(19) United States
(12) Patent Application Publication
(10) Pub. No.: US 2019/0374606 A1
(43) Pub. Date: Dec. 12, 2019

(54) NEUROTROPHIN MUTANTS FOR TREATING HEARING LOSS AND OTHER OTIC DISORDERS

(71) Applicant: OTONOMY, INC., San Diego, CA (US)

(72) Inventors: Alan FOSTER, San Diego, CA (US); Stephanie SZOBOTA, San Diego, CA (US); Horacio Uri SARAGOVI, Montreal (CA)

(21) Appl. No.: 16/481,245

(22) PCT Filed: Jan. 26, 2018
(86) PCT No.: PCT/US18/15548

§ 371 Date: Jul. 26, 2019

Related U.S. Application Data
(60) Provisional application No. 62/451,570, filed on Jan. 27, 2017.

(51) Int. Cl. A61K 38/18

(52) U.S. Cl. CPC A61K 38/185; A61K 45/06

(57) ABSTRACT

Disclosed herein are compositions and methods for the treatment of otic diseases or conditions with a non-natural neurotrophic agent compositions and formulations administered to an individual afflicted with an otic disease or condition, through direct application of these compositions and formulations onto or via perfusion into the targeted auris structure(s).

### CROSS-REFERENCE

This application claims the benefit of U.S. Provisional Application No. 62/451,570, filed on Jan. 27, 2017, which is incorporated herein by reference in its entirety.

### BACKGROUND OF THE DISCLOSURE

Vertebrates have a pair of ears, placed symmetrically on opposite sides of the head. The ear serves as both the sense organ that detects sound and the organ that maintains balance and body position. The ear is generally divided into three portions: the outer ear, auris media (or middle ear), and the auris interna (or inner ear).

### SUMMARY OF THE DISCLOSURE

Described herein, in certain embodiments, are methods of treating an otic condition in a subject in need thereof, comprising administering to the subject in need thereof an otic composition comprising a therapeutically effective amount of a non-natural neurotrophic agent; and a pharmaceutically acceptable carrier. In some embodiments, the non-natural neurotrophic agent comprises a modification at amino acid residue position equivalent to amino acid residue 15 set forth in SEQ ID NO: 2, and optionally comprises a modification at one or more amino acid residue positions equivalent to amino acid residues 11, 51, 68, 87, 103, 114, or 115 set forth in SEQ ID NO: 2. In some embodiments, the non-natural neurotrophic agent comprises nerve growth factor (NGF), a pro-form of nerve growth factor (proNGF), neurotrophin-3 (NT-3), a pro-form of neurotrophin-3 (proNT-3), neurotrophin-4 (NT-4), a pro-form of neurotrophin-4 (proNT-4), neurotrophin-5 (NT-5), a pro-form of neurotrophin-5 (proNT-5), brain-derived neurotrophic factor (BDNF), a pro-form of brain-derived neurotrophic factor (proBDNF), a pan-neurotrophin (PNT), or PNT-1. In some embodiments, the non-natural neurotrophic agent is NT-3 and wherein NT-3 comprises a modification at amino acid residue position equivalent to amino acid residue 15 set forth in SEQ ID NO: 2, and optionally comprises a modification at one or more amino acid residue positions equivalent to amino acid residues 11, 51, 68, 87, 103, 114, or 115 set forth in SEQ ID NO: 2. In some embodiments, the non-natural neurotrophic agent comprises about 70%, 80%, 85%, 90%, 91%, 92%, 93%, 94%, 95%, 96%, 97%, 98%, or 99% sequence identity to SEQ ID NOs: 22-34. In some embodiments, the non-natural neurotrophic agent comprises modifications at amino acid residue positions equivalent to amino acid residues 7 and 103 set forth in SEQ ID NO: 1. In some embodiments, the non-natural neurotrophic agent further comprises a modification at an amino acid residue position equivalent to amino acid residue 84 set forth in SEQ ID NO: 1. In some embodiments, the non-natural neurotrophic agent further comprises a modification at an amino acid residue position equivalent to amino acid residue 45 set forth in SEQ ID NO: 1. In some embodiments, the non-natural neurotrophic agent further comprises modifications at amino acid residue positions equivalent to amino acid residues 32, 34, and 35 set forth in SEQ ID NO: 1. In some embodiments, wherein the non-natural neurotrophic agent comprises nerve growth factor (NGF), neurotrophin-3 (NT-3), neurotrophin-4 (NT-4), neurotrophin-5 (NT-5), or brain-derived neurotrophic factor (BDNF). In some embodiments, the non-natural neurotrophic agent comprises a pro-form of nerve growth factor (proNGF), neurotrophin-3 (proNT-3), neurotrophin-4 (proNT-4), neurotrophin-5 (proNT-5), or brain-derived neurotrophic factor (proBDNF). In some embodiments, the non-natural neurotrophic agent comprises NGF. In some embodiments, the NGF comprises modifications at amino acid residue positions equivalent to amino acid residue 7, 84, and 103 set forth in SEQ ID NO: 1. In some embodiments, the NGF comprises modifications at amino acid residue positions equivalent to amino acid residue 7, 45, and 103 set forth in SEQ ID NO: 1. In some embodiments, the NGF further comprises modifications at amino acid residue positions equivalent to amino acid residues 32, 34, and 35 set forth in SEQ ID NO: 1. In some embodiments, the modification comprises a mutation to a non-polar residue. In some embodiments, the modification comprises a mutation to a polar residue. In some embodiments, the modification comprises a mutation to a charged residue. In some embodiments, the modification comprises a mutation to alanine. In some embodiments, the modification comprises a mutation to a conservative amino acid. In some embodiments, the modification comprises a mutation to a non-conservative amino acid. In some embodiments, the modification comprises a mutation to a semi-conservative amino acid. In some embodiments, the NGF comprises about 70%, 80%, 85%, 90%, 91%, 92%, 93%, 94%, 95%, 96%, 97%, 98% or 99% sequence identity to SEQ ID NO: 8, 9, 10 or 11. In some embodiments, NGF comprises the amino acid sequence set forth in SEQ ID NO: 8, 9, 10, or 11. In some embodiments, the non-natural neurotrophic agent comprises NT-3. In some embodiments, the non-natural neurotrophic agent comprises NT-4 or NT-5. In some embodiments, the non-natural neurotrophic agent comprises BDNF. In some embodiments, the non-natural neurotrophic agent is a pan-neurotrophin (PNT). In some embodiments, the non-natural neurotrophic agent is PNT-1. In some embodiments, the non-natural neurotrophic agent comprises a sequence set forth in SEQ ID NO: 6. In some embodiments, the non-natural neurotrophic agent comprises a sequence set forth in SEQ ID NO: 7. In some embodiments, the non-natural neurotrophic agent comprises at least two modifications selected from amino acid residue positions equivalent to amino acid residues 74, 75, and 77 set forth in SEQ ID NO: 5. In some embodiments, the non-natural neurotrophic agent further comprises a modification at an amino acid residue position equivalent to amino acid residue 32, 34, 115, or 116 set forth in SEQ ID NO: 5. In some embodiments, the non-natural neurotrophic agent comprises nerve growth factor (NGF), neurotrophin-3 (NT-3), neurotrophin-4 (NT-4), neurotrophin-5 (NT-5), or brain-derived neurotrophic factor (BDNF). In some embodiments, the non-natural neurotrophic agent is a pan-neurotrophin (PNT). In some embodiments, comprises modifications at amino acid residue positions equivalent to amino acid residues 74, 75, and 77 set forth in SEQ ID NO: 5. In some embodiments, PNT further comprises modifications at amino acid residue positions equivalent to amino acid residues 32, 34, 115, or 116 set forth in SEQ ID NO: 5. In some embodiments, the modification comprises a mutation to a non-polar residue. In some embodiments, the modification comprises a mutation to a polar residue. In some embodiments, the modification comprises a mutation to a charged residue. In some embodiments, the modification comprises a mutation to alanine. In some embodiments, the non-natural neurotrophic agent comprises about 70%, 80%, 85%, 90%, 91%, 92%, 93%, 94%, 95%, 96%, 97%, 98%, or 99% sequence identity to SEQ ID NO: 14 or 16. In some embodiments, the non-natural neurotrophic agent comprises the amino acid sequence set forth in SEQ ID NO: 14 or 16. In some embodiments, the non-natural neurotrophic agent comprises NGF. In some embodiments, the non-natural neurotrophic agent comprises NT-3. In some embodiments, the non-natural neurotrophic agent comprises NT-4 or NT-5. In some embodiments, the non-natural neurotrophic agent comprises BDNF. In some embodiments, the non-natural neurotrophic agent comprises a sequence set forth in SEQ ID NO: 6. In some embodiments, the non-natural neurotrophic agent comprises a sequence set forth in SEQ ID NO: 7. In some embodiments, the non-natural neurotrophic agent comprises modifications at amino acid residue positions equivalent to amino acid residues 32 and 34 set forth in SEQ ID NO: 5, wherein the non-natural neurotrophic agent comprises NGF, NT-4, NT-5, BDNF, or PNT. In some embodiments, the non-natural neurotrophic agent further comprises a modification at an amino acid residue position equivalent to amino acid residue 74, 75, 77, 115, or 116 set forth in SEQ ID NO: 5. In some embodiments, the non-natural neurotrophic agent is PNT. In some embodiments, PNT comprises modifications at amino acid residue positions equivalent to amino acid residues 32 and 34 set forth in SEQ ID NO: 5. In some embodiments, PNT comprises further comprises modifications at amino acid residue positions equivalent to amino acid residues 74, 75, 77, 115, and 116 set forth in SEQ ID NO: 5. In some embodiments, the modification comprises a mutation to a non-polar residue. In some embodiments, the modification comprises a mutation to a polar residue. In some embodiments, the modification comprises a mutation to a charged residue. In some embodiments, the modification comprises a mutation to alanine. In some embodiments, the non-natural neurotrophic agent comprises about 70%, 80%, 85%, 90%, 91%, 92%, 93%, 94%, 95%, 96%, 97%, 98% or 99% sequence identity to SEQ ID NO: 13 or 16. In some embodiments, the non-natural neurotrophic agent comprises the amino acid sequence set forth in SEQ ID NO: 13 or 16. In some embodiments, the non-natural neurotrophic agent comprises NGF. In some embodiments, the non-natural neurotrophic agent comprises NT-4 or NT-5. In some embodiments, the non-natural neurotrophic agent comprises BDNF. In some embodiments, the non-natural neurotrophic agent comprises a sequence set forth in SEQ ID NO: 6. In some embodiments, the non-natural neurotrophic agent comprises a sequence set forth in SEQ ID NO: 7. In some embodiments, the non-natural neurotrophic agent comprises modifications at amino acid residue positions equivalent to amino acid residues 115 and 116 set forth in SEQ ID NO: 5, wherein the non-natural neurotrophic agent comprises NGF, NT-4, NT-5, BDNF, or PNT. In some embodiments, the non-natural neurotrophic agent further comprises a modification at an amino acid residue position equivalent to amino acid residue 32, 34, 74, 75, or 77 set forth in SEQ ID NO: 5. In some embodiments, the non-natural neurotrophic agent is PNT. In some embodiments, PNT comprises modifications at amino acid residue positions equivalent to amino acid residues 115 and 116 set forth in SEQ ID NO: 5. In some embodiments, PNT further comprises modifications at amino acid residue positions equivalent to amino acid residues 32, 34, 74, 75, and 77 set forth in SEQ ID NO: 5. In some embodiments, the modification comprises a mutation to a non-polar residue. In some embodiments, the modification comprises a mutation to a polar residue. In some embodiments, the modification comprises a mutation to a charged residue. In some embodiments, the modification comprises a mutation to alanine. In some embodiments, the non-natural neurotrophic agent comprises about 70%, 80%, 85%, 90%, 91%, 92%, 93%, 94%, 95%, 96%, 97%, 98%, or 99% sequence identity to SEQ ID NO: 15 or 16. In some embodiments, the non-natural neurotrophic agent comprises the amino acid sequence set forth in SEQ ID NO: 15 or 16. In some embodiments, the non-natural neurotrophic agent comprises NGF. In some embodiments, the non-natural neurotrophic agent comprises NT-4 or NT-5. In some embodiments, the non-natural neurotrophic agent comprises BDNF. In some embodiments, the non-natural neurotrophic agent comprises a sequence set forth in SEQ ID NO: 6. In some embodiments, the non-natural neurotrophic agent comprises a sequence set forth in SEQ ID NO: 7. In some embodiments, the non-natural neurotrophic agent comprises modifications at amino acid residue positions equivalent to amino acid residues 61 and 100 set forth in SEQ ID NO: 5, wherein the non-natural neurotrophic agent comprises NT-3, NT-4, NT-5, BDNF, or PNT. In some embodiments, the non-natural neurotrophic agent is PNT. In some embodiments, PNT comprises modifications at amino acid residue positions equivalent to amino acid residues 61 and 100 set forth in SEQ ID NO: 5. In some embodiments, the modification comprises a mutation to a non-polar residue. In some embodiments, the modification comprises a mutation to a polar residue. In some embodiments, the modification comprises a mutation to a charged residue. In some embodiments, the modification at amino acid residue 61 comprises a mutation from proline to serine. In some embodiments, the modification at amino acid residue 100 comprises a mutation from arginine to glutamic acid. In some embodiments, the non-natural neurotrophic agent comprises about 70%, 80%, 85%, 90%, 91%, 92%, 93%, 94%, 95%, 96%, 97%, 98%, or 99% sequence identity to SEQ ID NO: 20. In some embodiments, the non-natural neurotrophic agent comprises the amino acid sequence set forth in SEQ ID NO: 20. In some embodiments, the non-natural neurotrophic agent is NT-3. In some embodiments, the non-natural neurotrophic agent comprises NT-4 or NT-5. In some embodiments, the non-natural neurotrophic agent comprises BDNF. In some embodiments, the non-natural neurotrophic agent comprises a sequence set forth in SEQ ID NO: 6. In some embodiments, the non-natural neurotrophic agent comprises a sequence set forth in SEQ ID NO: 7. In some embodiments, the non-natural neurotrophic agent comprises a modification at amino acid residue position equivalent to amino acid residue 118 set forth in SEQ ID NO: 5, wherein the non-natural neurotrophic agent comprises NT-3, NT-4, NT-5, BDNF, or PNT. In some embodiments, the non-natural neurotrophic agent is PNT. In some embodiments, PNT comprises a modification at amino acid residue positions equivalent to amino acid residue 118 set forth in SEQ ID NO: 5. In some embodiments, the modification comprises a mutation to a non-polar residue. In some embodiments, the modification comprises a mutation to a polar residue. In some embodiments, the modification comprises a mutation to a charged residue. In some embodiments, the modification comprises a mutation to alanine. In some embodiments, the non-natural neurotrophic agent comprises about 70%, 80%, 85%, 90%, 91%, 92%, 93%, 94%, 95%, 96%, 97%, 98%, or 99% sequence identity to SEQ ID NO: 21. In some embodiments, the non-natural neurotrophic agent comprises the amino acid sequence set forth in SEQ ID NO: 21. In some embodiments, the non-natural neurotrophic agent comprises NT-3. In some embodiments, the non-natural neurotrophic agent comprises NT-4 or NT-5. In some embodiments, the non-natural neurotrophic agent comprises BDNF. In some embodiments, the non-natural neurotrophic agent comprises a sequence set forth in SEQ ID NO: 6. In some embodiments, the non-natural neurotrophic agent comprises a sequence set forth in SEQ ID NO: 7. In some embodiments, the non-natural neurotrophic agent comprises a sequence set forth in SEQ ID NOs: 17-19. In some embodiments, the non-natural neurotrophic agent recognizes one or more tropomyosin receptor kinase (Trk) receptors. In some embodiments, the non-natural neurotrophic agent recognizes a TrkA receptor. In some embodiments, the non-natural neurotrophic agent recognizes a TrkB receptor. In some embodiments, the non-natural neurotrophic agent recognizes a TrkC receptor. In some embodiments, the non-natural neurotrophic agent is a pan-neurotrophin. In some embodiments, the non-natural neurotrophic agent has an increased binding affinity to one or more Trk receptors relative to the binding affinity of an equivalent wild type neurotrophin. In some embodiments, the increase in binding affinity is about 20%, 30%, 40%, 50%, 60%, 70%, 80%, 85%, 90%, 95%, 96%, 97%, 98%, 99%, or more, relative to the binding affinity of an equivalent wild type neurotrophin. In some embodiments, the non-natural neurotrophic agent has a decreased binding affinity to p75^{NTR} relative to the binding affinity of an equivalent wild type neurotrophin. In some embodiments, the decrease in binding affinity is about 30%, 40%, 50%, 60%, 70%, 80%, 85%, 90%, 95%, 96%, 97%, 98%, 99%, or more, relative to the binding affinity of an equivalent wild type neurotrophin. In some embodiments, the non-natural neurotrophic agent has an impaired ability to activate p75^{NTR} or does not activate p75^{NTR} relative to the p75^{NTR} activation by an equivalent wild type neurotrophin. In some embodiments, p75^{NTR} activation is reduced by 30%, 40%, 50%, 60%, 70%, 80%, 85%, 90%, 95%, 96%, 97%, 98%, 99%, or more, relative to the p75^{NTR} activation by an equivalent wild type neurotrophin. In some embodiments, the otic composition further comprises two or more characteristics selected from: (i) from about 0.0001% to about 60% by weight of the non-natural TrkB or TrkC agonist, or pharmaceutically acceptable prodrug or salt thereof; (ii) from about 14% to about 21% by weight of a polyoxyethylene-polyoxypropylene triblock copolymer; (iii) sterile water, q.s., buffered to provide a pH from about 5.5 to about 8.0; (iv) a gelation temperature between about 19° C. to about 42° C.; and (v) an apparent viscosity from about 100,000 cP to about 500,000 cP. In some embodiments, the non-natural neurotrophic agent is released from the composition for a period of at least 1 day or at least 5 days. In some embodiments, the otic condition is selected from a group consisting of ototoxicity, chemotherapy induced hearing loss, excitotoxicity, sensorineural hearing loss, noise induced hearing loss, hidden hearing loss, Meniere's Disease/Syndrome, endolymphatic hydrops, labyrinthitis, Ramsay Hunt's Syndrome, vestibular neuronitis, tinnitus, age-related hearing loss (presbycusis), microvascular compression syndrome, genetic hearing loss, and balance disorder. In some embodiments, administering the otic composition comprising the non-natural neurotrophic agent treats sensorineural hearing loss by inducing auris neuronal cell growth. In some embodiments, administering the otic composition comprising the non-natural neurotrophic agent mediates at least one of synaptic modulation, afferent fiber growth, restoration of ribbon synapses, and increased neuronal connection to hair cells. In some embodiments, the otic condition is characterized by damaged ribbon synapse, neurodegeneration, hair cell loss, or synaptopathy. In some embodiments, the subject is a human.

Described herein, in certain embodiments, are otic pharmaceutical compositions comprising a therapeutically effective amount of a non-natural neurotrophic agent and a pharmaceutically acceptable carrier. In some embodiments, the non-natural neurotrophic agent comprises modifications at amino acid residue positions equivalent to amino acid residues 7 and 103 set forth in SEQ ID NO: 1. In some embodiments, the non-natural neurotrophic agent further comprises a modification at an amino acid residue position equivalent to amino acid residue 84 set forth in SEQ ID NO: 1. In some embodiments, the non-natural neurotrophic agent further comprises a modification at an amino acid residue position equivalent to amino acid residue 45 set forth in SEQ ID NO: 1. In some embodiments, the non-natural neurotrophic agent further comprises modifications at amino acid residue positions equivalent to amino acid residues 32, 34, and 35 set forth in SEQ ID NO: 1. In some embodiments, the non-natural neurotrophic agent comprises at least two modifications selected from amino acid residue positions equivalent to amino acid residues 74, 75, and 77 set forth in SEQ ID NO: 5. In some embodiments, the non-natural neurotrophic agent further comprises a modification at an amino acid residue position equivalent to amino acid residue 32, 34, 115, or 116 set forth in SEQ ID NO: 5. In some embodiments, the non-natural neurotrophic agent comprises modifications at amino acid residue positions equivalent to amino acid residues 32 and 34 set forth in SEQ ID NO: 5, wherein the non-natural neurotrophic agent comprises NGF, NT-4, NT-5, BDNF, or PNT. In some embodiments, the non-natural neurotrophic agent further comprises a modification at an amino acid residue position equivalent to amino acid residue 74, 75, 77, 115, or 116 set forth in SEQ ID NO: 5. In some embodiments, the non-natural neurotrophic agent comprises modifications at amino acid residue positions equivalent to amino acid residues 115 and 116 set forth in SEQ ID NO: 5, wherein the non-natural neurotrophic agent comprises NGF, NT-4, NT-5, BDNF, or PNT. In some embodiments, the non-natural neurotrophic agent further comprises a modification at an amino acid residue position equivalent to amino acid residue 32, 34, 74, 75, or 77 set forth in SEQ ID NO: 5. In some embodiments, the non-natural neurotrophic agent comprises modifications at amino acid residue positions equivalent to amino acid residues 61 and 100 set forth in SEQ ID NO: 5, wherein the non-natural neurotrophic agent comprises NT-3, NT-4, NT-5, BDNF, or PNT. In some embodiments, the non-natural neurotrophic agent comprises a modification at amino acid residue position equivalent to amino acid residue 118 set forth in SEQ ID NO: 5, wherein the non-natural neurotrophic agent comprises NT-3, NT-4, NT-5, BDNF, or PNT. In some embodiments, the non-natural neurotrophic agent comprises a sequence set forth in SEQ ID NOs: 17-19. In some embodiments, further comprises two or more characteristics selected from: (i) from about 0.0001% to about 60% by weight of the non-natural TrkB or TrkC agonist, or pharmaceutically acceptable prodrug or salt thereof; (ii) from about 14% to about 21% by weight of a polyoxyethylene-polyoxypropylene triblock copolymer; (iii) sterile water, q.s., buffered to provide a pH from about 5.5 to about 8.0; (iv) a gelation temperature from about 19° C. to about 42° C.; and (v) an apparent viscosity from about 100,000 cP to about 500,000 cP. In some embodiments, the pharmaceutical composition is an auris-acceptable thermoreversible gel.

Described herein, in certain embodiments, are otic pharmaceutical compositions comprising a therapeutically effective amount of a non-natural neurotrophic agent and triglycerides comprising medium chain fatty acids and/or long chain fatty acids. In some embodiments, the non-natural neurotrophic agent further comprises a modification at an amino acid residue position equivalent to amino acid residue 84 set forth in SEQ ID NO: 1. In some embodiments, the non-natural neurotrophic agent further comprises a modification at an amino acid residue position equivalent to amino acid residue 45 set forth in SEQ ID NO: 1. In some embodiments, the non-natural neurotrophic agent further comprises modifications at amino acid residue positions equivalent to amino acid residues 32, 34, and 35 set forth in SEQ ID NO: 1. In some embodiments, the non-natural neurotrophic agent comprises at least two modifications selected from amino acid residue positions equivalent to amino acid residues 74, 75, and 77 set forth in SEQ ID NO: 5. In some embodiments, the non-natural neurotrophic agent further comprises a modification at an amino acid residue position equivalent to amino acid residue 32, 34, 115, or 116 set forth in SEQ ID NO: 5. In some embodiments, the non-natural neurotrophic agent comprises modifications at amino acid residue positions equivalent to amino acid residues 32 and 34 set forth in SEQ ID NO: 5, wherein the non-natural neurotrophic agent comprises NGF, NT-4, NT-5, BDNF, or PNT. In some embodiments, the non-natural neurotrophic agent further comprises a modification at an amino acid residue position equivalent to amino acid residue 74, 75, 77, 115, or 116 set forth in SEQ ID NO: 5. In some embodiments, the non-natural neurotrophic agent comprises modifications at amino acid residue positions equivalent to amino acid residues 115 and 116 set forth in SEQ ID NO: 5, wherein the non-natural neurotrophic agent comprises NGF, NT-4, NT-5, BDNF, or PNT. In some embodiments, the non-natural neurotrophic agent further comprises a modification at an amino acid residue position equivalent to amino acid residue 32, 34, 74, 75, or 77 set forth in SEQ ID NO: 5. In some embodiments, the non-natural neurotrophic agent comprises modifications at amino acid residue positions equivalent to amino acid residues 61 and 100 set forth in SEQ ID NO: 5, wherein the non-natural neurotrophic agent is selected from NT-3, NT-4, NT-5, BDNF, or PNT. In some embodiments, the non-natural neurotrophic agent comprises a modification at amino acid residue position equivalent to amino acid residue 118 set forth in SEQ ID NO: 5, wherein the non-natural neurotrophic agent comprises NT-3, NT-4, NT-5, BDNF, or PNT. In some embodiments, the non-natural neurotrophic agent comprises a sequence set forth in SEQ ID NOs: 17-19. In some embodiments, the triglycerides are derived from glycerol and medium chain fatty acids. In some embodiments, each medium chain fatty acid independently comprises 6 to 12 carbon atoms in the carbon chain. In some embodiments, each medium chain fatty acid independently comprises 8 to 12 carbon atoms in the carbon chain. In some embodiments, the medium chain fatty acids are saturated medium chain fatty acids, unsaturated medium chain fatty acids, or any combination thereof. In some embodiments, the medium chain fatty acids are caproic acid (hexanoic acid), enanthic acid (heptanoic acid), caprylic acid (octanoic acid), pelargonic acid (nonanoic acid), capric acid (decanoic acid), undecylenic acid (undec-10-enoic acid), lauric acid (dodecanoic acid), or any combination thereof. In some embodiments, the triglycerides comprising medium chain fatty acids are balassee oil, coconut oil, cohune oil, palm kernel oil, tucum oil, or any combination thereof. In some embodiments, the triglycerides are derived from glycerol and long chain fatty acids. In some embodiments, each long chain fatty acid independently comprises greater than 12 carbon atoms in the carbon chain. In some embodiments, each long chain fatty acid independently comprises 13 to 38 carbon atoms in the carbon chain. In some embodiments, each long chain fatty acid independently comprises 13 to 24 carbon atoms in the carbon chain. In some embodiments, the long chain fatty acids are saturated long chain fatty acids, unsaturated long chain fatty acids, or any combination thereof. In some embodiments, the long chain fatty acids are tridecylic acid (tridecanoic acid), myristic acid (tetradecanoic acid), pentadecylic acid (pentadecanoic acid), palmitic acid (hexadecanoic acid), margaric acid (heptadecanoic acid), stearic acid (octadecanoic acid), nonadecylic acid (nonadecanoic acid), arachidic acid (eicosanoic acid), heneicosylic acid (heneicosanoic acid), behenic acid (docosanoic acid), tricosylic acid (tricosanoic acid), lignoceric acid (tetracosanoic acid), pentacosylic acid (pentacosanoic acid), cerotic acid (hexacosanoic acid), heptacosylic acid (heptacosanoic acid), montanic acid (octacosanoic acid), nonacosylic acid (nonacosanoic acid), melissic acid (triacontanoic acid), henatriacontylic acid (henatriacontanoic acid), lacceroic acid (dotriacontanoic acid), psyllic acid (tritriacontanoic acid), geddic acid (tetratriacontanoic acid), ceroplastic acid (pentatriacontanoic acid), hexatriacontylic acid (hexatriacontanoic acid), heptatriacontanoic acid (heptatriacontanoic acid), octatriacontanoic acid, or any combination thereof. In some embodiments, the long chain fatty acids are α-linolenic acid, stearidonic acid, eicosapentaenoic acid, docosahexaenoic acid, linoleic acid, γ-linolenic acid, dihomo-γ-linolenic acid, arachidonic acid, docosatetraenoic acid, palmitoleic acid, vaccenic acid, paullinic acid, oleic acid, elaidic acid, gondoic acid, erucic acid, nervonic acid, mead acid, or any combination thereof. In some embodiments, the triglycerides comprising long chain fatty acids are corn oil, peanut oil, safflower oil, soybean oil, sunflower seed oil, sesame oil, olive oil, castor oil, cotton seed oil, fish oil, or any combination thereof. In some embodiments, the otic pharmaceutical formulation comprises from about 5% to about 99.9%, from about 10% to about 99.9%, from about 15% to about 99.9%, from about 20% to about 99.9%, from about 25% to about 99.9%, from about 30% to about 99.9%, from about 35% to about 99.9%, from about 40% to about 99.9%, from about 45% to about 99.9%, from about 50% to about 99.9%, from about 55% to about 99.9%, from about 60% to about 99.9%, from about 65% to about 99.9%, from about 70% to about 99.9%, from about 75% to about 99.9%, from about 85% to about 99.9%, or from about 80% to about 99.9% by weight of the triglycerides.

Described herein, in certain embodiments, are methods of treating an otic condition in a subject in need thereof, comprising: administering to the subject in need thereof an otic composition comprising: i) a therapeutically effective amount of a non-natural neurotrophic agent comprising modifications at amino residue positions equivalent to amino acid residues 15, 114, and 115 set forth in SEQ ID NO: 2; and ii) a pharmaceutically acceptable carrier; wherein the non-natural neurotrophic agent comprises NGF, NT-4, NT-5, BDNF, or PNT. In some embodiments, the non-natural neurotrophic agent further comprises a modification at an amino residue position equivalent to at least one of amino acid residue 11, 68, 87, and 103 set forth in SEQ ID NO: 2.

Described herein, in certain embodiments, are methods of treating an otic condition in a subject in need thereof, comprising: administering to the subject in need thereof an otic composition comprising: i) a therapeutically effective amount of a non-natural neurotrophic agent comprising modifications at amino residue positions equivalent to amino acid residues 15 and 103 set forth in SEQ ID NO: 2; and ii) a pharmaceutically acceptable carrier; wherein the non-natural neurotrophic agent comprises NGF, NT-4, NT-5, BDNF, or PNT.

Described herein, in certain embodiments, are methods of treating an otic condition in a subject in need thereof, comprising: administering to the subject in need thereof an otic composition comprising: i) a therapeutically effective amount of a non-natural neurotrophic agent comprising modifications at amino residue positions equivalent to amino acid residues 15, 51, and 103 set forth in SEQ ID NO: 2; and ii) a pharmaceutically acceptable carrier; wherein the non-natural neurotrophic agent comprises NGF, NT-4, NT-5, BDNF, or PNT.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates the anatomy of the ear.

FIG. 2 illustrates respective levels of trophic support to rat spiral ganglion neurons by exemplary NT-3 mutants D15A-NT-3, R114A/K115A-NT-3 and D15A/R114A/K115A-NT-3 in culture.

FIG. 3A-FIG. 3B illustrate perilymph concentrations of BDNF and NT-3 following a single IT injection in rats. BDNF was administered at a dose of 0.1% (1.05 mg/ml) (FIG. 3A) and NT-3 was administered at a dose of 0.1% (1.05 mg/ml) (FIG. 3B).

FIG. 4 illustrates perilymph concentrations of BDNF following a single IT injection in rats in poloxamer 407 formulation. BDNF was administered at the indicated doses. Perilymph BDNF is in arbitrary units.

FIG. 5A-FIG. 5C illustrate perilymph concentrations of BDNF following a single IT injection in rats in MCT formulations. BDNF was administered at a dose of 0.15% (1.5 mg/ml) in the MCT formulation containing different polymers: PVP (polyvinylpyrrolidone) at 2 or 10% (FIG. 5A); 0.15% carbomer (FIG. 5B); or P407 (poloxamer 407) at 10% (FIG. 5C). Perilymph BDNF is in arbitrary units.

FIG. 6 illustrates EC₅₀ values for mutant neurotrophins compared with NT-3 in a human TrkC receptor assay.

FIG. 7 illustrates maximum effect values for mutant neurotrophins compared with NT-3 in a human TrkC receptor assay.

### DETAILED DESCRIPTION OF THE DISCLOSURE

Provided herein are otic compositions for treating or ameliorating hearing loss or reduction resulting from destroyed, stunted, malfunctioning, damaged, fragile, or missing hair cells, neurons, and their connections in the inner ear. In one embodiment, the otic composition comprises a therapeutically-effective amount of at least one non-natural Trk receptor (e.g., TrkA, TrkB, or TrkC) agonist and an auris-acceptable pharmaceutical excipient. Further disclosed herein are otic compositions and formulations comprising a non-natural neurotrophic agent to treat ototoxicity, chemotherapy-induced hearing loss, excitotoxicity, sensorineural hearing loss, noise-induced hearing loss, hidden hearing loss, Meniere's Disease/Syndrome, endolymphatic hydrops, labyrinthitis, Ramsay Hunt's Syndrome, vestibular neuronitis, tinnitus, age-related hearing loss (presbycusis), microvascular compression syndrome, genetic hearing loss, or balance disorder. In some embodiments, otic compositions and formulations comprising a non-natural neurotrophic agent are used in conjunction with other treatments for otic diseases or disorders. For example, otic compositions and formulations comprising a non-natural neurotrophic agent are used in conjunction with cochlear implants.

In certain embodiments, disclosed herein are compositions, formulations, methods of use, kits, and delivery devices for treating an otic condition. In some embodiments, the otic condition is ototoxicity, chemotherapy-induced hearing loss, excitotoxicity, sensorineural hearing loss, noise-induced hearing loss, hidden hearing loss, Meniere's Disease/Syndrome, endolymphatic hydrops, labyrinthitis, Ramsay Hunt's Syndrome, vestibular neuronitis, tinnitus, age-related hearing loss (presbycusis), microvascular compression syndrome, genetic hearing loss, or balance disorder. In certain embodiments, disclosed herein are compositions, formulations, methods, uses, kits, and delivery devices for treating otic conditions which need repair of damaged ribbon synapses.

Also disclosed herein, are controlled-release otic compositions and formulations for treating otic conditions, including but not limited to ototoxicity, chemotherapy-induced hearing loss, excitotoxicity, sensorineural hearing loss, noise-induced hearing loss, hidden hearing loss, Meniere's Disease/Syndrome, endolymphatic hydrops, labyrinthitis, Ramsay Hunt's Syndrome, vestibular neuronitis, tinnitus, age-related hearing loss (presbycusis), microvascular compression syndrome, genetic hearing loss, and balance disorder. The formulations described herein provide a constant, sustained, extended, or delayed rate of release of a non-natural neurotrophic agent into the otic environment and thus avoid any variability in drug exposure in treatment of ototoxicity, chemotherapy-induced hearing loss, excitotoxicity, sensorineural hearing loss, noise-induced hearing loss, hidden hearing loss, Meniere's Disease/Syndrome, endolymphatic hydrops, labyrinthitis, Ramsay Hunt's Syndrome, vestibular neuronitis, tinnitus, age-related hearing loss (presbycusis), microvascular compression syndrome, genetic hearing loss, or balance disorder.

Further provided herein are otic formulations that are sterilized with stringent sterility requirements and are suitable for otic administration. In some embodiments, the auris-compatible compositions described herein are substantially free of pyrogens and/or microbes.

Provided herein are otic formulations that meet certain criteria for pH, osmolarity, ionic balance, sterility, endotoxin, and/or pyrogen levels. The otic compositions described herein are compatible with the otic environment and are suitable for administration to humans.

By way of non-limiting example, the use of the following commonly used solvents should be limited, reduced, or eliminated when formulating agents for administration to the ear: alcohols, propylene glycol, and cyclohexane. Thus, in some embodiments, an otic composition or formulation disclosed herein is free or substantially free of alcohols, propylene glycol, and cyclohexane. In some embodiments, an otic composition or formulation disclosed herein comprises less than about 50 ppm of each of alcohols, propylene glycol, and cyclohexane. In some embodiments, an otic composition or formulation disclosed herein comprises less than about 25 ppm of each of alcohols, propylene glycol, and cyclohexane. In some embodiments, an otic composition or formulation disclosed herein comprises less than about 20 ppm of each of alcohols, propylene glycol, and cyclohexane. In some embodiments, an otic composition or formulation disclosed herein comprises less than about 10 ppm of each of alcohols, propylene glycol, and cyclohexane. In some embodiments, an otic composition or formulation disclosed herein comprises less than about 5 ppm of each of alcohols, propylene glycol, and cyclohexane. In some embodiments, an otic composition or formulation disclosed herein comprises less than about 1 ppm of each of alcohols, propylene glycol, and cyclohexane.

Further, otic preparations require particularly low concentrations of several potentially common contaminants that are known to be ototoxic. Other dosage forms, while seeking to limit the contamination attributable to these compounds, do not require the stringent precautions that otic preparations require. For example, the following contaminants should be absent or nearly absent from otic preparations: arsenic, lead, mercury, and tin. Thus, in some embodiments, an otic composition or formulation disclosed herein is free or substantially free of arsenic, lead, mercury, and tin. In some embodiments, an otic composition or formulation disclosed herein comprises less than about 50 ppm of each of arsenic, lead, mercury, and tin. In some embodiments, an otic composition or formulation disclosed herein comprises less than about 25 ppm of each of arsenic, lead, mercury, and tin. In some embodiments, an otic composition or formulation disclosed herein comprises less than about 20 ppm of each of arsenic, lead, mercury, and tin. In some embodiments, an otic composition or formulation disclosed herein comprises less than about 10 ppm of each of arsenic, lead, mercury, and tin. In some embodiments, an otic composition or formulation disclosed herein comprises less than about 5 ppm of each of arsenic, lead, mercury, and tin. In some embodiments, an otic composition or formulation disclosed herein comprises less than about 1 ppm of each of arsenic, lead, mercury, and tin.

### Anatomy of the Ear

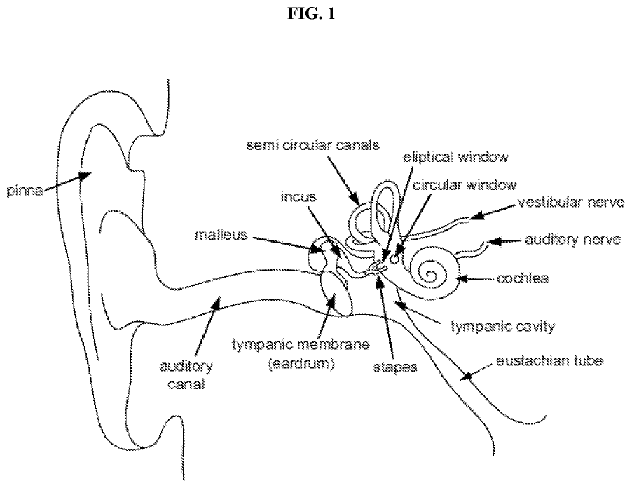

As shown in FIG. 1, the outer ear is the external portion of the organ and is composed of the pinna (auricle), the auditory canal (external auditory meatus), and the outward facing portion of the tympanic membrane, also known as the ear drum. The pinna, which is the fleshy part of the external ear that is visible on the side of the head, collects sound waves and directs them toward the auditory canal. Thus, the function of the outer ear, in part, is to collect and direct sound waves towards the tympanic membrane and the middle ear.

The middle ear is an air-filled cavity, called the tympanic cavity, behind the tympanic membrane. The tympanic membrane, also known as the ear drum, is a thin membrane that separates the external ear from the middle ear. The middle ear lies within the temporal bone and includes within this space the three ear bones (auditory ossicles): the malleus, the incus, and the stapes. The auditory ossicles are linked together via tiny ligaments, which form a bridge across the space of the tympanic cavity. The malleus, which is attached to the tympanic membrane at one end, is linked to the incus at its anterior end, which in turn is linked to the stapes. The stapes is attached to the oval window, one of two windows located within the tympanic cavity. A fibrous tissue layer, known as the annular ligament connects the stapes to the oval window. Sound waves from the outer ear first cause the tympanic membrane to vibrate. The vibration is transmitted across to the cochlea through the auditory ossicles and oval window, which transfers the motion to the fluids in the auris interna. Thus, the auditory ossicles are arranged to provide a mechanical linkage between the tympanic membrane and the oval window of the fluid-filled auris interna, where sound is transformed and transduced to the auris interna for further processing. Stiffness, rigidity, or loss of movement of the auditory ossicles, tympanic membrane, or oval window leads to hearing loss, e.g. otosclerosis, or rigidity of the stapes bone.

The tympanic cavity also connects to the throat via the eustachian tube. The eustachian tube provides the ability to equalize the pressure between the outside air and the middle ear cavity. The round window, a component of the auris interna but which is also accessible within the tympanic cavity, opens into the cochlea of the auris interna. The round window is covered by round window membrane, which consists of three layers: an external or mucous layer, an intermediate or fibrous layer, and an internal membrane, which communicates directly with the cochlear fluid. The round window, therefore, has direct communication with the auris interna via the internal membrane.

Movements in the oval and round window are interconnected, i.e. as the stapes bone transmits movement from the tympanic membrane to the oval window to move inward against the auris interna fluid, the round window (round window membrane) is correspondingly pushed out and away from the cochlear fluid. This movement of the round window allows movement of fluid within the cochlea, which leads in turn to movement of the cochlear inner hair cells, allowing hearing signals to be transduced. Stiffness and rigidity in round window membrane leads to hearing loss because of the lack of ability of movement in the cochlear fluid. Recent studies have focused on implanting mechanical transducers onto the round window, which bypasses the normal conductive pathway through the oval window and provides amplified input into the cochlear chamber.

Auditory signal transduction takes place in the auris interna. The fluid-filled auris interna, or inner ear, consists of two major components: the cochlear and the vestibular apparatus. The auris interna is located in part within the osseous or bony labyrinth, an intricate series of passages in the temporal bone of the skull. The vestibular apparatus is the organ of balance and consists of the three semi-circular canals and the vestibule. The three semi-circular canals are arranged relative to each other such that movement of the head along the three orthogonal planes in space can be detected by the movement of the fluid and subsequent signal processing by the sensory organs of the semi-circular canals, called the crista ampullaris. The crista ampullaris contains hair cells and supporting cells, and is covered by a dome-shaped gelatinous mass called the cupula. The hairs of the hair cells are embedded in the cupula. The semi-circular canals detect dynamic equilibrium, the equilibrium of rotational or angular movements.

When the head turns rapidly, the semicircular canals move with the head, but endolymph fluid located in the membranous semi-circular canals tends to remain stationary. The endolymph fluid pushes against the cupula, which tilts to one side. As the cupula tilts, it bends some of the hairs on the hair cells of the crista ampullaris, which triggers a sensory impulse. Because each semicircular canal is located in a different plane, the corresponding crista ampullaris of each semi-circular canal responds differently to the same movement of the head. This creates a mosaic of impulses that are transmitted to the central nervous system on the vestibular branch of the vestibulocochlear nerve. The central nervous system interprets this information and initiates the appropriate responses to maintain balance. Of importance in the central nervous system is the cerebellum, which mediates the sense of balance and equilibrium.

The vestibule is the central portion of the auris interna and contains mechanoreceptors bearing hair cells that ascertain static equilibrium, or the position of the head relative to gravity. Static equilibrium plays a role when the head is motionless or moving in a straight line. The membranous labyrinth in the vestibule is divided into two sac-like structures, the utricle and the saccule. Each structure in turn contains a small structure called a macula, which is responsible for maintenance of static equilibrium. The macula consists of sensory hair cells, which are embedded in a gelatinous mass (similar to the cupula) that covers the macula. Grains of calcium carbonate, called otoliths, are embedded on the surface of the gelatinous layer.

When the head is in an upright position, the hairs are straight along the macula. When the head tilts, the gelatinous mass and otoliths tilts correspondingly, bending some of the hairs on the hair cells of the macula. This bending action initiates a signal impulse to the central nervous system, which travels via the vestibular branch of the vestibulocochlear nerve, which in turn relays motor impulses to the appropriate muscles to maintain balance.

The cochlea is the portion of the auris interna related to hearing. The cochlea is a tapered tube-like structure which is coiled into a shape resembling a snail. The inside of the cochlea is divided into three regions, which is further defined by the position of the vestibular membrane and the basilar membrane. The portion above the vestibular membrane is the scala vestibuli, which extends from the oval window to the apex of the cochlea and contains perilymph fluid, an aqueous liquid low in potassium and high in sodium content. The basilar membrane defines the scala tympani region, which extends from the apex of the cochlea to the round window and also contains perilymph. The basilar membrane contains thousands of stiff fibers, which gradually increase in length from the round window to the apex of the cochlea. The fibers of the basement membrane vibrate when activated by sound. In between the scala vestibuli and the scala tympani is the cochlear duct, which ends as a closed sac at the apex of the cochlea. The cochlear duct contains endolymph fluid, which is similar to cerebrospinal fluid and is high in potassium.

The organ of Corti, the sensory organ for hearing, is located on the basilar membrane and extends upward into the cochlear duct. The organ of Corti contains hair cells, which have hairlike projections that extend from their free surface, and contacts a gelatinous surface called the tectorial membrane. Although hair cells have no axons, they are surrounded by sensory nerve fibers that form the cochlear branch of the vestibulocochlear nerve (cranial nerve VIII).

As discussed, the oval window, also known as the elliptical window communicates with the stapes to relay sound waves that vibrate from the tympanic membrane. Vibrations transferred to the oval window increase pressure inside the fluid-filled cochlea via the perilymph and scala vestibuli/scala tympani, which in turn causes the round window membrane to expand in response. The concerted inward pressing of the oval window/outward expansion of the round window allows for the movement of fluid within the cochlea without a change of intra-cochlear pressure. However, as vibrations travel through the perilymph in the scala vestibuli, they create corresponding oscillations in the vestibular membrane. These corresponding oscillations travel through the endolymph of the cochlear duct and transfer to the basilar membrane. When the basilar membrane oscillates, or moves up and down, the organ of Corti moves along with it. The hair cell receptors in the Organ of Corti then move against the tectorial membrane, causing a mechanical deformation in the stereocilia of the hair cells. The deflection of stereocilia produces depolarization of the hair cells and a graded release of the neurotransmitter glutamate at the hair cell ribbon synapses. Glutamate activates receptors on the cochlea afferent fibers that connect to inner hair cells as part of ribbon synapses. The afferent fibers are dendrites from spiral ganglion neurons and their depolarization by glutamate is carried along the afferent fibers to the cell bodies where an action potential can be evoked. Action potentials in spiral ganglion neurons are transmitted via their axons which form the auditory (VIII^{th} cranial) nerve to the central nervous system where these signals are perceived as sound. In this way, sound produces a mechanical stimulus within the cochlea that is transduced to an electrical signal by the organ of Corti that is perceived as sound by the central nervous system.

### Diseases

Described herein, in some embodiments, are otic compositions comprising a non-natural neurotrophic agent for treating an otic disease or disorder. In some embodiments, the agonist is a non-natural neurotrophic agent. In some embodiments, the non-natural neurotrophic agent comprises NGF, NT-3, NT-4, NT-5, BDNF, a pan-neurotrophin, or a chimeric neurotrophin. Otic disorders produce symptoms which include but are not limited to hearing loss, nystagmus, vertigo, tinnitus, inflammation, infection, and congestion. The otic disorders which are treated with the compositions disclosed herein are numerous and include ototoxicity, chemotherapy-induced hearing loss, excitotoxicity, sensorineural hearing loss, noise-induced hearing loss, hidden hearing loss, Meniere's Disease/Syndrome, endolymphatic hydrops, labyrinthitis, Ramsay Hunt's Syndrome, vestibular neuronitis, tinnitus, age-related hearing loss (presbycusis), microvascular compression syndrome, genetic hearing loss, and balance disorder. In some cases, genetic hearing loss comprises a hearing loss associated with or due to a genetic mutation. In some instances, the genetic hearing loss comprises disorders associated with vestibular system (e.g., central vestibular disorders and/or peripheral vestibular disorders). In other instances, genetic hearing loss comprises congenital hearing loss, hereditary hearing loss, autosomal dominant hearing loss, autosomal recessive hearing loss, or X-linked hearing loss. In some embodiments, otic compositions and formulations comprising a non-natural Trk receptor agonist are used in conjunction with other treatments for otic diseases or disorders. For example, otic compositions and formulations comprising a non-natural neurotrophic agent are used in conjunction with cochlear implants.

### Excitotoxicity

Excitotoxicity refers to the death or damaging of neurons and/or otic hair cells by glutamate and/or similar substances.

Glutamate is the most abundant excitatory neurotransmitter in the central nervous system. Pre-synaptic neurons release glutamate upon stimulation. It flows across the synapse, binds to receptors located on post-synaptic neurons, and activates these neurons. The glutamate receptors include the NMDA, AMPA, and kainate receptors. Glutamate transporters are tasked with removing extracellular glutamate from the synapse. Certain events (e.g. ischemia or stroke) can damage the transporters. This results in excess glutamate accumulating in the synapse. Excess glutamate in synapses results in the over-activation of the glutamate receptors.

The AMPA receptor is activated by the binding of both glutamate and AMPA. Activation of certain isoforms of the AMPA receptor results in the opening of ion channels located in the plasma membrane of the neuron. When the channels open, N⁺ and Ca²⁺ ions flow into the neuron and K⁺ ions flow out of the neuron.

The NMDA receptor is activated by the binding of both glutamate and NMDA. Activation of the NMDA receptor, results in the opening of ion channels located in the plasma membrane of the neuron. However, these channels are blocked by Mg²⁺ ions. Activation of the AMPA receptor results in the expulsion of Mg²⁺ ions from the ion channels into the synapse. When the ion channels open, and the Mg²⁺ ions evacuate the ion channels, Na⁺ and Ca²⁺ ions flow into the neuron, and K⁺ ions flow out of the neuron.

Excitotoxicity occurs when the NMDA receptor and AMPA receptors are over-activated by the binding of excessive amounts of ligands, for example, abnormal amounts of glutamate. The over-activation of these receptors causes excessive opening of the ion channels under their control. This allows abnormally high levels of Ca²⁺ and Na⁺ to enter the neuron. The influx of these levels of Ca²⁺ and Na⁺ into the neuron causes the neuron to fire more often, resulting in a rapid buildup of free radicals and inflammatory compounds within the cell. The free radicals eventually damage the mitochondria, depleting the cell's energy stores. Furthermore, excess levels of Ca²⁺ and Na⁺ ions activate excess levels of enzymes including, but not limited to, phospholipases, endonucleases, and proteases. The over-activation of these enzymes results in damage to the cytoskeleton, plasma membrane, mitochondria, and DNA of the sensory neuron. In some embodiments, a non-natural neurotrophic agent is a functional glutamate receptor antagonist that reduces or inhibits excessive neuronal firing and/or neuronal cell death by modulating glutamate receptor responses and/or modifying the expression of glutamate receptors and/or their associated proteins. Disclosed herein, in certain embodiments, is a pharmaceutical composition for use in the treatment of a disease of the ear characterized by the dysfunction of an NMDA receptor.

### Tinnitus

As used herein, “tinnitus” refers to a disorder characterized by the perception of sound in the absence of any external stimuli. In certain instances, tinnitus occurs in one or both ears, continuously or sporadically, and is most often described as a ringing sound. It is most often used as a diagnostic symptom for other diseases. There are two types of tinnitus: objective and subjective. The former is a sound created in the body which is audible to anyone. The latter is audible only to the affected individual. Studies estimate that over 50 million Americans experience some form of tinnitus. Of those 50 million, about 12 million experience severe tinnitus.

There are several treatments for tinnitus. Lidocaine, administered by IV, reduces or eliminates the noise associated with tinnitus in about 60-80% of sufferers. Selective neurotransmitter reuptake inhibitors, such as nortriptyline, sertraline, and paroxetine, have also demonstrated efficacy against tinnitus. Benzodiazepines are also prescribed to treat tinnitus. In some embodiments, a non-natural neurotrophic agent reduces or inhibits auris sensory cell damage and/or death associated with tinnitus.

### Sensorineural Hearing Loss

Sensorineural hearing loss is a type of hearing loss which results from defects (congenital and acquired) in the vestibulocochlear nerve (also known as cranial nerve VIII), or sensory cells of the inner ear. The majority of defects of the inner ear are defects of otic hair cells and sensory neurons.

Aplasia of the cochlea, chromosomal defects, and congenital cholesteatoma are examples of congenital defects which can result in sensorineural hearing loss. By way of non-limiting example, inflammatory diseases (e.g. suppurative labyrinthitis, meningitis, mumps, measles, viral syphilis, and autoimmune disorders), Meniere's Disease, exposure to ototoxic drugs (e.g. aminoglycosides, loop diuretics, antimetabolites, salicylates, and cisplatin), physical trauma, presbyacusis, and acoustic trauma (prolonged exposure to sound in excess of 90 dB) can all result in acquired sensorineural hearing loss.

If the defect resulting in sensorineural hearing loss is a defect in the auditory pathways, the sensorineural hearing loss is called central hearing loss. If the defect resulting in sensorineural hearing loss is a defect in the auditory pathways, the sensorineural hearing loss is called cortical deafness. In some embodiments, a non-natural Trk receptor agonist is a non-natural neurotrophic agent (e.g., BDNF, GDNF) that promotes growth of auris sensory cells and their processes and connections and reduces or reverses sensorineural hearing loss.

### Noise-Induced Hearing Loss

Noise-induced hearing loss (NIHL) is caused upon exposure to sounds that are too loud or loud sounds that last an extended period of time. Long, repeated, or impulse exposure to sounds at or above 85 decibels can cause hearing loss. Hearing loss may also occur from prolonged exposure to loud noises, such as loud music, heavy equipment or machinery, airplanes, gunfire, or other human-based noises. NIHL causes damage to the hair cells and/or the auditory nerve. The hair cells are small sensory cells that convert sound energy into electrical signals that travel to the brain. Impulse sound can result in immediate hearing loss that may be permanent. This kind of hearing loss may be accompanied by tinnitus—a ringing, buzzing, or roaring in the ears or head which may subside over time. Hearing loss and tinnitus may be experienced in one or both ears, and tinnitus may continue constantly or occasionally throughout a lifetime. Continuous exposure to loud noise also damages the structure of hair cells and sensory neurons, resulting in permanent hearing loss and tinnitus, although the process occurs more gradually than for impulse noise.

In some embodiments, an otoprotectant can reverse, reduce, or ameliorate NIHL. Examples of otoprotectants that treat or prevent NIHL include, but are not limited to, otoprotectants described herein.

Disclosed herein, in some embodiments, is use of a non-natural neurotrophic agent described herein for the treatment of noise-induced hearing loss.

### Ototoxicity

Ototoxicity refers to hearing loss caused by a toxin. The hearing loss may be due to trauma to otic hair cells, the cochlea, and/or the cranial nerve VIII. Multiple drugs are known to be ototoxic. Often ototoxicity is dose-dependent. It may be permanent or reversible upon withdrawal of the drug.

Known ototoxic drugs include, but are not limited to, the aminoglycoside class of antibiotics (e.g. gentamicin and amikacin), some members of the macrolide class of antibiotics (e.g erythromycin), some members of the glycopeptide class of antibiotics (e.g. vancomycin), salicylic acid, nicotine, some chemotherapeutic agents (e.g. actinomycin, bleomycin, cisplatin, carboplatin and vincristine), some members of the loop diuretic family of drugs (e.g. furosemide), 6-hydroxy dopamine (6-OH DPAT), 6,7-dinitroquinoxaline-2,3-dione (DNQX), or the like.

Chemotherapeutic agents and the aminoglycoside class of antibiotics induce the production of reactive oxygen species (“ROS”). ROS can damage cells directly by damaging DNA, polypeptides, and/or lipids. Antioxidants prevent damage of ROS by preventing their formation or scavenging free radicals before they can damage the cell. Both chemotherapeutic agents and the aminoglycoside class of antibiotics are also thought to damage the ear by binding melanin in the stria vascularis of the inner ear. In some instances, hearing loss induced by chemotherapy agents such as cisplatin, actinomycin, bleomycin, carboplatin, oxaliplatin, and vincristine is referred to as chemotherapy-induced hearing loss.

Salicylic acid is classified as ototoxic as it inhibits the function of the polypeptide prestin. Prestin mediates outer otic hair cell motility by controlling the exchange of chloride and carbonate across the plasma membrane of outer otic hair cells. It is only found in the outer otic hair cells, not the inner otic hair cells. Accordingly, disclosed herein is the use of controlled-release auris compositions comprising otoprotectants (e.g. antioxidants) with a non-natural neurotrophic agent described herein, to prevent, ameliorate, or lessen ototoxic effects of chemotherapy, including, but not limited to, cisplatin treatment, aminoglycoside or salicylic acid administration, or other ototoxic agents.

### Endolymphatic Hydrops

Endolymphatic hydrops refers to an increase in the hydraulic pressure within the endolymphatic system of the inner ear. The endolymph and perilymph are separated by thin membranes which contain multiple nerves. Fluctuation in pressure stresses the membranes and the nerves they house. If the pressure is great enough, disruptions may form in these membranes. This results in a mixing of the fluids which can lead to a depolarization blockade and transient loss of function. Changes in the rate of vestibular nerve firing often lead to vertigo. Further, the organ of Corti may also be affected. Distortions of the basilar membrane and the inner and outer hair cells can lead to hearing loss and/or tinnitus.

Causes of endolymphatic hydrops include metabolic disturbances, hormonal imbalances, autoimmune disease, and viral, bacterial, or fungal infections. Symptoms include hearing loss, vertigo, tinnitus, and aural fullness. Nystagmus may also be present. Treatment includes systemic administration of benzodiazepine, diuretics (to decrease the fluid pressure), corticosteroids, and/or anti-bacterial, anti-viral, or anti-fungal agents.

Disclosed herein, in some embodiments, is use of a non-natural neurotrophic agent described herein for the treatment of endolymphatic hydrops.

### Labyrinthitis

Labyrinthitis is an inflammation of the labyrinths of the ear which contain the vestibular system of the inner ear. Causes include bacterial, viral, and fungal infections. It may also be caused by a head injury or allergies. Symptoms of labyrinthitis include difficulty maintaining balance, dizziness, vertigo, tinnitus, and hearing loss. Recovery may take one to six weeks; however, chronic symptoms may be present for years.

There are several treatments for labyrinthitis. Prochlorperazine is often prescribed as an antiemetic. Serotonin-reuptake inhibitors have been shown to stimulate new neural growth within the inner ear. Additionally, treatment with antibiotics is prescribed if the cause is a bacterial infection, and treatment with corticosteroids and antivirals is recommended if the condition is caused by a viral infection.

In some embodiments, disclosed herein include use of a non-natural neurotrophic agent described herein for the treatment of labyrinthitis.

### Meniere's Disease

Meniere's Disease is an idiopathic condition characterized by sudden attacks of vertigo, nausea, and vomiting that may last for 3 to 24 hours, and may subside gradually. Progressive hearing loss, tinnitus, and a sensation of pressure in the ears accompanies the disease through time. The cause of Meniere's disease is likely related to an imbalance of inner ear fluid homeostasis, including an increase in production or a decrease in reabsorption of inner ear fluid.

Studies of the vasopressin (VP)-mediated aquaporin 2 (AQP2) system in the inner ear suggest a role for VP in inducing endolymph production, thereby increasing pressure in the vestibular and cochlear structures. VP levels were found to be upregulated in endolymphatic hydrops (Meniere's Disease) cases, and chronic administration of VP in guinea pigs was found to induce endolymphatic hydrops. Treatment with VP antagonists, including infusion of OPC-31260 (a competitive antagonist of V2-R) into the scala tympani resulted in a marked reduction of Meniere's disease symptoms. Other VP antagonists include WAY-140288, CL-385004, tolvaptan, conivaptan, SR 121463A, and VPA 985. (Sanghi et al. Eur. Heart J. (2005) 26:538-543; Palm et al. Nephrol. Dial Transplant (1999) 14:2559-2562).

Other studies suggest a role for estrogen-related receptor beta/NR3B2 (ERR/Nr3b2) in regulating endolymph production and therefore pressure in the vestibular/cochlear apparatus. Knock-out studies in mice demonstrate the role of the polypeptide product of the Nr3b2 gene in regulating endolymph fluid production. Nr3b2 expression has been localized in the endolymph-secreting strial marginal cells and vestibular dark cells of the cochlea and vestibular apparatus, respectively. Moreover, conditional knockout of the Nr3b2 gene results in deafness and diminished endolymphatic fluid volume. Treatment with antagonists to ERR/Nr3b2 may assist in reducing endolymphatic volume and thus alter pressure in the auris interna structures.

Other treatments may be aimed at dealing with the immediate symptoms and prevention of recurrence. Low-sodium diets as well as avoidance of caffeine, alcohol, and tobacco have been advocated. Medications that may temporarily relieve vertigo attacks include antihistamines (including meclizine and other antihistamines) and central nervous system agents, including barbiturates and/or benzodiazepines, including lorazepam or diazepam. Other examples of drugs that may be useful in relieving symptoms include muscarinic antagonists, including scopolamine. Nausea and vomiting may be relieved by suppositories containing antipsychotic agents, including the phenothiazine agent proclorperazine.

Surgical procedures that have been used to relieve symptoms include the destruction of vestibular and/or cochlear function to relieve vertigo symptoms. These procedures aim to either reduce fluid pressure in the inner ear and/or to destroy inner ear balance function. An endolymphatic shunt procedure, which relieves fluid pressure, may be placed in the inner ear to relieve symptoms of vestibular dysfunction. Other treatments include gentamicin application, which when injected into the eardrum destroys sensory hair cell function, thereby eradicating inner ear balance function. Severing of the vestibular nerve may also be employed which, while preserving hearing, may control vertigo. In some embodiments, an auris sensory cell modulator promotes growth of hair cells and allows a subject to regain inner ear balance function.

In some embodiments, disclosed herein comprises use of a neurotrophic agent described herein for the treatment of Meniere's Disease.

### Meniere's Syndrome

Meniere's Syndrome, which displays similar symptoms as Meniere's disease, is attributed as a secondary affliction to another disease process, e.g. thyroid disease or inner ear inflammation due to syphilis infection. Meniere's syndrome, thus, are secondary effects to various processes that interfere with normal production or resorption of endolymph including endocrine abnormalities, electrolyte imbalance, autoimmune dysfunction, medications, infections (e.g. parasitic infections), or hyperlipidemia. Treatment of patients afflicted with Meniere's Syndrome is similar to Meniere's Disease.

### Ramsay Hunt's Syndrome (Herpes Zoster Infection)

Ramsay Hunt's Syndrome is caused by a herpes zoster infection of the auditory nerve. The infection may cause severe ear pain, hearing loss, vertigo, as well as blisters on the outer ear, in the ear canal, as well as on the skin of the face or neck supplied by the nerves. Facial muscles may also become paralyzed if the facial nerves are compressed by the swelling. Hearing loss may be temporary or permanent, with vertigo symptoms usually lasting from several days to weeks.

Treatment of Ramsay Hunt's syndrome includes administration of antiviral agents, including acyclovir. Other antiviral agents include famciclovir and valacyclovir. Combination of antiviral and corticosteroid therapy may also be employed to ameliorate herpes zoster infection. Analgesics or narcotics may also be administered to relieve the pain, and diazepam or other central nervous system agents to suppress vertigo. Capsaicin, lidocaine patches, and nerve blocks are optionally used. Surgery may also be performed on compressed facial nerves to relieve facial paralysis.

In some embodiments, disclosed herein comprises use of a non-natural neurotrophic agent described herein for the treatment of Ramsay Hunt's Syndrome.

### Microvascular Compression Syndrome

Microvascular compression syndrome (MCS), also called “vascular compression” or “neurovascular compression”, is a disorder characterized by vertigo and tinnitus. It is caused by the irritation of Cranial Nerve VIII by a blood vessel. Other symptoms found in subjects with MCS include, but are not limited to, severe motion intolerance, and neuralgic like “quick spins”. MCS is treated with carbamazepine, TRILEPTAL®, and baclofen. It can also be surgically treated.

In some embodiments, disclosed herein comprises use of a non-natural neurotrophic agent described herein for the treatment of microvascular compression syndrome.

### Vestibular Neuronitis

Vestibular neuronitis, or vestibular neuropathy, is an acute, sustained dysfunction of the peripheral vestibular system. It is theorized that vestibular neuronitis is caused by a disruption of afferent neuronal input from one or both of the vestibular apparatuses. Sources of this disruption include viral infection and acute localized ischemia of the vestibular nerve and/or labyrinth.

The most significant finding when diagnosing vestibular neuronitis is spontaneous, unidirectional, horizontal nystagmus. It is often accompanied by nausea, vomiting, and vertigo. It is, however, generally not accompanied by hearing loss or other auditory symptoms.

There are several treatments for vestibular neuronitis. H1-receptor antagonists, such as dimenhydrinate, diphenhydramine, meclizine, and promethazine, diminish vestibular stimulation and depress labyrinthine function through anticholinergic effects. Benzodiazepines, such as diazepam and lorazepam, are also used to inhibit vestibular responses due to their effects on the GABAA receptor. Anticholinergics, for example scopolamine, are also prescribed. They function by suppressing conduction in the vestibular cerebellar pathways. Finally, corticosteroids (i.e. prednisone) are prescribed to ameliorate the inflammation of the vestibular nerve and associated apparatus.

In some embodiments, disclosed herein comprises use of a non-natural neurotrophic agent described herein for the treatment of vestibular neuronitis.

### Presbycusis

Age-related hearing loss (presbycusis) is the loss of hearing that gradually occurs with ageing. It is one of the most common conditions affecting older and elderly adults. Approximately one in three people in the United States between the ages of 65 and 74 has hearing loss, and nearly half of those older than 75 have difficulty hearing. Having trouble hearing can make it hard to understand and follow a doctor's advice, respond to warnings, and hear phones, doorbells, and smoke alarms. Hearing loss can also make it hard to enjoy talking with family and friends, leading to feelings of isolation. Age-related hearing loss most often occurs in both ears, affecting them equally.

There are many causes of age-related hearing loss. Most commonly, it arises from changes in the inner ear as one ages, but it can also result from changes in the middle ear, or from complex changes along the nerve pathways from the ear to the brain. Certain medical conditions and medications may also play a role. Presbycusis may result from a gradual loss of spiral ganglion neuron afferent fibers and their synapses with hair cells (ribbon synapses), causing a disconnection between the sensory cells that detect sound and the auditory nerve that transmits this information to the auditory brain. Loss of spiral ganglion neurons and hair cells also occurs. Prior exposure to loud noise or other otic insults may exacerbate this ageing process, leading to an accelerated loss of hearing. Presbycusis also involves “hidden hearing loss”, an inability to detect sound against a background noise (“speech-in-noise”) despite a lack of marked changes in hearing thresholds. These more subtle decrements in hearing have been associated with a loss of spiral ganglion neuron afferent fibers and their synaptic connections with hair cells (ribbon synapses).

In some embodiments, disclosed herein comprises use of a non-natural neurotrophic agent described herein for the treatment of presbycusis (age-related hearing loss).

### Pharmaceutical Agents

Provided herein are otic compositions or formulations, comprising non-natural Trk receptor agonists, that modulate the degeneration of auris sensory cells (e.g., neurons and their processes and connections and/or hair cells of the auris) and promote their reconnection. In some embodiments, otic compositions or formulations, comprising non-natural Trk receptor agonists, described herein reduce, delay, or reverse the degeneration of auris sensory cells (e.g., neurons and their processes and connections and/or cells of the auris). Also disclosed herein are controlled-release otic compositions, comprising non-natural Trk receptor agonists, for treating or ameliorating hearing loss or reduction resulting from destroyed, stunted, malfunctioning, damaged, fragile, or missing hair cells in the inner ear. Additionally provided herein are otic compositions or formulations that promote the growth and/or regeneration of auris sensory cells (e.g., neurons and their processes and connections and/or hair cells of the auris). In some embodiments, non-natural Trk receptor agonists are otoprotectants and reduce, reverse, or delay damage to auris sensory cells (e.g., neurons and their processes and connections and/or hair cells of the auris). In some embodiments, non-natural Trk receptor agonists repair damage to the afferent sensory fibers and their ribbon synapses.

Otic and vestibular disorders have causes and symptoms that are responsive to the non-natural Trk receptor agonists disclosed herein.

The non-natural Trk receptor agonist comprising otic compositions or formulations disclosed herein are optionally targeted directly to otic structures where treatment is needed; for example, one embodiment contemplated is the direct application of the formulations disclosed herein onto the round window membrane or the crista fenestrae cochlea of the auris interna, allowing direct access and treatment of the auris interna, or inner ear components. In other embodiments, the formulation disclosed herein is applied directly to the oval window. In yet other embodiments, direct access is obtained through microinjection directly into the auris interna, for example, through cochlear microperfusion. Such embodiments also optionally comprise a drug delivery device, wherein the drug delivery device delivers the non-natural Trk receptor agonist formulations through use of a needle and syringe, a pump, a microinjection device, an auris-acceptable in situ forming spongy material, or any combination thereof.

In some embodiments, the non-natural Trk receptor agonist formulations disclosed herein further comprise otoprotectants that reduce, inhibit, or ameliorate the ototoxicity of pharmaceutical agents disclosed herein, or reduce, inhibit, or ameliorate the effects of other environmental factors, including excessive noise and the like. Examples of otoprotectants include, and are not limited to, otoprotectants described herein, thiols and/or thiol derivatives and/or pharmaceutically acceptable salts, or derivatives (e.g. prodrugs) thereof.

Moreover, some pharmaceutical excipients, diluents, or carriers are potentially ototoxic. For example, benzalkonium chloride, a common preservative, is ototoxic and therefore potentially harmful if introduced into the vestibular or cochlear structures. In formulating a controlled-release non-natural Trk receptor agonist formulation, it is advised to avoid or combine the appropriate excipients, diluents, or carriers to lessen or eliminate potential ototoxic components from the formulation, or to decrease the amount of such excipients, diluents, or carriers. Optionally, a controlled-release non-natural Trk receptor agonist formulation includes otoprotective agents, such as antioxidants, alpha lipoic acid, calcium, fosfomycin, or iron chelators, to counteract potential ototoxic effects that may arise from the use of specific therapeutic agents or excipients, diluents, or carriers.

### Tropomyosin Receptor Kinase (Trk) Agonists

Trk tyrosine kinase receptors are multi-domain single-transmembrane receptors that play an important role in a wide spectrum of neuronal responses including survival, differentiation, growth, and regeneration. Trk receptors are widely distributed in the central nervous system and the peripheral nervous system, and play a key role in neuronal survival, differentiation, and maintenance of proper function.

There are three members of the Trk family: TrkA, TrkB, and TrkC, encoded, respectively, by the genes Ntrk1, Ntrk2, and Ntrk3 in rat or mouse genomic nomenclature, and by NTRK1, NTRK2, and NTRK3 in human genomic nomenclature. The extracellular domains of native TrkA, TrkB, and TrkC receptors have five functional domains that have been defined with reference to homologous or otherwise similar structures identified in various other proteins. The domains have been designated starting at the N-terminus of the amino acid sequence of the mature Trk receptors as 1) a first cysteine-rich domain extending from amino acid position 1 to about amino acid position 32 of human TrkA, from amino acid position 1 to about amino acid position 36 of human TrkB, and from amino acid position 1 to about amino acid position 48 of human TrkC; 2) a leucine-rich domain stretching from about amino acid 33 to about amino acid to about amino acid 104 in TrkA; from about amino acid 37 to about amino acid 108 in TrkB, and from about amino acid 49 to about amino acid 120 in TrkC; 3) a second cysteine-rich domain from about amino acid 105 to about amino acid 157 in TrkA: from about amino acid 109 to about amino acid 164 in TrkB; and from about amino acid 121 to about amino acid 177 in TrkC; 4) a first immunoglobulin-like domain stretching from about amino acid 176 to about amino acid 234 in TrkA, from about amino acid 183 to about amino acid 239 in TrkB, and from about amino acid 196 to about amino acid 257 in TrkC; and 5) a second immunoglobulin-like domain extending from about amino acid 264 to about amino acid 330 in TrkA, from about amino acid 270 to about amino acid 334 in TrkB, and from about amino acid 288 to about amino acid 351 in TrkC.

In some instances, the tropomyosin receptor kinases are characterized as high affinity receptors for naturally occurring neurotrophins, a family of protein growth factors which includes nerve growth factor (NGF), brain derived neurotrophic factor (BDNF), neurotrophin-3 (NT-3), and neurotrophins-4/5 (NT-4/5). In some cases, NT-3, BDNF, and NGF are referred to as essential growth factors for the development and maintenance of the nervous system.

In some embodiments, Trk receptor ectodomain termed D5 comprises the main neurotrophin binding site and is required for ligand-dependent receptor activation. In some instances, the Trk receptor ectodomain D4 further mediates ligand-dependent receptor activation. In additional cases, the Trk receptor ectodomain D1 is involved in mediating ligand-dependent receptor activation. Such receptor sites that define ligand binding and functional activation are termed “hot spots”.

Mature neurotrophins bind a selective Trk receptor with relatively high affinity (e.g. TrkB-BDNF, TrkA-NGF, and TrkC-NT-3). In some cases, TrkC is the preferred receptor for NT-3 and mediates the multiple effects of NT-3, including neuronal death or survival, and cellular differentiation. The Trk receptor has tyrosine kinase catalytic activity that is associated with the survival and differentiation of neurotrophic signals. Neurotrophin-induced Trk activity affords trophic (growth/survival) responses via MAPK and AKT, whereas PLC-γ and fibroblast growth factor receptor substrate-2 (FRS-2) activity are involved in differentiation.

In some embodiments, the mature neurotrophins (e.g., NGF, NT-3, NT-4, NT-5, and BDNF) also bind to p75^{NTR}, a neurotrophin receptor which binds all neurotrophins with lower affinity but, in complex with the ubiquitous protein sortilin, makes a high-affinity receptor for precursor of mature neurotrophins or proneurotrophins. p75^{NTR} is not a receptor protein-tyrosine kinase and recruits intracellular signaling different from that activated by Trks. p75^{NTR} signaling is generally atrophic, promoting apoptosis, inhibiting neurite growth, promoting inflammatory cascades, and depressing synaptic strength. The p75^{NTR} is expressed on glial cells as well as on neurons. In the peripheral nervous system, p75^{NTR} is expressed on Schwann cells after axotomy. When p75^{NTR} is expressed in non-neuronal cells, its activation can cause pro-inflammatory responses. When p75^{NTR} is expressed in neuronal cells together with Trks, the p75^{NTR} receptor can affect Trk-ligand binding or Trk function, although the mechanism is not fully understood. It has been shown that p75^{NTR} can unmask a cryptic “hot spot” of Trk receptors, suggesting the notion of allosteric regulation.

Described herein in some embodiments, are otic compositions comprising non-natural agonists for Trk receptors. In some embodiments, the non-natural agonists for Trk receptors comprise a non-natural neurotrophic agent. In some instances, the non-natural neurotrophic agent is a mutant of at least one of NGF, BDNF, NT-3, NT-4, and NT-5. In some cases, the non-natural neurotrophic agent comprises a proneurotrophin form. In other cases, the non-natural neurotrophic agent comprises a mature neurotrophin form.

### Neurotrophic Agents

Described herein, in some embodiments, are otic compositions comprising a non-natural Trk receptor agonist, wherein the agonist is a non-natural neurotrophic agent. In some embodiments, the non-natural Trk receptor agonist is a non-natural neurotrophic agent that selectively binds to TrkA receptor. In some embodiments, the non-natural Trk receptor agonist is a non-natural neurotrophic agent that selectively binds to TrkB receptor. In some embodiments, the non-natural Trk receptor agonist is a non-natural neurotrophic agent that selectively binds to TrkC receptor. In some embodiments, the non-natural neurotrophic agent binds one or more Trk receptors. In some embodiments, the non-natural Trk receptor agonist is a non-natural neurotrophic agent that does not bind to the neurotrophic receptor p75^{NTR}, a non-natural neurotrophic agent that binds to p75^{NTR} with reduced efficacy, or a non-natural neurotrophic agent that activates p75^{NTR} with reduced efficacy. In some embodiments, the non-natural Trk receptor agonist is a non-natural neurotrophic agent that binds to the neurotrophic receptor p75^{NTR} at a reduced affinity. In some embodiments, the non-natural neurotrophic agent recognizes an isoform of at least one of TrkA, TrkB, TrkC, and p75^{NTR}.

In some embodiments, a non-natural neurotrophic agent is an agent that promotes the growth of tissue and/or neurons and their processes and connections and/or hair cells of the auris. In some embodiments, the non-natural neurotrophic agent is an agent that promotes the survival of neurons and their processes and connections and otic hair cells, and/or the growth of neurons and their processes and connections and otic hair cells. In some embodiments, the non-natural neurotrophic agent which promotes the survival of otic hair cells is a growth factor. In some embodiments, the growth factor is a neurotrophin. In certain instances, neurotrophins are growth factors which prevent cell death, prevent cell damage, repair damaged neurons and their processes and connections and otic hair cells, and/or induce differentiation in progenitor cells. In some embodiments, the neurotrophin is nerve growth factor (NGF), brain-derived neurotrophic factor (BDNF), ciliary neurotrophic factor (CNTF), glial cell-line derived neurotrophic factor (GDNF), neurotrophin-3 (NT-3), neurotrophin-4 (NT-4), neurotrophin-5 (NT-5), a pan-neurotrophin, or a chimeric neurotrophin. In some embodiments, the neurotrophin is a pro-form or precursor of NGF, BDNF, NT-3, NT-4, or NT-5. In some embodiments, the neurotrophin is an isoform of NGF, BDNF, NT-3, NT-4, or NT-5. In some embodiments, the neurotrophin is NGF. In some embodiments, the neurotrophin is BDNF. In some embodiments, the neurotrophin is NT-3. In some embodiments, the neurotrophin is NT-4. In some embodiments, the neurotrophin is NT-5. In some embodiments, the neurotrophin is a pan-neurotrophin. In some embodiments, the neurotrophin is a chimeric neurotrophin. Exemplary sequences of neurotrophins described herein are illustrated in Table 1.

TABLE 1

| | | SEQ |
|---|---|---|
| Protein | | ID NO: |
| Nerve Growth | SSSHPIFHRGEFSVCDSVSVWVGDKTTATDIKGKEVMV | |
| Factor | LGEVNINNSVFKQYFFETKCRDPNPVDSGCRGIDSKHW | |
| (mature form) | NSYCTTTHTFVKALTMDGKQAAWRFIRIDTACVCVL SR | 1 |
| | KAVRRA | |
| Neurotrophin 3 | YAEHKSHRGEYSVCDSESLWVTDKSSAIDIRGHQVTVL | |
| (mature form) | GEIKTGNSPVKQYFYETRCKEARPVKNGCRGIDDKHW | 2 |
| | NSQCKTSQTYVRALTSENNKLVGWRWIRIDTSCVCALS | |
| | RKIGRT | |
| Neurotrophin 4/5 | GVSETAPASRRGELAVCDAVSGWVTDRRTAVDLRGRE | |
| (mature form) | VEVLGEVPAAGGSPLRQYFFETRCKADNAEEGGPGAG | 3 |
| | GGGCRGVDRRHWVSECKAKQSYVRALTADAQGRVG | |
| | WRWIRIDTACVCTLLSRTGRA | |
| Brain-derived | HSDPARRGELSVCDSISEWVTAADKKTAVDMSGGTVT | |
| neurotrophic | VLEKVPVSKGQLKQYFYETKCNPMGYTKEGCRGIDKR | |
| factor | HWNSQCRTTQSYVRALTMDSKKRIGWRFIRIDTSCVCT | 4 |
| (mature form) | LTIKRGR | |
| PNT 1 | SSSHPIFHRGEYSVCDSESLWVTDKSSAIDIRGHQVTVL | |
| | GEIKTGNSPVKQYFYETRCKEARPVKNGCRGIDDKHW | |
| | NSQCKTSQTYVRALTSESKKRIGWRWIRIDTSCVCALS | 5 |
| | RKIGRT | |
| NGF/BDNF | SSSHPIFHRGEFSVCDSVSVWVGDKTTATDIKGKEVMV | |
| chimera 1 | LGEVNINNSVFKQYFFETKCNPMGYTKEGCRGIDSKH | |
| (3 + 4 + 5) | WNSQCRTTQSYVRALTMDSKKRIGWRFIRIDTACVCVL | 6 |
| | SRKAVRRA | |
| NGF/BDNF | SSSHPIFHRGEFSVCDSVSVWVTAADKKTAVDMSGGT | |
| chimera 2 | VMVLGEVNINNSVFKQYFFETKCNPMGYTKEGCRGIDS | |
| (1 + 3 + 4 + 5) | KHWNSQCRTTQSYVRALTMDSKKRIGWRFIRIDTACV | 7 |
| | CVLSRKAVRRA | |

Described herein, in some embodiments, are otic compositions comprising a non-natural Trk receptor agonist, wherein the agonist is a non-natural neurotrophic agent. In some embodiments, the non-natural neurotrophic agent is derived from NGF, NT-3, NT-4, NT-5, BDNF, a pan-neurotrophin, or a chimeric neurotrophin. In some embodiments, the non-natural neurotrophic agent comprises at most 1 amino acid modification, at most 2 amino acid modifications, at most 3 amino acid modifications, at most 4 amino acid modifications, at most 5 amino acid modifications, at most 6 amino acid modifications, at most 7 amino acid modifications, at most 8 amino acid modifications, at most 9 amino acid modifications, at most 10 amino acid modifications, or another suitable number of modifications as compared to a neurotrophic agent comprising a sequence set forth in SEQ ID NO: 1, 2, 3, 4, 5, 6, or 7. In some embodiments, the non-natural neurotrophic agent comprises at least 1 amino acid modification, at least 2 amino acid modifications, at least 3 amino acid modifications, at least 4 amino acid modifications, at least 5 amino acid modifications, at least 6 amino acid modifications, at least 7 amino acid modifications, at least 8 amino acid modifications, at least 9 amino acid modifications, at least 10 amino acid modifications, or another suitable number of modifications as compared to a neurotrophic agent comprising a sequence set forth in SEQ ID NO: 1, 2, 3, 4, 5, 6, or 7.

In some embodiments, a non-natural neurotrophic agent described herein is derived from NGF. In some embodiments, the non-natural neurotrophic agent is derived from a pro-form of NGF. In some instances, the non-natural neurotrophic agent is derived from a mature form of NGF. In additional instances, the non-natural neurotrophic agent is derived from an isoform of NGF. In some embodiments, the non-natural neurotrophic agent comprises the sequence set forth in SEQ ID NO: 1. In some embodiments, the non-natural neurotrophic agent further comprises at most 1 amino acid modification, at most 2 amino acid modifications, at most 3 amino acid modifications, at most 4 amino acid modifications, at most 5 amino acid modifications, at most 6 amino acid modifications, at most 7 amino acid modifications, at most 8 amino acid modifications, at most 9 amino acid modifications, at most 10 amino acid modifications, or another suitable number of modifications. In some embodiments, the non-natural neurotrophic agent further comprises at least 1 amino acid modification, at least 2 amino acid modifications, at least 3 amino acid modifications, at least 4 amino acid modifications, at least 5 amino acid modifications, at least 6 amino acid modifications, at least 7 amino acid modifications, at least 8 amino acid modifications, at least 9 amino acid modifications, at least 10 amino acid modifications, or another suitable number of modifications. In some instances, the non-natural neurotrophic agent consists of the sequence set forth in SEQ ID NO: 1. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent described herein is derived from neurotrophin-3 (NT-3). In some embodiments, neurotrophin-3 promotes the survival of existing neurons and their processes and connections and otic hair cells, and promotes the differentiation of neural and otic hair cell progenitors. Further, in some embodiments, neurotrophin-3 protects cranial nerve VIII from degeneration. In some embodiments, the non-natural neurotrophic agent is derived from a pro-form of NT-3. In some instances, the non-natural neurotrophic agent is derived from a mature form of NT-3. In other instances, the non-natural neurotrophic agent is derived from an isoform of NT-3. In some embodiments, the non-natural neurotrophic agent comprises the sequence set forth in SEQ ID NO: 2. In some embodiments, the non-natural neurotrophic agent further comprises at most 1 amino acid modification, at most 2 amino acid modifications, at most 3 amino acid modifications, at most 4 amino acid modifications, at most 5 amino acid modifications, at most 6 amino acid modifications, at most 7 amino acid modifications, at most 8 amino acid modifications, at most 9 amino acid modifications, at most 10 amino acid modifications, or another suitable number of modifications. In some embodiments, the non-natural neurotrophic agent further comprises at least 1 amino acid modification, at least 2 amino acid modifications, at least 3 amino acid modifications, at least 4 amino acid modifications, at least 5 amino acid modifications, at least 6 amino acid modifications, at least 7 amino acid modifications, at least 8 amino acid modifications, at least 9 amino acid modifications, at least 10 amino acid modifications, or another suitable number of modifications. In some cases, the non-natural neurotrophic agent consists of the sequence set forth in SEQ ID NO: 2. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, the non-natural neurotrophic agent comprises a naturally occurring neurotrophin-3 with one or more mutations which comprise NT-3₍₁₋₁₁₉₎ or NT-3₍₁₋₁₁₇₎ as described in PCT Pub. No. WO9803546.

In some embodiments, the non-natural neurotrophic agent comprises a naturally occurring neurotrophin-3 with one or more mutations that comprises a NT-3 mutant described in Urfer, et al., “The binding epitopes of neurotrophin-3 to its receptors TrkC and gp75 and the design of a multifunctional human neurotrophin,” EMBO 13(24): 5896-5909 (1994).

In some embodiments, a non-natural neurotrophic agent described herein is derived from neurotrophin-4 (NT-4). In some embodiments, the non-natural neurotrophic agent is derived from a pro-form of NT-4. In some instances, the non-natural neurotrophic agent is derived from a mature form of NT-4. In other instances, the non-natural neurotrophic agent is derived from an isoform of NT-4. In some embodiments, the non-natural neurotrophic agent comprises the sequence set forth in SEQ ID NO: 3. In some embodiments, the non-natural neurotrophic agent further comprises at most 1 amino acid modification, at most 2 amino acid modifications, at most 3 amino acid modifications, at most 4 amino acid modifications, at most 5 amino acid modifications, at most 6 amino acid modifications, at most 7 amino acid modifications, at most 8 amino acid modifications, at most 9 amino acid modifications, at most 10 amino acid modifications, or another suitable number of modifications. In some embodiments, the non-natural neurotrophic agent further comprises at least 1 amino acid modification, at least 2 amino acid modifications, at least 3 amino acid modifications, at least 4 amino acid modifications, at least 5 amino acid modifications, at least 6 amino acid modifications, at least 7 amino acid modifications, at least 8 amino acid modifications, at least 9 amino acid modifications, at least 10 amino acid modifications, or another suitable number of modifications. In some cases, the non-natural neurotrophic agent consists of the sequence set forth in SEQ ID NO: 3. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent described herein is derived from a neurotrophin-5 (NT-5). In some embodiments, the non-natural neurotrophic agent is derived from a pro-form of NT-5. In some instances, the non-natural neurotrophic agent is derived from a mature form of NT-5. In other instances, the non-natural neurotrophic agent is derived from an isoform of NT-5. In some embodiments, the non-natural neurotrophic agent comprises of the sequence set forth in SEQ ID NO: 3. In some embodiments, the non-natural neurotrophic agent further comprises at most 1 amino acid modification, at most 2 amino acid modifications, at most 3 amino acid modifications, at most 4 amino acid modifications, at most 5 amino acid modifications, at most 6 amino acid modifications, at most 7 amino acid modifications, at most 8 amino acid modifications, at most 9 amino acid modifications, at most 10 amino acid modifications, or another suitable number of modifications. In some embodiments, the non-natural neurotrophic agent further comprises at least 1 amino acid modification, at least 2 amino acid modifications, at least 3 amino acid modifications, at least 4 amino acid modifications, at least 5 amino acid modifications, at least 6 amino acid modifications, at least 7 amino acid modifications, at least 8 amino acid modifications, at least 9 amino acid modifications, at least 10 amino acid modifications, or another suitable number of modifications. In some cases, the non-natural neurotrophic agent consists of the sequence set forth in SEQ ID NO: 3. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent described herein is derived from BDNF. In some embodiments, the non-natural neurotrophic agent is derived from a pro-form of BDNF. In some instances, the non-natural neurotrophic agent is derived from a mature form of BDNF. In other instances, the non-natural neurotrophic agent is derived from an isoform of BDNF. In some embodiments, the non-natural neurotrophic agent comprises the sequence set forth in SEQ ID NO: 4. In some embodiments, the non-natural neurotrophic agent further comprises at most 1 amino acid modification, at most 2 amino acid modifications, at most 3 amino acid modifications, at most 4 amino acid modifications, at most 5 amino acid modifications, at most 6 amino acid modifications, at most 7 amino acid modifications, at most 8 amino acid modifications, at most 9 amino acid modifications, at most 10 amino acid modifications, or another suitable number of modifications. In some embodiments, the non-natural neurotrophic agent further comprises at least 1 amino acid modification, at least 2 amino acid modifications, at least 3 amino acid modifications, at least 4 amino acid modifications, at least 5 amino acid modifications, at least 6 amino acid modifications, at least 7 amino acid modifications, at least 8 amino acid modifications, at least 9 amino acid modifications, at least 10 amino acid modifications, or another suitable number of modifications. In some cases, the non-natural neurotrophic agent consists of the sequence set forth in SEQ ID NO: 4. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent described herein is a pan-neurotrophin (PNT). In some instances, a pan-neurotrophin is a synthetic trophic factor engineered by combining one or more domains of nerve growth factor (NGF), brain-derived neurotrophic factor (BDNF), and/or neurotrophin 3 (NT-3). In some instances, a pan-neurotrophin recognizes or binds to TrkA, TrkB, and TrkC receptors. In some instances, a pan-neurotrophin is pan-neurotrophin 1 (PNT-1), described in Ilag, et al., “Pan-neurotrophin 1: A genetically engineered neurotrophic factor displaying multiple specificities in peripheral neurons in vitro and in vivo,” PNAS 92: 607-611 (1995). In some cases, a pan-neurotrophin is a pan-neurotrophin described in Ibanez, et al, “An extended surface of binding to Trk tyrosine kinase receptors in NGF and BDNF allows the engineering of a multifunctional pan-neurotrophin,” EMBO 12(6): 2281-2293 (1993).

In some embodiments, the non-natural neurotrophic agent comprises PNT. In some embodiments, the non-natural neurotrophic agent comprises the sequence set forth in SEQ ID NO: 5. In some embodiments, the non-natural neurotrophic agent further comprises at most 1 amino acid modification, at most 2 amino acid modifications, at most 3 amino acid modifications, at most 4 amino acid modifications, at most 5 amino acid modifications, at most 6 amino acid modifications, at most 7 amino acid modifications, at most 8 amino acid modifications, at most 9 amino acid modifications, at most 10 amino acid modifications, or another suitable number of modifications. In some embodiments, the non-natural neurotrophic agent further comprises at least 1 amino acid modification, at least 2 amino acid modifications, at least 3 amino acid modifications, at least 4 amino acid modifications, at least 5 amino acid modifications, at least 6 amino acid modifications, at least 7 amino acid modifications, at least 8 amino acid modifications, at least 9 amino acid modifications, at least 10 amino acid modifications, or another suitable number of modifications. In some cases, the non-natural neurotrophic agent consists of the sequence set forth in SEQ ID NO: 5 (PNT-1). In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some instances, a non-natural neurotrophic agent described herein is a chimeric neurotrophin. In some instances, the chimeric neurotrophin recognizes two or more Trk receptors. In some embodiments, the non-natural neurotrophic agent is a chimera of NGF and BDNF. In some cases, a chimeric neurotrophic agent comprises, for example, one or more domains of nerve growth factor (NGF) and one or more domains of brain-derived neurotrophic factor (BDNF). In some instances, the non-natural neurotrophic agent is a chimera of NGF and BDNF described in Ibáñez, et al., “Chimeric molecules with multiple neurotrophic activities reveal structural elements determining the specificities of NGF and BDNF,” EMBO 10(8): 2105-2110, 1991; Rydén, et al., “Functional analysis of mutant neurotrophins deficient in low-affinity binding reveals a role for p75LNGFR in NT-4 signalling,” EMBO 14(9): 1979-1990, 1995; and/or Ibáñez, et al., “An extended surface of binding to Trk tyrosine kinase receptors in NGF and BDNF allows the engineering of a multifunctional pan-neurotrophin,” EMBO 12(6): 2281-2293, 1993.

In some embodiments, a chimeric neurotrophin described herein is a neurotrophin that binds to two or more Trk receptors. In some embodiments, the chimeric neurotrophin binds to two or more Trk receptors and further has a reduced binding affinity to p75^{NTR}, or activates p75^{NTR} with a reduced efficacy. In some embodiments, the chimeric neurotrophin binds to two or more Trk receptors but does not bind to p75^{NTR}. In some embodiments, the chimeric neurotrophin is further characterized with an improved neural or non-neural survival, differentiation, growth, regeneration compared to a non-chimeric neurotrophin, or a combination thereof, relative to a non-chimeric neurotrophin. In some embodiments, the chimeric neurotrophin has an increased binding affinity, efficacy, potency, or a combination thereof, relative to a non-chimeric neurotrophin.

In some embodiments, the chimera of NGF and BDNF comprises the amino acid sequence set forth in SEQ ID NO: 6 or 7. In some embodiments, the non-natural neurotrophic agent further comprises at most 1 amino acid modification, at most 2 amino acid modifications, at most 3 amino acid modifications, at most 4 amino acid modifications, at most 5 amino acid modifications, at most 6 amino acid modifications, at most 7 amino acid modifications, at most 8 amino acid modifications, at most 9 amino acid modifications, at most 10 amino acid modifications, or another suitable number of modifications. In some embodiments, the non-natural neurotrophic agent further comprises at least 1 amino acid modification, at least 2 amino acid modifications, at least 3 amino acid modifications, at least 4 amino acid modifications, at least 5 amino acid modifications, at least 6 amino acid modifications, at least 7 amino acid modifications, at least 8 amino acid modifications, at least 9 amino acid modifications, at least 10 amino acid modifications, or another suitable number of modifications. In some instances, the non-natural neurotrophic agent consists of the sequence set forth in SEQ ID NO: 6. In some instances, the non-natural neurotrophic agent consists of the sequence set forth in SEQ ID NO: 7. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent described herein comprises a modification at an amino acid residue position equivalent to at least one of an amino acid residue 7, 84, 103, 45, 32, 34, and 35 set forth in SEQ ID NO: 1. In some embodiments, the non-natural neurotrophic agent comprises modifications at amino acid residue positions equivalent to amino acid residues 7 and 103 set forth in SEQ ID NO: 1. In some cases, the non-natural neurotrophic agent further comprises a modification at an amino acid residue position equivalent to amino acid residue 45 or 84 set forth in SEQ ID NO: 1. In some cases, the non-natural neurotrophic agent further comprises a modification at an amino acid residue position equivalent to amino acid residue 84 set forth in SEQ ID NO: 1. In some embodiments, the non-natural neurotrophic agent further comprises a modification at an amino acid residue position equivalent to amino acid residue 45 set forth in SEQ ID NO: 1. In some embodiments, the non-natural neurotrophic agent further comprises modifications at amino acid residue positions equivalent to amino acid residues 32, 34, and 35 set forth in SEQ ID NO: 1. In some embodiments, the non-natural neurotrophic agent comprises modifications at amino acid residue positions equivalent to amino acid residues 7, 84, and 103 set forth in SEQ ID NO: 1. In some embodiments, the non-natural neurotrophic agent comprises modifications at amino acid residue positions equivalent to amino acid residues 7, 45, and 103 set forth in SEQ ID NO: 1. In some embodiments, the non-natural neurotrophic agent comprises modifications at amino acid residue positions equivalent to amino acid residues 7, 32, 34, 35, 45, and 103 set forth in SEQ ID NO: 1. In some embodiments, the non-natural neurotrophic agent comprises modifications at amino acid residue positions equivalent to amino acid residues 7, 32, 34, 35, 84, and 103 set forth in SEQ ID NO: 1. In some instances, the non-natural neurotrophic agent is derived from NGF, NT-3, NT-4, NT-5, BDNF, a pan-neurotrophic agent, or a chimeric neurotrophin. In some instances, the chimeric neurotrophin comprises a sequence set forth in SEQ ID NO: 6 or 7. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent is derived from NGF. In some embodiments, NGF comprises a modification at an amino acid residue position equivalent to at least one of an amino acid residue 7, 84, 103, 45, 32, 34, and 35 set forth in SEQ ID NO: 1. In some embodiments, NGF comprises modifications at amino acid residue positions equivalent to amino acid residues 7 and 103 set forth in SEQ ID NO: 1. In some cases, NGF further comprises a modification at an amino acid residue position equivalent to amino acid residue 45 or 84 set forth in SEQ ID NO: 1. In some cases, NGF further comprises a modification at an amino acid residue position equivalent to amino acid residue 84 set forth in SEQ ID NO: 1. In some embodiments, NGF further comprises a modification at an amino acid residue position equivalent to amino acid residue 45 set forth in SEQ ID NO: 1. In some embodiments, NGF further comprises modifications at amino acid residue positions equivalent to amino acid residues 32, 34, and 35 set forth in SEQ ID NO: 1. In some embodiments, NGF comprises modifications at amino acid residue positions equivalent to amino acid residues 7, 84, and 103 set forth in SEQ ID NO: 1. In some embodiments, NGF comprises modifications at amino acid residue positions equivalent to amino acid residues 7, 45, and 103 set forth in SEQ ID NO: 1. In some embodiments, NGF comprises modifications at amino acid residue positions equivalent to amino acid residues 7, 32, 34, 35, 45, and 103 set forth in SEQ ID NO: 1. In some embodiments, NGF comprises modifications at amino acid residue positions equivalent to amino acid residues 7, 32, 34, 35, 84, and 103 set forth in SEQ ID NO: 1. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent is derived from the mature form of NGF. In some embodiments, the non-natural neurotrophic agent has a modification at at least one of an amino acid residue 7, 84, 103, 45, 32, 34, and 35 set forth in SEQ ID NO: 1. In some embodiments, the non-natural neurotrophic agent comprises modifications at amino acid residues 7 and 103 set forth in SEQ ID NO: 1. In some cases, the non-natural neurotrophic agent further comprises a modification at amino acid residue 45 or 84 set forth in SEQ ID NO: 1. In some cases, the non-natural neurotrophic agent further comprises a modification at amino acid residue 84 set forth in SEQ ID NO: 1. In some embodiments, the non-natural neurotrophic agent further comprises a modification at amino acid residue 45 set forth in SEQ ID NO: 1. In some embodiments, the non-natural neurotrophic agent further comprises modifications at amino acid residues 32, 34, and 35 set forth in SEQ ID NO: 1. In some embodiments, the non-natural neurotrophic agent comprises modifications at amino acid residues 7, 84, and 103 set forth in SEQ ID NO: 1. In some embodiments, the non-natural neurotrophic agent comprises modifications at amino acid residues 7, 45, and 103 set forth in SEQ ID NO: 1. In some embodiments, the non-natural neurotrophic agent comprises modifications at amino acid residues 7, 32, 34, 35, 45, and 103 set forth in SEQ ID NO: 1. In some embodiments, the non-natural neurotrophic agent comprises modifications at amino acid residues 7, 32, 34, 35, 84, and 103 set forth in SEQ ID NO: 1. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent described herein comprises a modification at an amino acid residue position equivalent to at least one of an amino acid residue 32, 34, 61, 74, 75, 77, 100, 115, 116, and 118 set forth in SEQ ID NO: 5. In some embodiments, the non-natural neurotrophic agent comprises at least two modifications selected from amino acid residue positions equivalent to amino acid residues 74, 75 and 77 set forth in SEQ ID NO: 5. In some embodiments, the non-natural neurotrophic agent further comprises a modification at an amino acid residue position equivalent to amino acid residue 32, 34, 115 or 116 set forth in SEQ ID NO: 5. In some embodiments, the non-natural neurotrophic agent further comprises modifications at amino acid residue positions equivalent to amino acid residues 32, 34, 115 and 116 set forth in SEQ ID NO: 5. In some embodiments, the non-natural neurotrophic agent comprises modifications at amino acid residue positions equivalent to amino acid residues 32, 34, 74, 75, 77, 115 and 116 set forth in SEQ ID NO: 5. In some instances, the non-natural neurotrophic agent is NGF, NT-3, NT-4, NT-5, BDNF, a pan-neurotrophic agent, or a chimeric neurotrophin. In some instances, the chimeric neurotrophin comprises a sequence set forth in SEQ ID NO: 6 or 7. In some instances, the modification is a mutation. For example, the mutation is a mutation to at least one of a non-polar residue, a polar residue, and a charged residue. In some instances, the mutation is to alanine. In some cases, the mutation is a conservative mutation, a semi-conservative mutation, or a non-conservative mutation.

In some embodiments, a non-natural neurotrophic agent is PNT. In some embodiments, PNT comprises a modification at an amino acid residue position equivalent to at least one of an amino acid residue 32, 34, 61, 74, 75, 77, 100, 115, 116, and 118 set forth in SEQ ID NO: 5. In some embodiments, PNT comprises at least two modifications selected from amino acid residue positions equivalent to amino acid residues 74, 75, and 77 set forth in SEQ ID NO: 5. In some embodiments, PNT further comprises a modification at an amino acid residue position equivalent to amino acid residue 32, 34, 115, or 116 set forth in SEQ ID NO: 5. In some embodiments, PNT further comprises modifications at amino acid residue positions equivalent to amino acid residues 32, 34, 115, and 116 set forth in SEQ ID NO: 5. In some embodiments, PNT comprises modifications at amino acid residue positions equivalent to amino acid residue 32, 34, 74, 75, 77, 115, and 116 set forth in SEQ ID NO: 5. In some instances, the modification is a mutation. For example, the mutation is a mutation to at least one of a non-polar residue, a polar residue, and a charged residue. In some instances, the mutation is to alanine. In some cases, the mutation is a conservative mutation, a semi-conservative mutation, or a non-conservative mutation.

In some embodiments, a non-natural neurotrophic agent comprises PNT-1. In some embodiments, the non-natural neurotrophic agent comprises a modification at at least one of an amino acid residue 32, 34, 61, 74, 75, 77, 100, 115, 116, and 118 set forth in SEQ ID NO: 5. In some embodiments, the non-natural neurotrophic agent comprises at least two modifications selected from amino acid residues 74, 75, and 77 set forth in SEQ ID NO: 5. In some embodiments, the non-natural neurotrophic agent further comprises a modification at amino acid residue 32, 34, 115, or 116 set forth in SEQ ID NO: 5. In some embodiments, the non-natural neurotrophic agent further comprises modifications at amino acid residue 32, 34, 115, or 116 set forth in SEQ ID NO: 5. In some embodiments, the non-natural neurotrophic agent comprises modifications at amino acid residue 32, 34, 74, 75, 77, 115, and 116 set forth in SEQ ID NO: 5. In some instances, the modification is a mutation. For example, the mutation is a mutation to at least one of a non-polar residue, a polar residue, and a charged residue. In some instances, the mutation is to alanine. In some cases, the mutation is a conservative mutation, a semi-conservative mutation, or a non-conservative mutation.

In some embodiments, a non-natural neurotrophic agent comprises modifications at amino acid residue positions equivalent to amino acid residues 32 and 34 set forth in SEQ ID NO: 5. In some embodiments, the non-natural neurotrophic agent further comprises a modification at an amino acid residue position equivalent to amino acid residue 74, 75, 77, 115, or 116 set forth in SEQ ID NO: 5. In some embodiments, the non-natural neurotrophic agent further comprises modifications at amino acid residue positions equivalent to amino acid residues 74, 75, 77, 115, and 116 set forth in SEQ ID NO: 5. In some embodiments, the non-natural neurotrophic agent is selected from NGF, NT-4, NT-5, BDNF, PNT, or a chimeric neurotrophin. In some instances, the chimeric neurotrophin comprises a sequence set forth in SEQ ID NO: 6 or 7. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent is PNT. In some embodiments, PNT comprises modifications at amino acid residue positions equivalent to amino acid residues 32 and 34 set forth in SEQ ID NO: 5. In some embodiments, PNT further comprises a modification at an amino acid residue position equivalent to amino acid residue 74, 75, 77, 115, or 116 set forth in SEQ ID NO: 5. In some embodiments, PNT further comprises modifications at amino acid residue positions equivalent to amino acid residues 74, 75, 77, 115, and 116 set forth in SEQ ID NO: 5. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent comprises PNT-1. In some embodiments, the non-natural neurotrophic agent comprises modifications at amino acid residues 32 and 34 set forth in SEQ ID NO: 5. In some embodiments, the non-natural neurotrophic agent further comprises a modification at amino acid residue 74, 75, 77, 115, or 116 set forth in SEQ ID NO: 5. In some embodiments, the non-natural neurotrophic agent further comprises modifications at amino acid residues 74, 75, 77, 115, and 116 set forth in SEQ ID NO: 5. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent comprises modifications at amino acid residue positions equivalent to amino acid residues 115 and 116 set forth in SEQ ID NO: 5. In some embodiments, the non-natural neurotrophic agent further comprises a modification at an amino acid residue position equivalent to amino acid residue 32, 34, 74, 75, or 77 set forth in SEQ ID NO: 5. In some embodiments, the non-natural neurotrophic agent further comprises modifications at amino acid residue positions equivalent to amino acid residues 32, 34, 74, 75, and 77 set forth in SEQ ID NO: 5. In some embodiments, the non-natural neurotrophic agent is selected from NGF, NT-4, NT-5, BDNF, PNT, or a chimeric neurotrophin. In some instances, the chimeric neurotrophin comprises a sequence set forth in SEQ ID NO: 6 or 7. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent is PNT. In some embodiments, PNT comprises modifications at amino acid residue positions equivalent to amino acid residues 115 and 116 set forth in SEQ ID NO: 5. In some embodiments, PNT further comprises a modification at an amino acid residue position equivalent to amino acid residue 32, 34, 74, 75, or 77 set forth in SEQ ID NO: 5. In some embodiments, PNT further comprises modifications at amino acid residue positions equivalent to amino acid residues 32, 34, 74, 75, and 77 set forth in SEQ ID NO: 5. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent comprises PNT-1. In some embodiments, the non-natural neurotrophic agent comprises modifications at amino acid residues 115 and 116 set forth in SEQ ID NO: 5. In some embodiments, the non-natural neurotrophic agent further comprises a modification at amino acid residue 32, 34, 74, 75, or 77 set forth in SEQ ID NO: 5. In some embodiments, the non-natural neurotrophic agent further comprises modifications at amino acid residues 32, 34, 74, 75, and 77 set forth in SEQ ID NO: 5. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent comprises modifications at amino acid residue positions equivalent to amino acid residues 61 and 100 set forth in SEQ ID NO: 5. In some embodiments, the non-natural neurotrophic agent is derived from NT-3, NT-4, NT-5, BDNF, PNT, or a chimeric neurotrophin. In some instances, the chimeric neurotrophin comprises a sequence set forth in SEQ ID NO: 6 or 7. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent is PNT. In some embodiments, PNT comprises modifications at amino acid residue positions equivalent to amino acid residues 61 and 100 set forth in SEQ ID NO: 5. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent comprises PNT-1. In some embodiments, the non-natural neurotrophic agent comprises modifications at amino acid residue positions equivalent to amino acid residues 61 and 100 set forth in SEQ ID NO: 5. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation. In some instances, the modification at amino acid residue 61 comprises a mutation from proline to serine. In some cases, the modification at amino acid residue 100 comprises a mutation from arginine to glutamic acid.

In some embodiments, a non-natural neurotrophic agent comprises a modification at amino acid residue position equivalent to amino acid residue 118 set forth in SEQ ID NO: 5. In some embodiments, the non-natural neurotrophic agent is derived from NT-3, NT-4, NT-5, BDNF, PNT, or a chimeric neurotrophin. In some instances, the chimeric neurotrophin comprises a sequence set forth in SEQ ID NO: 6 or 7. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent is PNT. In some instances, PNT comprises a modification at amino acid residue position equivalent to amino acid residue 118 set forth in SEQ ID NO: 5. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent comprises PNT-1. In some embodiments, a non-natural neurotrophic agent comprises a modification at amino acid residue 118 set forth in SEQ ID NO: 5. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent comprises modifications at amino acid residue positions equivalent to amino acid residues 32, 34, and 35 set forth in SEQ ID NO: 6 or 7. In some embodiments, the non-natural neurotrophic agent comprises modifications at amino acid residue positions equivalent to amino acid residues 32, 34, and 35 set forth in SEQ ID NO: 6. In some embodiments, the non-natural neurotrophic agent comprises modifications at amino acid residue positions equivalent to amino acid residues 32, 34, and 35 set forth in SEQ ID NO: 7. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent comprises a modification at an amino acid residue position equivalent to at least one of an amino acid residue 3, 4, 6, 7, 8, 10, 11, 15, 17, 19, 22, 23, 24, 25, 26, 28, 31, 33, 36, 38, 40, 42, 43, 45, 46, 47, 48, 49, 51, 54, 56, 58, 59, 61, 63, 64, 65, 68, 71, 72, 73, 74, 76, 78, 80, 83, 87, 89, 91, 92, 93, 94, 95, 96, 97, 103, 105, 114, and 115 set forth in SEQ ID NO: 2. In some embodiments, the modification is at a single amino acid position set forth in SEQ ID NO: 2. In some embodiments, the modification is at multiple amino acid positions set forth in SEQ ID NO: 2. For example, the modification is at amino acid residue positions equivalent to amino acid residues 15 and 103 set forth in SEQ ID NO: 2. In some embodiments, the modification is at amino acid residue positions equivalent to amino acid residue 15, 114, and 115 set forth in SEQ ID NO: 2. In some embodiments, the modification is at amino acid residue positions equivalent to amino acid residue 11, 15, 68, 87, 114, and 115 set forth in SEQ ID NO: 2. In some embodiments, the modification is at amino acid residue positions equivalent to amino acid residue 15, 68, 87, 114, and 115 set forth in SEQ ID NO: 2. In some embodiments, the modification is at amino acid residue positions equivalent to amino acid residue 11, 15, 87, 114, and 115 set forth in SEQ ID NO: 2. In some embodiments, the modification is at amino acid residue positions equivalent to amino acid residue 15, 87, 114, and 115 set forth in SEQ ID NO: 2. In some embodiments, the modification is at amino acid residue positions equivalent to amino acid residue 11, 15, 114, and 115 set forth in SEQ ID NO: 2. In some embodiments, the modification is at amino acid residue positions equivalent to amino acid residue 15, 103, and 51 set forth in SEQ ID NO: 2. In some embodiments, the modification is at amino acid residue positions equivalent to amino acid residue 15, 103, 114, and 115 set forth in SEQ ID NO: 2. In some embodiments, the modification is at amino acid residue positions equivalent to amino acid residue 31 set forth in SEQ ID NO: 2. In some embodiments, the modification is at amino acid residue positions equivalent to amino acid residue 40 set forth in SEQ ID NO: 2. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent comprises a modification at an amino acid residue position equivalent to at least one of an amino acid residue 11, 15, 31, 40, 51, 68, 87, 103, 114, and 115 set forth in SEQ ID NO: 2. In some embodiments, the modification is at a single amino acid position set forth in SEQ ID NO: 2. In some embodiments, the modification is at multiple amino acid positions set forth in SEQ ID NO: 2. For example, the modification is at amino acid residue positions equivalent to amino acid residues 15 and 103 set forth in SEQ ID NO: 2. In some embodiments, the modification is at amino acid residue positions equivalent to amino acid residue 15, 114, and 115 set forth in SEQ ID NO: 2. In some embodiments, the modification is at amino acid residue positions equivalent to amino acid residue 11, 15, 68, 87, 114, and 115 set forth in SEQ ID NO: 2. In some embodiments, the modification is at amino acid residue positions equivalent to amino acid residue 15, 68, 87, 114, and 115 set forth in SEQ ID NO: 2. In some embodiments, the modification is at amino acid residue positions equivalent to amino acid residue 11, 15, 87, 114, and 115 set forth in SEQ ID NO: 2. In some embodiments, the modification is at amino acid residue positions equivalent to amino acid residue 15, 87, 114, and 115 set forth in SEQ ID NO: 2. In some embodiments, the modification is at amino acid residue positions equivalent to amino acid residue 11, 15, 114, and 115 set forth in SEQ ID NO: 2. In some embodiments, the modification is at amino acid residue positions equivalent to amino acid residue 15, 103, and 51 set forth in SEQ ID NO: 2. In some embodiments, the modification is at amino acid residue positions equivalent to amino acid residue 15, 103, 114, and 115 set forth in SEQ ID NO: 2. In some embodiments, the modification is at amino acid residue positions equivalent to amino acid residue 31 set forth in SEQ ID NO: 2. In some embodiments, the modification is at amino acid residue positions equivalent to amino acid residue 41 set forth in SEQ ID NO: 2. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent is derived from NT-3. In some instances, NT-3 comprises a modification at an amino acid residue position equivalent to at least one of an amino acid residue 11, 15, 31, 40, 51, 68, 87, 103, 114, and 115 set forth in SEQ ID NO: 2. In some embodiments, the modification is at a single amino acid position set forth in SEQ ID NO: 2. In some embodiments, the modification is at multiple amino acid positions set forth in SEQ ID NO: 2. For example, the modification is at amino acid residue positions equivalent to amino acid residues 15 and 103 set forth in SEQ ID NO: 2. In some embodiments, the modification is at amino acid residue positions equivalent to amino acid residue 15, 114, and 115 set forth in SEQ ID NO: 2. In some embodiments, the modification is at amino acid residue positions equivalent to amino acid residue 11, 15, 68, 87, 114, and 115 set forth in SEQ ID NO: 2. In some embodiments, the modification is at amino acid residue positions equivalent to amino acid residue 15, 68, 87, 114, and 115 set forth in SEQ ID NO: 2. In some embodiments, the modification is at amino acid residue positions equivalent to amino acid residue 11, 15, 87, 114, and 115 set forth in SEQ ID NO: 2. In some embodiments, the modification is at amino acid residue positions equivalent to amino acid residue 15, 87, 114, and 115 set forth in SEQ ID NO: 2. In some embodiments, the modification is at amino acid residue positions equivalent to amino acid residue 11, 15, 114, and 115 set forth in SEQ ID NO: 2. In some embodiments, the modification is at amino acid residue positions equivalent to amino acid residue 15, 103, and 51 set forth in SEQ ID NO: 2. In some embodiments, the modification is at amino acid residue positions equivalent to amino acid residue 15, 103, 114, and 115 set forth in SEQ ID NO: 2. In some embodiments, the modification is at amino acid residue positions equivalent to amino acid residue 31 set forth in SEQ ID NO: 2. In some embodiments, the modification is at amino acid residue positions equivalent to amino acid residue 40 set forth in SEQ ID NO: 2. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent is derived from the mature form of NT-3. In some embodiments, the mature form of NT-3 comprises a modification at an amino acid residue 11, 15, 31, 40, 51, 68, 87, 103, 114, and 115 set forth in SEQ ID NO: 2. In some embodiments, the modification is at a single amino acid position set forth in SEQ ID NO: 2. In some embodiments, the modification is at multiple amino acid positions set forth in SEQ ID NO: 2. For example, the modification is at amino acid residues 15 and 103 set forth in SEQ ID NO: 2. In some embodiments, the modification is at amino acid residues 15, 114, and 115 set forth in SEQ ID NO: 2. In some embodiments, the modification is at amino acid residues 11, 15, 68, 87, 114, and 115 set forth in SEQ ID NO: 2. In some embodiments, the modification is at amino acid residues 15, 68, 87, 114, and 115 set forth in SEQ ID NO: 2. In some embodiments, the modification is at amino acid residues 11, 15, 87, 114, and 115 set forth in SEQ ID NO: 2. In some embodiments, the modification is at amino acid residues 15, 87, 114, and 115 set forth in SEQ ID NO: 2. In some embodiments, the modification is at amino acid residues 11, 15, 114, and 115 set forth in SEQ ID NO: 2. In some embodiments, the modification is at amino acid residues 15, 103, and 51 set forth in SEQ ID NO: 2. In some embodiments, the modification is at amino acid residues 15, 103, 114, and 115 set forth in SEQ ID NO: 2. In some embodiments, the modification is at amino acid residues 31 set forth in SEQ ID NO: 2. In some embodiments, the modification is at amino acid residues 40 set forth in SEQ ID NO: 2. In some instances, the modification is a mutation. For example, the mutation is a mutation to at least one of a non-polar residue, a polar residue, and a charged residue. In some cases, the mutation is a conservative mutation, a semi-conservative mutation, or a non-conservative mutation.

In some embodiments, a non-natural neurotrophic agent described herein comprises about 50%, 55%, 60%, 65%, 70%, 75%, 80%, 85%, 90%, 95%, 96%, 97%, 98%, 99%, or 100% sequence identity to the amino acid sequence set forth in SEQ ID NO: 8, 9, 10, or 11. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 8, 9, 10, or 11. In some cases, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 8. In some cases, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 9. In some cases, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 10. In some cases, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 11. In some embodiments, a non-natural neurotrophic agent described herein comprises an amino acid sequence set forth in SEQ ID NO: 8, 9, 10, or 11.

In some embodiments, a non-natural neurotrophic agent comprises about 50%, 55%, 60%, 65%, 70%, 75%, 80%, 85%, 90%, 95%, 96%, 97%, 98%, 99%, or 100% sequence identity to the amino acid sequence set forth in SEQ ID NO: 13, 14, 15, or 16. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 13, 14, 15, or 16. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 13. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 14. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 15. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 16. In some embodiments, a non-natural neurotrophic agent described herein comprises the amino acid sequence set forth in SEQ ID NO: 13, 14, 15, or 16.

In some embodiments, a non-natural neurotrophic agent comprises about 50%, 55%, 60%, 65%, 70%, 75%, 80%, 85%, 90%, 95%, 96%, 97%, 98%, 99%, or 100% sequence identity to the amino acid sequence set forth in SEQ ID NO: 17 or 18. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 17 or 18. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 17. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 18. In some embodiments, a non-natural neurotrophic agent described herein comprises the amino acid sequence set forth in SEQ ID NO: 17 or 18.

In some embodiments, a non-natural neurotrophic agent comprises about 50%, 55%, 60%, 65%, 70%, 75%, 80%, 85%, 90%, 95%, 96%, 97%, 98%, 99%, or 100% sequence identity to the amino acid sequence set forth in SEQ ID NO: 19, 20, or 21. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 19, 20, or 21. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 19. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 20. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 21. In some embodiments, a non-natural neurotrophic agent described herein comprises the amino acid sequence set forth in SEQ ID NO: 19, 20, or 21.

In some embodiments, a non-natural neurotrophic agent comprises about 50%, 55%, 60%, 65%, 70%, 75%, 80%, 85%, 90%, 95%, 96%, 97%, 98%, 99%, or 100% sequence identity to the amino acid sequence set forth in SEQ ID NO: 22, 23, 24, 25, 26, 27, 28, 29, 30, 31, 32, 33, or 34. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 22, 23, 24, 25, 26, 27, 28, 29, 30, 31, 32, 33, or 34. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 22. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 23. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 24. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 25. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 26. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 27. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 28. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 29. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 30. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 31. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 32. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 33. In some embodiments, the non-natural neurotrophic agent comprises about 70%, 80%, 85%, 90%, 91%, 92%, 93%, 94%, 95%, 96%, 97%, 98%, or 99% sequence identity to the amino acid sequence set forth in SEQ ID NO: 34. In some embodiments, a non-natural neurotrophic agent described herein comprises the amino acid sequence set forth in SEQ ID NO: 22, 23, 24, 25, 26, 27, 28, 29, 30, 31, 32, 33, or 34.

In some embodiments, a non-natural neurotrophic agent comprises a sequence as illustrated in Table 2. In some embodiments, the non-natural neurotrophic agent consists of a sequence as illustrated in Table 2.

TABLE 2

| | | | SEQ |
|---|---|---|---|
| Mutein name | mutations | Sequence | ID NO: |
| 7-84-103 | F7A/H84A/R103A | SSSHPIAHRGEFSVCDSVSVWVGDKTTATD | 8 |
| | | IKGKEVMVLGEVNINNSVFKQYFFETKCRD | |
| | | PNPVDSGCRGIDSKHWNSYCTTTATFVKAL | |
| | | TMDGKQAAWRFIAIDTACVCVLSRKAVRR | |
| | | A | |
| 7-45-103 | F7A/N45A/R103A | SSSHPIAHRGEFSVCDSVSVWVGDKTTATD | 9 |
| | | IKGKEVMVLGEVNIANSVFKQYFFETKCRD | |
| | | PNPVDSGCRGIDSKHWNSYCTTTHTFVKAL | |
| | | TMDGKQAAWRFIAIDTACVCVLSRKAVRR | |
| | | A | |
| K84 | K32A/K34A/E35A | SSSHPIAHRGEFSVCDSVSVWVGDKTTATD | 10 |
| | F7A/H84A/R103A | IAGAAVMVLGEVNINNSVFKQYFFETKCRD | |
| | | PNPVDSGCRGIDSKHWNSYCTTTATFVKAL | |
| | | TMDGKQAAWRFIAIDTACVCVLSRKAVRR | |
| | | A | |
| K45 | K32A/K34A/E35A | SSSHPIAHRGEFSVCDSVSVWVGDKTTATD | 11 |
| | F7A/N45A/R103A | IAGAAVMVLGEVNIANSVFKQYFFETKCRD | |
| | | PNPVDSGCRGIDSKHWNSYCTTTHTFVKAL | |
| | | TMDGKQAAWRFIAIDTACVCVLSRKAVRR | |
| | | A | |
| K9 | K32A/K34A/E35A | SSSHPIFHVCDSVSVWVGDKTTATDIAGAA | 12 |
| | Δ9-13 | VMVLGEVNINNSVFKQYFFETKCRDPNPV | |
| | | DSGCRGIDSKHWNSYCTTTHTFVKALTMD | |
| | | GKQAAWRFIRIDTACVCVLSRKAVRRA | |
| Ki-PNT-1 | R32A/H34A | SSSHPIFHRGEYSVCDSESLWVTDKSSAIDI | 13 |
| | | AGAQVTVLGEIKTGNSPVKQYFYETRCKEA | |
| | | RPVKNGCRGIDDKHWNSQCKTSQTYVRAL | |
| | | TSESKKRIGWRWIRIDTSCVCALSRKIGRT | |
| K2-PNT-1 | K74A/H75A/N77A | SSSHPIFHRGEYSVCDSESLWVTDKSSAIDI | 14 |
| | | RGHQVTVLGEIKTGNSPVKQYFYETRCKEA | |
| | | RPVKNGCRGIDDAAWASQCKTSQTYVRAL | |
| | | TSESKKRIGWRWIRIDTSCVCALSRKIGRT | |
| K3-PNT-1 | R115A/K116A | SSSHPIFHRGEYSVCDSESLWVTDKSSAIDI | 15 |
| | | RGHQVTVLGEIKTGNSPVKQYFYETRCKEA | |
| | | RPVKNGCRGIDDKHWNSQCKTSQTYVRAL | |
| | | TSESKKRIGWRWIRIDTSCVCALSAAIGRT | |
| K4-PNT-1 | R32A/H34A/ | SSSHPIFHRGEYSVCDSESLWVTDKSSAIDI | 16 |
| | K74A/H75A/ | AGAQVTVLGEIKTGNSPVKQYFYETRCKEA | |
| | N77A/R115A/ | RPVKNGCRGIDDAAWASQCKTSQTYVRAL | |
| | K116A | TSESKKRIGWRWIRIDTSCVCALSAAIGRT | |
| NGF/BDNF | K32A/K34A/E35A | SSSHPIFHRGEFSVCDSVSVWVGDKTTATDI | 17 |
| chimera 3 | | AGAAVMVLGEVNINNSVFKQYFFETKCNP | |
| (3 + 4 + 5 | | MGYTKEGCRGIDSKHWNSQCRTTQSYVRA | |
| and KKE) | | LTMDSKKRIGWRFIRIDTACVCVLSRKAVR | |
| | | RA | |
| NGF/BDNF | K32A/K34A/E35A | SSSHPIFHRGEFSVCDSVSVWVTAADKKTA | 18 |
| chimera 4 | | VDMAGAAVMVLGEVNINNSVFKQYFFETK | |
| (3 + 4 + 5 | | CNPMGYTKEGCRGIDSKHWNSQCRTTQSY | |
| and KKE) | | VRALTMDSKKRIGWRFIRIDTACVCVLSRK | |
| | | AVRRA | |
| PNT1 + KKE | R32A/H34A/Q35A | SSSHPIFHRGEYSVCDSESLWVTDKSSAIDI | 19 |
| | | AGAAVTVLGEIKTGNSPVKQYFYETRCKEA | |
| | | RPVKNGCRGIDDKHWNSQCKTSQTYVRAL | |
| | | TSESKKRIGWRWIRIDTSCVCALSRKIGRT | |
| PNT-1 mutant | A61S/R101E | SSSHPIFHRGEYSVCDSESLWVTDKSSAIDI | 20 |
| 5 | | RGHQVTVLGEIKTGNSPVKQYFYETRCKES | |
| | | RPVKNGCRGIDDKHWNSQCKTSQTYVRAL | |
| | | TSESKKRIGWEWIRIDTSCVCALSRKIGRT | |
| PNT-1 mutant | G118A | SSSHPIFHRGEYSVCDSESLWVTDKSSAIDI | 21 |
| 6 | | RGHQVTVLGEIKTGNSPVKQYFYETRCKEA | |
| | | RPVKNGCRGIDDKHWNSQCKTSQTYVRAL | |
| | | TSESKKRIGWRWIRIDTSCVCALSRKIART | |
| NT-3 mutant 1 | D15A/R103A | YAEHKSHRGEYSVCASESLWVTDKSSAIDI | 22 |
| | | RGHQVTVLGEIKTGNSPVKQYFYETRCKEA | |
| | | RPVKNGCRGIDDKHWNSQCKTSQTYVRAL | |
| | | TSENNKLVGWRWIAIDTSCVCALSRKIGRT | |
| NT-3 mutant 2 | D15A/R114A/ | YAEHKSHRGEYSVCASESLWVTDKSSAIDI | 23 |
| | K115A | RGHQVTVLGEIKTGNSPVKQYFYETRCKEA | |
| | | RPVKNGCRGIDDKHWNSQCKTSQTYVRAL | |
| | | TSENNKLVGWRWIRIDTSCVCALSAAIGRT | |
| NT-3 mutant 3 | Y11A/D15A/R68A/ | YAEHKSHRGEASVCASESLWVTDKSSAIDI | 24 |
| | R87A/R114A/ | RGHQVTVLGEIKTGNSPVKQYFYETRCKEA | |
| | K115A | RPVKNGCAGIDDKHWNSQCKTSQTYVAAL | |
| | | TSENNKLVGWRWIRIDTSCVCALSAAIGRT | |
| NT-3 mutant 4 | D15A/R68A/R87A/ | YAEHKSHRGEYSVCASESLWVTDKSSAIDI | 25 |
| | R114A/K115A | RGHQVTVLGEIKTGNSPVKQYFYETRCKEA | |
| | | RPVKNGCAGIDDKHWNSQCKTSQTYVAAL | |
| | | TSENNKLVGWRWIRIDTSCVCALSAAIGRT | |
| NT-3 | Y11A/D15A/R87A/ | YAEHKSHRGEASVCASESLWVTDKSSAIDI | 26 |
| mutant 5 | R114A/K115A | RGHQVTVLGEIKTGNSPVKQYFYETRCKEA | |
| | | RPVKNGCRGIDDKHWNSQCKTSQTYVAAL | |
| | | TSENNKLVGWRWIRIDTSCVCALSAAIGRT | |
| NT-3 mutant 6 | D15A/R87A/ | YAEHKSHRGEYSVCASESLWVTDKSSAIDI | 27 |
| | R114A/K115A | RGHQVTVLGEIKTGNSPVKQYFYETRCKEA | |
| | | RPVKNGCRGIDDKHWNSQCKTSQTYVAAL | |
| | | TSENNKLVGWRWIRIDTSCVCALSAAIGRT | |
| NT-3 mutant 7 | Y11A/D15A/ | YAEHKSHRGEASVCASESLWVTDKSSAIDI | 28 |
| | R114A/K115A | RGHQVTVLGEIKTGNSPVKQYFYETRCKEA | |
| | | RPVKNGCRGIDDKHWNSQCKTSQTYVRAL | |
| | | TSENNKLVGWRWIRIDTSCVCALSAAIGRT | |
| NT-3 mutant 8 | D15A/R103A/ | YAEHKSHRGEYSVCASESLWVTDKSSAIDI | 29 |
| | Y51A | RGHQVTVLGEIKTGNSPVKQAFYETRCKEA | |
| | | RPVKNGCRGIDDKHWNSQCKTSQTYVRAL | |
| | | TSENNKLVGWRWIAIDTSCVCALSRKIGRT | |
| NT-3 mutant 9 | D15A/R103A/ | YAEHKSHRGEYSVCASESLWVTDKSSAIDI | 30 |
| | R114A/K115A | RGHQVTVLGEIKTGNSPVKQYFYETRCKEA | |
| | | RPVKNGCRGIDDKHWNSQCKTSQTYVRAL | |
| | | TSENNKLVGWRWIAIDTSCVCALSAAIGRT | |
| NT-3 mutant 10 | D15A | YAEHKSHRGEYSVCASESLWVTDKSSAIDI | 31 |
| | | RGHQVTVLGEIKTGNSPVKQYFYETRCKEA | |
| | | RPVKNGCRGIDDKHWNSQCKTSQTYVRAL | |
| | | TSENNKLVGWRWIRIDTSCVCALSRKIGRT | |
| NT-3 mutant 11 | R114A/K115A | YAEHKSHRGEYSVCDSESLWVTDKSSAIDI | 32 |
| | | RGHQVTVLGEIKTGNSPVKQYFYETRCKEA | |
| | | RPVKNGCRGIDDKHWNSQCKTSQTYVRAL | |
| | | TSENNKLVGWRWIRIDTSCVCALSAAIGRT | |
| NT-3 mutant 12 | R31A | YAEHKSHRGEYTSSVCASESLWVTDKSSAIDI | 33 |
| | | AGHQVTVLGEIKTGNSPVKQYFYETRCKEA | |
| | | RPVKNGCRGIDDKHWNSQCKTSQTYVRAL | |
| | | ENNKLVGWRWIRIDTSCVCALSRKIGRT | |
| NT-3 mutant 13 | E40A | YAEHKSHRGEYSVCASESLWVTDKSSAIDI | 34 |
| | | AGHQVTVLGAIKTGNSPVKQYFYETRCKE | |
| | | ARPVKNGCRGIDDKHWNSQCKTSQTYVRA | |
| | | LTSENNKLVGWRWIRIDTSCVCALSRKIGR | |
| | | T | |

In some embodiments, a non-natural neurotrophic agent is derived from GDNF. In certain instances, GDNF and BDNF are neurotrophic agents that promote the survival of existing neurons and their processes and connections (e.g. spiral ganglion neurons) and otic hair cells by repairing damaged cells, inhibiting the production of ROS, and/or inhibiting cell death. In some embodiments, the non-natural neurotrophic agent also promotes the differentiation of neural and otic hair cell progenitors. Further, in some embodiments, the non-natural neurotrophic agent protects the Cranial Nerve VIII from degeneration. In some embodiments, cells treated with exogenous GDNF have higher survival rates after trauma than untreated cells. In some embodiments, the neurotrophic agent BDNF is administered in conjunction with fibroblast growth factor. In some cases, GDNF comprises a naturally occurring GDNF with one or more mutations or modifications (e.g., chemical modifications such as phosphorylation, incorporation of unnatural amino acids, biotinylation, cyclisation, and the like) in amino acid residues.

In some embodiments, a non-natural neurotrophic agent is derived from CNTF. In some embodiments, CNTF promotes the synthesis of neurotransmitters and the growth of neurites. In some embodiments, CNTF is administered in conjunction with BDNF. In some cases, CNTF comprises a naturally occurring CNTF with one or more mutations or modifications (e.g., chemical modifications such as phosphorylation, incorporation of unnatural amino acids, biotinylation, cyclisation, and the like) in amino acid residues.

In some embodiments, binding of a non-natural Trk receptor agonist to a Trk receptor results in increased levels of phosphorylated Trk receptor, phosphorylated MAPK, phosphorylated Akt, phosphorylated ERK1/2, and phosphorylated phospholipase C-γ. In some embodiments, binding of a non-natural Trk receptor agonist to Trk receptor leads to improved neuronal survival. In some embodiments, administration of an otic composition comprising a non-natural Trk receptor agonist that binds to Trk receptor leads to improved neuronal survival and treats or prevents an otic condition. In some embodiments, administration of an otic composition comprising a non-natural Trk receptor agonist that binds to a Trk receptor leads to improved neuronal survival and treats or prevents an otic condition that requires reconnection of afferent sensory fibers and repair of ribbon synapses. In some embodiments, administration of an otic composition comprising a non-natural Trk receptor agonist that binds to a Trk receptor treats or prevents presbycusis (age related hearing loss). In some embodiments, administration of an otic composition comprising a non-natural Trk receptor agonist that binds to a Trk receptor leads to improved neuronal survival and treats sensorineural hearing loss.

In some embodiments, the binding affinity of a non-natural Trk receptor agonist to a Trk receptor is about 0.001 nM to about 60 μM. In some instances, the binding affinity of a non-natural Trk receptor agonist to a Trk receptor is about 0.001 nM to about 60 μM, 0.001 nM to about 50 μM, 0.001 nM to about 40 μM, 0.001 nM to about 30 μM, 0.001 nM to about 20 μM, 0.001 nM to about 10 μM, 0.001 nM to about 8 μM, 0.001 nM to about 5 μM 0.01 nM to about 50 μM, 0.001 nM to about 3 μM, 0.001 nM to about 1 μM, 0.01 nM to about 50 μM, 0.01 nM to about 40 μM, about 0.01 nM to about 30 μM, about 0.01 nM to about 20 μM, about 0.01 nM to about 10 μM, about 0.01 nM to about 8 μM, about 0.01 nM to about 5 μM, about 0.01 nM to about 3 μM, about 0.01 nM to about 1 μM, about 0.1 nM to about 50 μM, about 0.1 nM to about 40 μM, about 0.1 nM to about 30 μM, about 0.1 nM to about 20 μM, about 0.1 nM to about 10 μM, about 0.1 nM to about 8 μM, about 0.1 nM to about 5 μM, about 0.01 nM to about 1 μM, about 1 nM to about 50 μM, about 10 nM to about 30 μM, about 100 nM to about 20 μM, about 500 nM to about 10 μM, about 500 nM to about 1 μM, about 50 nM to about 5 μM, about 100 nM to about 1 μM, or about 1 nM to about 1 μM.

In some embodiments, the binding affinity of a non-natural Trk receptor agonist to a Trk receptor is about 0.10 to about 0.80 nM, about 0.15 to about 0.75 nM and about 0.18 to about 0.72 nM, about 1 nM to about 1.5 nM, about 2 nM to about 5 nM, about 10 nM to about 20 nM, about 30 nM to about 50 nM, about 75 nM to about 100 nM, about 125 nM to about 150 nM, about 160 nM to about 200 nM. In some embodiments, the binding affinity is about 2 pM, about 5 pM, about 10 pM, about 15 pM, about 20 pM, about 40 pM, or greater than about 40 pM. In some embodiments, the binding affinity is between about 2 pM and 22 pM. In some embodiments, the binding affinity is less than about 10 nM, about 5 nM, about 1 nM, about 900 pM, about 800 pM, about 700 pM, about 600 pM, about 500 pM, about 400 pM, about 300 pM, about 200 pM, about 150 pM, about 100 pM, about 90 pM, about 80 pM, about 70 pM, about 60 pM, about 50 pM, about 40 pM, about 30 pM, about 10 pM. In some embodiments, the binding affinity is about 10 nM. In some embodiments, the binding affinity is less than about 10 nM. In other embodiments, the binding affinity is about 0.1 nM or about 0.07 nM. In other embodiments, the binding affinity is less than about 0.1 nM or less than about 0.07 nM. In some embodiments, the binding affinity is any of about 10 nM, about 5 nM, about 1 nM, about 900 pM, about 800 pM, about 700 pM, about 600 pM, about 500 pM, about 400 pM, about 300 pM, about 200 pM, about 150 pM, about 100 pM, about 90 pM, about 80 pM, about 70 pM, about 60 pM, about 50 pM, about 40 pM, about 30 pM, about 10 pM to any of about 2 pM, about 5 pM, about 10 pM, about 15 pM, about 20 pM, or about 40 pM. In some embodiments, the binding affinity is any of about 10 nM, about 5 nM, about 1 nM, about 900 pM, about 800 pM, about 700 pM, about 600 pM, about 500 pM, about 400 pM, about 300 pM, about 200 pM, about 150 pM, about 100 pM, about 90 pM, about 80 pM, about 70 pM, about 60 pM, about 50 pM, about 40 pM, about 30 pM, about 10 pM. In some embodiments, the binding affinity is about 2 pM, about 5 pM, about 10 pM, about 15 pM, about 20 pM, about 40 pM, or greater than about 40 pM. In some embodiments, the binding affinity falls within any range bound by any of these values, for example, between about 175 nM and about 180 nM. In some embodiments, the binding affinity is 100 nM. In some embodiments, the binding affinity is 200 nM. In some embodiments, the Trk receptor is at least one of TrkA, TrkB, and TrkC.

In some embodiments, the off-rate (or k_{off}) of a non-natural Trk receptor agonist to Trk receptor is from about 10⁻¹ to about 10⁻⁶ s⁻¹. In some embodiments, the off-rate (or k_{off}) of a non-natural Trk receptor agonist to Trk receptor is from about 10⁻² to about 10⁻⁶ s⁻¹, from about 10⁻³ to about 10⁻⁶ s⁻¹, from about 10⁻⁴ to about 10⁻⁶ s⁻¹, from about 10⁻² to about 10⁻⁵ s⁻¹, from about 10⁻² to about 10⁻⁴ s⁻¹, from about 10⁻² to about 10⁻³ s⁻¹, from about 10⁻³ to about 10⁻⁵ s⁻¹, from about 10⁻³ to about 10⁻⁴ s⁻¹, from about 10⁻⁴ to about 10⁻⁵ s⁻¹, from about 10⁻¹ to about 10⁻⁵ s⁻¹, from about 10⁻¹ to about 10⁻⁴ s⁻¹, from about 10⁻¹ to about 10⁻³ s⁻¹, or from about 10⁻¹ to about 10⁻² s⁻¹. In some embodiments, the off-rate (or k_{off}) of a TrkA agonist to TrkA receptor is about 10⁻¹ s⁻¹, about 10⁻² s⁻¹, about 10⁻³ s⁻¹, about 10⁻⁴ s⁻¹, about 10⁻⁵ s⁻¹, or about 10⁻⁶ s⁻¹. In some embodiments, the Trk receptor is at least one of TrkA, TrkB, and TrkC.

In some embodiments, the potency of a non-natural Trk receptor agonist to a Trk receptor is from about 0.001 nM to about 60 μM. In some instances, the potency of a non-natural Trk receptor agonist to a Trk receptor is from about 0.001 nM to about 60 μM, 0.001 nM to about 50 μM, 0.001 nM to about 40 μM, 0.001 nM to about 30 μM, 0.001 nM to about 20 μM, 0.001 nM to about 10 μM, 0.001 nM to about 8 μM, 0.001 nM to about 5 μM 0.01 nM to about 50 μM, 0.001 nM to about 3 μM, 0.001 nM to about 1 μM, 0.01 nM to about 50 μM, from about 0.01 nM to about 40 μM, from about 0.01 nM to about 30 μM, from about 0.01 nM to about 20 μM, from about 0.01 nM to about 10 μM, from about 0.01 nM to about 8 μM, from about 0.01 nM to about 5 μM, from about 0.01 nM to about 3 μM, from about 0.01 nM to about 1 μM, from about 0.1 nM to about 50 μM, from about 0.1 nM to about 40 μM, from about 0.1 nM to about 30 μM, from about 0.1 nM to about 20 μM, from about 0.1 nM to about 10 μM, from about 0.1 nM to about 8 μM, from about 0.1 nM to about 5 μM, from about 0.01 nM to about 1 μM, from about 1 nM to about 50 μM, from about 10 nM to about 30 μM, from about 100 nM to about 20 μM, from about 500 nM to about 10 μM, from about 500 nM to about 1 μM, from about 50 nM to about 5 μM, from about 100 nM to about 1 μM, or from about 1 nM to about 1 μM. In some embodiments, the Trk receptor is at least one of TrkA, TrkB, and TrkC.

In some embodiments, the potency of a non-natural Trk receptor agonist to a Trk receptor is from about 0.10 to about 0.80 nM, from about 0.15 to about 0.75 nM, from about 0.18 to about 0.72 nM, from about 1 nM to about 1.5 nM, from about 2 nM to about 5 nM, from about 10 nM to about 20 nM, from about 30 nM to about 50 nM, from about 75 nM to about 100 nM, from about 125 nM to about 150 nM, or from about 160 nM to about 200 nM. In some embodiments, the potency is about 2 pM, about 5 pM, about 10 pM, about 15 pM, about 20 pM, about 40 pM, or greater than about 40 pM. In some embodiments, the potency is between about 2 pM and 22 pM. In some embodiments, the potency is less than about 10 nM, about 5 nM, about 1 nM, about 900 pM, about 800 pM, about 700 pM, about 600 pM, about 500 pM, about 400 pM, about 300 pM, about 200 pM, about 150 pM, about 100 pM, about 90 pM, about 80 pM, about 70 pM, about 60 pM, about 50 pM, about 40 pM, about 30 pM, about 10 pM. In some embodiments, the potency is about 10 nM. In some embodiments, the potency is less than about 10 nM. In other embodiments, the potency is about 0.1 nM or about 0.07 nM. In other embodiments, the potency is less than about 0.1 nM or less than about 0.07 nM. In some embodiments, the potency is any of about 10 nM, about 5 nM, about 1 nM, about 900 pM, about 800 pM, about 700 pM, about 600 pM, about 500 pM, about 400 pM, about 300 pM, about 200 pM, about 150 pM, about 100 pM, about 90 pM, about 80 pM, about 70 pM, about 60 pM, about 50 pM, about 40 pM, about 30 pM, about 10 pM, about 5 pM, or about 2 pM. In some embodiments, the potency is any of about 10 nM, about 5 nM, about 1 nM, about 900 pM, about 800 pM, about 700 pM, about 600 pM, about 500 pM, about 400 pM, about 300 pM, about 200 pM, about 150 pM, about 100 pM, about 90 pM, about 80 pM, about 70 pM, about 60 pM, about 50 pM, about 40 pM, about 30 pM, or about 10 pM. In some embodiments, the potency is about 2 pM, about 5 pM, about 10 pM, about 15 pM, about 20 pM, about 40 pM, about 40 pM or greater. In some embodiments, the potency falls within any range bound by any of these values, for example, from about 175 nM to about 180 nM. In some embodiments, the potency is 100 nM. In some embodiments, the potency is 200 nM. In some embodiments, the Trk receptor is at least one of TrkA, TrkB, and TrkC.

In some embodiments, a non-natural neurotrophic agent described herein has an increased binding affinity to one or more Trk receptors relative to the binding affinity of an equivalent wild type neurotrophin. In some embodiments, the Trk receptor is at least one of TrkA, TrkB, and TrkC. In some instances, the increase in binding affinity is about 20%, 30%, 40%, 50%, 60%, 70%, 80%, 85%, 90%, 95%, 96%, 97%, 98%, 99%, or more, relative to the binding affinity of an equivalent wild type neurotrophin. In some cases, the increase in binding affinity is about 20% or more, relative to the binding affinity of an equivalent wild type neurotrophin. In some cases, the increase in binding affinity is about 30% or more, relative to the binding affinity of an equivalent wild type neurotrophin. In some cases, the increase in binding affinity is about 40% or more, relative to the binding affinity of an equivalent wild type neurotrophin. In some cases, the increase in binding affinity is about 50% or more, relative to the binding affinity of an equivalent wild type neurotrophin. In some cases, the increase in binding affinity is about 60% or more, relative to the binding affinity of an equivalent wild type neurotrophin. In some cases, the increase in binding affinity is about 70% or more, relative to the binding affinity of an equivalent wild type neurotrophin. In some cases, the increase in binding affinity is about 80% or more, relative to the binding affinity of an equivalent wild type neurotrophin. In some cases, the increase in binding affinity is about 85% or more, relative to the binding affinity of an equivalent wild type neurotrophin. In some cases, the increase in binding affinity is about 90% or more, relative to the binding affinity of an equivalent wild type neurotrophin. In some cases, the increase in binding affinity is about 95% or more, relative to the binding affinity of an equivalent wild type neurotrophin. In some cases, the increase in binding affinity is about 96% or more, relative to the binding affinity of an equivalent wild type neurotrophin. In some cases, the increase in binding affinity is about 97% or more, relative to the binding affinity of an equivalent wild type neurotrophin. In some cases, the increase in binding affinity is about 98% or more, relative to the binding affinity of an equivalent wild type neurotrophin. In some cases, the increase in binding affinity is about 99% or more, relative to the binding affinity of an equivalent wild type neurotrophin.

In some embodiments, a non-natural neurotrophic agent described herein has a decrease in binding affinity to p75^{NTR} relative to the binding affinity of an equivalent wild-type neurotrophin. In some embodiments, the non-natural neurotrophic agent is derived from at least one of NGF, NT-3, NT-4, NT-5, BDNF, PNT, and a chimeric neurotrophic agent. In some embodiments, the decrease in binding affinity is about 30%, 40%, 50%, 60%, 70%, 80%, 85%, 90%, 95%, 96%, 97%, 98%, 99%, or more, relative to the binding affinity of an equivalent wild type neurotrophin. In some embodiments, the decrease in binding affinity is about 30% or more, relative to the binding affinity of an equivalent wild type neurotrophin. In some embodiments, the decrease in binding affinity is about 40% or more, relative to the binding affinity of an equivalent wild type neurotrophin. In some embodiments, the decrease in binding affinity is about 50% or more, relative to the binding affinity of an equivalent wild type neurotrophin. In some embodiments, the decrease in binding affinity is about 60% or more, relative to the binding affinity of an equivalent wild type neurotrophin. In some embodiments, the decrease in binding affinity is about 70% or more, relative to the binding affinity of an equivalent wild type neurotrophin. In some embodiments, the decrease in binding affinity is about 80% or more, relative to the binding affinity of an equivalent wild type neurotrophin. In some embodiments, the decrease in binding affinity is about 85% or more, relative to the binding affinity of an equivalent wild type neurotrophin. In some embodiments, the decrease in binding affinity is about 90% or more, relative to the binding affinity of an equivalent wild type neurotrophin. In some embodiments, the decrease in binding affinity is about 95% or more, relative to the binding affinity of an equivalent wild type neurotrophin. In some embodiments, the decrease in binding affinity is about 96% or more, relative to the binding affinity of an equivalent wild type neurotrophin. In some embodiments, the decrease in binding affinity is about 97% or more, relative to the binding affinity of an equivalent wild type neurotrophin. In some embodiments, the decrease in binding affinity is about 98% or more, relative to the binding affinity of an equivalent wild type neurotrophin. In some embodiments, the decrease in binding affinity is about 99% or more, relative to the binding affinity of an equivalent wild type neurotrophin. In some embodiments, the decrease in binding affinity is more than 99%, relative to the binding affinity of an equivalent wild type neurotrophin. In some embodiments, a non-natural neurotrophic agent described herein does not bind to p75^{NTR}, or activates p75^{NTR} with a reduced efficacy (e.g., the activation is reduced by 30%, 40%, 50%, 60%, 70%, 80%, 85%, 90%, 95%, 96%, 97%, 98%, 99%, or more, relative to the activation of p75^{NTR} by a wild-type neurotrophic agent). In some instances, the term p75^{NTR} encompasses one or more p75^{NTR} isoforms. In some embodiments, agonist activity is determined by one or more techniques well-known in the art. Suitable techniques include, but not limited to, Western blot (Wes Protein Analyzer, Protein Simple Inc.), AlphaLisa assay (Perkin Elmer Enspire plate reader), ELISA, and immunohistochemistry. In some instances, a protein modification such as protein phosphorylation is measured in cells. For example, p-ERK and p-AKT is measured. In some instances, cell survival assays are performed. In some embodiments, assays are performed to measure neurite outgrowth and branching. In some embodiments, techniques are used with at least one of cultured cells, primary cells, and tissue. In some instances, techniques are used with cells that heterologously express a Trk receptor. In some embodiments, EC₅₀ and maximal effect values such as percent efficacy are determined from dose-response curves using such techniques.

In some embodiments, the binding affinity is determined by one or more techniques well-known in the art. Suitable techniques include, e.g., surface plasmon resonance (Biacore3000™ surface plasmon resonance (SPR) system, Biacore, Inc.) equipped with pre-immobilized streptavidin sensor chips, which allows determination of the rate constants for binding (kₐ) and dissociation (k_{d}) of an agonist to a TrkC receptor; isothermal titration calorimetry (ITC); Octet® (ForteBio); KinExA® (Kinetic Exclusion Assay, KinExA 3000, Sapidyne Instruments, Inc.); flow cytometry and ELISA.

In some embodiments, a modification disclosed herein comprises a mutation to a natural or unnatural amino acid residue. For example, in some cases, the modification comprises a mutation of an amino acid residue to an alternative natural amino acid residue, for example a mutation of an arginine to an alanine. In some instances, the modification comprises a mutation of an amino acid residue to an unnatural amino acid residue, for example, a mutation of a cysteine to a seleno-L-cystine.

As used herein, an amino acid residue is a molecule containing both an amino group and a carboxyl group. Suitable amino acids for use in a non-natural neurotrophic agent described herein include, without limitation, both the D- and L-isomers of the naturally-occurring amino acids, as well as non-naturally occurring amino acids prepared by organic synthesis or other metabolic routes. In some instances, an amino acid is an α-amino acid, β-amino acid, natural amino acid, non-natural amino acid, or amino acid analog. A naturally occurring amino acid comprises any one of the twenty amino acids commonly found in peptides synthesized in nature, and known by the one letter abbreviations A, R, N, C, D, Q, E, G, H, I, L, K, M, F, P, S, T, W, Y, and V.

Exemplary unnatural amino acid residues comprise, for example, amino acid analogs such as β-amino acid analogs; racemic analogs; or analogs of amino acid residue alanine, valine, glycine, leucine, arginine, lysine, aspartic acid, glutamic acid, cysteine, methionine, tyrosine, phenylalanine, tryptophane, serine, threonine, or proline. Exemplary β-amino acid analogs include, but are not limited to, cyclic β-amino acid analogs, β-alanine, (R)-β-phenylalanine, (R)-1,2,3,4-tetrahydro-isoquinoline-3-acetic acid, (R)-3-amino-4-(1-naphthyl)-butyric acid, (R)-3-amino-4-(2,4-dichlorophenyl)butyric acid, (R)-3-amino-4-(2-chlorophenyl)-butyric acid, (R)-3-amino-4-(2-cyanophenyl)-butyric acid, (R)-3-amino-4-(2-fluorophenyl)-butyric acid, (R)-3-amino-4-(2-furyl)-butyric acid, (R)-3-amino-4-(2-methylphenyl)-butyric acid, (R)-3-amino-4-(2-naphthyl)-butyric acid, (R)-3-amino-4-(2-thienyl)-butyric acid, (R)-3-amino-4-(2-trifluoromethylphenyl)-butyric acid, (R)-3-amino-4-(3,4-dichlorophenyl)butyric acid, (R)-3-amino-4-(3,4-difluorophenyl)butyric acid, (R)-3-amino-4-(3-benzothienyl)-butyric acid, (R)-3-amino-4-(3-chlorophenyl)-butyric acid, (R)-3-amino-4-(3-cyanophenyl)-butyric acid, (R)-3-amino-4-(3-fluorophenyl)-butyric acid, (R)-3-amino-4-(3-methylphenyl)-butyric acid, (R)-3-amino-4-(3-pyridyl)-butyric acid, (R)-3-amino-4-(3-thienyl)-butyric acid, (R)-3-amino-4-(3-trifluoromethylphenyl)-butyric acid, (R)-3-amino-4-(4-bromophenyl)-butyric acid, (R)-3-amino-4-(4-chlorophenyl)-butyric acid, (R)-3-amino-4-(4-cyanophenyl)-butyric acid, (R)-3-amino-4-(4-fluorophenyl)-butyric acid, (R)-3-amino-4-(4-iodophenyl)-butyric acid, (R)-3-amino-4-(4-methylphenyl)-butyric acid, (R)-3-amino-4-(4-nitrophenyl)-butyric acid, (R)-3-amino-4-(4-pyridyl)-butyric acid, (R)-3-amino-4-(4-trifluoromethylphenyl)-butyric acid, (R)-3-amino-4-pentafluoro-phenylbutyric acid, (R)-3-amino-5-hexenoic acid, (R)-3-amino-5-hexynoic acid, (R)-3-amino-5-phenylpentanoic acid, (R)-3-amino-6-phenyl-5-hexenoic acid, (S)-1,2,3,4-tetrahydro-isoquinoline-3-acetic acid, (S)-3-amino-4-(1-naphthyl)-butyric acid, (S)-3-amino-4-(2,4-dichlorophenyl)butyric acid, (S)-3-amino-4-(2-chlorophenyl)-butyric acid, (S)-3-amino-4-(2-cyanophenyl)-butyric acid, (S)-3-amino-4-(2-fluorophenyl)-butyric acid, (S)-3-amino-4-(2-furyl)-butyric acid, (S)-3-amino-4-(2-methylphenyl)-butyric acid, (S)-3-amino-4-(2-naphthyl)-butyric acid, (S)-3-amino-4-(2-thienyl)-butyric acid, (S)-3-amino-4-(2-trifluoromethylphenyl)-butyric acid, (S)-3-amino-4-(3,4-dichlorophenyl)butyric acid, (S)-3-amino-4-(3,4-difluorophenyl)butyric acid, (S)-3-amino-4-(3-benzothienyl)-butyric acid, (S)-3-amino-4-(3-chlorophenyl)-butyric acid, (S)-3-amino-4-(3-cyanophenyl)-butyric acid, (S)-3-amino-4-(3-fluorophenyl)-butyric acid, (S)-3-amino-4-(3-methylphenyl)-butyric acid, (S)-3-amino-4-(3-pyridyl)-butyric acid, (S)-3-amino-4-(3-thienyl)-butyric acid, (S)-3-amino-4-(3-trifluoromethylphenyl)-butyric acid, (S)-3-amino-4-(4-bromophenyl)-butyric acid, (S)-3-amino-4-(4-chlorophenyl) butyric acid, (S)-3-amino-4-(4-cyanophenyl)-butyric acid, (S)-3-amino-4-(4-fluorophenyl) butyric acid, (S)-3-amino-4-(4-iodophenyl)-butyric acid, (S)-3-amino-4-(4-methylphenyl)-butyric acid, (S)-3-amino-4-(4-nitrophenyl)-butyric acid, (S)-3-amino-4-(4-pyridyl)-butyric acid, (S)-3-amino-4-(4-trifluoromethylphenyl)-butyric acid, (S)-3-amino-4-pentafluoro-phenylbutyric acid, (S)-3-amino-5-hexenoic acid, (S)-3-amino-5-hexynoic acid, (S)-3-amino-5-phenylpentanoic acid, (S)-3-amino-6-phenyl-5-hexenoic acid, 1,2,5,6-tetrahydropyridine-3-carboxylic acid, 1,2,5,6-tetrahydropyridine-4-carboxylic acid, 3-amino-3-(2-chlorophenyl)-propionic acid, 3-amino-3-(2-thienyl)-propionic acid, 3-amino-3-(3-bromophenyl)-propionic acid, 3-amino-3-(4-chlorophenyl)-propionic acid, 3-amino-3-(4-methoxyphenyl)-propionic acid, 3-amino-4,4,4-trifluoro-butyric acid, 3-aminoadipic acid, D-β-phenylalanine, β-leucine, L-β-homoalanine, L-β-homoaspartic acid γ-benzyl ester, L-β-homoglutamic acid δ-benzyl ester, L-β-homoisoleucine, L-β-homoleucine, L-β-homomethionine, L-β-homophenylalanine, L-β-homoproline, L-β-homotryptophan, L-β-homovaline, L-Nω-benzyloxycarbonyl-β-homolysine, Nω-L-β-homoarginine, O-benzyl-L-β-homohydroxyproline, O-benzyl-L-β-homoserine, O-benzyl-L-β-homothreonine, O-benzyl-L-β-homotyrosine, γ-trityl-L-β-homoasparagine, (R)-β-phenylalanine, L-β-homoaspartic acid γ-t-butyl ester, L-β-homoglutamic acid δ-t-butyl ester, L-Nω-β-homolysine, Nδ-trityl-L-β-homoglutamine, Nω-2,2,4,6,7-pentamethyl-dihydrobenzofuran-5-sulfonyl-L-β-homoarginine, O-t-butyl-L-β-homohydroxy-proline, O-t-butyl-L-β-homoserine, O-t-butyl-L-β-homothreonine, O-t-butyl-L-β-homotyrosine, 2-aminocyclopentane carboxylic acid, and 2-aminocyclohexane carboxylic acid.

In some instances, unnatural amino acid residues comprise a racemic mixture of amino acid analogs. For example, in some instances, the D isomer of the amino acid analog is used. In some cases, the L isomer of the amino acid analog is used. In some instances, the amino acid analog comprises chiral centers that are in the R or S configuration. Sometimes, the amino group(s) of a β-amino acid analog is substituted with a protecting group, e.g., tert-butyloxycarbonyl (BOC group), 9-fluorenylmethyloxycarbonyl (FMOC), tosyl, and the like. Sometimes, the carboxylic acid functional group of a β-amino acid analog is protected, e.g., as its ester derivative. In some cases, the salt of the amino acid analog is used.

In some cases, unnatural amino acid residues comprise analogs of amino acid residue alanine, valine, glycine, leucine, arginine, lysine, aspartic acid, glutamic acid, cysteine, methionine, tyrosine, phenylalanine, tryptophane, serine, threonine, or proline. Exemplary amino acid analogs of alanine, valine, glycine, and leucine include, but are not limited to, α-methoxyglycine, α-allyl-L-alanine, α-aminoisobutyric acid, α-methyl-leucine, β-(1-naphthyl)-D-alanine, β-(1-naphthyl)-L-alanine, β-(2-naphthyl)-D-alanine, β-(2-naphthyl)-L-alanine, β-(2-pyridyl)-D-alanine, β-(2-pyridyl)-L-alanine, β-(2-thienyl)-D-alanine, β-(2-thienyl)-L-alanine, β-(3-benzothienyl)-D-alanine, β-(3-benzothienyl)-L-alanine, β-(3-pyridyl)-D-alanine, β-(3-pyridyl)-L-alanine, β-(4-pyridyl)-D-alanine, β-(4-pyridyl)-L-alanine, β-chloro-L-alanine, β-cyano-L-alanin, β-cyclohexyl-D-alanine, β-cyclohexyl-L-alanine, β-cyclopenten-1-yl-alanine, β-cyclopentyl-alanine, β-cyclopropyl-L-Ala-OH.dicyclohexylammonium salt, β-t-butyl-D-alanine, β-t-butyl-L-alanine, γ-aminobutyric acid, L-α,β-diaminopropionic acid, 2,4-dinitro-phenylglycine, 2,5-dihydro-D-phenylglycine, 2-amino-4,4,4-trifluorobutyric acid, 2-fluoro-phenylglycine, 3-amino-4,4,4-trifluoro-butyric acid, 3-fluoro-valine, 4,4,4-trifluoro-valine, 4,5-dehydro-L-leu-OH.dicyclohexylammonium salt, 4-fluoro-D-phenylglycine, 4-fluoro-L-phenylglycine, 4-hydroxy-D-phenylglycine, 5,5,5-trifluoro-leucine, 6-aminohexanoic acid, cyclopentyl-D-Gly-OH.dicyclohexylammonium salt, cyclopentyl-Gly-OH.dicyclohexylammonium salt, D-α,β-diaminopropionic acid, D-α-aminobutyric acid, D-α-t-butylglycine, D-(2-thienyl)glycine, D-(3-thienyl)glycine, D-2-aminocaproic acid, D-2-indanylglycine, D-allylglycine-dicyclohexylammonium salt, D-cyclohexylglycine, D-norvaline, D-phenylglycine, β-aminobutyric acid, β-aminoisobutyric acid, (2-bromophenyl)glycine, (2-methoxyphenyl)glycine, (2-methylphenyl)glycine, (2-thiazoyl)glycine, (2-thienyl)glycine, 2-amino-3-(dimethylamino)-propionic acid, L-α,β-diaminopropionic acid, L-α-aminobutyric acid, L-α-t-butylglycine, L-(3-thienyl)glycine, L-2-amino-3-(dimethylamino)-propionic acid, L-2-aminocaproic acid dicyclohexyl-ammonium salt, L-2-indanylglycine, L-allylglycine.dicyclohexyl ammonium salt, L-cyclohexylglycine, L-phenylglycine, L-propargylglycine, L-norvaline, N-α-aminomethyl-L-alanine, D-α,γ-diaminobutyric acid, L-α,γ-diaminobutyric acid, β-cyclopropyl-L-alanine, (N-β-(2,4-dinitrophenyl))-L-α,β-diaminopropionic acid, (N-β-1-(4,4-dimethyl-2,6-dioxocyclohex-1-ylidene)ethyl)-D-α,β-diaminopropionic acid, (N-β-1-(4,4-dimethyl-2,6-dioxocyclohex-1-ylidene)ethyl)-L-α,β-diaminopropionic acid, (N-β-4-methyltrityl)-L-α,β-diaminopropionic acid, (N-β-allyloxycarbonyl)-L-α,β-diaminopropionic acid, (N-γ-1-(4,4-dimethyl-2,6-dioxocyclohex-1-ylidene)ethyl)-D-α,γ-diaminobutyric acid, (N-γ-1-(4,4-dimethyl-2,6-dioxocyclohex-1-ylidene)ethyl)-L-α,γ-diaminobutyric acid, (N-γ-4-methyltrityl)-D-α,γ-diaminobutyric acid, (N-γ-4-methyltrityl)-L-α,γ-diaminobutyric acid, (N-γ-allyloxycarbonyl)-L-α,γ-diaminobutyric acid, D-α,γ-diaminobutyric acid, 4,5-dehydro-L-leucine, cyclopentyl-D-Gly-OH, cyclopentyl-Gly-OH, D-allylglycine, D-homocyclohexylalanine, L-1-pyrenylalanine, L-2-aminocaproic acid, L-allylglycine, L-homocyclohexylalanine, and N-(2-hydroxy-4-methoxy-Bzl)-Gly-OH.

Exemplary amino acid analogs of arginine and lysine include, but are not limited to, citrulline, L-2-amino-3-guanidinopropionic acid, L-2-amino-3-ureidopropionic acid, L-citrulline, Lys(Me)₂-OH, Lys(N₃)—OH, Nδ-benzyloxycarbonyl-L-omithine, Nω-nitro-D-arginine, Nω-nitro-L-arginine, α-methyl-omithine, 2,6-diaminoheptanedioic acid, L-omithine, (Nδ-1-(4,4-dimethyl-2,6-dioxo-cyclohex-1-ylidene)ethyl)-D-omithine, (Nδ-1-(4,4-dimethyl-2,6-dioxo-cyclohex-1-ylidene)ethyl)-L-omithine, (Nδ-4-methyltrityl)-D-omithine, (Nδ-4-methyltrityl)-L-omithine, D-omithine, L-omithine, Arg(Me)(Pbf)-OH, Arg(Me)₂-OH (asymmetrical), Arg(Me)2-OH (symmetrical), Lys(ivDde)-OH, Lys(Me)2-OH.HCl, Lys(Me3)-OH chloride, Nω-nitro-D-arginine, and Nω-nitro-L-arginine.

Exemplary amino acid analogs of aspartic and glutamic acids include, but are not limited to, α-methyl-D-aspartic acid, α-methyl-glutamic acid, α-methyl-L-aspartic acid, γ-methylene-glutamic acid, (N-γ-ethyl)-L-glutamine, [N-α-(4-aminobenzoyl)]-L-glutamic acid, 2,6-diaminopimelic acid, L-α-aminosuberic acid, D-2-aminoadipic acid, D-α-aminosuberic acid, α-aminopimelic acid, iminodiacetic acid, L-2-aminoadipic acid, threo-β-methyl-aspartic acid, γ-carboxy-D-glutamic acid γ,γ-di-t-butyl ester, γ-carboxy-L-glutamic acid γ,γ-di-t-butyl ester, Glu(OAll)-OH, L-Asu(OtBu)-OH, and pyroglutamic acid.

Exemplary amino acid analogs of cysteine and methionine include, but are not limited to, Cys(farnesyl)-OH, Cys(farnesyl)-OMe, α-methyl-methionine, Cys(2-hydroxyethyl)-OH, Cys(3-aminopropyl)-OH, 2-amino-4-(ethylthio)butyric acid, buthionine, buthioninesulfoximine, ethionine, methionine methylsulfonium chloride, selenomethionine, cysteic acid, [2-(4-pyridyl)ethyl]-DL-penicillamine, [2-(4-pyridyl)ethyl]-L-cysteine, 4-methoxybenzyl-D-penicillamine, 4-methoxybenzyl-L-penicillamine, 4-methylbenzyl-D-penicillamine, 4-methylbenzyl-L-penicillamine, benzyl-D-cysteine, benzyl-L-cysteine, benzyl-DL-homocysteine, carbamoyl-L-cysteine, carboxyethyl-L-cysteine, carboxymethyl-L-cysteine, diphenylmethyl-L-cysteine, ethyl-L-cysteine, methyl-L-cysteine, t-butyl-D-cysteine, trityl-L-homocysteine, trityl-D-penicillamine, cystathionine, homocystine, L-homocystine, (2-aminoethyl)-L-cysteine, seleno-L-cystine, cystathionine, Cys(StBu)-OH, and acetamidomethyl-D-penicillamine.

Exemplary amino acid analogs of phenylalanine and tyrosine include, but are not limited to, β-methyl-phenylalanine, β-hydroxyphenylalanine, α-methyl-3-methoxy-DL-phenylalanine, α-methyl-D-phenylalanine, α-methyl-L-phenylalanine, 1,2,3,4-tetrahydroisoquinoline-3-carboxylic acid, 2,4-dichloro-phenylalanine, 2-(trifluoromethyl)-D-phenylalanine, 2-(trifluoromethyl)-L-phenylalanine, 2-bromo-D-phenylalanine, 2-bromo-L-phenylalanine, 2-chloro-D-phenylalanine, 2-chloro-L-phenylalanine, 2-cyano-D-phenylalanine, 2-cyano-L-phenylalanine, 2-fluoro-D-phenylalanine, 2-fluoro-L-phenylalanine, 2-methyl-D-phenylalanine, 2-methyl-L-phenylalanine, 2-nitro-D-phenylalanine, 2-nitro-L-phenylalanine, 2,4,5-trihydroxy-phenylalanine, 3,4,5-trifluoro-D-phenylalanine, 3,4,5-trifluoro-L-phenylalanine, 3,4-dichloro-D-phenylalanine, 3,4-dichloro-L-phenylalanine, 3,4-difluoro-D-phenylalanine, 3,4-difluoro-L-phenylalanine, 3,4-dihydroxy-L-phenylalanine, 3,4-dimethoxy-L-phenylalanine, 3,5,3′-triiodo-L-thyronine, 3,5-diiodo-D-tyrosine, 3,5-diiodo-L-tyrosine, 3,5-diiodo-L-thyronine, 3-(trifluoromethyl)-D-phenylalanine, 3-(trifluoromethyl)-L-phenylalanine, 3-amino-L-tyrosine, 3-bromo-D-phenylalanine, 3-bromo-L-phenylalanine, 3-chloro-D-phenylalanine, 3-chloro-L-phenylalanine, 3-chloro-L-tyrosine, 3-cyano-D-phenylalanine, 3-cyano-L-phenylalanine, 3-fluoro-D-phenylalanine, 3-fluoro-L-phenylalanine, 3-fluoro-tyrosine, 3-iodo-D-phenylalanine, 3-iodo-L-phenylalanine, 3-iodo-L-tyrosine, 3-methoxy-L-tyrosine, 3-methyl-D-phenylalanine, 3-methyl-L-phenylalanine, 3-nitro-D-phenylalanine, 3-nitro-L-phenylalanine, 3-nitro-L-tyrosine, 4-(trifluoromethyl)-D-phenylalanine, 4-(trifluoromethyl)-L-phenylalanine, 4-amino-D-phenylalanine, 4-amino-L-phenylalanine, 4-benzoyl-D-phenylalanine, 4-benzoyl-L-phenylalanine, 4-bis(2-chloroethyl)amino-L-phenylalanine, 4-bromo-D-phenylalanine, 4-bromo-L-phenylalanine, 4-chloro-D-phenylalanine, 4-chloro-L-phenylalanine, 4-cyano-D-phenylalanine, 4-cyano-L-phenylalanine, 4-fluoro-D-phenylalanine, 4-fluoro-L-phenylalanine, 4-iodo-D-phenylalanine, 4-iodo-L-phenylalanine, homophenylalanine, thyroxine, 3,3-diphenylalanine, thyronine, ethyl-tyrosine, and methyl-tyrosine.

Exemplary amino acid analogs of proline include 3,4-dehydro-proline, 4-fluoro-proline, cis-4-hydroxy-proline, thiazolidine-2-carboxylic acid, and trans-4-fluoro-proline.

Exemplary amino acid analogs of serine and threonine include 3-amino-2-hydroxy-5-methylhexanoic acid, 2-amino-3-hydroxy-4-methylpentanoic acid, 2-amino-3-ethoxybutanoic acid, 2-amino-3-methoxybutanoic acid, 4-amino-3-hydroxy-6-methylheptanoic acid, 2-amino-3-benzyloxypropionic acid, 2-amino-3-benzyloxypropionic acid, 2-amino-3-ethoxypropionic acid, 4-amino-3-hydroxybutanoic acid, and α-methylserine.

Exemplary amino acid analogs of tryptophan include, but are not limited to, α-methyl-tryptophan, β-(3-benzothienyl)-D-alanine, β-(3-benzothienyl)-L-alanine, 1-methyl-tryptophan, 4-methyl-tryptophan, 5-benzyloxy-tryptophan, 5-bromo-tryptophan, 5-chloro-tryptophan, 5-fluoro-tryptophan, 5-hydroxy-tryptophan, 5-hydroxy-L-tryptophan, 5-methoxy-tryptophan, 5-methoxy-L-tryptophan, 5-methyl-tryptophan, 6-bromo-tryptophan, 6-chloro-D-tryptophan, 6-chloro-tryptophan, 6-fluoro-tryptophan, 6-methyl-tryptophan, 7-benzyloxy-tryptophan, 7-bromo-tryptophan, 7-methyl-tryptophan, D-1,2,3,4-tetrahydro-norharman-3-carboxylic acid, 6-methoxy-1,2,3,4-tetrahydronorharman-1-carboxylic acid, 7-azatryptophan, L-1,2,3,4-tetrahydro-norharman-3-carboxylic acid, 5-methoxy-2-methyl-tryptophan, and 6-chloro-L-tryptophan.

In some instances, the modification comprises a mutation to a hydrophobic or nonpolar amino acid residue. In some cases, a hydrophobic or nonpolar amino acid includes small hydrophobic amino acids and large hydrophobic amino acids. Exemplary small hydrophobic amino acids include glycine, alanine, proline, and analogs thereof. Exemplary large hydrophobic amino acids include valine, leucine, isoleucine, phenylalanine, methionine, tryptophan, and analogs thereof. In some instances, a non-natural neurotrophic agent described herein comprises a mutation to a small hydrophobic amino acid (e.g., a mutation to glycine, alanine, proline, and analogs thereof). In some instances, a non-natural neurotrophic agent described herein comprises a mutation to a large hydrophobic amino acid (e.g., a mutation to valine, leucine, isoleucine, phenylalanine, methionine, tryptophan, and analogs thereof).

In some instances, the modification comprises a mutation to a polar amino acid residue. In some cases, the polar amino acid comprises serine, threonine, asparagine, glutamine, cysteine, tyrosine, and analogs thereof. In some instances, a non-natural neurotrophic agent described herein comprises a mutation to a polar amino acid residue (e.g., a mutation to serine, threonine, asparagine, glutamine, cysteine, tyrosine, and analogs thereof).

In additional instances, the modification comprises a mutation to a charged amino acid residue. In some cases, charged amino acids include lysine, arginine, histidine, aspartate, glutamate, or analog thereof. In some instances, a non-natural neurotrophic agent described herein comprises a mutation to a charged amino acid residue (e.g., a mutation to lysine, arginine, histidine, aspartate, glutamate, or analog thereof).

In some embodiments, a non-natural neurotrophic agent described herein comprise a modification to a non-essential amino acid. In some instances, a non-essential amino acid residue is a residue that is altered from the wild-type sequence of a polypeptide without abolishing or substantially altering its essential biological or biochemical activity (e.g., receptor binding or activation). In some cases, a non-natural neurotrophic agent provided herein comprises an essential amino acid. In some cases, an essential amino acid residue is a residue that, when altered from the wild-type sequence of the polypeptide, results in abolishing or substantially abolishing the polypeptide's essential biological or biochemical activity.

In some embodiments, a non-natural neurotrophic agent described herein comprises a conservative amino acid substitution. In some cases, a conservative amino acid substitution is one in which the amino acid residue is replaced with an amino acid residue having a similar side chain. Families of amino acid residues having similar side chains have been defined in the art. These families include, for example, amino acids with basic side chains (e.g., K, R, H), acidic side chains (e.g., D, E), uncharged polar side chains (e.g., G, N, Q, S, T, Y, C), nonpolar side chains (e.g., A, V, L, I, P, F, M, W), beta-branched side chains (e.g., T, V, I) and aromatic side chains (e.g., Y, F, W, H). Thus, a predicted nonessential amino acid residue in a polypeptide, for example, is replaced with another amino acid residue from the same side chain family. Other examples of acceptable substitutions include substitutions based on isosteric considerations (e.g., norleucine for methionine) or other properties (e.g., 2-thienylalanine for phenylalanine or 6-Cl-tryptophan for tryptophan).

In some embodiments, a non-natural neurotrophic agent described herein comprises a semi-conservative amino acid substitution. In some embodiments, the semi-conservative amino acid substitution involves substitution between amino acids within a same side chain family or amino acids in which the side chains share similar steric confirmations.

In some embodiments, a non-natural neurotrophic agent described herein comprises a non-conservative amino acid substitution. In some embodiments, the non-conservative amino acid substitution involves substitution between amino acids within a different side chain family.

In some embodiments, a non-natural neurotrophic agent described herein comprise a modification that is introduced by mutagenesis. In some embodiments, the mutagenesis is random. In some embodiments, mutagenesis is non-random. An example of non-random mutagenesis is site-directed mutagenesis. In some embodiments, the site-directed mutagenesis utilizes cassette mutagenesis, PCR-site-directed mutagenesis, whole plasmid mutagenesis, Kunkel's method, or in vivo site-directed mutagenesis method. In some instances, the mutagenesis is alanine screening mutagenesis.

In some embodiments, the non-natural neurotrophic agents described herein are chemically modified analogs of naturally occurring neurotrophic agents. Exemplary chemical modifications include, but are not limited to, phosphorylation or sulfurylation at serine, threonine, or tyrosine residues; by incorporating unnatural amino acids; by incorporating heavy amino acids; by incorporating D-amino acids; by biotinylation; by cyclisations; by acylation; by dimethylation; by amidation; by derivatization; by conjugation to carrier proteins; or by branching of peptide.

In some embodiments, administration of the otic composition comprising a non-natural neurotrophic agent as described herein ameliorates hearing loss or reduction resulting from destroyed, stunted, malfunctioning, damaged, fragile, or missing hair cells in the inner ear. In some embodiments, administration of the otic composition comprising a non-natural neurotrophic agent as described herein ameliorates hearing loss or reduction resulting from destroyed, stunted, malfunctioning, damaged, fragile or missing hair cells in the inner ear, wherein the non-natural neurotrophic agent is chemically modified. In some embodiments, the hearing loss is sensorineural hearing loss.

In some embodiments, administration of the otic composition comprising a non-natural neurotrophic agent as described herein protects against neuronal degeneration. In some embodiments, administration of the otic composition comprising a non-natural neurotrophic agent promotes neurite outgrowth. In some embodiments, administration of the otic composition comprising a non-natural neurotrophic agent as described herein improves function of a neural synapse. For example, the non-natural neurotrophic agent improves the neural synapse between hair cells and spiral ganglion neurons.

### Methods of Neurotropic Agent Production

In some embodiments, one or more of the non-natural neurotrophic agents described herein are produced, for example, in a host cell system or a cell-free system. In some embodiments, one or more of the neurotropic agents described herein are produced recombinantly through a host cell system. In some instances, the host cell is a eukaryotic cell (e.g., mammalian cell, insect cells, yeast cells, or plant cell) or a prokaryotic cell (e.g., gram-positive bacterium or a gram-negative bacterium).

In some embodiments, a eukaryotic host cell is a mammalian host cell. In some cases, a mammalian host cell is a stable cell line, or a cell line that has incorporated a genetic material of interest into its own genome and has the capability to express the product of the genetic material after many generations of cell division. In other cases, a mammalian host cell is a transient cell line, or a cell line that has not incorporated a genetic material of interest into its own genome and does not have the capability to express the product of the genetic material after many generations of cell division.

Exemplary mammalian host cells include 293T cell line, 293A cell line, 293FT cell line, 293F cells, 293 H cells, A549 cells, MDCK cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some embodiments, a eukaryotic host cell is an insect host cell. Exemplary insect host cell include Drosophila S2 cells, Sf9 cells, Sf21 cells, High Five™ cells, and expresSF+® cells.

In some embodiments, a eukaryotic host cell is a yeast host cell. Exemplary yeast host cells include Pichia pastoris yeast strains such as GS115, KM71H, SMD1168, SMD1168H, and X-33, and Saccharomyces cerevisiae yeast strain such as INVSc1.

In some embodiments, a eukaryotic host cell is a plant host cell. In some instances, the plant cells comprise a cell from algae. Exemplary plant cell lines include strains from Chlamydomonas reinhardtii 137c, or Synechococcus elongatus PPC 7942.

In some embodiments, a host cell is a prokaryotic host cell. Exemplary prokaryotic host cells include BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™.

In some instances, suitable polynucleic acid molecules or vectors for the production of a non-natural neurotrophic agent described herein include any suitable vectors derived from either a eukaryotic or prokaryotic sources. Exemplary polynucleic acid molecules or vectors include vectors from bacteria (e.g., E. coli), insects, yeast (e.g., Pichia pastoris), algae, or mammalian source. Bacterial vectors include, for example, pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Insect vectors include, for example, pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

Yeast vectors include, for example, Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichia pastoris vector, pGAPZA, B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Algae vectors include, for example, pChlamy-4 vector or MCS vector.

Mammalian vectors include, for example, transient expression vectors or stable expression vectors. Exemplary mammalian transient expression vectors include p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Exemplary mammalian stable expression vectors include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is used for the production of a non-natural neurotrophic agent described herein. In some cases, a cell-free system comprises a mixture of cytoplasmic and/or nuclear components from a cell and is suitable for in vitro nucleic acid synthesis. In some instances, a cell-free system utilizes prokaryotic cell components. In other instances, a cell-free system utilizes eukaryotic cell components. Nucleic acid synthesis is obtained in a cell-free system based on, for example, Drosophila cell, Xenopus egg, or HeLa cells. Exemplary cell-free systems include E. coli S30 Extract system, E. coli T7 S30 system, or PURExpress®.

In some embodiments, one or more non-natural neurotrophic agents described herein are chemically synthesized. Exemplary synthesis techniques include, for example, solid phase technique developed by R. B. Merrified which permits the peptide to be built residue by residue from the carboxyl terminal amino acid to the amino terminal amino acid either manually or with an automated, commercially available synthesizer, and techniques described in Stewart, J. M. et al., Solid Phase Peptide Synthesis (Pierce Chemical Co., 2d ed., 1984), and Bodanszky, M. et al., The Practice of Peptide Synthesis (Springer-Verlag, 1984).

### Combination Therapy

In some embodiments, otic composition or device described herein comprising non-natural Trk receptor agonists, further comprises one or more active agents and/or a second therapeutic agent including but not limited to anti-emetic agents, antimicrobial agents, antioxidants, anti-septic agents or the like.

### Otic Surgery and Implants

In some embodiments, the otic formulations, compositions or devices described herein are used in combination with (e.g., implantation, short-term use, long-term use, or removal of) implants (e.g., cochlear implants). As used herein, implants include auris-interna or auris-media medical devices, examples of which include cochlear implants, hearing sparing devices, hearing-improvement devices, short electrodes, micro-prostheses or piston-like prostheses, needles, stem cell transplants, drug delivery devices, any cell-based therapeutic, or the like. In some instances, the implants are used in conjunction with a patient experiencing hearing loss. In some instances, the hearing loss is present at birth. In some instances, the hearing loss is associated with conditions such as AIED, bacterial meningitis, or the like that lead to osteoneogenesis and/or nerve damage with rapid obliteration of cochlear structures and profound hearing loss.

In some instances, an implant is an immune cell or a stem cell transplant in the ear. In some instances, an implant is a small electronic device that has an external portion placed behind the ear, and a second portion that is surgically placed under the skin that helps provide a sense of hearing to a person who is profoundly deaf or severely hard-of-hearing. By way of example, such cochlear medical device implants bypass damaged portions of the ear and directly stimulate the auditory nerve. In some instances, cochlear implants are used in single-sided deafness. In some instances, cochlear implants are used for deafness in both ears.

In some embodiments, administration of a non-natural Trk receptor agonist composition or device described herein in combination with an otic intervention (e.g., an intratympanic injection, a stapedectomy, a medical device implant, or a cell-based transplant) delays or prevents collateral damage to auris structures, e.g., irritation, cell damage, cell death, osteoneogeneis, and/or further neuronal degeneration, caused by the external otic intervention (e.g., installation of an external device and/or cells in the ear). In some embodiments, administration of a non-natural Trk receptor agonist composition or device described herein in combination with an implant allows for a more effective restoration of hearing loss compared to an implant alone.

In some embodiments, administration of a non-natural Trk receptor agonist composition or device described herein reduces damage to cochlear structures caused by underlying conditions (e.g., bacterial meningitis, autoimmune ear disease (AIED)) allowing for successful cochlear device implantation. In some embodiments, administration of a non-natural Trk receptor agonist composition or device described herein, in conjunction with otic surgery, medical device implantation, and/or cell transplantation, reduces or prevents cell damage and/or death (e.g., auris sensory hair cell death and/or damage) associated with otic surgery, medical device implantation, and/or cell transplantation.

In some embodiments, administration of a non-natural Trk receptor agonist composition or device described herein (e.g., a composition or device comprising a growth factor) in conjunction with a cochlear implant or stem cell transplant has a trophic effect (e.g., promotes healthy growth of cells and/or healing of tissue in the area of an implant or transplant). In some embodiments, administration of a non-natural Trk receptor agonist composition or device described herein promotes at least one of differentiation, proliferation, and growth of stem cells such as hair stem cells and neuronal stem cells. In some instances, the stem cells are treated with the non-natural Trk receptor agonist ex vivo. In some embodiments, the stem cells are treated with the non-natural Trk receptor agonist ex vivo before implantation of the cochlear implant.

In some embodiments, a trophic effect is desirable during otic surgery or during intratympanic injection procedures. In some embodiments, atrophic effect is desirable after installation of a medical device or after a cell transplant. In some of such embodiments, the non-natural Trk receptor agonist compositions or devices described herein are administered via direct cochlear injection, through a chochleostomy, or via deposition on the round window.

In some embodiments, administration of an anti-inflammatory or immunosuppressant composition (e.g., a composition comprising an immunosuppressant such as a corticosteroid) reduces inflammation and/or infections associated with otic surgery, implantation of a medical device, or a cell transplant. In some instances, perfusion of a surgical area with an auris sensory cell modulator formulation described herein reduces or eliminates post-surgical and/or post-implantation complications (e.g., inflammation, hair cell damage, neuronal degeneration, osteoneogenesis, or the like). In some instances, perfusion of a surgical area with a formulation described herein reduces post-surgery or post-implantation recuperation time. In some embodiments, a medical device is coated with a composition described herein prior to implantation in the ear.

In one aspect, the formulations described herein, and modes of administration thereof, are applicable to methods of direct perfusion of the inner ear compartments. Thus, the formulations described herein are useful in combination with otic interventions. In some embodiments, an otic intervention is an implantation procedure (e.g., implantation of a hearing device in the cochlea). In some embodiments, an otic intervention is a surgical procedure including, by way of non-limiting examples, cochleostomy, labyrinthotomy, mastoidectomy, stapedectomy, stapedotomy, endolymphatic sacculotomy, tympanostomy, or the like. In some embodiments, the inner ear compartments are perfused with a formulation described herein prior to otic intervention, during otic intervention, after otic intervention, or any combination thereof.

In some embodiments, when perfusion is carried out in combination with otic intervention, the non-natural Trk receptor agonist compositions are immediate release compositions. In some of such embodiments, the immediate release formulations described herein are non-thickened compositions and are substantially free of extended release components (e.g., gelling components such as polyoxyethylene-polyoxypropylene copolymers). In some of such embodiments, the compositions contain less than 5% of the extended release components (e.g., gelling components such as polyoxyethylene-polyoxypropylene triblock copolymers) by weight of the formulation. In some of such embodiments, the compositions contain less than 2% of the extended release components (e.g., gelling components such as polyoxyethylene-polyoxypropylene triblock copolymers) by weight of the formulation. In some of such embodiments, the compositions contain less than 1% of the extended release components (e.g., gelling components such as polyoxyethylene-polyoxypropylene triblock copolymers) by weight of the formulation. In some of such embodiments, a composition described herein that is used for perfusion of a surgical area contains substantially no gelling component and is an immediate release composition.

In other embodiments, a non-natural Trk receptor agonist composition described herein is administered after an otic intervention (e.g., after implantation of a medical device or a cell-based therapeutic). In some of such embodiments, a non-natural Trk receptor agonist composition described herein that is administered after the otic intervention is an intermediate release or extended release composition and contains gelling components as described herein.

### General Methods of Sterilization

The environment of the inner ear is an isolated environment. The endolymph and the perilymph are static fluids and are not in contiguous contact with the circulatory system. The blood-labyrinth-barrier (BLB), which includes a blood-endolymph barrier and a blood-perilymph barrier, consists of tight junctions between specialized epithelial cells in the labyrinth spaces (i.e., the vestibular and cochlear spaces). The presence of the BLB limits delivery of active agents (e.g., non-natural Trk receptor agonists) to the isolated microenvironment of the inner ear. Auris hair cells are bathed in endolymphatic or perilymphatic fluids and cochlear recycling of potassium ions is important for hair cell function. When the inner ear is infected, there is an influx of leukocytes and/or immunoglobins (e.g. in response to a microbial infection) into the endolymph and/or the perilymph and the delicate ionic composition of inner ear fluids is upset by the influx of leukocytes and/or immunoglobins. In certain instances, a change in the ionic composition of inner ear fluids results in hearing loss, loss of balance, and/or ossification of auditory structures. In certain instances, even trace amounts of pyrogens and/or microbes can trigger infections and related physiological changes in the isolated microenvironment of the inner ear.

Provided herein are auris formulations comprising non-natural Trk receptor agonists that are manufactured with low bioburden or sterilized with stringent sterility requirements and are suitable for administration to the middle and/or inner ear. In some embodiments, the auris compatible compositions described herein are substantially free of pyrogens and/or microbes.

Provided herein are otic compositions comprising non-natural Trk receptor agonists that ameliorate or lessen otic disorders described herein. Further provided herein are methods comprising the administration of said otic compositions. In some embodiments, the compositions or devices are sterilized. Included within the embodiments disclosed herein are means and processes for sterilization of a pharmaceutical composition or device disclosed herein for use in humans. The goal is to provide a safe pharmaceutical product, relatively free of infection causing micro-organisms. The U.S. Food and Drug Administration has provided regulatory guidance in the publication “Guidance for Industry: Sterile Drug Products Produced by Aseptic Processing” which is incorporated herein by reference in its entirety.

As used herein, sterilization means a process used to destroy or remove microorganisms that are present in a product or packaging. Any suitable method available for sterilization of objects and compositions is used. Available methods for the inactivation of microorganisms include, but are not limited to, the application of extreme heat, lethal chemicals, or gamma radiation. In some embodiments, disclosed herein, is a process for the preparation of an otic therapeutic formulation comprising subjecting the formulation to a sterilization method selected from chemical sterilization, radiation sterilization, or filtration sterilization. The method used depends largely upon the nature of the device or composition to be sterilized. Detailed descriptions of many methods of sterilization are given in Chapter 40 of Remington: The Science and Practice of Pharmacy published by Lippincott, Williams & Wilkins, and is incorporated by reference with respect to this subject matter.

Chemical Sterilization

Chemical sterilization methods are an alternative for products that do not withstand the extremes of heat sterilization. In this method, a variety of gases and vapors with germicidal properties, such as ethylene oxide, chlorine dioxide, formaldehyde, or ozone are used as the anti-apoptotic agents. The germicidal activity of ethylene oxide, for example, arises from its ability to serve as a reactive alkylating agent. Thus, the sterilization process requires the ethylene oxide vapors to make direct contact with the product to be sterilized.

Radiation Sterilization

One advantage of radiation sterilization is the ability to sterilize many types of products without heat degradation or other damage. The radiation commonly employed is beta radiation or, alternatively, gamma radiation from a ⁶⁰Co source. The penetrating ability of gamma radiation allows its use in the sterilization of many product types, including solutions, compositions, and heterogeneous mixtures. The germicidal effects of irradiation arise from the interaction of gamma radiation with biological macromolecules. This interaction generates charged species and free radicals. Subsequent chemical reactions, such as rearrangements and cross-linking processes, result in the loss of normal function for these biological macromolecules. The formulations described herein are also optionally sterilized using beta irradiation. In some embodiments, the formulations described herein, comprising non-natural Trk receptor agonists are in the form of solution, and the solutions are sterilized using radiation sterilization methods.

Filtration

Filtration sterilization is a method used to remove but not destroy microorganisms from solutions. Membrane filters are used to filter heat-sensitive solutions. Such filters are thin, strong, homogenous polymers of mixed cellulosic esters (MCE), polyvinylidene fluoride (PVF; also known as PVDF), or polytetrafluoroethylene (PTFE) and have pore sizes ranging from 0.1 to 0.22 m. Solutions of various characteristics are optionally filtered using different filter membranes. For example, PVF and PTFE membranes are well suited to filtering organic solvents while aqueous solutions are filtered through PVF or MCE membranes. Filter apparatus are available for use on many scales ranging from the single point-of-use disposable filter attached to a syringe up to commercial scale filters for use in manufacturing plants. The membrane filters are sterilized by autoclave or chemical sterilization. Validation of membrane filtration systems is performed following standardized protocols (Microbiological Evaluation of Filters for Sterilizing Liquids, Vol 4, No. 3. Washington, D.C.: Health Industry Manufacturers Association, 1981) and involve challenging the membrane filter with a known quantity (ca. 10^{7/}cm²) of unusually small microorganisms, such as Brevundimonas diminuta (ATCC 19146). In some embodiments, the formulations described herein, comprising non-natural Trk receptor agonists, are in the form of solution, and the solutions are sterilized using filtration methods.

Pharmaceutical compositions are optionally sterilized by passing through membrane filters. Formulations comprising nanoparticles (U.S. Pat. No. 6,139,870) or multilamellar vesicles (Richard et al., International Journal of Pharmaceutics (2006), 312(1-2): 144-50) are amenable to sterilization by filtration through 0.22 μm filters without destroying their organized structure. In some embodiments, the formulations described herein, comprising non-natural Trk receptor agonists, are in the form of multilamellar vesicles, and the multilamellar vesicles are sterilized using filtration methods.

In some embodiments, the methods disclosed herein comprise sterilizing the formulation (or components thereof) by means of filtration sterilization. In another embodiment the auris-acceptable otic therapeutic agent formulation comprises a particle wherein the particle formulation is suitable for filtration sterilization. In a further embodiment said particle formulation comprises particles of less than 300 nm in size, of less than 200 nm in size, or of less than 100 nm in size. In another embodiment the auris-acceptable formulation comprises a particle formulation wherein the sterility of the particle is ensured by sterile filtration of the precursor component solutions. In another embodiment the auris-acceptable formulation comprises a particle formulation wherein the sterility of the particle formulation is ensured by low temperature sterile filtration. In a further embodiment, low temperature sterile filtration is carried out at a temperature from 0 to 30° C., from 0 to 20° C., from 0 to 10° C., from 10 to 20° C., or from 20 to 30° C.

In another embodiment is a process for the preparation of an auris-acceptable particle formulation comprising: filtering the aqueous solution containing the particle formulation at low temperature through a sterilization filter; lyophilizing the sterile solution; and reconstituting the particle formulation with sterile water prior to administration. In some embodiments, a formulation described herein is manufactured as a suspension in a single vial formulation containing the micronized active pharmaceutical ingredient. A single vial formulation is prepared by aseptically mixing a sterile poloxamer solution with sterile micronized active ingredient (e.g., ketamine) and transferring the formulation to sterile pharmaceutical containers. In some embodiments, a single vial containing a formulation described herein as a suspension is resuspended before dispensing and/or administration.

In specific embodiments, filtration and/or filling procedures are carried out at about 5° C. below the gel temperature (Tgel) of a formulation described herein and with viscosity below a theoretical value of 100 cP to allow for filtration in a reasonable time using a peristaltic pump.

In another embodiment the auris-acceptable otic therapeutic agent formulation comprises a nanoparticle formulation wherein the nanoparticle formulation is suitable for filtration sterilization. In a further embodiment the nanoparticle formulation comprises nanoparticles of less than 300 nm in size, of less than 200 nm in size, or of less than 100 nm in size. In another embodiment the auris-acceptable formulation comprises a microsphere formulation wherein the sterility of the microsphere is ensured by sterile filtration of the precursor organic solution and aqueous solutions. In another embodiment the auris-acceptable formulation comprises a thermoreversible gel formulation wherein the sterility of the gel formulation is ensured by low temperature sterile filtration. In a further embodiment, the low temperature sterile filtration occurs at a temperature from 0 to 30° C., or from 0 to 20° C., or from 0 to 10° C., or from 10 to 20° C., or from 20 to 30° C. In another embodiment is a process for the preparation of an auris-acceptable thermoreversible gel formulation comprising: filtering the aqueous solution containing the thermoreversible gel components at low temperature through a sterilization filter; lyophilizing the sterile solution; and reconstituting the thermoreversible gel formulation with sterile water prior to administration.

In certain embodiments, the active ingredients are dissolved in a suitable vehicle (e.g. a buffer) and sterilized separately (e.g. by heat treatment, filtration, or gamma radiation). In some instances, the active ingredients are sterilized separately in a dry state. In some instances, the active ingredients are sterilized as a suspension or as a colloidal suspension. The remaining excipients (e.g., fluid gel components present in auris formulations) are sterilized in a separate step by a suitable method (e.g. filtration and/or irradiation of a cooled mixture of excipients); the two solutions that are separately sterilized are then mixed aseptically to provide a final auris formulation. In some instances, the final aseptic mixing is performed just prior to administration of a formulation described herein.

In some instances, conventionally used methods of sterilization (e.g., heat treatment (e.g., in an autoclave), gamma irradiation, filtration) lead to irreversible degradation of polymeric components (e.g., thermosetting, gelling or mucoadhesive polymer components) and/or the active agent in the formulation. In some instances, sterilization of an auris formulation by filtration through membranes (e.g., 0.2 μM membranes) is not possible if the formulation comprises thixotropic polymers that gel during the process of filtration.

Accordingly, provided herein are methods for sterilization of auris formulations that prevent degradation of polymeric components (e.g., thermosetting and/or gelling and/or mucoadhesive polymer components) and/or the non-natural Trk receptor agonist during the process of sterilization. In some embodiments, degradation of the non-natural Trk receptor agonist (e.g., a non-natural neurotrophic agent described herein) is reduced or eliminated through the use of specific pH ranges for buffer components and specific proportions of gelling agents in the formulations. In some embodiments, the choice of an appropriate gelling agent and/or thermosetting polymer allows for sterilization of formulations described herein by filtration. In some embodiments, the use of an appropriate thermosetting polymer and an appropriate copolymer (e.g., a gelling agent) in combination with a specific pH range for the formulation allows for high temperature sterilization of formulations described with substantially no degradation of the therapeutic agent or the polymeric excipients. An advantage of the methods of sterilization provided herein is that, in certain instances, the formulations are subjected to terminal sterilization via autoclaving without any loss of the active agent and/or excipients and/or polymeric components during the sterilization step and are rendered substantially free of microbes and/or pyrogens.

Microorganisms

Provided herein are auris-acceptable compositions or devices that ameliorate or lessen otic disorders described herein. Further provided herein are methods comprising the administration of said otic compositions. In some embodiments, the compositions or devices are substantially free of microorganisms. Acceptable bioburden or sterility levels are based on applicable standards that define therapeutically acceptable compositions, including but not limited to United States Pharmacopeia Chapters <1111> et seq. For example, acceptable sterility (e.g., bioburden) levels include about 10 colony forming units (cfu) per gram of formulation, about 50 cfu per gram of formulation, about 100 cfu per gram of formulation, about 500 cfu per gram of formulation or about 1000 cfu per gram of formulation. In some embodiments, acceptable bioburden levels or sterility for formulations include less than 10 cfu/mL, less than 50 cfu/mL, less than 500 cfu/mL or less than 1000 cfu/mL microbial agents. In addition, acceptable bioburden levels or sterility include the exclusion of specified objectionable microbiological agents. By way of example, specified objectionable microbiological agents include but are not limited to Escherichia coli (E. coli), Salmonella sp., Pseudomonas aeruginosa (P. aeruginosa), and/or other specific microbial agents.

Sterility of the auris-acceptable otic therapeutic agent formulation is confirmed through a sterility assurance program in accordance with United States Pharmacopeia Chapters <61>, <62> and <71>. A key component of the sterility assurance quality control, quality assurance, and validation process is the method of sterility testing. Sterility testing, by way of example only, is performed by two methods. The first is direct inoculation wherein a sample of the composition to be tested is added to growth medium and incubated for a period of time up to 21 days. Turbidity of the growth medium indicates contamination. Drawbacks to this method include the small sampling size of bulk materials which reduces sensitivity and detection of microorganism growth based on a visual observation. An alternative method is membrane filtration sterility testing. In this method, a volume of product is passed through a small membrane filter paper. The filter paper is then placed into media to promote the growth of microorganisms. This method has the advantage of greater sensitivity as the entire bulk product is sampled. The commercially available Millipore Steritest sterility testing system is optionally used for determinations by membrane filtration sterility testing. For the filtration testing of creams or ointments Steritest filter system No. TLHVSL210 are used. For the filtration testing of emulsions or viscous products Steritest filter system No. TLAREM210 or TDAREM210 are used. For the filtration testing of pre-filled syringes Steritest filter system No. TTHASY210 are used. For the filtration testing of material dispensed as an aerosol or foam Steritest filter system No. TTHVA210 are used. For the filtration testing of soluble powders in ampoules or vials Steritest filter system No. TTHADA210 or TTHADV210 are used.

Testing for E. coli and Salmonella includes the use of lactose broths incubated at 30-35° C. for 24-72 hours, incubation in MacConkey and/or EMB agars for 18-24 hours, and/or the use of Rappaport medium. Testing for the detection of P. aeruginosa includes the use of NAC agar. United States Pharmacopeia Chapter <62> further enumerates testing procedures for specified objectionable microorganisms.

In certain embodiments, any controlled-release formulation described herein has less than about 60 colony forming units (CFU), less than about 50 colony forming units, less than about 40 colony forming units, or less than about 30 colony forming units of microbial agents per gram of formulation. In certain embodiments, the otic formulations described herein are formulated to be isotonic with the endolymph and/or the perilymph.

Endotoxins

Provided herein are otic compositions that ameliorate or lessen otic disorders described herein. Further provided herein are methods comprising the administration of said otic compositions. In some embodiments, the compositions or devices are substantially free of endotoxins. An additional aspect of the sterilization process is the removal of by-products from the killing of microorganisms (hereinafter, “Product”). The process of depyrogenation removes pyrogens from the sample. Pyrogens are endotoxins or exotoxins which induce an immune response. An example of an endotoxin is the lipopolysaccharide (LPS) molecule found in the cell wall of gram-negative bacteria. While sterilization procedures such as autoclaving or treatment with ethylene oxide kill the bacteria, the LPS residue induces a proinflammatory immune response, such as septic shock. Because the molecular size of endotoxins can vary widely, the presence of endotoxins is expressed in “endotoxin units” (EU). One EU is equivalent to 100 picograms of E. coli LPS. Humans can develop a response to as little as 5 EU/kg of body weight. The bioburden (e.g., microbial limit) and/or sterility (e.g., endotoxin level) is expressed in any units as recognized in the art. In certain embodiments, otic compositions described herein contain lower endotoxin levels (e.g. <4 EU/kg of body weight of a subject) when compared to conventionally acceptable endotoxin levels (e.g., 5 EU/kg of body weight of a subject). In some embodiments, the auris-acceptable otic therapeutic agent formulation has less than about 5 EU/kg of body weight of a subject. In other embodiments, the auris-acceptable otic therapeutic agent formulation has less than about 4 EU/kg of body weight of a subject. In additional embodiments, the auris-acceptable otic therapeutic agent formulation has less than about 3 EU/kg of body weight of a subject. In additional embodiments, the auris-acceptable otic therapeutic agent formulation has less than about 2 EU/kg of body weight of a subject.

In some embodiments, the auris-acceptable otic therapeutic agent formulation or device has less than about 5 EU/kg of formulation. In other embodiments, the auris-acceptable otic therapeutic agent formulation has less than about 4 EU/kg of formulation. In additional embodiments, the auris-acceptable otic therapeutic agent formulation has less than about 3 EU/kg of formulation. In some embodiments, the auris-acceptable otic therapeutic agent formulation has less than about 5 EU/kg Product. In other embodiments, the auris-acceptable otic therapeutic agent formulation has less than about 1 EU/kg Product. In additional embodiments, the auris-acceptable otic therapeutic agent formulation has less than about 0.2 EU/kg Product. In some embodiments, the auris-acceptable otic therapeutic agent formulation has less than about 5 EU/g of unit or Product. In other embodiments, the auris-acceptable otic therapeutic agent formulation has less than about 4 EU/g of unit or Product. In additional embodiments, the auris-acceptable otic therapeutic agent formulation has less than about 3 EU/g of unit or Product. In some embodiments, the auris-acceptable otic therapeutic agent formulation has less than about 5 EU/mg of unit or Product. In other embodiments, the auris-acceptable otic therapeutic agent formulation has less than about 4 EU/mg of unit or Product. In additional embodiments, the auris-acceptable otic therapeutic agent formulation has less than about 3 EU/mg of unit or Product. In certain embodiments, otic compositions described herein contain from about 1 to about 5 EU/mL of formulation. In certain embodiments, otic compositions described herein contain from about 2 to about 5 EU/mL of formulation, from about 3 to about 5 EU/mL of formulation, or from about 4 to about 5 EU/mL of formulation.

In certain embodiments, otic compositions or devices described herein contain lower endotoxin levels (e.g. <0.5 EU/mL of formulation) when compared to conventionally acceptable endotoxin levels (e.g., 0.5 EU/mL of formulation). In some embodiments, the auris-acceptable otic therapeutic agent formulation or device has less than about 0.5 EU/mL of formulation. In other embodiments, the auris-acceptable otic therapeutic agent formulation has less than about 0.4 EU/mL of formulation. In additional embodiments, the auris-acceptable otic therapeutic agent formulation has less than about 0.2 EU/mL of formulation.

Pyrogen detection, by way of example only, is performed by several methods. Suitable tests for sterility include tests described in United States Pharmacopoeia (USP) <71> Sterility Tests (23rd edition, 1995). The rabbit pyrogen test and the Limulus amebocyte lysate test are both specified in the United States Pharmacopeia Chapters <85> and <151> (USP23/NF 18, Biological Tests, The United States Pharmacopeial Convention, Rockville, Md., 1995). Alternative pyrogen assays have been developed based upon the monocyte activation-cytokine assay. Uniform cell lines suitable for quality control applications have been developed and have demonstrated the ability to detect pyrogenicity in samples that have passed the rabbit pyrogen test and the Limulus amebocyte lysate test (Taktak et al, J. Pharm. Pharmacol. (1990), 43:578-82). In an additional embodiment, the auris-acceptable otic therapeutic agent formulation is subject to depyrogenation. In a further embodiment, the process for the manufacture of the auris-acceptable otic therapeutic agent formulation comprises testing the formulation for pyrogenicity. In certain embodiments, the formulations described herein are substantially free of pyrogens.

### pH and Practical Osmolarity

Described herein, in some embodiments, are formulations comprising a non-natural Trk receptor agonist. As used herein, “practical osmolarity” means the osmolarity of a formulation that is measured by including the active agent and all excipients except the gelling and/or the thickening agent (e.g., polyoxyethylene-polyooxypropylene copolymers, carboxymethylcellulose or the like). The practical osmolarity of a formulation described herein is measured by any suitable method, e.g., a freezing point depression method as described in Viegas et. al., Int. J. Pharm., 1998, 160, 157-162. In some instances, the practical osmolarity of a composition described herein is measured by vapor pressure osmometry (e.g., vapor pressure depression method) that allows for determination of the osmolarity of a composition at higher temperatures. In some instances, vapor pressure depression method allows for determination of the osmolarity of a formulation comprising a gelling agent (e.g., a thermoreversible polymer) at a higher temperature wherein the gelling agent is in the form of a gel. The practical osmolality of an otic formulation described herein is from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, the formulations described herein have a practical osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L.

In some embodiments, the osmolarity at a target site of action (e.g., the perilymph) is about the same as the delivered osmolarity (i.e., osmolarity of materials that cross or penetrate the round window membrane) of any formulation described herein. In some embodiments, the formulations described herein have a deliverable osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L.

The main cation present in the endolymph is potassium. In addition the endolymph has a high concentration of positively charged amino acids. The main cation present in the perilymph is sodium. In certain instances, the ionic composition of the endolymph and perilymph regulate the electrochemical impulses of hair cells. In certain instances, any change in the ionic balance of the endolymph or perilymph results in a loss of hearing due to changes in the conduction of electrochemical impulses along otic hair cells. In some embodiments, a composition disclosed herein does not disrupt the ionic balance of the perilymph. In some embodiments, a composition disclosed herein has an ionic balance that is the same as or substantially the same as the perilymph. In some embodiments, a composition disclosed herein does not disrupt the ionic balance of the endolymph. In some embodiments, a composition disclosed herein has an ionic balance that is the same as or substantially the same as the endolymph. In some embodiments, an otic formulation described herein is formulated to provide an ionic balance that is compatible with inner ear fluids (e.g., endolymph and/or perilymph).

The endolymph and the perilymph have a pH that is close to the physiological pH of blood. The endolymph has a pH range of about 7.2-7.9; the perilymph has a pH range of about 7.2-7.4. The in situ pH of the proximal endolymph is about 7.4 while the pH of distal endolymph is about 7.9.

In some embodiments, the pH of a composition described herein is adjusted (e.g., by use of a buffer) to an endolymph-compatible pH range of about 5.5 to 9.0. In specific embodiments, the pH of a composition described herein is adjusted to a perilymph-suitable pH range of about 5.5 to about 9.0. In some embodiments, the pH of a composition described herein is adjusted to a perilymph-suitable range of about 5.5 to about 8.0, about 6 to about 8.0 or about 6.6 to about 8.0. In some embodiments, the pH of a composition described herein is adjusted to a perilymph-suitable pH range of about 7.0-7.6.

In some embodiments, useful formulations also include one or more pH adjusting agents or buffering agents. Suitable pH adjusting agents or buffers include, but are not limited to acetate, bicarbonate, ammonium chloride, citrate, phosphate, pharmaceutically acceptable salts thereof, and any combinations or mixtures thereof.

In one embodiment, when one or more buffers are utilized in the formulations of the present disclosure, they are combined, e.g., with a pharmaceutically acceptable vehicle and are present in the final formulation, e.g., in an amount ranging from about 0.1% to about 20%, from about 0.5% to about 10%. In certain embodiments of the present disclosure, the amount of buffer included in the gel formulations is an amount such that the pH of the gel formulation does not interfere with the body's natural buffering system.

In one embodiment, diluents are also used to stabilize compounds because they can provide a more stable environment. Salts dissolved in buffered solutions (which also can provide pH control or maintenance) are utilized as diluents in the art, including, but not limited to, a phosphate buffered saline solution.

In some embodiments, any gel formulation described herein has a pH that allows for sterilization (e.g, by filtration, aseptic mixing, heat treatment, and/or autoclaving (e.g., terminal sterilization)) of a gel formulation without degradation of the pharmaceutical agent (e.g., non-natural Trk receptor agonist) or the polymers comprising the gel. In order to reduce hydrolysis and/or degradation of the otic agent and/or the gel polymer during sterilization, the buffer pH is designed to maintain pH of the formulation in the 7-8 range during the process of sterilization (e.g., high temperature autoclaving).

In specific embodiments, any gel formulation described herein has a pH that allows for terminal sterilization (e.g, by heat treatment and/or autoclaving) of a gel formulation or the polymers comprising the gel. For example, in order to reduce hydrolysis and/or degradation of the otic agent and/or the gel polymer during autoclaving, the buffer pH is designed to maintain pH of the formulation in the 7-8 range at elevated temperatures. Any appropriate buffer is used depending on the otic agent used in the formulation. In some instances, since pKa of TRIS decreases as temperature increases at approximately −0.03/° C. and pKa of PBS increases as temperature increases at approximately 0.003/° C., autoclaving at 250° F. (121° C.) results in a significant downward pH shift (i.e. more acidic) in the TRIS buffer whereas a relatively much less upward pH shift in the PBS buffer and therefore much increased hydrolysis and/or degradation of an otic agent in TRIS than in PBS. Degradation of an otic agent is reduced by the use of an appropriate combination of a buffer and polymeric additives (e.g. CMC) as described herein.

In some embodiments, a formulation pH of from about 5.0 to about 9.0, from about 5.5 to about 8.5, from about 6.0 to about 7.6, from about 7 to about 7.8, from about 7.0 to about 7.6, from about 7.2 to 7.6, or from about 7.2 to about 7.4 is suitable for sterilization (e.g, by filtration or aseptic mixing or heat treatment and/or autoclaving (e.g., terminal sterilization)) of auris formulations described herein. In specific embodiments a formulation pH of about 6.0, about 6.5, about 7.0, about 7.1, about 7.2, about 7.3, about 7.4, about 7.5, or about 7.6 is suitable for sterilization (e.g, by filtration or aseptic mixing or heat treatment and/or autoclaving (e.g., terminal sterilization)) of any composition described herein.

In some embodiments, the formulations have a pH as described herein and include a thickening agent (e.g, a viscosity enhancing agent) such as, by way of non-limiting example, a cellulose based thickening agent described herein. In some instances, the addition of a secondary polymer (e.g., a thickening agent) and a pH of formulation as described herein, allows for sterilization of a formulation described herein without any substantial degradation of the otic agent and/or the polymer components in the otic formulation. In some embodiments, the ratio of a thermoreversible poloxamer to a thickening agent in a formulation that has a pH as described herein is about 40:1, about 35:1, about 30:1, about 25:1, about 20:1, about 15:1, about 10:1, or about 5:1. For example, in certain embodiments, a sustained and/or extended release formulation described herein comprises a combination of poloxamer 407 (pluronic F127) and carboxymethylcellulose (CMC) in a ratio of about 40:1, about 35:1, about 30:1, about 25:1, about 20:1, about 15:1, about 10:1, or about 5:1.

In some embodiments, the amount of thermoreversible polymer in any formulation described herein is about 10%, about 15%, about 20%, about 25%, about 30%, about 35%, or about 40% of the total weight of the formulation. In some embodiments, the amount of thermoreversible polymer in any formulation described herein is about 10%, about 11%, about 12%, about 13%, about 14%, about 15%, about 16%, about 17%, about 18%, about 19%, about 20%, about 21%, about 22%, about 23%, about 24%, or about 25% of the total weight of the formulation. In some embodiments, the amount of thermoreversible polymer (e.g., pluronic F127) in any formulation described herein is about 7.5% of the total weight of the formulation. In some embodiments, the amount of thermoreversible polymer (e.g., pluronic F127) in any formulation described herein is about 10% of the total weight of the formulation. In some embodiments, the amount of thermoreversible polymer (e.g., pluronic F127) in any formulation described herein is about 11% of the total weight of the formulation. In some embodiments, the amount of thermoreversible polymer (e.g., pluronic F127) in any formulation described herein is about 12% of the total weight of the formulation. In some embodiments, the amount of thermoreversible polymer (e.g., pluronic F127) in any formulation described herein is about 13% of the total weight of the formulation. In some embodiments, the amount of thermoreversible polymer (e.g., pluronic F127) in any formulation described herein is about 14% of the total weight of the formulation. In some embodiments, the amount of thermoreversible polymer (e.g., pluronic F127) in any formulation described herein is about 15% of the total weight of the formulation. In some embodiments, the amount of thermoreversible polymer (e.g., pluronic F127) in any formulation described herein is about 16% of the total weight of the formulation. In some embodiments, the amount of thermoreversible polymer (e.g., pluronic F127) in any formulation described herein is about 17% of the total weight of the formulation. In some embodiments, the amount of thermoreversible polymer (e.g., pluronic F127) in any formulation described herein is about 18% of the total weight of the formulation. In some embodiments, the amount of thermoreversible polymer (e.g., pluronic F127) in any formulation described herein is about 19% of the total weight of the formulation. In some embodiments, the amount of thermoreversible polymer (e.g., pluronic F127) in any formulation described herein is about 20% of the total weight of the formulation. In some embodiments, the amount of thermoreversible polymer (e.g., pluronic F127) in any formulation described herein is about 21% of the total weight of the formulation. In some embodiments, the amount of thermoreversible polymer (e.g., pluronic F127) in any formulation described herein is about 23% of the total weight of the formulation. In some embodiments, the amount of thermoreversible polymer (e.g., pluronic F127) in any formulation described herein is about 25% of the total weight of the formulation.

In some embodiments, the amount of thickening agent (e.g., a gelling agent) in any formulation described herein is about 0.1%, about 0.2%, about 0.3%, about 0.4%, about 0.5%, about 0.6%, about 0.7%, about 0.8%, about 0.9%, about 1%, about 5%, about 10%, or about 15% of the total weight of the formulation. In some embodiments, the amount of thickening agent (e.g., a gelling agent) in any formulation described herein is about 0.1%, 0.5%, about 1%, about 1.5%, about 2%, about 2.5%, about 3%, about 3.5%, about 4%, about 4.5%, or about 5% of the total weight of the formulation.

In some embodiments, the pharmaceutical formulations described herein are stable with respect to pH over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 1 month, at least about 2 months, at least about 3 months, at least about 4 months, at least about 5 months, or at least about 6 months. In other embodiments, the formulations described herein are stable with respect to pH over a period of at least about 1 week. Also described herein are formulations that are stable with respect to pH over a period of at least about 1 month.

Tonicity Agents

In general, the endolymph has a higher osmolality than the perilymph. For example, the endolymph has an osmolality of about 304 mOsm/kg H₂O while the perilymph has an osmolality of about 294 mOsm/kg H₂O. In certain embodiments, tonicity agents are added to the formulations described herein in an amount as to provide a practical osmolality of an otic formulation of about 100 mOsm/kg to about 1000 mOsm/kg, from about 200 mOsm/kg to about 800 mOsm/kg, from about 250 mOsm/kg to about 500 mOsm/kg, from about 250 mOsm/kg to about 350 mOsm/kg, or from about 280 mOsm/kg to about 320 mOsm/kg. In some embodiments, the formulations described herein have a practical osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 280 mOsm/L to about 320 mOsm/L, or about 250 mOsm/L to about 320 mOsm/L.

In some embodiments, the deliverable osmolarity of any formulation described herein is designed to be isotonic with the targeted otic structure (e.g., endolymph, perilymph, or the like). In specific embodiments, auris compositions described herein are formulated to provide a delivered perilymph-suitable osmolarity at the target site of action of about 250 to about 320 mOsm/L and preferably about 270 to about 320 mOsm/L. In specific embodiments, auris compositions described herein are formulated to provide a delivered perilymph-suitable osmolality at the target site of action of about 250 to about 320 mOsm/kg H₂O or an osmolality of about 270 to about 320 mOsm/kg H₂O. In specific embodiments, the deliverable osmolarity/osmolality of the formulations (i.e., the osmolarity/osmolality of the formulation in the absence of gelling or thickening agents (e.g., thermoreversible gel polymers) is adjusted, for example, by the use of appropriate salt concentrations (e.g., concentration of potassium or sodium salts) or the use of tonicity agents which renders the formulations endolymph-compatible and/or perilymph-compatible (i.e. isotonic with the endolymph and/or perilymph) upon delivery at the target site. The osmolarity of a formulation comprising a thermoreversible gel polymer is an unreliable measure due to the association of varying amounts of water with the monomeric units of the polymer. The practical osmolarity of a formulation (i.e., osmolarity in the absence of a gelling or thickening agent (e.g. a thermoreversible gel polymer) is a reliable measure and is measured by any suitable method (e.g., freezing point depression method, vapor depression method). In some instances, the formulations described herein provide a deliverable osmolarity (e.g., at a target site (e.g., perilymph) that causes minimal disturbance to the environment of the inner ear and causes minimum discomfort (e.g., vertigo and/or nausea) to a mammal upon administration.

In some embodiments, any formulation described herein is isotonic with the perilymph and/or endolymph. Isotonic formulations are provided by the addition of a tonicity agent. Suitable tonicity agents include, but are not limited to any pharmaceutically acceptable sugar, salt, or any combinations or mixtures thereof, such as, but not limited to dextrose, glycerin, mannitol, sorbitol, sodium chloride, and other electrolytes.

Useful auris compositions include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

In some embodiments, the formulations described herein have a pH and/or practical osmolarity as described herein, and have a concentration of active pharmaceutical ingredient between about 1 μM and about 10 μM, between about 1 mM and about 100 mM, between about 0.1 mM and about 100 mM, between about 0.1 mM and about 100 nM. In some embodiments, the formulations described herein have a pH and/or practical osmolarity as described herein, and have a concentration of active pharmaceutical ingredient from about 0.0001% to about 60%, from about 0.001% to about 40%, from about 0.01% to about 20%, from about 0.01% to about 10%, from about 0.01% to about 7.5%, from about 0.01% to about 6%, from about 0.01 to about 5%, from about 0.1 to about 10%, or from about 0.1 to about 6% of the active ingredient by weight of the formulation. In some embodiments, the formulations described herein have a pH and/or practical osmolarity as described herein, and have a concentration of active pharmaceutical ingredient from about 0.1 mg/mL to about 100 mg/mL, from about 1 mg/mL to about 100 mg/mL, from about 1 mg/mL to about 80 mg/mL, from about 1 mg/mL to about 60 mg/mL, from about 1 mg/mL to about 50 mg/mL, from about 1 mg/mL to about 50 mg/mL, from about 1 mg/mL to about 20 mg/mL, from about 1 mg/mL to about 10 mg/mL, -from about 1 mg/mL to about 5 mg/mL, or from about 0.5 mg/mL to about 5 mg/mL of the active agent by volume of the formulation. In some embodiments, the formulations described herein have a pH and/or practical osmolarity as described herein, and have a concentration of active pharmaceutical ingredient from about 1 μg/mL to about 500 μg/mL, from about 1 μg/mL to about 250 μg/mL, from about 1 μg to about 100 μg/mL, from about 1 μg/mL to about 50 μg/mL, or from about 1 μg/mL to about 20 μg/mL of the active agent by volume of the formulation.

In some embodiments, the active pharmaceutical ingredient comprises a non-natural Trk receptor agonist. In some cases, the formulations described herein have a pH and/or practical osmolarity as described herein and have a concentration of a non-natural Trk receptor agonist from about 1 μM to about 10 μM, from about 1 mM to about 100 mM, from about 0.1 mM to about 100 mM, from about 0.1 mM to about 100 nM. In some embodiments, the formulations described herein have a pH and/or practical osmolarity as described herein, and have a concentration of a non-natural Trk receptor agonist from about 0.0001% to about 60%, from about 0.001% to about 40%, from about 0.01% to about 20%, from about 0.01% to about 10%, from about 0.01% to about 7.5%, from about 0.01% to 6%, from about 0.01 to 5%, from about 0.1 to about 10%, or from about 0.1 to about 6% of the active ingredient by weight of the formulation. In some embodiments, the formulations described herein have a pH and/or practical osmolarity as described herein, and have a concentration of a non-natural Trk receptor agonist from about 0.1 mg/mL to about 100 mg/mL, from about 1 mg/mL to about 100 mg/mL, from about 1 mg/mL to about 80 mg/mL, from about 1 mg/mL to about 60 mg/mL, from about 1 mg/mL to about 50 mg/mL, from about 1 mg/mL to about 50 mg/mL, from about 1 mg/mL to about 20 mg/mL, from about 1 mg/mL to about 10 mg/mL, -from about 1 mg/mL to about 5 mg/mL, or from about 0.5 mg/mL to about 5 mg/mL of the non-natural Trk receptor agonist by volume of the formulation. In some embodiments, the formulations described herein have a pH and/or practical osmolarity as described herein, and have a concentration of a non-natural Trk receptor agonist from about 1 μg/mL to about 500 μg/mL, from about 1 μg/mL to about 250 μg/mL, from about 1 μg to about 100 μg/mL, from about 1 μg/mL to about 50 μg/mL, or from about 1 μg/mL to about 20 μg/mL of the non-natural Trk receptor agonist by volume of the formulation.

### Tunable Release Characteristics

The release of non-natural Trk receptor agonists from any formulation, composition, or device described herein is optionally tunable to the desired release characteristics. In some embodiments, a composition described herein is a solution that is substantially free of gelling components. In such instances, the composition provides essentially immediate release of the non-natural Trk receptor agonists. In some of such embodiments, the composition is useful in perfusion of otic structures, e.g., during surgery.

In some of such embodiments, the composition provides release of the non-natural Trk receptor agonist from about 2 days to about 4 days.

In some embodiments, a composition described herein comprising a non-natural Trk receptor agonist, further comprises a gelling agent (e.g., poloxamer 407) and provides release of the non-natural Trk receptor agonist over a period of from about 1 day to about 3 days. In some embodiments, a composition described herein comprising a non-natural Trk receptor agonist, further comprises a gelling agent (e.g., poloxamer 407) and provides release of the non-natural Trk receptor agonist over a period of from about 1 day to about 5 days. In some embodiments, a composition described herein comprising a non-natural Trk receptor agonist, further comprises a gelling agent (e.g., poloxamer 407) and provides release of the non-natural Trk receptor agonist over a period of from about 2 days to about 7 days.

In some embodiments, a composition described herein comprising a non-natural Trk receptor agonist, further comprises from about 14 to about 17% of a gelling agent (e.g., poloxamer 407), and provides extended sustained release over a period of from about 1 week to about 3 weeks. In some embodiments, a composition described herein comprising a non-natural Trk receptor agonist, further comprises from about 18 to about 21% of a gelling agent (e.g., poloxamer 407) and, provides extended sustained release over a period of from about 3 weeks to about 6 weeks.

In some embodiments, the viscosity of any formulation described herein comprising a non-natural Trk receptor agonist, is designed to provide a suitable rate of release from an auris compatible gel. In some embodiments, the concentration of a thickening agent (e.g., gelling components such as polyoxyethylene-polyoxypropylene copolymers) allows for a tunable mean dissolution time (MDT). The MDT is inversely proportional to the release rate of an active agent from a composition or device described herein. Experimentally, the released non-natural Trk receptor agonist is optionally fitted to the Korsmeyer-Peppas equation

$$\frac{Q}{{Q}_{α}} = {kt}^{n} + b$$

where Q is the amount of otic agent released at time t, Qₐ is the overall released amount of otic agent, k is a release constant of the nth order, n is a dimensionless number related to the dissolution mechanism, and b is the axis intercept, characterizing the initial burst release mechanism wherein n=1 characterizes an erosion controlled mechanism. The mean dissolution time (MDT) is the sum of different periods of time the drug molecules stay in the matrix before release, divided by the total number of molecules, and is optionally calculated by:

$$MDT = \frac{{nk}^{- 1 / n}}{n + 1}$$

For example, a linear relationship between the mean dissolution time (MDT) of a composition or device and the concentration of the gelling agent (e.g., poloxamer) indicates that the non-natural Trk receptor agonist is released due to the erosion of the polymer gel (e.g., poloxamer) and not via diffusion. In another example, a non-linear relationship indicates release of otic agent via a combination of diffusion and/or polymer gel degradation. In another example, a faster gel elimination time course of a composition or device (a faster release of the non-natural Trk receptor agonist) indicates lower mean dissolution time (MDT). The concentration of gelling components and/or active agent in a composition are tested to determine suitable parameters for MDT. In some embodiments, injection volumes are also tested to determine suitable parameters for preclinical and clinical studies. The gel strength and concentration of the non-natural Trk receptor agonist affects release kinetics of the non-natural Trk receptor agonist from the composition. At low poloxamer concentration, elimination rate is accelerated (MDT is lower). An increase in the non-natural Trk receptor agonist concentration in the composition or device prolongs residence time and/or MDT of the non-natural Trk receptor agonist in the ear.

In some embodiments, the MDT for poloxamer from a composition or device described herein is at least 6 hours. In some embodiments, the MDT for poloxamer from a composition or device described herein is at least 10 hours.

In some embodiments, the MDT for a non-natural Trk receptor agonist from a composition or device described herein is from about 30 hours to about 48 hours. In some embodiments, the MDT for a non-natural Trk receptor agonist from a composition or device described herein is from about 30 hours to about 96 hours. In some embodiments, the MDT for a non-natural Trk receptor agonist from a composition or device described herein is from about 30 hours to about 1 week. In some embodiments, the MDT for a non-natural Trk receptor agonist from a composition or device described herein is from about 1 week to about 6 weeks.

In certain embodiments, any controlled-release otic composition described herein increases the exposure of a non-natural Trk receptor agonist and increases the Area Under the Curve (AUC) in otic fluids (e.g., endolymph and/or perilymph) by about 30%, about 40%, about 50%, about 60%, about 70%, about 80%, about 90%, about 100%, or higher than 100%, compared to an otic composition that is not a controlled-release otic composition. In certain embodiments, any controlled-release otic composition described herein increases the exposure time of a non-natural Trk receptor agonist and decreases the Cₘₐₓ in otic fluids (e.g., endolymph and/or perilymph) by about 40%, about 30%, about 20%, or about 10%, compared to a formulation that is not a controlled-release otic composition. In certain embodiments, any controlled-release otic composition described herein alters (e.g. reduces) the ratio of Cₘₐₓ to Cₘᵢₙ compared to a formulation that is not a controlled-release otic composition. In certain embodiments, any controlled-release otic composition described herein increases the exposure of a non-natural Trk receptor agonist and increases the length of time that the concentration of the non-natural Trk receptor agonist is above Cₘᵢₙ by about 30%, about 40%, about 50%, about 60%, about 70%, about 80%, or about 90% compared to a formulation that is not a controlled-release otic composition. In certain embodiments, the increase in exposure of a non-natural Trk receptor agonist and the increase in the length of time that the concentration of the non-natural Trk receptor agonist is above Cₘᵢₙ by a controlled-release otic composition described herein is greater than 100% compared to a formulation that is not a controlled-release otic composition. In certain instances, controlled-release otic compositions described herein delay the time to Cₘₐₓ. In certain instances, the controlled steady release of a drug prolongs the time the concentration of the non-natural Trk receptor agonist will stay above the Cₘᵢₙ. In some embodiments, otic compositions described herein prolong the residence time of a non-natural Trk receptor agonist in the inner ear and provide a stable drug exposure profile. In some instances, an increase in concentration of a non-natural Trk receptor agonist in the otic composition saturates the clearance process and allows for a more rapid and stable steady state to be reached.

In certain instances, once exposure to a non-natural Trk receptor agonist (e.g., concentration in the endolymph or perilymph) reaches steady state, the concentration of the non-natural Trk receptor agonist in the endolymph or perilymph stays at or about the therapeutic dose for an extended period of time (e.g., one day, 2 days, 3 days, 4 days, 5 days, 6 days, 1 week, 3 weeks, 6 weeks, or 2 months). In some embodiments, the steady state concentration of a non-natural Trk receptor agonist released from a controlled-release otic composition described herein is from about 20 to about 50 times the steady state concentration of a non-natural Trk receptor agonist released from a formulation that is not a controlled-release otic composition.

### Pharmaceutical Formulations

Provided herein are otic pharmaceutical compositions or devices that include at least one non-natural Trk receptor agonist and a pharmaceutically acceptable diluent(s), excipient(s), or carrier(s). In some embodiments, the pharmaceutical compositions include other medicinal or pharmaceutical agents; carriers; adjuvants; preserving, stabilizing, wetting or emulsifying agents; solution promoters; salts for regulating the osmotic pressure; and/or buffers. In some embodiments, the otic composition or device comprises (i) a non-natural Trk receptor agonist, (ii) a gelling and viscosity enhancing agent, (iii) a pH adjusting agent, and (iv) sterile water.

In other embodiments, the otic pharmaceutical compositions also contain other therapeutic substances.

In some embodiments, the otic pharmaceutical compositions or devices described herein include a dye to help enhance the visualization of the gel when applied. In some embodiments, dyes that are compatible with the auris-acceptable compositions or devices described herein include Evans blue (e.g., 0.5% of the total weight of an otic formulation), Methylene blue (e.g., 1% of the total weight of an otic formulation), Isosulfan blue (e.g., 1% of the total weight of an otic formulation), Trypan blue (e.g., 0.15% of the total weight of an otic formulation), and/or indocyanine green (e.g., 25 mg/vial). Other common dyes, e.g, FD&C red 40, FD&C red 3, FD&C yellow 5, FD&C yellow 6, FD&C blue1, FD&C blue2, FD&C green 3, fluorescence dyes (e.g., Fluorescein isothiocyanate, rhodamine, Alexa Fluors, DyLight Fluors), and/or dyes that are visualizable in conjunction with non-invasive imaging techniques such as MRI, CAT scans, PET scans, or the like. Gadolinium-based MRI dyes, iodine-base dyes, barium-based dyes, or the like are also contemplated for use with any otic composition described herein. Other dyes that are compatible with any formulation or composition described herein are listed in the Sigma-Aldrich catalog under dyes (which is included herein by reference for such disclosure).

In some embodiments, mechanical or imaging devices are used to monitor or survey the hearing, balance, or other auris disorder. For example, magnetic resonance imaging (MRI) devices are specifically contemplated within the scope of the embodiments, wherein the MRI devices (for example, 3 Tesla MRI devices) are capable of evaluating Meniere Disease progression and subsequent treatment with the pharmaceutical formulations disclosed herein. Gadolinium-based dyes, iodine-base dyes, barium-based dyes, or the like are also contemplated for use with any auris-compatible composition or device described herein and/or with any mechanical or imaging devices described herein. In certain embodiments, gadolinium hydrate is used in combination with MRI and/or any pharmaceutical composition or device described herein to evaluate disease severity (e.g., size of endolymphatic hydrops), formulation penetration into the inner ear, and/or therapeutic effectiveness of the pharmaceutical formulations/devices in the otic diseases described herein (e.g., Meniere's disease).

Any otic pharmaceutical composition or device described herein is administered by locating the composition or device in contact with the crista fenestrae cochlea, the round window, the tympanic cavity, the tympanic membrane, the auris media, or the auris externa.

In one specific embodiment of the auris-acceptable controlled-release non-natural Trk receptor agonist pharmaceutical formulations described herein, the non-natural Trk receptor agonist is provided in a gel matrix, also referred to herein as “auris acceptable gel formulations”, “auris interna-acceptable gel formulations”, “auris media-acceptable gel formulations”, “auris externa-acceptable gel formulations”, “auris gel formulations”, or variations thereof. All of the components of the gel formulation must be compatible with the targeted auris structure. Further, the gel formulations provide controlled-release of the non-natural Trk receptor agonist to the desired site within the targeted auris structure; in some embodiments, the gel formulation also has an immediate or rapid release component for delivery of the non-natural Trk receptor agonist to the desired target site. In other embodiments, the gel formulation has a sustained release component for delivery of the non-natural Trk receptor agonist. In some embodiments, the auris gel formulations are biodegradable. In other embodiments, the auris gel formulations include a mucoadhesive excipient to allow adhesion to the external mucous layer of the round window membrane. In yet other embodiments, the auris gel formulations include a penetration enhancer excipient; in further embodiments, the auris gel formulation contains a viscosity enhancing agent sufficient to provide a viscosity of from about 500 and 1,000,000 centipoise; from about 750 to about 1,000,000 centipoise; from about 1000 to about 1,000,000 centipoise; from about 1000 to about 400,000 centipoise; from about 2000 to about 100,000 centipoise; from about 3000 to about 50,000 centipoise; from about 4000 to about 25,000 centipoise; from about 5000 to about 20,000 centipoise; or from about 6000 to about 15,000 centipoise. In some embodiments, the auris gel formulation contains a viscosity enhancing agent sufficient to provide a viscosity of from about 50,0000 to about 1,000,000 centipoise.

In some embodiments, the otic pharmaceutical compositions or devices described herein are low viscosity compositions or devices at body temperature. In some embodiments, low viscosity compositions or devices contain from about 1% to about 10% of a viscosity enhancing agent (e.g., gelling components such as polyoxyethylene-polyoxypropylene copolymers). In some embodiments, low viscosity compositions or devices contain from about 2% to about 10% of a viscosity enhancing agent (e.g., gelling components such as polyoxyethylene-polyoxypropylene copolymers). In some embodiments, low viscosity compositions or devices contain from about 5% to about 10% of a viscosity enhancing agent (e.g., gelling components such as polyoxyethylene-polyoxypropylene copolymers). In some embodiments, low viscosity compositions or devices are substantially free of a viscosity enhancing agent (e.g., gelling components such as polyoxyethylene-polyoxypropylene copolymers). In some embodiments, a low viscosity non-natural Trk receptor agonist composition or device described herein provides an apparent viscosity of from about 100 cP to about 10,000 cP. In some embodiments, a low viscosity non-natural Trk receptor agonist composition or device described herein provides an apparent viscosity of from about 500 cP to about 10,000 cP. In some embodiments, a low viscosity non-natural Trk receptor agonist composition or device described herein provides an apparent viscosity of from about 1000 cP to about 10,000 cP. In some of such embodiments, a low viscosity non-natural Trk receptor agonist composition or device is administered in combination with an external otic intervention, e.g., a surgical procedure including but not limited to middle ear surgery, inner ear surgery, typanostomy, cochleostomy, labyrinthotomy, mastoidectomy, stapedectomy, stapedotomy, endolymphatic sacculotomy, or the like. In some of such embodiments, a low viscosity non-natural Trk receptor agonist composition or device is administered during an otic intervention. In other such embodiments, a low viscosity non-natural Trk receptor agonist composition or device is administered before the otic intervention.

In some embodiments, the otic pharmaceutical compositions or devices described herein are high viscosity compositions or devices at body temperature. In some embodiments, high viscosity compositions or devices contain from about 10% to about 25% of a viscosity enhancing agent (e.g., gelling components such as polyoxyethylene-polyoxypropylene copolymers). In some embodiments, high viscosity compositions or devices contain from about 14% to about 22% of a viscosity enhancing agent (e.g., gelling components such as polyoxyethylene-polyoxypropylene copolymers). In some embodiments, high viscosity compositions or devices contain from about 15% to about 21% of a viscosity enhancing agent (e.g., gelling components such as polyoxyethylene-polyoxypropylene copolymers). In some embodiments, a high viscosity non-natural Trk receptor agonist composition or device described herein provides an apparent viscosity of from about 100,000 cP to about 1,000,000 cP. In some embodiments, a high viscosity non-natural Trk receptor agonist composition or device described herein provides an apparent viscosity of from about 150,000 cP to about 500,000 cP. In some embodiments, a high viscosity non-natural Trk receptor agonist composition or device described herein provides an apparent viscosity of from about 250,000 cP to about 500,000 cP. In some of such embodiments, a high viscosity composition or device is a liquid at room temperature and gels at about between room temperature and body temperature (including an individual with a serious fever, e.g., up to about 42° C.). In some embodiments, an otic high viscosity composition or device comprising a non-natural Trk receptor agonist is administered as monotherapy for treatment of an otic disease or condition described herein. In some embodiments, an otic high viscosity composition or device comprising a non-natural Trk receptor agonist is administered in combination with an external otic intervention, e.g., a surgical procedure including but not limited to middle ear surgery, inner ear surgery, typanostomy, cochleostomy, labyrinthotomy, mastoidectomy, stapedectomy, stapedotomy, endolymphatic sacculotomy, or the like. In some of such embodiments, a high viscosity otic composition or device comprising a non-natural Trk receptor agonist is administered after the otic intervention. In other such embodiments, a high viscosity non-natural Trk receptor agonist composition or device is administered before the otic intervention.

In other embodiments, the otic pharmaceutical formulations described herein further provide an auris-acceptable hydrogel; in yet other embodiments, the otic pharmaceutical formulations provide an auris-acceptable microsphere or microparticle; in still other embodiments, the otic pharmaceutical formulations provide an auris-acceptable liposome. In some embodiments, the otic pharmaceutical formulations provide an auris-acceptable foam; in yet other embodiments, the otic pharmaceutical formulations provide an auris-acceptable paint; in still further embodiments, otic pharmaceutical formulations provide an auris-acceptable in situ forming spongy material. In some embodiments, the otic pharmaceutical formulations provide an auris-acceptable solvent release gel. In some embodiments, the otic pharmaceutical formulations provide an actinic radiation curable gel. Further embodiments include a thermoreversible gel in the otic pharmaceutical formulation, such that upon preparation of the gel at room temperature or below, the formulation is a fluid, but upon application of the gel into or near the auris interna and/or auris media target site, including the tympanic cavity, round window membrane, or the crista fenestrae cochleae, the otic-pharmaceutical formulation stiffens or hardens into a gel-like substance.

In further or alternative embodiments, the otic gel formulations are capable of being administered on or near the round window membrane via intratympanic injection. In other embodiments, the otic gel formulations are administered on or near the round window or the crista fenestrae cochleae through entry via a post-auricular incision and surgical manipulation into or near the round window or the crista fenestrae cochleae area. Alternatively, the otic gel formulation is applied via syringe and needle, wherein the needle is inserted through the tympanic membrane and guided to the area of the round window or crista fenestrae cochleae. The otic gel formulations are then deposited on or near the round window or crista fenestrae cochleae for localized treatment of autoimmune otic disorders. In other embodiments, the otic gel formulations are applied via microcathethers implanted into the patient, and in yet further embodiments the formulations are administered via a pump device onto or near the round window membrane. In still further embodiments, the otic gel formulations are applied at or near the round window membrane via a microinjection device. In yet other embodiments, the otic gel formulations are applied in the tympanic cavity. In some embodiments, the otic gel formulations are applied on the tympanic membrane. In still other embodiments, the otic gel formulations are applied onto or in the auditory canal.

### Controlled-Release Formulations

In general, controlled-release drug formulations impart control over the release of drug with respect to site of release and time of release within the body. As discussed herein, controlled release refers to immediate release, delayed release, sustained release, extended release, variable release, pulsatile release and bi-modal release. Many advantages are offered by controlled release. First, controlled release of a pharmaceutical agent allows less frequent dosing and thus minimizes repeated treatment. Second, controlled release treatment results in more efficient drug utilization and less of the compound remains as a residue. Third, controlled release offers the possibility of localized drug delivery by placement of a delivery device or formulation at the site of disease. Still further, controlled release offers the opportunity to administer and release two or more different drugs, each having a unique release profile, or to release the same drug at different rates or for different durations, by means of a single dosage unit.

Accordingly, one aspect of the embodiments disclosed herein is to provide a controlled-release non-natural Trk receptor agonist auris-acceptable composition or device for the treatment of autoimmune disorders and/or inflammatory disorders. The controlled release aspect of the compositions and/or formulations and/or devices disclosed herein is imparted through a variety of agents, including but not limited to excipients, agents, or materials that are acceptable for use in the auris interna or other otic structure. By way of example only, such excipients, agents, or materials include an auris-acceptable polymer, an auris-acceptable viscosity enhancing agent, an auris-acceptable gel, an auris-acceptable paint, an auris-acceptable foam, an auris-acceptable xerogel, an auris-acceptable microsphere or microparticle, an auris-acceptable hydrogel, an auris-acceptable in situ forming spongy material, an auris-acceptable actinic radiation curable gel, an auris-acceptable solvent release gel, an auris-acceptable liposome, an auris-acceptable nanocapsule or nanosphere, an auris-acceptable thermoreversible gel, or any combination thereof.

### Auris-Acceptable Gels

Described herein, in some embodiments, are otic formulations comprising a non-natural Trk receptor agonist that is an auris-acceptable gel. Gels, sometimes referred to as jellies, have been defined in various ways. For example, the United States Pharmacopoeia defines gels as semisolid systems consisting of either suspensions made up of small inorganic particles or large organic molecules interpenetrated by a liquid. Gels include a single-phase or a two-phase system. A single-phase gel consists of organic macromolecules distributed uniformly throughout a liquid in such a manner that no apparent boundaries exist between the dispersed macromolecules and the liquid. Some single-phase gels are prepared from synthetic macromolecules (e.g., carbomer) or from natural gums (e.g., tragacanth). In some embodiments, single-phase gels are generally aqueous but will also be made using alcohols and oils. Two-phase gels consist of a network of small discrete particles.

Gels can also be classified as being hydrophobic or hydrophilic. In certain embodiments, the base of a hydrophobic gel consists of a liquid paraffin with polyethylene or fatty oils gelled with colloidal silica or aluminum or zinc soaps. In contrast, the base of hydrophilic gels usually consists of water, glycerol, or propylene glycol gelled with a suitable gelling agent (e.g., tragacanth, starch, cellulose derivatives, carboxyvinylpolymers, and magnesium-aluminum silicates). In certain embodiments, the rheology of the compositions or devices disclosed herein is pseudo plastic, plastic, thixotropic, or dilatant.

In one embodiment the enhanced viscosity auris-acceptable formulation described herein is not a liquid at room temperature. In certain embodiments, the enhanced viscosity formulation is characterized by a phase transition between room temperature and body temperature (including an individual with a serious fever, e.g., up to about 42° C.). In some embodiments, the phase transition occurs at about 1° C. below body temperature, at about 2° C. below body temperature, at about 3° C. below body temperature, at about 4° C. below body temperature, at about 6° C. below body temperature, at about 8° C. below body temperature, or at about 10° C. below body temperature. In some embodiments, the phase transition occurs at about 15° C. below body temperature, at about 20° C. below body temperature, or at about 25° C. below body temperature. In specific embodiments, the gelation temperature (Tgel) of a formulation described herein is about 20° C., about 25° C., or about 30° C. In certain embodiments, the gelation temperature (Tgel) of a formulation described herein is about 35° C. or about 40° C. In one embodiment, administration of any formulation described herein at about body temperature reduces or inhibits vertigo associated with intratympanic administration of otic formulations. Included within the definition of body temperature is the body temperature of a healthy individual or an unhealthy individual, including an individual with a fever (up to ˜42° C.). In some embodiments, the pharmaceutical compositions or devices described herein are liquids at about room temperature and are administered at or about room temperature, reducing or ameliorating side effects such as, for example, vertigo.

Polymers composed of polyoxypropylene and polyoxyethylene form thermoreversible gels when incorporated into aqueous solutions. These polymers have the ability to change from the liquid state to the gel state at temperatures close to body temperature, therefore allowing useful formulations that are applied to the targeted auris structure(s). The liquid state-to-gel state phase transition is dependent on the polymer concentration and the ingredients in the solution.

Poloxamer 407 (PF-127) is a nonionic surfactant composed of polyoxyethylene-polyoxypropylene copolymers. Other poloxamers include 188 (F-68 grade), 237 (F-87 grade), and 338 (F-108 grade). Aqueous solutions of poloxamers are stable in the presence of acids, alkalis, and metal ions. PF-127 is a commercially available polyoxyethylene-polyoxypropylene triblock copolymer of general formula E106 P70 E106, with an average molar mass of 13,000. The polymer can be further purified by suitable methods that will enhance gelation properties of the polymer. It contains approximately 70% ethylene oxide, which accounts for its hydrophilicity. It is one of the series of poloxamer ABA block copolymers, whose members share the chemical formula shown below.

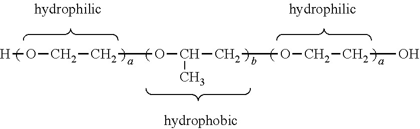

PF-127 is of particular interest since concentrated solutions (>20% w/w) of the copolymer are transformed from low viscosity transparent solutions to solid gels on heating to body temperature. This phenomenon, therefore, suggests that when placed in contact with the body, the gel preparation will form a semi-solid structure and a sustained release depot. Furthermore, PF-127 has good solubilizing capacity, low toxicity and is, therefore, considered a good medium for drug delivery systems.

In an alternative embodiment, the thermogel is a PEG-PLGA-PEG triblock copolymer (Jeong et al, Nature (1997), 388:860-2; Jeong et al, J. Control. Release (2000), 63:155-63; Jeong et al, Adv. Drug Delivery Rev. (2002), 54:37-51). The polymer exhibits sol-gel behavior over a concentration of about 5% w/w to about 40% w/w. Depending on the properties desired, the lactide/glycolide molar ratio in the PLGA copolymer ranges from about 1:1 to about 20:1. The resulting copolymers are soluble in water and form a free-flowing liquid at room temperature but form a hydrogel at body temperature. A commercially available PEG-PLGA-PEG triblock copolymer is RESOMER RGP t50106 manufactured by Boehringer Ingelheim. This material is composed of a PGLA copolymer of 50:50 poly(DL-lactide-co-glycolide), is 10% w/w of PEG, and has a molecular weight of about 6000.

ReGel® is a tradename of MacroMed Incorporated for a class of low molecular weight, biodegradable block copolymers having reverse thermal gelation properties as described in U.S. Pat. Nos. 6,004,573, 6,117,949, 6,201,072, and 6,287,588. It also includes biodegradable polymeric drug carriers disclosed in pending U.S. patent application Ser. Nos. 09/906,041, 09/559,799 and 10/919,603. The biodegradable drug carrier comprises ABA-type or BAB-type triblock copolymers, or mixtures thereof, wherein the A-blocks are relatively hydrophobic and comprise biodegradable polyesters or poly(orthoester)s, and the B-blocks are relatively hydrophilic and comprise polyethylene glycol (PEG), said copolymers having a hydrophobic content of between 50.1 to 83% by weight and a hydrophilic content of between 17 to 49.9% by weight, and an overall block copolymer molecular weight of between 2000 and 8000 Daltons. The drug carriers exhibit water solubility at temperatures below normal mammalian body temperatures and undergo reversible thermal gelation to then exist as a gel at temperatures equal to physiological mammalian body temperatures. The biodegradable, hydrophobic A polymer block comprises a polyester or poly(ortho ester), in which the polyester is synthesized from monomers selected from the group consisting of D,L-lactide, D-lactide, L-lactide, D,L-lactic acid, D-lactic acid, L-lactic acid, glycolide, glycolic acid, ε-caprolactone, ε-hydroxyhexanoic acid, γ-butyrolactone, γ-hydroxybutyric acid, δ-valerolactone, δ-hydroxyvaleric acid, hydroxybutyric acids, malic acid, and copolymers thereof and having an average molecular weight of between about 600 and 3000 Daltons. The hydrophilic B-block segment is preferably polyethylene glycol (PEG) having an average molecular weight of between about 500 and 2200 Daltons.

Additional biodegradable thermoplastic polyesters include AtriGel® (provided by Atrix Laboratories, Inc.) and/or those disclosed, e.g., in U.S. Pat. Nos. 5,324,519; 4,938,763; 5,702,716; 5,744,153; and 5,990,194; wherein the suitable biodegradable thermoplastic polyester is disclosed as a thermoplastic polymer. Examples of suitable biodegradable thermoplastic polyesters include polylactides, polyglycolides, polycaprolactones, copolymers thereof, terpolymers thereof, and any combinations thereof. In some such embodiments, the suitable biodegradable thermoplastic polyester is a polylactide, a polyglycolide, a copolymer thereof, a terpolymer thereof, or any combination thereof. In one embodiment, the biodegradable thermoplastic polyester is 50/50 poly(DL-lactide-co-glycolide) having a carboxy terminal group; is present in about 30 wt. % to about 40 wt. % of the composition; and has an average molecular weight of about 23,000 to about 45,000. Alternatively, in another embodiment, the biodegradable thermoplastic polyester is 75/25 poly (DL-lactide-co-glycolide) without a carboxy terminal group; is present in about 40 wt. % to about 50 wt. % of the composition; and has an average molecular weight of about 15,000 to about 24,000. In further or alternative embodiments, the terminal groups of the poly(DL-lactide-co-glycolide) are either hydroxyl, carboxyl, or ester depending upon the method of polymerization. Polycondensation of lactic or glycolic acid provides a polymer with terminal hydroxyl and carboxyl groups. Ring-opening polymerization of the cyclic lactide or glycolide monomers with water, lactic acid, or glycolic acid provides polymers with the same terminal groups. However, ring-opening of the cyclic monomers with a monofunctional alcohol such as methanol, ethanol, or 1-dodecanol provides a polymer with one hydroxyl group and one ester terminal groups. Ring-opening polymerization of the cyclic monomers with a diol such as 1,6-hexanediol or polyethylene glycol provides a polymer with only hydroxyl terminal groups.

Since the polymer systems of thermoreversible gels dissolve more completely at reduced temperatures, methods of solubilization include adding the required amount of polymer to the amount of water to be used at reduced temperatures. Generally after wetting the polymer by shaking, the mixture is capped and placed in a cold chamber or in a thermostatic container at about 0-10° C. in order to dissolve the polymer. The mixture is stirred or shaken to bring about a more rapid dissolution of the thermoreversible gel polymer. The non-natural Trk receptor agonist and various additives such as buffers, salts, and preservatives are subsequently added and dissolved. In some instances the non-natural Trk receptor agonist and/or other pharmaceutically active agent is suspended if it is insoluble in water. The pH is modulated by the addition of appropriate buffering agents. Round window membrane mucoadhesive characteristics are optionally imparted to a thermoreversible gel by incorporation of round window membrane mucoadhesive carbomers, such as Carbopol® 934P, to the composition (Majithiya et al, AAPS PharmSciTech (2006), 7(3), p. E1; EP0551626, both of which is incorporated herein by reference for such disclosure).

In one embodiment are auris-acceptable pharmaceutical gel formulations which do not require the use of an added viscosity enhancing agent. Such gel formulations incorporate at least one pharmaceutically acceptable buffer. In one aspect is a gel formulation comprising a non-natural Trk receptor agonist and a pharmaceutically acceptable buffer. In another embodiment, the pharmaceutically acceptable excipient or carrier is a gelling agent.

In other embodiments, useful non-natural Trk receptor agonist auris-acceptable pharmaceutical formulations also include one or more pH adjusting agents or buffering agents to provide an endolymph or perilymph suitable pH. Suitable pH adjusting agents or buffers include, but are not limited to acetate, bicarbonate, ammonium chloride, citrate, phosphate, pharmaceutically acceptable salts thereof, and combinations or mixtures thereof. Such pH adjusting agents and buffers are included in an amount required to maintain pH of the composition from a pH of about 5 to about 9, in one embodiment a pH from about 6.5 to about 7.5, and in yet another embodiment at a pH of about 6.5, 6.6, 6.7, 6.8, 6.9, 7.0, 7.1, 7.2, 7.3, 7.4, 7.5, 7.6, 7.7, 7.8, 7.9, or 8.0. In one embodiment, when one or more buffers are utilized in the formulations of the present disclosure, they are combined, e.g., with a pharmaceutically acceptable vehicle and are present in the final formulation, e.g., in an amount ranging from about 0.1% to about 20%, from about 0.5% to about 10%. In certain embodiments of the present disclosure, the amount of buffer included in the gel formulations are an amount such that the pH of the gel formulation does not interfere with the auris media or auris interna's natural buffering system, or does not interfere with the natural pH of the endolymph or perilymph, depending on where in the cochlea the non-natural Trk receptor agonist formulation is targeted. In some embodiments, from about 10 μM to about 200 mM concentration of a buffer is present in the gel formulation. In certain embodiments, from about a 5 mM to about a 200 mM concentration of a buffer is present. In certain embodiments, from about a 20 mM to about a 100 mM concentration of a buffer is present. In one embodiment is a buffer such as acetate or citrate at slightly acidic pH. In one embodiment the buffer is a sodium acetate buffer having a pH of about 4.5 to about 6.5. In one embodiment the buffer is a sodium citrate buffer having a pH of about 5.0 to about 8.0, or about 5.5 to about 7.0.

In an alternative embodiment, the buffer used is tris(hydroxymethyl)aminomethane, bicarbonate, carbonate, or phosphate at slightly basic pH. In one embodiment, the buffer is a sodium bicarbonate buffer having a pH of about 6.5 to about 8.5, or about 7.0 to about 8.0. In another embodiment the buffer is a sodium phosphate dibasic buffer having a pH of about 6.0 to about 9.0.

Also described herein are controlled-release formulations or devices comprising a non-natural Trk receptor agonist and a viscosity enhancing agent. Suitable viscosity-enhancing agents include by way of example only, gelling agents and suspending agents. In one embodiment, the enhanced viscosity formulation does not include a buffer. In other embodiments, the enhanced viscosity formulation includes a pharmaceutically acceptable buffer. Sodium chloride or other tonicity agents are optionally used to adjust tonicity, if necessary.

By way of example only, the auris-acceptable viscosity agent includes hydroxypropyl methylcellulose, hydroxyethyl cellulose, polyvinylpyrrolidone, carboxymethyl cellulose, polyvinyl alcohol, sodium chondroitin sulfate, sodium hyaluronate. Other viscosity enhancing agents compatible with the targeted auris structure include, but are not limited to, acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or combinations thereof. In specific embodiments, the viscosity-enhancing excipient is a combination of MCC and CMC. In another embodiment, the viscosity-enhancing agent is a combination of carboxymethylated chitosan, or chitin, and alginate. The combination of chitin and alginate with the non-natural Trk receptor agonists disclosed herein acts as a controlled-release formulation, restricting the diffusion of the non-natural Trk receptor agonists from the formulation. Moreover, the combination of carboxymethylated chitosan and alginate is optionally used to assist in increasing the permeability of the non-natural Trk receptor agonists through the round window membrane.

In some embodiments is an enhanced viscosity formulation, comprising from about 0.1 mM and about 100 mM of a non-natural Trk receptor agonist, a pharmaceutically acceptable viscosity agent, and water for injection, the concentration of the viscosity agent in the water being sufficient to provide an enhanced viscosity formulation with a final viscosity from about 100 to about 100,000 cP. In certain embodiments, the viscosity of the gel is in the range from about 100 to about 50,000 cP, about 100 cP to about 1,000 cP, about 500 cP to about 1500 cP, about 1000 cP to about 3000 cP, about 2000 cP to about 8,000 cP, about 4,000 cP to about 50,000 cP, about 10,000 cP to about 500,000 cP, about 15,000 cP to about 1,000,000 cP. In other embodiments, when an even more viscous medium is desired, the biocompatible gel comprises at least about 35%, at least about 45%, at least about 55%, at least about 65%, at least about 70%, at least about 75%, or even at least about 80% or so by weight of the non-natural Trk receptor agonist. In highly concentrated samples, the biocompatible enhanced viscosity formulation comprises at least about 25%, at least about 35%, at least about 45%, at least about 55%, at least about 65%, at least about 75%, at least about 85%, at least about 90%, at least about 95%, or more by weight of the non-natural Trk receptor agonist.

In some embodiments, the viscosity of the gel formulations presented herein are measured by any means described. For example, in some embodiments, an LVDV-II+CP Cone Plate Viscometer and a Cone Spindle CPE-40 is used to calculate the viscosity of the gel formulation described herein. In other embodiments, a Brookfield (spindle and cup) viscometer is used to calculate the viscosity of the gel formulation described herein. In some embodiments, the viscosity ranges referred to herein are measured at room temperature. In other embodiments, the viscosity ranges referred to herein are measured at body temperature (e.g., at the average body temperature of a healthy human).

In one embodiment, the pharmaceutically acceptable enhanced viscosity auris-acceptable formulation comprises at least one non-natural Trk receptor agonist and at least one gelling agent. Suitable gelling agents for use in preparation of the gel formulation include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum, and any combinations or mixtures thereof. In some other embodiments, hydroxypropylmethylcellulose (Methocel®) is utilized as the gelling agent. In certain embodiments, the viscosity enhancing agents described herein are also utilized as the gelling agent for the gel formulations presented herein.

In some embodiments, the non-natural Trk receptor agonists disclosed herein are dispensed as an auris-acceptable paint. As used herein, paints (also known as film formers) are solutions comprised of a solvent, a monomer or polymer, an active agent, and optionally one or more pharmaceutically-acceptable excipients. After application to a tissue, the solvent evaporates leaving behind a thin coating comprised of the monomers or polymers, and the non-natural Trk receptor agonist. The coating protects the non-natural Trk receptor agonists and maintains them in an immobilized state at the site of application. This decreases the amount of non-natural Trk receptor agonists which may be lost and correspondingly increases the amount delivered to the subject. By way of non-limiting example, paints include collodions (e.g. Flexible Collodion, USP), and solutions comprising saccharide siloxane copolymers and a cross-linking agent. Collodions are ethyl ether/ethanol solutions containing pyroxylin (a nitrocellulose). After application, the ethyl ether/ethanol solution evaporates leaving behind a thin film of pyroxylin. In solutions comprising saccharide siloxane copolymers, the saccharide siloxane copolymers form the coating after evaporation of the solvent initiates the cross-linking of the saccharide siloxane copolymers. For additional disclosures regarding paints, see Remington: The Science and Practice of Pharmacy which is hereby incorporated with respect to this subject matter. The paints contemplated for use herein are flexible such that they do not interfere with the propagation of pressure waves through the ear. Further, the paints may be applied as a liquid (i.e. solution, suspension, or emulsion), a semisolid (i.e. a gel, foam, paste, or jelly), or an aerosol.

In some embodiments, the non-natural Trk receptor agonists disclosed herein are dispensed as a controlled-release foam. Examples of suitable foamable carriers for use in the compositions disclosed herein include, but are not limited to, alginate and derivatives thereof; carboxymethylcellulose and derivatives thereof; collagen; polysaccharides, including, for example, dextran, dextran derivatives; pectin; starch; modified starches such as starches having additional carboxyl and/or carboxamide groups and/or having hydrophilic side-chains; cellulose and derivatives thereof; agar and derivatives thereof, such as agar stabilized with polyacrylamide; polyethylene oxides; glycol methacrylates; gelatin; gums such as xanthum, guar, karaya, gellan, arabic, tragacanth and locust bean gum; or any combinations thereof. Also suitable are the salts of the aforementioned carriers, for example, sodium alginate. The formulation optionally further comprises a foaming agent, which promotes the formation of the foam, including a surfactant or external propellant. Examples of suitable foaming agents include cetrimide, lecithin, soaps, silicones, and the like. Commercially available surfactants such as Tween® are also suitable.

In some embodiments, other gel formulations are useful depending upon the particular non-natural Trk receptor agonists, other pharmaceutical agent, or excipients/additives used, and as such are considered to fall within the scope of the present disclosure. For example, other commercially-available glycerin-based gels, glycerin-derived compounds, conjugated or crosslinked gels, matrices, hydrogels, and polymers, as well as gelatins and their derivatives, alginates, alginate-based gels, and even various native and synthetic hydrogel and hydrogel-derived compounds are all expected to be useful in the non-natural Trk receptor agonist formulations described herein. In some embodiments, auris-acceptable gels include, but are not limited to, alginate hydrogels SAF®-Gel (ConvaTec, Princeton, N.J.), Duoderm® Hydroactive Gel (ConvaTec), Nu-gel® (Johnson & Johnson Medical, Arlington, Tex.), Carrasyn® (V) Acemannan Hydrogel (Carrington Laboratories, Inc., Irving, Tex.) glycerin gels Elta® Hydrogel (Swiss-American Products, Inc., Dallas, Tex.), and K-Y® Sterile (Johnson & Johnson). In further embodiments, biodegradable biocompatible gels also represent compounds present in auris-acceptable formulations disclosed and described herein.

In some formulations developed for administration to a mammal, and for compositions formulated for human administration, the auris-acceptable gel comprises substantially all of the weight of the composition. In other embodiments, the auris-acceptable gel comprises as much as about 98% or about 99% of the composition by weight. This is desirable when a substantially non-fluid or substantially viscous formulation is needed. In a further embodiment, when slightly less viscous, or slightly more fluid auris-acceptable pharmaceutical gel formulations are desired, the biocompatible gel portion of the formulation comprises at least about 50% by weight, at least about 60% by weight, at least about 70% by weight, or even at least about 80% or 90% by weight of the compound. All intermediate integers within these ranges are contemplated to fall within the scope of this disclosure, and in some alternative embodiments, even more fluid (and consequently less viscous) auris-acceptable gel compositions are formulated, such as for example, those in which the gel or matrix component of the mixture comprises not more than about 50% by weight, not more than about 40% by weight, not more than about 30% by weight, or even those than comprise not more than about 15% or about 20% by weight of the composition.

### Auris-Acceptable Suspending Agents

In one embodiment, at least one non-natural Trk receptor agonist is included in a pharmaceutically acceptable enhanced viscosity formulation wherein the formulation further comprises at least one suspending agent, wherein the suspending agent assists in imparting controlled-release characteristics to the formulation. In some embodiments, suspending agents also serve to increase the viscosity of the auris-acceptable non-natural Trk receptor agonist formulations and compositions.

Suspending agents include, by way of example only, compounds such as polyvinylpyrrolidone, e.g., polyvinylpyrrolidone K12, polyvinylpyrrolidone K17, polyvinylpyrrolidone K25, or polyvinylpyrrolidone K30; vinyl pyrrolidone/vinyl acetate copolymer (S630); sodium carboxymethylcellulose; methylcellulose; hydroxypropylmethylcellulose (hypromellose); hydroxymethylcellulose acetate stearate; polysorbate-80; hydroxyethylcellulose; sodium alginate; gums, such as, e.g., gum tragacanth and gum acacia, guar gum; xanthans, including xanthan gum; sugars; cellulosics, such as, e.g., sodium carboxymethylcellulose, methylcellulose, sodium carboxymethylcellulose, hydroxypropylmethylcellulose, hydroxyethylcellulose; polysorbate-80; sodium alginate; polyethoxylated sorbitan monolaurate; polyethoxylated sorbitan monolaurate; povidone; and the like. In some embodiments, useful aqueous suspensions also contain one or more polymers as suspending agents. Useful polymers include water-soluble polymers such as cellulosic polymers, e.g., hydroxypropyl methylcellulose, and water-insoluble polymers such as cross-linked carboxyl-containing polymers.

In one embodiment, the present disclosure provides auris-acceptable gel compositions comprising a therapeutically effective amount of a non-natural Trk receptor agonist in a hydroxyethyl cellulose gel. Hydroxyethyl cellulose (HEC) is obtained as a dry powder which is reconstituted in water or an aqueous buffer solution to give the desired viscosity (generally about 200 cps to about 30,000 cps, corresponding to about 0.2% to about 10% HEC). In one embodiment the concentration of HEC is between about 1% and about 15%, about 1% and about 2%, or about 1.5% to about 2%.

In other embodiments, the auris-acceptable formulations, including gel formulations and viscosity-enhanced formulations, further include excipients; other medicinal or pharmaceutical agents; carriers; adjuvants; preserving, stabilizing, wetting or emulsifying agents; solution promoters; salts; solubilizers; an antifoaming agent; an antioxidant; a dispersing agent; a wetting agent; a surfactant; and any combinations thereof.

### Auris-Acceptable Actinic Radiation Curable Gel

In other embodiments, the gel is an actinic radiation curable gel, such that following administration to or near the targeted auris structure, use of actinic radiation (or light, including UV light, visible light, or infrared light) the desired gel properties are formed. By way of example only, fiber optics are used to provide the actinic radiation so as to form the desired gel properties. In some embodiments, the fiber optics and the gel administration device form a single unit. In other embodiments, the fiber optics and the gel administration device are provided separately.

### Auris-Acceptable Solvent Release Gel

In some embodiments, the gel is a solvent release gel such that the desired gel properties are formed after administration to or near the targeted auris structure, that is, as the solvent in the injected gel formulation diffuses out the gel, a gel having the desired gel properties is formed. For example, a formulation that comprises sucrose acetate isobutyrate, a pharmaceutically acceptable solvent, one or more additives, and the non-natural Trk receptor agonist is administered at or near the round window membrane; diffusion of the solvent out of the injected formulation provides a depot having the desired gel properties. For example, use of a water soluble solvent provides a high viscosity depot when the solvent diffuses rapidly out of the injected formulation. On the other hand, use of a hydrophobic solvent (e.g., benzyl benzoate) provides a less viscous depot. One example of an auris-acceptable solvent release gel formulation is the SABER™ Delivery System marketed by DURECT Corporation.

### Auris-Acceptable In Situ Forming Spongy Material

Also contemplated within the scope of the embodiments is the use of a spongy material, formed in situ in the auris interna or auris media. In some embodiments, the spongy material is formed from hyaluronic acid or its derivatives. The spongy material is impregnated with a non-natural Trk receptor agonist and placed within the auris media so as to provide controlled release of the non-natural Trk receptor agonist within the auris media or in contact with the round window membrane, so as to provide controlled release of the non-natural Trk receptor agonist into the auris interna. In some embodiments, the spongy material is biodegradable.

### Round Window Membrane Mucoadhesives

Also contemplated within the scope of the embodiments is the addition of a round window membrane mucoadhesive with the non-natural Trk receptor agonist formulations and compositions and devices disclosed herein. The term ‘mucoadhesion’ is used for materials that bind to the mucin layer of a biological membrane, such as the external membrane of the 3-layered round window membrane. To serve as round window membrane mucoadhesive polymers, the polymers possess some general physiochemical features such as predominantly anionic hydrophilicity with numerous hydrogen bond forming groups, suitable surface property for wetting mucus/mucosal tissue surfaces, or sufficient flexibility to penetrate the mucus network.

Round window membrane mucoadhesive agents that are used with the auris-acceptable formulations include, but are not limited to, at least one soluble polyvinylpyrrolidone polymer (PVP); a water-swellable, but water-insoluble, fibrous, cross-linked carboxy-functional polymer; a crosslinked poly(acrylic acid) (e.g. Carbopol® 947P); a carbomer homopolymer; a carbomer copolymer; a hydrophilic polysaccharide gum; maltodextrin; a cross-linked alginate gum gel; a water-dispersible polycarboxylated vinyl polymer; at least two particulate components selected from the group consisting of titanium dioxide, silicon dioxide, and clay; or a mixture thereof. The round window membrane mucoadhesive agent is optionally used in combination with an auris-acceptable viscosity increasing excipient, or used alone to increase the interaction of the composition with the mucosal layer target otic component. In one non-limiting example, the mucoadhesive agent is maltodextrin. In some embodiments, the mucoadhesive agent is an alginate gum. When used, the round window membrane mucoadhesive character imparted to the composition is at a level that is sufficient to deliver an effective amount of the non-natural Trk receptor agonist composition to, for example, the mucosal layer of round window membrane or the crista fenestrae cochleae in an amount that coats the mucosal membrane, and thereafter deliver the composition to the affected areas, including by way of example only, the vestibular and/or cochlear structures of the auris interna. When used, the mucoadhesive characteristics of the compositions provided herein are determined and, using this information (along with the other teachings provided herein), the appropriate amounts are determined. One method for determining sufficient mucoadhesiveness includes monitoring changes in the interaction of the composition with a mucosal layer, including, but not limited to, measuring changes in residence or retention time of the composition in the absence and presence of the mucoadhesive excipient.

Mucoadhesive agents have been described, for example, in U.S. Pat. Nos. 6,638,521; 6,562,363; 6,509,028; 6,348,502; 6,319,513; 6,306,789; 5,814,330; and 4,900,552, each of which is hereby incorporated by reference for such disclosure.

In another non-limiting example, a mucoadhesive agent is, for example, at least two particulate components selected from titanium dioxide, silicon dioxide, and clay, wherein the composition is not further diluted with any liquid prior to administration and the level of silicon dioxide, if present, is from about 3% to about 15%, by weight of the composition. Silicon dioxide, if present, includes fumed silicon dioxide, precipitated silicon dioxide, coacervated silicon dioxide, gel silicon dioxide, and mixtures thereof. Clay, if present, includes kaolin minerals, serpentine minerals, smectites, illite, or a mixture thereof. For example, clay includes laponite, bentonite, hectorite, saponite, montmorillonites, or a mixture thereof.

In one non-limiting example, the round window membrane mucoadhesive agent is maltodextrin. Maltodextrin is a carbohydrate produced by the hydrolysis of starch that is optionally derived from corn, potato, wheat, or other plant products. Maltodextrin is optionally used either alone or in combination with other round window membrane mucoadhesive agents to impart mucoadhesive characteristics on the compositions disclosed herein. In one embodiment, a combination of maltodextrin and a carbopol polymer are used to increase the round window membrane mucoadhesive characteristics of the compositions or devices disclosed herein.

In another embodiment, the round window membrane mucoadhesive agent is an alkyl-glycoside and/or a saccharide alkyl ester. As used herein, an “alkyl-glycoside” means a compound comprising any hydrophilic saccharide (e.g. sucrose, maltose, or glucose) linked to a hydrophobic alkyl. In some embodiments, the round window membrane mucoadhesive agent is an alkyl-glycoside wherein the alkyl-glycoside comprises a sugar linked to a hydrophobic alkyl (e.g., an alkyl comprising about 6 to about 25 carbon atoms) by an amide linkage, an amine linkage, a carbamate linkage, an ether linkage, a thioether linkage, an ester linkage, a thioester linkage, a glycosidic linkage, a thioglycosidic linkage, and/or a ureide linkage. In some embodiments, the round window membrane mucoadhesive agent is a hexyl-, heptyl-, octyl-, nonyl-, decyl-, undecyl-, dodecyl-, tridecyl-, tetradecyl, pentadecyl-, hexadecyl-, heptadecyl-, octadecyl α-D-, or octadecyl β-D-maltoside; hexyl-, heptyl-, octyl-, nonyl-, decyl-, undecyl-, dodecyl-, tridecyl-, tetradecyl, pentadecyl-, hexadecyl-, heptadecyl-, octadecyl α-D-, or octadecyl β-D-glucoside; hexyl-, heptyl-, octyl-, nonyl-, decyl-, undecyl-, dodecyl-, tridecyl-, tetradecyl, pentadecyl-, hexadecyl-, heptadecyl-, octadecyl α-D-, or octadecyl β-D-sucroside; hexyl-, heptyl-, octyl-, dodecyl-, tridecyl-, or tetradecyl-β-D-thiomaltoside; dodecyl maltoside; heptyl- or octyl-1-thio-α- or β-D-glucopyranoside; alkyl thiosucroses; alkyl maltotriosides; long chain aliphatic carbonic acid amides of sucrose β-amino-alkyl ethers; derivatives of palatinose or isomaltamine linked by an amide linkage to an alkyl chain and derivatives of isomaltamine linked by urea to an alkyl chain; long chain aliphatic carbonic acid ureides of sucrose β-amino-alkyl ethers and long chain aliphatic carbonic acid amides of sucrose β-amino-alkyl ethers, or any combination thereof. In some embodiments, the round window membrane mucoadhesive agent is an alkyl-glycoside wherein the alkyl glycoside is maltose, sucrose, glucose, or a combination thereof linked by a glycosidic linkage to an alkyl chain of 9-16 carbon atoms (e.g., nonyl-, decyl-, dodecyl- and tetradecyl sucroside; nonyl-, decyl-, dodecyl- and tetradecyl glucoside; and nonyl-, decyl-, dodecyl- and tetradecyl maltoside). In some embodiments, the round window membrane mucoadhesive agent is an alkyl-glycoside wherein the alkyl glycoside is dodecylmaltoside, tridecylmaltoside, and tetradecylmaltoside.

In some embodiments, the round window membrane mucoadhesive agent is an alkyl-glycoside wherein the alkyl-glycoside is a disaccharide with at least one glucose. In some embodiments, the auris acceptable penetration enhancer is a surfactant comprising α-D-glucopyranosyl-β-glycopyranoside, n-Dodecyl-4-O-α-D-glucopyranosyl-β-glycopyranoside, and/or n-tetradecyl-4-O-α-D-glucopyranosyl-β-glycopyranoside. In some embodiments, the round window membrane mucoadhesive agent is an alkyl-glycoside wherein the alkyl-glycoside has a critical micelle concentration (CMC) of less than about 1 mM in pure water or in aqueous solutions. In some embodiments, the round window membrane mucoadhesive agent is an alkyl-glycoside wherein an oxygen atom within the alkyl-glycoside is substituted with a sulfur atom. In some embodiments, the round window membrane mucoadhesive agent is an alkyl-glycoside wherein the alkylglycoside is the β anomer. In some embodiments, the round window membrane mucoadhesive agent is an alkyl-glycoside wherein the alkylglycoside comprises 90%, 91%, 92%, 93%, 94%, 95%, 96%, 97%, 98%, 99%, 99.1%, 99.5%, or 99.9% of the 0 anomer.

### Auris-Acceptable Controlled-Release Particles

Non-natural Trk receptor agonists and/or other pharmaceutical agents disclosed herein are optionally incorporated within controlled-release particles, lipid complexes, liposomes, nanoparticles, microparticles, microspheres, coacervates, nanocapsules, or other agents which enhance or facilitate the localized delivery of the non-natural Trk receptor agonists. In some embodiments, a single enhanced viscosity formulation is used, in which at least one non-natural Trk receptor agonist is present, while in other embodiments, a pharmaceutical formulation that comprises a mixture of two or more distinct enhanced viscosity formulations is used, in which at least a non-natural Trk receptor agonist is present. In some embodiments, combinations of sols, gels, and/or biocompatible matrices is also employed to provide desirable characteristics of the controlled-release non-natural Trk receptor agonist compositions or formulations. In certain embodiments, the controlled-release non-natural Trk receptor agonist formulations or compositions are cross-linked by one or more agents to alter or improve the properties of the composition.

Examples of microspheres relevant to the pharmaceutical formulations disclosed herein include: Luzzi, L. A., J. Pharm. Psy. 59:1367 (1970); U.S. Pat. No. 4,530,840; Lewis, D. H., “Controlled Release of Bioactive Agents from Lactides/Glycolide Polymers” in Biodegradable Polymers as Drug Delivery Systems, Chasin, M. and Langer, R., eds., Marcel Decker (1990); U.S. Pat. No. 4,675,189; Beck et al., “Poly(lactic acid) and Poly(lactic acid-co-glycolic acid) Contraceptive Delivery Systems,” in Long Acting Steroid Contraception, Mishell, D. R., ed., Raven Press (1983); U.S. Pat. Nos. 4,758,435; 3,773,919; 4,474,572. Examples of protein therapeutics formulated as microspheres include: U.S. Pat. Nos. 6,458,387; 6,268,053; 6,090,925; 5,981,719; and 5,578,709, and are herein incorporated by reference for such disclosure.

Microspheres usually have a spherical shape, although irregularly-shaped microparticles are possible. Microspheres may vary in size, ranging from submicron to 1000 micron diameters. Microspheres suitable for use with the auris-acceptable formulations disclosed herein are submicron to 250 micron diameter microspheres, allowing administration by injection with a standard gauge needle. The auris-acceptable microspheres are prepared by any method which produces microspheres in a size range acceptable for use in an injectable composition. Injection is optionally accomplished with standard gauge needles used for administering liquid compositions.

Suitable examples of polymeric matrix materials for use in the auris-acceptable controlled-release particles herein include poly(glycolic acid), poly-d,l-lactic acid, poly-l-lactic acid, copolymers of the foregoing, poly(aliphatic carboxylic acids), copolyoxalates, polycaprolactone, polydioxonene, poly(orthocarbonates), poly(acetals), poly(lactic acid-caprolactone), polyorthoesters, poly(glycolic acid-caprolactone), polydioxonene, polyanhydrides, polyphosphazines, and natural polymers including albumin, casein, and some waxes, such as, glycerol mono- and distearate, and the like. Various commercially available poly (lactide-co-glycolide) materials (PLGA) are optionally used in the method disclosed herein. For example, poly (d,l-lactic-co-glycolic acid) is commercially available from Boehringer-Ingelheim as RESOMER RG 503 H. This product has a mole percent composition of 50% lactide and 50% glycolide. These copolymers are available in a wide range of molecular weights and ratios of lactic acid to glycolic acid. One embodiment includes the use of the polymer poly(d,l-lactide-co-glycolide). The molar ratio of lactide to glycolide in such a copolymer includes the range of from about 95:5 to about 50:50.

The molecular weight of the polymeric matrix material is of some importance. The molecular weight should be high enough so that it forms satisfactory polymer coatings, i.e., the polymer should be a good film former. Usually, a satisfactory molecular weight is in the range of 5,000 to 500,000 daltons. The molecular weight of a polymer is also important from the point of view that molecular weight influences the biodegradation rate of the polymer. For a diffusional mechanism of drug release, the polymer should remain intact until all of the drug is released from the microparticles and then degrade. The non-natural Trk receptor agonist is also released from the microparticles as the polymeric excipient bioerodes. By an appropriate selection of polymeric materials a microsphere formulation is made such that the resulting microspheres exhibit both diffusional release and biodegradation release properties. This is useful in affording multiphasic release patterns.

A variety of methods are known by which compounds are encapsulated in microspheres. In these methods, the non-natural Trk receptor agonist is generally dispersed or emulsified, using stirrers, agitators, or other dynamic mixing techniques, in a solvent containing a wall-forming material. Solvent is then removed from the microspheres, and thereafter the microsphere product is obtained.

In one embodiment, controlled-release non-natural Trk receptor agonist formulations are made through the incorporation of the non-natural Trk receptor agonists and/or other pharmaceutical agents into ethylene-vinyl acetate copolymer matrices. (See U.S. Pat. No. 6,083,534, incorporated herein for such disclosure). In another embodiment, non-natural Trk receptor agonists are incorporated into poly (lactic-glycolic acid) or poly-L-lactic acid microspheres. Id. In yet another embodiment, the non-natural Trk receptor agonists are encapsulated into alginate microspheres. (See U.S. Pat. No. 6,036,978, incorporated herein for such disclosure). Biocompatible methacrylate-based polymers to encapsulate the non-natural Trk receptor agonist compounds or compositions are optionally used in the formulations and methods disclosed herein. A wide range of methacrylate-based polymer systems are commercially available, such as the EUDRAGIT polymers marketed by Evonik. One useful aspect of methacrylate polymers is that the properties of the formulation are varied by incorporating various co-polymers. For example, poly(acrylic acid-co-methylmethacrylate) microparticles exhibit enhanced mucoadhesion properties as the carboxylic acid groups in the poly(acrylic acid) form hydrogen bonds with mucin (Park et al, Pharm. Res. (1987) 4(6):457-464). Variation of the ratio between acrylic acid and methylmethacrylate monomers serves to modulate the properties of the co-polymer. Methacrylate-based microparticles have also been used in protein therapeutic formulations (Naha et al, Journal of Microencapsulation 4 Feb. 2008 (online publication)). In one embodiment, the enhanced viscosity auris-acceptable formulations described herein comprises non-natural Trk receptor agonist microspheres wherein the microspheres are formed from a methacrylate polymer or copolymer. In an additional embodiment, the enhanced viscosity formulation described herein comprises non-natural Trk receptor agonist microspheres wherein the microspheres are mucoadhesive. Other controlled-release systems, including incorporation or deposit of polymeric materials or matrices onto solid or hollow spheres containing non-natural Trk receptor agonists, are also explicitly contemplated within the embodiments disclosed herein. The types of controlled-release systems available without significantly losing activity of the non-natural Trk receptor agonists are determined using the teachings, examples, and principles disclosed herein

An example of a conventional microencapsulation process for pharmaceutical preparations is shown in U.S. Pat. No. 3,737,337, incorporated herein by reference for such disclosure. The non-natural Trk receptor agonist substances to be encapsulated or embedded are dissolved or dispersed in the organic solution of the polymer (phase A), using conventional mixers, including (in the preparation of dispersion) vibrators and high-speed stirrers, etc. The dispersion of phase (A), containing the core material in solution or in suspension, is carried out in the aqueous phase (B), again using conventional mixers, such as high-speed mixers, vibration mixers, or even spray nozzles, in which case the particle size of the microspheres will be determined not only by the concentration of phase (A), but also by the emulsate or microsphere size. With conventional techniques for the microencapsulation of non-natural Trk receptor agonists, the microspheres form when the solvent containing an active agent and a polymer is emulsified or dispersed in an immiscible solution by stirring, agitating, vibrating, or some other dynamic mixing technique, often for a relatively long period of time.

Methods for the construction of microspheres are also described in U.S. Pat. Nos. 4,389,330, and 4,530,840, incorporated herein by reference for such disclosure. The desired non-natural Trk receptor agonist is dissolved or dispersed in an appropriate solvent. To the agent-containing medium is added the polymeric matrix material in an amount relative to the active ingredient which gives a product of the desired loading of non-natural Trk receptor agonist. Optionally, all of the ingredients of the non-natural Trk receptor agonist microsphere product can be blended in the solvent medium together. Suitable solvents for the agonist and the polymeric matrix material include organic solvents such as acetone; halogenated hydrocarbons such as chloroform, methylene chloride, and the like; aromatic hydrocarbon compounds; halogenated aromatic hydrocarbon compounds; cyclic ethers; alcohols; ethyl acetate; and the like.

The mixture of ingredients in the solvent is emulsified in a continuous-phase processing medium; the continuous-phase medium being such that a dispersion of microdroplets containing the indicated ingredients is formed in the continuous-phase medium. Naturally, the continuous-phase processing medium and the organic solvent must be immiscible and includes water although nonaqueous media such as xylene and toluene and synthetic oils and natural oils are optionally used. Optionally, a surfactant is added to the continuous-phase processing medium to prevent the microparticles from agglomerating and to control the size of the solvent microdroplets in the emulsion. A preferred surfactant-dispersing medium combination is a 1 to 10 wt. % poly (vinyl alcohol) in water mixture. The dispersion is formed by mechanical agitation of the mixed materials. An emulsion is optionally formed by adding small drops of the non-natural Trk receptor agonist-wall forming material solution to the continuous phase processing medium. The temperature during the formation of the emulsion is not especially critical but influences the size and quality of the microspheres and the solubility of the drug in the continuous phase. It is desirable to have as little of the non-natural Trk receptor agonist in the continuous phase as possible. Moreover, depending on the solvent and continuous-phase processing medium employed, the temperature must not be too low or the solvent and processing medium will solidify or the processing medium will become too viscous for practical purposes, or too high that the processing medium will evaporate, or that the liquid processing medium will not be maintained. Moreover, the temperature of the medium cannot be so high that the stability of the particular agent being incorporated in the microspheres is adversely affected. Accordingly, the dispersion process is conducted at any temperature which maintains stable operating conditions, which preferred temperature being about 15° C. to 60° C., depending upon the drug and excipient selected.

The dispersion which is formed is a stable emulsion and from this dispersion the organic solvent immiscible fluid is optionally partially removed in the first step of the solvent removal process. The solvent is removed by techniques such as heating, the application of a reduced pressure or a combination of both. The temperature employed to evaporate solvent from the microdroplets is not critical, but should not be that high that it degrades the non-natural Trk receptor agonist employed in the preparation of a given microparticle, nor should it be so high as to evaporate solvent at such a rapid rate to cause defects in the wall forming material. Generally, from 5 to 75%, of the solvent is removed in the first solvent removal step.

After the first stage, the dispersed microparticles in the solvent immiscible fluid medium are isolated from the fluid medium by any convenient means of separation. Thus, for example, the fluid is decanted from the microsphere or the microsphere suspension is filtered. Still other various combinations of separation techniques are used if desired.

Following the isolation of the microspheres from the continuous-phase processing medium, the remainder of the solvent in the microspheres is removed by extraction. In this step, the microspheres are suspended in the same continuous-phase processing medium used in step one, with or without surfactant, or in another liquid. The extraction medium removes the solvent from the microspheres and yet does not dissolve the microspheres. During the extraction, the extraction medium with dissolved solvent is optionally removed and replaced with fresh extraction medium. This is best done on a continual basis. The rate of extraction medium replenishment of a given process is a variable which is determined at the time the process is performed and, therefore, no precise limits for the rate must be predetermined. After the majority of the solvent has been removed from the microspheres, the microspheres are dried by exposure to air or by other conventional drying techniques such as vacuum drying, drying over a desiccant, or the like. This process is very efficient in encapsulating the non-natural Trk receptor agonist since core loadings of up to 80 wt. %, preferably up to 60 wt. %, are obtained.

Alternatively, controlled-release microspheres containing non-natural Trk receptor agonist are prepared through the use of static mixers. Static or motionless mixers consist of a conduit or tube in which is received a number of static mixing agents. Static mixers provide homogeneous mixing in a relatively short length of conduit, and in a relatively short period of time. With static mixers, the fluid moves through the mixer, rather than some part of the mixer, such as a blade, moving through the fluid.

A static mixer is optionally used to create an emulsion. When using a static mixer to form an emulsion, several factors determine emulsion particle size, including the density and viscosity of the various solutions or phases to be mixed, volume ratio of the phases, interfacial tension between the phases, static mixer parameters (conduit diameter; length of mixing element; number of mixing elements), and linear velocity through the static mixer. Temperature is a variable because it affects density, viscosity, and interfacial tension. The controlling variables are linear velocity, sheer rate, and pressure drop per unit length of static mixer.

In order to create microspheres containing non-natural Trk receptor agonist using a static mixer process, an organic phase and an aqueous phase are combined. The organic and aqueous phases are largely or substantially immiscible, with the aqueous phase constituting the continuous phase of the emulsion. The organic phase includes non-natural Trk receptor agonist as well as a wall-forming polymer or polymeric matrix material. The organic phase is prepared by dissolving a non-natural Trk receptor agonist in an organic or other suitable solvent, or by forming a dispersion or an emulsion containing the non-natural Trk receptor agonist. The organic phase and the aqueous phase are pumped so that the two phases flow simultaneously through a static mixer, thereby forming an emulsion which comprises microspheres containing the non-natural Trk receptor agonist encapsulated in the polymeric matrix material. The organic and aqueous phases are pumped through the static mixer into a large volume of quench liquid to extract or remove the organic solvent. Organic solvent is optionally removed from the microspheres while they are washing or being stirred in the quench liquid. After the microspheres are washed in a quench liquid, they are isolated, as through a sieve, and dried.

In one embodiment, microspheres are prepared using a static mixer. The process is not limited to the solvent extraction technique discussed above, but is used with other encapsulation techniques. For example, the process is optionally used with a phase separation encapsulation technique. To do so, an organic phase is prepared that comprises a non-natural Trk receptor agonist suspended or dispersed in a polymer solution. The non-solvent second phase is free from solvents for the polymer and active agent. A preferred non-solvent second phase is silicone oil. The organic phase and the non-solvent phase are pumped through a static mixer into a non-solvent quench liquid, such as heptane. The semi-solid particles are quenched for complete hardening and washing. The process of microencapsulation includes spray drying, solvent evaporation, a combination of evaporation and extraction, and melt extrusion.

In another embodiment, the microencapsulation process involves the use of a static mixer with a single solvent. This process is described in detail in U.S. application Ser. No. 08/338,805, herein incorporated by reference for such disclosure. An alternative process involves the use of a static mixer with co-solvents. In this process, biodegradable microspheres comprising a biodegradable polymeric binder and a non-natural Trk receptor agonist are prepared, which comprises a blend of at least two substantially non-toxic solvents, free of halogenated hydrocarbons to dissolve both the agent and the polymer. The solvent blend containing the dissolved agent and polymer is dispersed in an aqueous solution to form droplets. The resulting emulsion is then added to an aqueous extraction medium preferably containing at least one of the solvents of the blend, whereby the rate of extraction of each solvent is controlled, whereupon the biodegradable microspheres containing the non-natural Trk receptor agonist are formed. This process has the advantage that less extraction medium is required because the solubility of one solvent in water is substantially independent of the other and solvent selection is increased, especially with solvents that are particularly difficult to extract.

Nanoparticles are also contemplated for use with the non-natural Trk receptor agonists disclosed herein. Nanoparticles are material structures of about 100 nm or less in size. One use of nanoparticles in pharmaceutical formulations is the formation of suspensions as the interaction of the particle surface with solvent is strong enough to overcome differences in density. Nanoparticle suspensions are sterilized as the nanoparticles are small enough to be subjected to sterilizing filtration (see, e.g., U.S. Pat. No. 6,139,870, herein incorporated by reference for such disclosure). Nanoparticles comprise at least one hydrophobic, water-insoluble and water-indispersible polymer or copolymer emulsified in a solution or aqueous dispersion of surfactants, phospholipids, or fatty acids. The non-natural Trk receptor agonist is optionally introduced with the polymer or the copolymer into the nanoparticles.

Lipid nanocapsules as controlled-release structures, as well for penetrating the round window membrane and reaching auris interna and/or auris media targets, is also contemplated herein. Lipid nanocapsules are optionally formed by emulsifying capric and caprylic acid triglycerides (Labrafac WL 1349; avg. mw 512), soybean lecithin (LIPOID® S75-3; 69% phosphatidylcholine and other phospholipids), surfactant (for example, Solutol HS 15), a mixture of polyethylene glycol 660 hydroxystearate and free polyethylene glycol 660, NaCl, and water. The mixture is stirred at room temperature to obtain an oil emulsion in water. After progressive heating at a rate of 4° C./min under magnetic stirring, a short interval of transparency should occur close to 70° C., and the inverted phase (water droplets in oil) obtained at 85° C. Three cycles of cooling and heating is then applied between 85° C. and 60° C. at the rate of 4° C./min, and a fast dilution in cold water at a temperature close to 0° C. to produce a suspension of nanocapsules. To encapsulate the non-natural Trk receptor agonist, the agonist is optionally added just prior to the dilution with cold water.

Non-natural Trk receptor agonists are also inserted into the lipid nanocapsules by incubation for 90 minutes with an aqueous micellar solution of the non-natural Trk receptor agonist. The suspension is then vortexed every 15 minutes and then quenched in an ice bath for 1 minute.

Suitable auris-acceptable surfactants are, by way of example, cholic acid or taurocholic acid salts. Taurocholic acid, the conjugate formed from cholic acid and taurine, is a fully metabolizable sulfonic acid surfactant. An analog of taurocholic acid, tauroursodeoxycholic acid (TUDCA), is a naturally occurring bile acid and is a conjugate of taurine and ursodeoxycholic acid (UDCA). Other naturally occurring anionic (e.g., galactocerebroside sulfate), neutral (e.g., lactosylceramide), or zwitterionic surfactants (e.g., sphingomyelin, phosphatidyl choline, palmitoyl carnitine) are optionally used to prepare nanoparticles.

The auris-acceptable phospholipids are chosen, by way of example, from natural, synthetic, or semi-synthetic phospholipids; lecithins (phosphatidylcholine) such as, for example, purified egg or soya lecithins (lecithin E100, lecithin E80 and phospholipons, for example phospholipon 90); phosphatidylethanolamine; phosphatidylserine; phosphatidylinositol; phosphatidylglycerol; dipalmitoylphosphatidylcholine; dipalmitoylglycerophosphatidylcholine; dimyristoylphosphatidylcholine; distearoylphosphatidylcholine; phosphatidic acid; or mixtures thereof are used more particularly.

Fatty acids for use with the auris-acceptable formulations are chosen from, by way of example, lauric acid, mysristic acid, palmitic acid, stearic acid, isostearic acid, arachidic acid, behenic acid, oleic acid, myristoleic acid, palmitoleic acid, linoleic acid, alpha-linoleic acid, arachidonic acid, eicosapentaenoic acid, erucic acid, docosahexaenoic acid, and the like.

Suitable auris-acceptable surfactants are selected from known organic and inorganic pharmaceutical excipients. Such excipients include various polymers, low molecular weight oligomers, natural products, and surfactants. Preferred surface modifiers include nonionic and ionic surfactants. Two or more surface modifiers are used in combination.

Representative examples of auris-acceptable surfactants include cetyl pyridinium chloride, gelatin, casein, lecithin (phosphatides), dextran, glycerol, gum acacia, cholesterol, tragacanth, stearic acid, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, dodecyl trimethyl ammonium bromide, polyoxyethylenestearates, colloidal silicon dioxide, phosphates, sodium dodecylsulfate, carboxymethylcellulose calcium, hydroxypropyl cellulose (HPC, HPC-SL, and HPC-L), hydroxypropyl methylcellulose (HPMC), carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol (PVA), polyvinylpyrrolidone (PVP), 4-(1,1,3,3-tetaamethylbutyl)-phenol polymer with ethylene oxide and formaldehyde (also known as tyloxapol, superione, and triton), poloxamers, poloxamnines, a charged phospholipid such as dimyristoyl phosphatidyl glycerol, dioctylsulfosuccinate (DOSS), Tetronic® 1508, dialkylesters of sodium sulfosuccinic acid, Duponol P, Tritons X-200, Crodestas F-110, p-isononylphenoxypoly-(glycidol), Crodestas SL-40 (Croda, Inc.), SA9OHCO (which is C₁₈ H₃₇ CH₂ (CON(CH₃)—CH₂ (CHOH)₄ (CH₂ OH)₂ (Eastman Kodak Co.)), decanoyl-N-methylglucamide, n-decyl β-D-glucopyranoside, n-decyl β-D-maltopyranoside, n-dodecyl β-D-glucopyranoside, n-dodecyl β-D-maltoside, heptanoyl-N-methylglucamide, n-heptyl-β-D-glucopyranoside, n-heptyl β-D-thioglucoside, n-hexyl β-D-glucopyranoside, nonanoyl-N-methylglucamide, n-noyl β-D-glucopyranoside, octanoyl-N-methylglucamide, n-octyl-β-D-glucopyranoside, octyl β-D-thioglucopyranoside, and the like. Most of these surfactants are known pharmaceutical excipients and are described in detail in the Handbook of Pharmaceutical Excipients, published jointly by the American Pharmaceutical Association and The Pharmaceutical Society of Great Britain (The Pharmaceutical Press, 1986), specifically incorporated by reference for such disclosure.

The hydrophobic, water-insoluble, and water-indispersible polymer or copolymer may be chosen from biocompatible and biodegradable polymers, for example lactic or glycolic acid polymers and copolymers thereof, or polylactic/polyethylene (or polypropylene) oxide copolymers, preferably with molecular weights of between 1000 and 200,000, polyhydroxybutyric acid polymers, polylactones of fatty acids containing at least 12 carbon atoms, or polyanhydrides.

The nanoparticles may be obtained by coacervation, or by the technique of evaporation of solvent, from an aqueous dispersion or solution of phospholipids and of an oleic acid salt into which is added an immiscible organic phase comprising the active principle and the hydrophobic, water-insoluble and water-indispersible polymer or copolymer. The mixture is pre-emulsified and then subjected to homogenization and evaporation of the organic solvent to obtain an aqueous suspension of very small-sized nanoparticles.

A variety of methods are optionally employed to fabricate the non-natural Trk receptor agonist nanoparticles that are within the scope of the embodiments. These methods include vaporization methods, such as free jet expansion, laser vaporization, spark erosion, electro explosion, and chemical vapor deposition; physical methods involving mechanical attrition (e.g., “pearlmilling” technology, Elan Nanosystems), super critical CO2 and interfacial deposition following solvent displacement. In one embodiment, the solvent displacement method is used. The size of nanoparticles produced by this method is sensitive to the concentration of polymer in the organic solvent; the rate of mixing; and to the surfactant employed in the process. Continuous flow mixers provide the necessary turbulence to ensure small particle size. One type of continuous flow mixing device that is optionally used to prepare nanoparticles has been described (Hansen et al J Phys Chem 92, 2189-96, 1988). In other embodiments, ultrasonic devices, flow through homogenizers, or supercritical CO2 devices may be used to prepare nanoparticles.

If suitable nanoparticle homogeneity is not obtained on direct synthesis, then size-exclusion chromatography is used to produce highly uniform drug-containing particles that are freed of other components involved in their fabrication. Size-exclusion chromatography (SEC) techniques, such as gel-filtration chromatography, is used to separate particle-bound non-natural Trk receptor agonist or other pharmaceutical compound from free non-natural Trk receptor agonist or other pharmaceutical compound, or to select a suitable size range of non-natural Trk receptor agonist—containing nanoparticles. Various SEC media, such as Superdex 200, Superose 6, Sephacryl 1000 are commercially available and are employed for the size-based fractionation of such mixtures. Additionally, nanoparticles are optionally purified by centrifugation, membrane filtration and by use of other molecular sieving devices, crosslinked gels/materials and membranes.

### Auris-Acceptable Cyclodextrin and Other Stabilizing Formulations

In a specific embodiment, the auris-acceptable formulations alternatively comprises a cyclodextrin. Cyclodextrins are cyclic oligosaccharides containing 6, 7, or 8 glucopyranose units, referred to as as α-cyclodextrin, β-cyclodextrin, or γ-cyclodextrin respectively. Cyclodextrins have a hydrophilic exterior, which enhances water-soluble, and a hydrophobic interior which forms a cavity. In an aqueous environment, hydrophobic portions of other molecules often enter the hydrophobic cavity of cyclodextrin to form inclusion compounds. Additionally, cyclodextrins are also capable of other types of nonbonding interactions with molecules that are not inside the hydrophobic cavity. Cyclodextrins have three free hydroxyl groups for each glucopyranose unit, or 18 hydroxyl groups on α-cyclodextrin, 21 hydroxyl groups on β-cyclodextrin, and 24 hydroxyl groups on γ-cyclodextrin. One or more of these hydroxyl groups can be reacted with any of a number of reagents to form a large variety of cyclodextrin derivatives, including hydroxypropyl ethers, sulfonates, and sulfoalkylethers. Shown below is the structure of β-cyclodextrin and the hydroxypropyl-β-cyclodextrin (HPβCD).

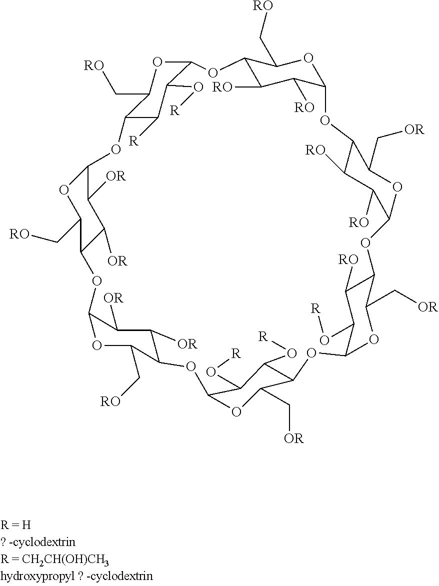

In some embodiments, the use of cyclodextrins in the pharmaceutical compositions described herein improves the solubility of the drug. Inclusion compounds are involved in many cases of enhanced solubility; however other interactions between cyclodextrins and insoluble compounds also improves solubility. Hydroxypropyl-β-cyclodextrin (HPβCD) is commercially available as a pyrogen free product. It is a nonhygroscopic white powder that readily dissolves in water. HPβCD is thermally stable and does not degrade at neutral pH. Thus, cyclodextrins improve the solubility of a therapeutic agent in a composition or formulation. Accordingly, in some embodiments, cyclodextrins are included to increase the solubility of the auris-acceptable non-natural Trk receptor agonists within the formulations described herein. In other embodiments, cyclodextrins in addition serve as controlled-release excipients within the formulations described herein.

By way of example only, cyclodextrin derivatives for use include α-cyclodextrin, β-cyclodextrin, γ-cyclodextrin, hydroxyethyl-β-cyclodextrin, hydroxypropyl γ-cyclodextrin, sulfated β-cyclodextrin, sulfated α-cyclodextrin, and sulfobutyl ether β-cyclodextrin.

The concentration of the cyclodextrin used in the compositions and methods disclosed herein varies according to the physiochemical properties, pharmacokinetic properties, side effect or adverse events, formulation considerations, or other factors associated with the therapeutically active agent, or a salt or prodrug thereof, or with the properties of other excipients in the composition. Thus, in certain circumstances, the concentration or amount of cyclodextrin used in accordance with the compositions and methods disclosed herein will vary, depending on the need. When used, the amount of cyclodextrins needed to increase solubility of the non-natural Trk receptor agonist and/or function as a controlled-release excipient in any of the formulations described herein is selected using the principles, examples, and teachings described herein.

Other stabilizers that are useful in the auris-acceptable formulations disclosed herein include, for example, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, and combinations thereof. In some embodiments, amide analogues of stabilizers are also used. In further embodiments, the chosen stabilizer changes the hydrophobicity of the formulation (e.g., oleic acid, waxes), or improves the mixing of various components in the formulation (e.g., ethanol), controls the moisture level in the formula (e.g., PVP or polyvinyl pyrrolidone), controls the mobility of the phase (substances with melting points higher than room temperature such as long chain fatty acids, alcohols, esters, ethers, amides etc. or mixtures thereof; waxes), and/or improves the compatibility of the formula with encapsulating materials (e.g., oleic acid or wax). In another embodiment some of these stabilizers are used as solvents/co-solvents (e.g., ethanol). In other embodiments, stabilizers are present in sufficient amounts to inhibit the degradation of the non-natural Trk receptor agonist. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v polysorbate 20, (h) arginine, (i) heparin, (j) dextran sulfate, (k) cyclodextrins, (l) pentosan polysulfate and other heparinoids, (m) divalent cations such as magnesium and zinc, or (n) combinations thereof.

Additional useful non-natural Trk receptor agonist auris-acceptable formulations include one or more anti-aggregation additives to enhance stability of non-natural Trk receptor agonist formulations by reducing the rate of protein aggregation. The anti-aggregation additive selected depends upon the nature of the conditions to which the non-natural Trk receptor agonist, for example non-natural Trk receptor agonist antibodies are exposed. For example, certain formulations undergoing agitation and thermal stress require a different anti-aggregation additive than a formulation undergoing lyophilization and reconstitution. Useful anti-aggregation additives include, by way of example only, urea, guanidinium chloride, simple amino acids such as glycine or arginine, sugars, polyalcohols, polysorbates, polymers such as polyethylene glycol and dextrans, alkyl saccharides, such as alkyl glycoside, and surfactants.

Other useful formulations optionally include one or more auris-acceptable antioxidants to enhance chemical stability where required. Suitable antioxidants include, by way of example only, ascorbic acid, methionine, sodium thiosulfate and sodium metabisulfite. In one embodiment, antioxidants are selected from metal chelating agents, thiol containing compounds and other general stabilizing agents.

Still other useful compositions include one or more auris-acceptable surfactants to enhance physical stability or for other purposes. Suitable nonionic surfactants include, but are not limited to, polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

In some embodiments, the auris-acceptable pharmaceutical formulations described herein are stable with respect to compound degradation over a period of any of at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 7 weeks, at least about 8 weeks, at least about 3 months, at least about 4 months, at least about 5 months, or at least about 6 months. In other embodiments, the formulations described herein are stable with respect to compound degradation over a period of at least about 1 week. Also described herein are formulations that are stable with respect to compound degradation over a period of at least about 1 month.

In other embodiments, an additional surfactant (co-surfactant) and/or buffering agent is combined with one or more of the pharmaceutically acceptable vehicles previously described herein so that the surfactant and/or buffering agent maintains the product at an optimal pH for stability. Suitable co-surfactants include, but are not limited to: a) natural and synthetic lipophilic agents, e.g., phospholipids, cholesterol, and cholesterol fatty acid esters and derivatives thereof; b) nonionic surfactants, which include for example, polyoxyethylene fatty alcohol esters, sorbitan fatty acid esters (Spans), polyoxyethylene sorbitan fatty acid esters (e.g., polyoxyethylene (20) sorbitan monooleate (Tween 80), polyoxyethylene (20) sorbitan monostearate (Tween 60), polyoxyethylene (20) sorbitan monolaurate (Tween 20) and other Tweens, sorbitan esters, glycerol esters, e.g., Myrj and glycerol triacetate (triacetin), polyethylene glycols, cetyl alcohol, cetostearyl alcohol, stearyl alcohol, polysorbate 80, poloxamers, poloxamines, polyoxyethylene castor oil derivatives (e.g., Cremophor® RH40, Cremphor A25, Cremphor A20, Cremophor® EL) and other Cremophors, sulfosuccinates, alkyl sulphates (SLS); PEG glyceryl fatty acid esters such as PEG-8 glyceryl caprylate/caprate (Labrasol), PEG-4 glyceryl caprylate/caprate (Labrafac Hydro WL 1219), PEG-32 glyceryl laurate (Gelucire 444/14), PEG-6 glyceryl mono oleate (Labrafil M 1944 CS), PEG-6 glyceryl linoleate (Labrafil M 2125 CS); propylene glycol mono- and di-fatty acid esters, such as propylene glycol laurate, propylene glycol caprylate/caprate; Brij® 700, ascorbyl-6-palmitate, stearylamine, sodium lauryl sulfate, polyoxethyleneglycerol triiricinoleate, and any combinations or mixtures thereof, c) anionic surfactants include, but are not limited to, calcium carboxymethylcellulose, sodium carboxymethylcellulose, sodium sulfosuccinate, dioctyl, sodium alginate, alkyl polyoxyethylene sulfates, sodium lauryl sulfate, triethanolamine stearate, potassium laurate, bile salts, and any combinations or mixtures thereof, and d) cationic surfactants such as cetyltrimethylammonium bromide, and lauryldimethylbenzyl-ammonium chloride.

In a further embodiment, when one or more co-surfactants are utilized in the auris-acceptable formulations of the present disclosure, they are combined, e.g., with a pharmaceutically acceptable vehicle and is present in the final formulation, e.g., in an amount ranging from about 0.1% to about 20%, from about 0.5% to about 10%.

In one embodiment, the surfactant has an HLB value of 0 to 20. In additional embodiments, the surfactant has an HLB value of 0 to 3, of 4 to 6, of 7 to 9, of 8 to 18, of 13 to 15, of 10 to 18.

In one embodiment, diluents are also used to stabilize the non-natural Trk receptor agonist or other pharmaceutical compounds because they provide a more stable environment. Salts dissolved in buffered solutions (which also can provide pH control or maintenance) are utilized as diluents, including, but not limited to a phosphate buffered saline solution. In other embodiments, the gel formulation is isotonic with the endolymph or the perilymph: depending on the portion of the cochlea that the non-natural Trk receptor agonist formulation is targeted. Isotonic formulations are provided by the addition of a tonicity agent. Suitable tonicity agents include, but are not limited to any pharmaceutically acceptable sugar, salt or any combinations or mixtures thereof, such as, but not limited to dextrose and sodium chloride. In further embodiments, the tonicity agents are present in an amount from about 100 mOsm/kg to about 500 mOsm/kg. In some embodiments, the tonicity agent is present in an amount from about 200 mOsm/kg to about 400 mOsm/kg, from about 280 mOsm/kg to about 320 mOsm/kg. The amount of tonicity agents will depend on the target structure of the pharmaceutical formulation, as described herein.

Useful tonicity compositions also include one or more salts in an amount required to bring osmolality of the composition into an acceptable range for the perilymph or the endolymph. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite, and ammonium sulfate.

In some embodiments, the auris-acceptable gel formulations disclosed herein alternatively or additionally contains preservatives to prevent microbial growth. Suitable auris-acceptable preservatives for use in the enhanced viscosity formulations described herein include, but are not limited to benzoic acid, boric acid, p-hydroxybenzoates, alcohols, quarternary compounds, stabilized chlorine dioxide, mercurials, such as merfen and thiomersal, mixtures of the foregoing and the like.

In a further embodiment, the preservative is, by way of example only, an antimicrobial agent, within the auris-acceptable formulations presented herein. In one embodiment, the formulation includes a preservative such as by way of example only, methyl paraben, sodium bisulfite, sodium thiosulfate, ascorbate, chlorobutanol, thimerosal, parabens, benzyl alcohol, phenylethanol, and others. In another embodiment, the methyl paraben is at a concentration of about 0.05% to about 1.0%, about 0.1% to about 0.2%. In a further embodiment, the gel is prepared by mixing water, methylparaben, hydroxyethylcellulose and sodium citrate. In a further embodiment, the gel is prepared by mixing water, methylparaben, hydroxyethylcellulose, and sodium acetate. In a further embodiment, the mixture is sterilized by autoclaving at 120° C. for about 20 minutes, and tested for pH, methylparaben concentration and viscosity before mixing with the appropriate amount of the non-natural Trk receptor agonist disclosed herein.

Suitable auris-acceptable water soluble preservatives which are employed in the drug delivery vehicle include sodium bisulfite, sodium thiosulfate, ascorbate, chlorobutanol, thimerosal, parabens, benzyl alcohol, Butylated hydroxytoluene (BHT), phenylethanol, and others. These agents are present, generally, in amounts of about 0.001% to about 5% by weight and, preferably, in the amount of about 0.01 to about 2% by weight. In some embodiments, auris-compatible formulations described herein are free of preservatives.

### Round Window Membrane Penetration Enhancers

In another embodiment, the formulation further comprises one or more round window membrane penetration enhancers. Penetration across the round window membrane is enhanced by the presence of round window membrane penetration enhancers. Round window membrane penetration enhancers are chemical entities that facilitate transport of coadministered substances across the round window membrane. Round window membrane penetration enhancers are grouped according to chemical structure. Surfactants, both ionic and non-ionic, such as sodium lauryl sulfate, sodium laurate, polyoxyethylene-20-cetyl ether, laureth-9, sodium dodecylsulfate, dioctyl sodium sulfosuccinate, polyoxyethylene-9-lauryl ether (PLE), Tween® 80, nonylphenoxypolyethylene (NP-POE), polysorbates and the like, function as round window membrane penetration enhancers. Bile salts (such as sodium glycocholate, sodium deoxycholate, sodium taurocholate, sodium taurodihydrofusidate, sodium glycodihydrofusidate and the like), fatty acids and derivatives (such as oleic acid, caprylic acid, mono- and di-glycerides, lauric acids, acylcholines, caprylic acids, acylcarnitines, sodium caprates and the like), chelating agents (such as EDTA, citric acid, salicylates and the like), sulfoxides (such as dimethyl sulfoxide (DMSO), decylmethyl sulfoxide and the like), and alcohols (such as ethanol, isopropanol, glycerol, propanediol and the like) also function as round window membrane penetration enhancers.

In some embodiments, the auris acceptable penetration enhancer is a surfactant comprising an alkyl-glycoside wherein the alkyl glycoside is tetradecyl-β-D-maltoside. In some embodiments, the auris acceptable penetration enhancer is a surfactant comprising an alkyl-glycoside wherein the alkyl glycoside is dodecyl-maltoside. In certain instances, the penetration enhancing agent is a hyaluronidase. In certain instances, a hyaluronidase is a human or bovine hyaluronidase. In some instances, a hyaluronidase is a human hyaluronidase (e.g., hyaluronidase found in human sperm, PH20 (Halozyme), Hyelenex® (Baxter International, Inc.)). In some instances, a hyaluronidase is a bovine hyaluronidase (e.g., bovine testicular hyaluronidase, Amphadase® (Amphastar Pharmaceuticals), Hydase® (PrimaPharm, Inc). In some instances, a hyluronidase is an ovine hyaluronidase, Vitrase® (ISTA Pharmaceuticals). In certain instances, a hyaluronidase described herein is a recombinant hyaluronidase. In some instances, a hyaluronidase described herein is a humanized recombinant hyaluronidase. In some instances, a hyaluronidase described herein is a pegylated hyaluronidase (e.g., PEGPH20 (Halozyme)). In addition, the peptide-like penetration enhancers described in U.S. Pat. Nos. 7,151,191; 6,221,367; and 5,714,167, herein incorporated by references for such disclosure, are contemplated as an additional embodiment. These penetration enhancers are amino-acid and peptide derivatives and enable drug absorption by passive transcellular diffusion without affecting the integrity of membranes or intercellular tight junctions.

### Round Window Membrane Permeable Liposomes

Liposomes or lipid particles may also be employed to encapsulate the non-natural Trk receptor agonist formulations or compositions. Phospholipids that are gently dispersed in an aqueous medium form multilayer vesicles with areas of entrapped aqueous media separating the lipid layers. Sonication, or turbulent agitation, of these multilayer vesicles results in the formation of single layer vesicles, commonly referred to as liposomes, with sizes of about 10-1000 nm. These liposomes have many advantages as non-natural Trk receptor agonists or other pharmaceutical agent carriers. They are biologically inert, biodegradable, non-toxic and non-antigenic. Liposomes are formed in various sizes and with varying compositions and surface properties. Additionally, they are able to entrap a wide variety of agents and release the agent at the site of liposome collapse.

Suitable phospholipids for use in auris-acceptable liposomes here are, for example, phosphatidyl cholines, ethanolamines and serines, sphingomyelins, cardiolipins, plasmalogens, phosphatidic acids and cerebrosides, in particular those which are soluble together with the non-natural Trk receptor agonists herein in non-toxic, pharmaceutically acceptable organic solvents. Preferred phospholipids are, for example, phosphatidyl choline, phosphatidyl ethanolamine, phosphatidyl serine, phosphatidyl inositol, lysophosphatidyl choline, phosphatidyl glycerol and the like, and mixtures thereof especially lecithin, e.g. soya lecithin. The amount of phospholipid used in the present formulation range from about 10 to about 30%, preferably from about 15 to about 25% and in particular is about 20%.

Lipophilic additives may be employed advantageously to modify selectively the characteristics of the liposomes. Examples of such additives include by way of example only, stearylamine, phosphatidic acid, tocopherol, cholesterol, cholesterol hemisuccinate and lanolin extracts. The amount of lipophilic additive used range from 0.5 to 8%, preferably from 1.5 to 4% and in particular is about 2%. Generally, the ratio of the amount of lipophilic additive to the amount of phospholipid ranges from about 1:8 to about 1:12 and in particular is about 1:10. Said phospholipid, lipophilic additive and the non-natural Trk receptor agonist and other pharmaceutical compounds are employed in conjunction with a non-toxic, pharmaceutically acceptable organic solvent system which dissolve said ingredients. Said solvent system not only must dissolve the non-natural Trk receptor agonist completely, but it also has to allow the formulation of stable single bilayered liposomes. The solvent system comprises dimethylisosorbide and tetraglycol (glycofurol, tetrahydrofurfuryl alcohol polyethylene glycol ether) in an amount of about 8 to about 30%. In said solvent system, the ratio of the amount of dimethylisosorbide to the amount of tetraglycol range from about 2:1 to about 1:3, in particular from about 1:1 to about 1:2.5 and preferably is about 1:2. The amount of tetraglycol in the final composition thus vary from 5 to 20%, in particular from 5 to 15% and preferably is approximately 10%. The amount of dimethylisosorbide in the final composition thus range from 3 to 10%, in particular from 3 to 7% and preferably is approximately 5%.

The term “organic component” as used hereinafter refers to mixtures comprising said phospholipid, lipophilic additives and organic solvents. The non-natural Trk receptor agonist may be dissolved in the organic component, or other means to maintain full activity of the agent. The amount of non-natural Trk receptor agonist in the final formulation may range from 0.1 to 5.0%. In addition, other ingredients such as anti-oxidants may be added to the organic component. Examples include tocopherol, butylated hydroxyanisole, butylated hydroxytoluene, ascorbyl palmitate, ascorbyl oleate and the like.

Liposomal formulations are alternatively prepared, for non-natural Trk receptor agonist or other pharmaceutical agents that are moderately heat-resistant, by (a) heating the phospholipid and the organic solvent system to about 60-80° C. in a vessel, dissolving the active ingredient, then adding any additional formulating agents, and stirring the mixture until complete dissolution is obtained; (b) heating the aqueous solution to 90-95° C. in a second vessel and dissolving the preservatives therein, allowing the mixture to cool and then adding the remainder of the auxiliary formulating agents and the remainder of the water, and stirring the mixture until complete dissolution is obtained; thus preparing the aqueous component; (c) transferring the organic phase directly into the aqueous component, while homogenizing the combination with a high performance mixing apparatus, for example, a high-shear mixer; and (d) adding a viscosity enhancing agent to the resulting mixture while further homogenizing. The aqueous component is optionally placed in a suitable vessel which is equipped with a homogenizer and homogenization is effected by creating turbulence during the injection of the organic component. Any mixing means or homogenizer which exerts high shear forces on the mixture may be employed. Generally, a mixer capable of speeds from about 1,500 to 20,000 rpm, in particular from about 3,000 to about 6,000 rpm may be employed. Suitable viscosity enhancing agents for use in process step (d) are for example, xanthan gum, hydroxypropyl cellulose, hydroxypropyl methylcellulose or mixtures thereof. The amount of viscosity enhancing agent depends on the nature and the concentration of the other ingredients and in general ranges from about 0.5 to 2.0%, or approximately 1.5%. In order to prevent degradation of the materials used during the preparation of the liposomal formulation, it is advantageous to purge all solutions with an inert gas such as nitrogen or argon, and to conduct all steps under an inert atmosphere. Liposomes prepared by the above described method usually contain most of the active ingredient bound in the lipid bilayer and separation of the liposomes from unencapsulated material is not required.

In other embodiments, the auris-acceptable formulations, including gel formulations and viscosity-enhanced formulations, further include excipients, other medicinal or pharmaceutical agents, carriers, adjuvants, such as preserving, stabilizing, wetting or emulsifying agents, solution promoters, salts, solubilizers, an antifoaming agent, an antioxidant, a dispersing agent, a wetting agent, a surfactant, and combinations thereof.

Suitable carriers for use in an auris-acceptable formulation described herein include, but are not limited to, any pharmaceutically acceptable solvent compatible with the targeted auris structure's physiological environment. In other embodiments, the base is a combination of a pharmaceutically acceptable surfactant and solvent.

In some embodiments, other excipients include, sodium stearyl fumarate, diethanolamine cetyl sulfate, isostearate, polyethoxylated castor oil, nonoxyl 10, octoxynol 9, sodium lauryl sulfate, sorbitan esters (sorbitan monolaurate, sorbitan monooleate, sorbitan monopalmitate, sorbitan monostearate, sorbitan sesquioleate, sorbitan trioleate, sorbitan tristearate, sorbitan laurate, sorbitan oleate, sorbitan palmitate, sorbitan stearate, sorbitan dioleate, sorbitan sesqui-isostearate, sorbitan sesquistearate, sorbitan tri-isostearate), lecithin pharmaceutical acceptable salts thereof and combinations or mixtures thereof.

In other embodiments, the carrier is a polysorbate. Polysorbates are nonionic surfactants of sorbitan esters. Polysorbates useful in the present disclosure include, but are not limited to polysorbate 20, polysorbate 40, polysorbate 60, polysorbate 80 (Tween 80) and any combinations or mixtures thereof. In further embodiments, polysorbate 80 is utilized as the pharmaceutically acceptable carrier.

In one embodiment, water-soluble glycerin-based auris-acceptable enhanced viscosity formulations utilized in the preparation of pharmaceutical delivery vehicles comprise at least one non-natural Trk receptor agonist containing at least about 0.1% of the water-soluble glycerin compound or more. In some embodiments, the percentage of non-natural Trk receptor agonist is varied between about 1% and about 95%, between about 5% and about 80%, between about 10% and about 60% or more of the weight or volume of the total pharmaceutical formulation. In some embodiments, the amount of the compound(s) in each therapeutically useful non-natural Trk receptor agonist formulation is prepared in such a way that a suitable dosage will be obtained in any given unit dose of the compound. Factors such as solubility, bioavailability, biological half-life, route of administration, product shelf life, as well as other pharmacological considerations are contemplated herein.

If desired, the auris-acceptable pharmaceutical gels also contain co-solvents, preservatives, cosolvents, ionic strength and osmolality adjustors and other excipients in addition to buffering agents. Suitable auris-acceptable water soluble buffering agents are alkali or alkaline earth metal carbonates, phosphates, bicarbonates, citrates, borates, acetates, succinates and the like, such as sodium phosphate, citrate, borate, acetate, bicarbonate, carbonate and tromethamine (TRIS). These agents are present in amounts sufficient to maintain the pH of the system at 7.4±0.2 and preferably, 7.4. As such, the buffering agent is as much as 5% on a weight basis of the total composition.

Cosolvents are used to enhance non-natural Trk receptor agonist solubility, however, some non-natural Trk receptor agonist or other pharmaceutical compounds are insoluble. These are often suspended in the polymer vehicle with the aid of suitable suspending or viscosity enhancing agents.

Moreover, some pharmaceutical excipients, diluents or carriers are potentially ototoxic. For example, benzalkonium chloride, a common preservative, is ototoxic and therefore potentially harmful if introduced into the vestibular or cochlear structures. In formulating a controlled-release non-natural Trk receptor agonist formulation, it is advised to avoid or combine the appropriate excipients, diluents or carriers to lessen or eliminate potential ototoxic components from the formulation, or to decrease the amount of such excipients, diluents or carriers. Optionally, a controlled-release non-natural Trk receptor agonist formulation includes otoprotective agents, such as antioxidants, alpha lipoic acid, calcium, fosfomycin or iron chelators, to counteract potential ototoxic effects that may arise from the use of specific therapeutic agents or excipients, diluents or carriers.

Therapeutically Acceptable Otic Formulations:

| Example Formulation | Example Characteristics |
|---|---|
| Chitosan | tunable degradation of matrix in vitro |
| glycerophosphate | tunable TACE inhibitor release in vitro: |
| (CGP) | e.g., ~50% of drug released after 24 hrs |
| | biodegradable |
| | compatible with drug delivery to the inner ear |
| | suitable for macromolecules and hydrophobic |
| | drugs |
| PEG-PLGA-PEG | tunable high stability: e.g., maintains |
| triblock | mechanical integrity >1 month in vitro |
| polymers | tunable fast release of hydrophilic drugs: |
| | e.g., ~50% of drug released after 24 hrs, and |
| | remainder released over ~5 days |
| | tunable slow release of hydrophobic drugs: |
| | e.g., ~80% released after 8 weeks |
| | biodegradable |
| | subcutaneous injection of solution: e.g., |
| | gel forms within seconds and is intact |
| | after 1 month |
| PEO-PPO-PEO | Tunable sol-gel transition temperature: e.g., |
| triblock copolymers | decreases with increasing F127 concentration |
| (e.g., Pluronic | |
| or Poloxameres) | |
| (e.g., F127) | |
| Chitosan | CGP formulation tolerates liposomes: e.g., up to |
| glycerophosphate | 15 uM/ml liposomes. |
| with drug-loaded | liposomes tunably reduce drug release time |
| liposomes | (e.g., up to 2 weeks in vitro). |
| | increase in liposome diameter optionally reduces |
| | drug release kinetics (e.g., liposome size |
| | between 100 and 300 nm) |
| | release parameters are controlled by changing |
| | composition of liposomes |

The formulations disclosed herein alternatively encompass an otoprotectant agent in addition to the at least one non-natural Trk receptor agonist and/or excipients, including but not limited to such agents as antioxidants, alpha lipoic acid, calcium, fosfomycin or iron chelators, to counteract potential ototoxic effects that may arise from the use of specific therapeutic agents or excipients, diluents or carriers.

### Triglycerides

Provided herein, in some embodiments, are otic formulations and compositions comprising a non-natural Trk receptor agonist and triglycerides. Triglycerides are esters derived from glycerol and three fatty acids. In some instances, these fatty acids are saturated fatty acids, unsaturated fatty acids, or a combination thereof.

In some instances, these triglycerides are medium chain triglycerides (MCTs) or long chain triglycerides (LCTs). In some embodiments, these triglycerides comprise medium chain fatty acids or long chain fatty acids. In some embodiments, these triglycerides comprise medium chain fatty acids and long chain fatty acids.

In some embodiments, the triglycerides are derived from glycerol and medium chain fatty acids. In some embodiments, each medium chain fatty acid independently comprises 6 to 12 carbon atoms in the carbon chain. In some embodiments, each medium chain fatty acid independently comprises 8 to 12 carbon atoms in the carbon chain. In some embodiments, each medium chain fatty acid independently comprises 6, 7, 8, 9, 10, 11, or 12 carbon atoms in the carbon chain. In some embodiments, each medium chain fatty acid independently comprises 8 or 10 carbon atoms in the carbon chain. In some embodiments, the medium chain fatty acids are caproic acid (hexanoic acid), enanthic acid (heptanoic acid), caprylic acid (octanoic acid), pelargonic acid (nonanoic acid), capric acid (decanoic acid), undecylenic acid (undec-10-enoic acid), lauric acid (dodecanoic acid), or a combination thereof. In some embodiments, the medium chain fatty acids are caprylic acid (octanoic acid), capric acid (decanoic acid), or a combination thereof.

In some embodiments, the triglycerides comprising medium chain fatty acids are balassee oil, coconut oil, cohune oil, palm kernel oil, tucum oil, or combinations thereof. In some embodiments, the triglycerides comprising medium chain fatty acids are balassee oil. In some embodiments, the triglycerides comprising medium chain fatty acids are coconut oil. In some embodiments, the triglycerides comprising medium chain fatty acids are cohune oil. In some embodiments, the triglycerides comprising medium chain fatty acids are palm kernel oil. In some embodiments, the triglycerides comprising medium chain fatty acids are tucum oil.

In some embodiments, the triglycerides are derived from glycerol and long chain fatty acids. In some embodiments, each long chain fatty acid independently comprises greater than 12 carbon atoms in the carbon chain. In some embodiments, each long chain fatty acid independently comprises 13 to 38 carbon atoms in the carbon chain. In some embodiments, each long chain fatty acid independently comprises 13 to 24 carbon atoms in the carbon chain. In some embodiments, each long chain fatty acid comprises 13, 14, 15, 16, 17, 18, 19, 20, 21, 22, 23, 24, 25, 26, 27, 28, 29, 30, 31, 32, 33, 34, 35, 36, 37, or 38 carbon atoms in the carbon chain. In some embodiments, the long chain fatty acids are saturated long chain fatty acids, unsaturated long chain fatty acids, or a combination thereof.

In some embodiments, the long chain fatty acids are tridecylic acid (tridecanoic acid), myristic acid (tetradecanoic acid), pentadecylic acid (pentadecanoic acid), palmitic acid (hexadecanoic acid), margaric acid (heptadecanoic acid), stearic acid (octadecanoic acid), nonadecylic acid (nonadecanoic acid), arachidic acid (eicosanoic acid), heneicosylic acid (heneicosanoic acid), behenic acid (docosanoic acid), tricosylic acid (tricosanoic acid), lignoceric acid (tetracosanoic acid), pentacosylic acid (pentacosanoic acid), cerotic acid (hexacosanoic acid), heptacosylic acid (heptacosanoic acid), montanic acid (octacosanoic acid), nonacosylic acid (nonacosanoic acid), melissic acid (triacontanoic acid), henatriacontylic acid (henatriacontanoic acid), lacceroic acid (dotriacontanoic acid), psyllic acid (tritriacontanoic acid), geddic acid (tetratriacontanoic acid), ceroplastic acid (pentatriacontanoic acid), hexatriacontylic acid (hexatriacontanoic acid), heptatriacontanoic acid (heptatriacontanoic acid), octatriacontanoic acid or a combination thereof. In some embodiments, the long chain fatty acids are α-linolenic acid, stearidonic acid, eicosapentaenoic acid, docosahexaenoic acid, linoleic acid, γ-linolenic acid, dihomo-γ-linolenic acid, arachidonic acid, docosatetraenoic acid, palmitoleic acid, vaccenic acid, paullinic acid, oleic acid, elaidic acid, gondoic acid, erucic acid, nervonic acid, mead acid, or a combination thereof. In some embodiments, the long chain fatty acids are tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, heptatriacontanoic acid, octatriacontanoic acid, α-linolenic acid, stearidonic acid, eicosapentaenoic acid, docosahexaenoic acid, linoleic acid, γ-linolenic acid, dihomo-γ-linolenic acid, arachidonic acid, docosatetraenoic acid, palmitoleic acid, vaccenic acid, paullinic acid, oleic acid, elaidic acid, gondoic acid, erucic acid, nervonic acid, mead acid, or a combination thereof.

In some embodiments, the triglycerides comprising long chain fatty acids are corn oil, peanut oil, safflower oil, soybean oil, sunflower seed oil, sesame oil, olive oil, castor oil, cotton seed oil, and fish oil, or combinations thereof. In some embodiments, the triglycerides comprising long chain fatty acids are corn oil. In some embodiments, the triglycerides comprising long chain fatty acids are peanut oil. In some embodiments, the triglycerides comprising long chain fatty acids are safflower oil. In some embodiments, the triglycerides comprising long chain fatty acids are soybean oil. In some embodiments, the triglycerides comprising long chain fatty acids are sunflower seed oil. In some embodiments, the triglycerides comprising long chain fatty acids are sesame oil. In some embodiments, the triglycerides comprising long chain fatty acids are olive oil. In some embodiments, the triglycerides comprising long chain fatty acids are castor oil. In some embodiments, the triglycerides comprising long chain fatty acids are cotton seed oil. In some embodiments, the triglycerides comprising long chain fatty acids are fish oil.

In some embodiments, the otic pharmaceutical formulation has triglycerides in an amount that is sufficient to stabilize the therapeutic agent for injection into the ear. In some embodiments, the otic pharmaceutical formulation has triglycerides in an amount that is sufficient to provide sufficient retention time in the ear. In some embodiments, the ear is the outer ear, middle ear, or inner ear. In some embodiments, the otic pharmaceutical formulation has triglycerides in an amount that is sufficient to provide sustained release of the therapeutic agent.

In some embodiments, the otic pharmaceutical formulation comprises between about 50% to about 99.9% by weight of the triglycerides. In some embodiments, the otic pharmaceutical formulation comprises between about 55% to about 99.9% by weight of the triglycerides. In some embodiments, the otic pharmaceutical formulation comprises between about 60% to about 99.9% by weight of the triglycerides. In some embodiments, the otic pharmaceutical formulation comprises between about 65% to about 99.9% by weight of the triglycerides. In some embodiments, the otic pharmaceutical formulation comprises between about 70% to about 99.9% by weight of the triglycerides. In some embodiments, the otic pharmaceutical formulation comprises between about 75% to about 99.9% by weight of the triglycerides. In some embodiments, the otic pharmaceutical formulation comprises between about 80% to about 99.9% by the weight of the triglycerides. In some embodiments, the otic pharmaceutical formulation comprises between about 85% to about 99.9% by the weight of the triglycerides. In some embodiments, the otic pharmaceutical formulation comprises between about 90% to about 99.9% by the weight of the triglycerides. In some embodiments, the otic pharmaceutical formulation comprises between about 95% to about 99.9% by the weight of the triglycerides.

In some embodiments, the otic pharmaceutical formulation comprises between about 50% to about 99.99% by weight of the triglycerides. In some embodiments, the otic pharmaceutical formulation comprises between about 55% to about 99.99% by weight of the triglycerides. In some embodiments, the otic pharmaceutical formulation comprises between about 60% to about 99.99% by weight of the triglycerides. In some embodiments, the otic pharmaceutical formulation comprises between about 65% to about 99.99% by weight of the triglycerides. In some embodiments, the otic pharmaceutical formulation comprises between about 70% to about 99.99% by weight of the triglycerides. In some embodiments, the otic pharmaceutical formulation comprises between about 75% to about 99.99% by weight of the triglycerides. In some embodiments, the otic pharmaceutical formulation comprises between about 80% to about 99.99% by weight of the triglycerides. In some embodiments, the otic pharmaceutical formulation comprises between about 85% to about 99.99% by weight of the triglycerides. In some embodiments, the otic pharmaceutical formulation comprises between about 90% to about 99.99% by weight of the triglycerides. In some embodiments, the otic pharmaceutical formulation comprises between about 95% to about 99.99% by weight of the triglycerides.

In some embodiments, the otic pharmaceutical formulation comprises between about 50% to about 95% by weight of the triglycerides. In some embodiments, the otic pharmaceutical formulation comprises between about 55% to about 95% by weight of the triglycerides. In some embodiments, the otic pharmaceutical formulation comprises between about 60% to about 95% by weight of the triglycerides. In some embodiments, the otic pharmaceutical formulation comprises between about 65% to about 95% by weight of the triglycerides. In some embodiments, the otic pharmaceutical formulation comprises between about 70% to about 95% by weight of the triglycerides. In some embodiments, the otic pharmaceutical formulation comprises between about 75% to about 95% by weight of the triglycerides. In some embodiments, the otic pharmaceutical formulation comprises between about 80% to about 95% by weight of the triglycerides. In some embodiments, the otic pharmaceutical formulation comprises between about 85% to about 95% by weight of the triglycerides. In some embodiments, the otic pharmaceutical formulation comprises between about 90% to about 95% by weight of the triglycerides.

In some embodiments, the otic pharmaceutical formulation comprises about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% about 99% by weight of the triglycerides.

In some embodiments, the triglycerides in any one of the otic formulations and compositions described herein are replaced with at least one of the following components in the corresponding amounts of triglyceride in the formulation or composition disclosed herein: mineral oil or any corresponding higher alkanes; Vaseline (petroleum jelly); silicone oil (polydimethylsiloxane) in different molecular weights; beeswax dissolved in any of the oils disclosed herein.

### Dosing Methods and Schedules

Drugs delivered to the inner ear have been administered systemically via oral, intravenous or intramuscular routes. However, systemic administration for pathologies local to the inner ear increases the likelihood of systemic toxicities and adverse side effects and creates a non-productive distribution of drug in which high levels of drug are found in the serum and correspondingly lower levels are found at the inner ear.

Intratympanic injection of therapeutic agents is the technique of injecting a therapeutic agent behind the tympanic membrane into the middle and/or inner ear. In one embodiment, the non-natural Trk receptor agonist formulations described herein are administered directly onto the round window membrane via transtympanic injection. In another embodiment, the non-natural Trk receptor agonist auris-acceptable formulations described herein are administered onto the round window membrane via a non-transtympanic approach to the inner ear. In additional embodiments, the non-natural Trk receptor agonist auris-acceptable formulation described herein is administered onto the round window membrane via a surgical approach to the round window membrane comprising modification of the crista fenestrae cochleae.

In one embodiment the delivery system is a syringe and needle apparatus that is capable of piercing the tympanic membrane and directly accessing the round window membrane or crista fenestrae cochleae of the auris interna. In some embodiments, the needle on the syringe is wider than a 18 gauge needle. In another embodiment, the needle gauge is from 18 gauge to 31 gauge. In a further embodiment, the needle gauge is from 25 gauge to 30 gauge. Depending upon the thickness or viscosity of the non-natural Trk receptor agonist compositions or formulations, the gauge level of the syringe or hypodermic needle may be varied accordingly. In another embodiment, the internal diameter of the needle can be increased by reducing the wall thickness of the needle (commonly referred as thin wall or extra thin wall needles) to reduce the possibility of needle clogging while maintaining an adequate needle gauge.

In another embodiment, the needle is a hypodermic needle used for instant delivery of the gel formulation. The hypodermic needle may be a single use needle or a disposable needle. In some embodiments, a syringe may be used for delivery of the pharmaceutically acceptable gel-based non-natural Trk receptor agonist-containing compositions as disclosed herein wherein the syringe has a press-fit (Luer) or twist-on (Luer-lock) fitting. In one embodiment, the syringe is a hypodermic syringe. In another embodiment, the syringe is made of plastic or glass. In yet another embodiment, the hypodermic syringe is a single use syringe. In a further embodiment, the glass syringe is capable of being sterilized. In yet a further embodiment, the sterilization occurs through an autoclave. In another embodiment, the syringe comprises a cylindrical syringe body wherein the gel formulation is stored before use. In other embodiments, the syringe comprises a cylindrical syringe body wherein the non-natural Trk receptor agonist pharmaceutically acceptable gel-based compositions as disclosed herein is stored before use which conveniently allows for mixing with a suitable pharmaceutically acceptable buffer. In other embodiments, the syringe may contain other excipients, stabilizers, suspending agents, diluents or a combination thereof to stabilize or otherwise stably store the non-natural Trk receptor agonist or other pharmaceutical compounds contained therein.

In some embodiments, the syringe comprises a cylindrical syringe body wherein the body is compartmentalized in that each compartment is able to store at least one component of the auris-acceptable non-natural Trk receptor agonist gel formulation. In a further embodiment, the syringe having a compartmentalized body allows for mixing of the components prior to injection into the auris media or auris interna. In other embodiments, the delivery system comprises multiple syringes, each syringe of the multiple syringes contains at least one component of the gel formulation such that each component is pre-mixed prior to injection or is mixed subsequent to injection. In a further embodiment, the syringes disclosed herein comprise at least one reservoir wherein the at least one reservoir comprises a non-natural Trk receptor agonist, or a pharmaceutically acceptable buffer, or a viscosity enhancing agent, such as a gelling agent or a combination thereof. Commercially available injection devices are optionally employed in their simplest form as ready-to-use plastic syringes with a syringe barrel, needle assembly with a needle, plunger with a plunger rod, and holding flange, to perform an intratympanic injection.

In some embodiments, the delivery device is an apparatus designed for administration of therapeutic agents to the middle and/or inner ear. By way of example only: GYRUS Medical Gmbh offers micro-otoscopes for visualization of and drug delivery to the round window niche; Arenberg has described a medical treatment device to deliver fluids to inner ear structures in U.S. Pat. Nos. 5,421,818; 5,474,529; and 5,476,446, each of which is incorporated by reference herein for such disclosure. U.S. patent application Ser. No. 08/874,208, which is incorporated herein by reference for such disclosure, describes a surgical method for implanting a fluid transfer conduit to deliver therapeutic agents to the inner ear. U.S. Patent Application Publication 2007/0167918, which is incorporated herein by reference for such disclosure, further describes a combined otic aspirator and medication dispenser for intratympanic fluid sampling and medicament application.

The auris-acceptable compositions or formulations containing the non-natural Trk receptor agonists described herein are administered for prophylactic and/or therapeutic treatments. In therapeutic applications, the non-natural Trk receptor agonist compositions are administered to a patient already suffering from a disease, condition or disorder, in an amount sufficient to cure or at least partially arrest the symptoms of the disease, disorder or condition. Amounts effective for this use will depend on the severity and course of the disease, disorder or condition, previous therapy, the patient's health status and response to the drugs, and the judgment of the treating physician.

### Frequency of Administration

In some embodiments, a composition disclosed herein is administered to an individual in need thereof once. In some embodiments, a composition disclosed herein is administered to an individual in need thereof more than once. In some embodiments, a first administration of a composition disclosed herein is followed by a second administration of a composition disclosed herein. In some embodiments, a first administration of a composition disclosed herein is followed by a second and third administration of a composition disclosed herein. In some embodiments, a first administration of a composition disclosed herein is followed by a second, third, and fourth administration of a composition disclosed herein. In some embodiments, a first administration of a composition disclosed herein is followed by a second, third, fourth, and fifth administration of a composition disclosed herein. In some embodiments, a first administration of a composition disclosed herein is followed by a drug holiday.

The number of times a composition is administered to an individual in need thereof depends on the discretion of a medical professional, the disorder, the severity of the disorder, and the individual's response to the formulation. In some embodiments, a composition disclosed herein is administered once to an individual in need thereof with a mild acute condition. In some embodiments, a composition disclosed herein is administered more than once to an individual in need thereof with a moderate or severe acute condition. In the case wherein the patient's condition does not improve, upon the doctor's discretion the administration of an auris sensory cell modulator may be administered chronically, that is, for an extended period of time, including throughout the duration of the patient's life in order to ameliorate or otherwise control or limit the symptoms of the patient's disease or condition.

In the case wherein the patient's condition does not improve, upon the doctor's discretion the administration of the non-natural Trk receptor agonist compounds may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the non-natural Trk receptor agonist is given continuously; alternatively, the dose of drug being administered is temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's otic conditions has occurred, a maintenance non-natural Trk receptor agonist dose is administered if necessary. Subsequently, the dosage or the frequency of administration, or both, is optionally reduced, as a function of the symptoms, to a level at which the improved disease, disorder or condition is retained. In certain embodiments, patients require intermittent treatment on a long-term basis upon any recurrence of symptoms.

The amount of non-natural Trk receptor agonist that will correspond to such an amount will vary depending upon factors such as the particular compound, disease condition and its severity, according to the particular circumstances surrounding the case, including, e.g., the specific non-natural Trk receptor agonist being administered, the route of administration, the autoimmune condition being treated, the target area being treated, and the subject or host being treated. In general, however, doses employed for adult human treatment will typically be in the range of 0.02-50 mg per administration, preferably 1-15 mg per administration. The desired dose is presented in a single dose or as divided doses administered simultaneously (or over a short period of time) or at appropriate intervals.

In some embodiments, the initial administration is a particular non-natural Trk receptor agonist and the subsequent administration a different formulation of a non-natural Trk receptor agonist.

### Pharmacokinetics of Controlled-Release Formulations

In one embodiment, the formulations disclosed herein additionally provides an immediate release of a non-natural Trk receptor agonist from the composition, or within 1 minute, or within 5 minutes, or within 10 minutes, or within 15 minutes, or within 30 minutes, or within 60 minutes or within 90 minutes. In other embodiments, a therapeutically effective amount of at least one non-natural Trk receptor agonist is released from the composition immediately, or within 1 minute, or within 5 minutes, or within 10 minutes, or within 15 minutes, or within 30 minutes, or within 60 minutes or within 90 minutes. In certain embodiments the composition comprises an auris-pharmaceutically acceptable gel formulation providing immediate release of at least one non-natural Trk receptor agonist. Additional embodiments of the formulation also includes an agent that enhances the viscosity of the formulations included herein.

In other or further embodiments, the formulation provides an extended release formulation of at least one non-natural Trk receptor agonist. In certain embodiments, diffusion of at least one non-natural Trk receptor agonist from the formulation occurs for a time period exceeding 5 minutes, or 15 minutes, or 30 minutes, or 1 hour, or 4 hours, or 6 hours, or 12 hours, or 18 hours, or 1 day, or 2 days, or 3 days, or 4 days, or 5 days, or 6 days, or 7 days, or 10 days, or 12 days, or 14 days, or 18 days, or 21 days, or 25 days, or 30 days, or 45 days, or 2 months or 3 months or 4 months or 5 months or 6 months or 9 months or 1 year. In other embodiments, a therapeutically effective amount of at least one non-natural Trk receptor agonist is released from the formulation for a time period exceeding 5 minutes, or 15 minutes, or 30 minutes, or 1 hour, or 4 hours, or 6 hours, or 12 hours, or 18 hours, or 1 day, or 2 days, or 3 days, or 4 days, or 5 days, or 6 days, or 7 days, or 10 days, or 12 days, or 14 days, or 18 days, or 21 days, or 25 days, or 30 days, or 45 days, or 2 months or 3 months or 4 months or 5 months or 6 months or 9 months or 1 year.

In other embodiments, the formulation provides both an immediate release and an extended release formulation of a non-natural Trk receptor agonist. In yet other embodiments, the formulation contains a 0.25:1 ratio, or a 0.5:1 ratio, or a 1:1 ratio, or a 1:2 ratio, or a 1:3, or a 1:4 ratio, or a 1:5 ratio, or a 1:7 ratio, or a 1:10 ratio, or a 1:15 ratio, or a 1:20 ratio of immediate release and extended release formulations. In a further embodiment the formulation provides an immediate release of a first non-natural Trk receptor agonist and an extended release of a second non-natural Trk receptor agonist or other therapeutic agent. In yet other embodiments, the formulation provides an immediate release and extended release formulation of at least one non-natural Trk receptor agonist, and at least one other therapeutic agent. In some embodiments, the formulation provides a 0.25:1 ratio, or a 0.5:1 ratio, or a 1:1 ratio, or a 1:2 ratio, or a 1:3, or a 1:4 ratio, or a 1:5 ratio, or a 1:7 ratio, or a 1:10 ratio, or a 1:15 ratio, or a 1:20 ratio of immediate release and extended release formulations of a first non-natural Trk receptor agonist and second therapeutic agent, respectively.

In a specific embodiment the formulation provides a therapeutically effective amount of at least one non-natural Trk receptor agonist at the site of disease with essentially no systemic exposure. In an additional embodiment the formulation provides a therapeutically effective amount of at least one non-natural Trk receptor agonist at the site of disease with essentially no detectable systemic exposure. In other embodiments, the formulation provides a therapeutically effective amount of at least one non-natural Trk receptor agonist at the site of disease with little or no detectable systemic exposure.

The combination of immediate release, delayed release and/or extended release non-natural Trk receptor agonist compositions or formulations may be combined with other pharmaceutical agents, as well as the excipients, diluents, stabilizers, tonicity agents and other components disclosed herein. As such, depending upon the non-natural Trk receptor agonist used, the thickness or viscosity desired, or the mode of delivery chosen, alternative aspects of the embodiments disclosed herein are combined with the immediate release, delayed release and/or extended release embodiments accordingly.

In certain embodiments, the pharmacokinetics of the non-natural Trk receptor agonist formulations described herein are determined by injecting the formulation on or near the round window membrane of a test animal (including by way of example, a guinea pig, rat, rabbit, cat, or a chinchilla). At a determined period of time (e.g., 6 hours, 12 hours, 1 day, 2 days, 3 days, 4 days, 5 days, 6 days, and 7 days for testing the pharmacokinetics of a formulation over a 1 week period), the test animal is euthanized and a 5 mL sample of the perilymph fluid is tested. The inner ear removed and tested for the presence of the non-natural Trk receptor agonist. As needed, the level of non-natural Trk receptor agonist is measured in other organs. In addition, the systemic level of the non-natural Trk receptor agonist is measured by withdrawing a blood sample from the test animal. In order to determine whether the formulation impedes hearing, the hearing of the test animal is optionally tested.

Alternatively, an inner ear is provided (as removed from a test animal) and the migration of the non-natural Trk receptor agonist is measured. As yet another alternative, an in vitro model of a round window membrane is provided and the migration of the non-natural Trk receptor agonist is measured.

### Kits/Articles of Manufacture

The disclosure also provides kits for preventing, treating or ameliorating the symptoms of a disease or disorder in a mammal. Such kits generally will comprise one or more of the non-natural Trk receptor agonist controlled-release compositions or devices disclosed herein, and instructions for using the kit. The disclosure also contemplates the use of one or more of the non-natural Trk receptor agonist controlled-release compositions, in the manufacture of medicaments for treating, abating, reducing, or ameliorating the symptoms of a disease, dysfunction, or disorder in a mammal, such as a human that has, is suspected of having, or at risk for developing an inner ear disorder.

In some embodiments, kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In other embodiments, the containers are formed from a variety of materials such as glass or plastic.

The articles of manufacture provided herein contain packaging materials. Packaging materials for use in packaging pharmaceutical products are also presented herein. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of non-natural Trk receptor agonist formulations compositions provided herein are contemplated as are a variety of treatments for any disease, disorder, or condition that would benefit by controlled release administration of a non-natural Trk receptor agonist to the inner ear.

In some embodiments, a kit includes one or more additional containers, each with one or more of various materials (such as reagents, optionally in concentrated form, and/or devices) desirable from a commercial and user standpoint for use of a formulation described herein. Non-limiting examples of such materials include, but not limited to, buffers, diluents, filters, needles, syringes; carrier, package, container, vial and/or tube labels listing contents and/or instructions for use and package inserts with instructions for use. A set of instructions is optionally included. In a further embodiment, a label is on or associated with the container. In yet a further embodiment, a label is on a container when letters, numbers or other characters forming the label are attached, molded or etched into the container itself, a label is associated with a container when it is present within a receptacle or carrier that also holds the container, e.g., as a package insert. In other embodiments a label is used to indicate that the contents are to be used for a specific therapeutic application. In yet another embodiment, a label also indicates directions for use of the contents, such as in the methods described herein.

In certain embodiments, the pharmaceutical compositions are presented in a pack or dispenser device which contains one or more unit dosage forms containing a compound provided herein. In another embodiment, the pack for example contains metal or plastic foil, such as a blister pack. In a further embodiment, the pack or dispenser device is accompanied by instructions for administration. In yet a further embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. In another embodiment, such notice, for example, is the labeling approved by the U.S. Food and Drug Administration for prescription drugs, or the approved product insert. In yet another embodiment, compositions containing a compound provided herein formulated in a compatible pharmaceutical carrier are also prepared, placed in an appropriate container, and labeled for treatment of an indicated condition.

### Certain Definitions

The term “auris-acceptable” with respect to a formulation, composition or ingredient, as used herein, includes having no persistent detrimental effect on the auris interna (or inner ear) of the subject being treated. By “auris-pharmaceutically acceptable,” as used herein, refers to a material, such as a carrier or diluent, which does not abrogate the biological activity or properties of the compound in reference to the auris interna (or inner ear), and is relatively or is reduced in toxicity to the auris interna (or inner ear), i.e., the material is administered to an individual without causing undesirable biological effects or interacting in a deleterious manner with any of the components of the composition in which it is contained.

As used herein, amelioration or lessening of the symptoms of a particular otic disease, disorder or condition by administration of a particular compound or pharmaceutical composition refers to any decrease of severity, delay in onset, slowing of progression, or shortening of duration, whether permanent or temporary, lasting or transient that is attributed to or associated with administration of the compound or composition.

“Antioxidants” are auris-pharmaceutically acceptable antioxidants, and include, for example, butylated hydroxytoluene (BHT), sodium ascorbate, ascorbic acid, sodium metabisulfite and tocopherol. In certain embodiments, antioxidants enhance chemical stability where required. Antioxidants are also used to counteract the ototoxic effects of certain therapeutic agents, including agents that are used in combination with the non-natural Trk receptor agonists disclosed herein.

“Auris interna” refers to the inner ear, including the cochlea and the vestibular labyrinth, and the round window that connects the cochlea with the middle ear.

“Auris-interna bioavailability” refers to the percentage of the administered dose of compounds disclosed herein that becomes available in the inner ear of the animal or human being studied.

“Auris media” refers to the middle ear, including the tympanic cavity, auditory ossicles and oval window, which connects the middle ear with the inner ear.

“Balance disorder” refers to a disorder, illness, or condition which causes a subject to feel unsteady, or to have a sensation of movement. Included in this definition are dizziness, vertigo, disequilibrium, and pre-syncope. Diseases which are classified as balance disorders include, but are not limited to, Ramsay Hunt's Syndrome, Meniere's Disease, mal de debarquement, benign paroxysmal positional vertigo, and labyrinthitis.

“Blood plasma concentration” refers to the concentration of compounds provided herein in the plasma component of blood of a subject.

“Carrier materials” are excipients that are compatible with the non-natural Trk receptor agonist, the auris interna and the release profile properties of the auris-acceptable pharmaceutical formulations. Such carrier materials include, e.g., binders, suspending agents, disintegration agents, filling agents, surfactants, solubilizers, stabilizers, lubricants, wetting agents, diluents, and the like. “Auris-pharmaceutically compatible carrier materials” include, but are not limited to, acacia, gelatin, colloidal silicon dioxide, calcium glycerophosphate, calcium lactate, maltodextrin, glycerine, magnesium silicate, polyvinylpyrrolidone (PVP), cholesterol, cholesterol esters, sodium caseinate, soy lecithin, taurocholic acid, phosphatidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like.

The term “diluent” refers to chemical compounds that are used to dilute the non-natural Trk receptor agonist prior to delivery and which are compatible with the auris interna.

“Dispersing agents,” and/or “viscosity modulating agents” are materials that control the diffusion and homogeneity of the non-natural Trk receptor agonist through liquid media. Examples of diffusion facilitators/dispersing agents include but are not limited to hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol has a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose are also be used as dispersing agents. Dispersing agents useful in liposomal dispersions and self-emulsifying dispersions of the non-natural Trk receptor agonists disclosed herein are dimyristoyl phosphatidyl choline, natural phosphatidyl choline from eggs, natural phosphatidyl glycerol from eggs, cholesterol and isopropyl myristate.

“Drug absorption” or “absorption” refers to the process of movement of the non-natural Trk receptor agonists from the localized site of administration, by way of example only, the round window membrane of the inner ear, and across a barrier (the round window membranes, as described below) into the auris interna or inner ear structures. The terms “co-administration” or the like, as used herein, are meant to encompass administration of the non-natural Trk receptor agonists to a single patient, and are intended to include treatment regimens in which the non-natural Trk receptor agonists are administered by the same or different route of administration or at the same or different time.

The terms “effective amount” or “therapeutically effective amount,” as used herein, refer to a sufficient amount of the non-natural Trk receptor agonist being administered that would be expected to relieve to some extent one or more of the symptoms of the disease or condition being treated. For example, the result of administration of the non-natural Trk receptor agonist disclosed herein is reduction and/or alleviation of the signs, symptoms, or causes of tinnitus or balance disorders. For example, an “effective amount” for therapeutic uses is the amount of non-natural Trk receptor agonist, including a formulation as disclosed herein required to provide a decrease or amelioration in disease symptoms without undue adverse side effects. The term “therapeutically effective amount” includes, for example, a prophylactically effective amount. An “effective amount” of a modulator of neuron and/or hair cells of the auris composition disclosed herein is an amount effective to achieve a desired pharmacologic effect or therapeutic improvement without undue adverse side effects. It is understood that “an effective amount” or “a therapeutically effective amount” varies, in some embodiments, from subject to subject, due to variation in metabolism of the compound administered, age, weight, general condition of the subject, the condition being treated, the severity of the condition being treated, and the judgment of the prescribing physician. It is also understood that “an effective amount” in an extended-release dosing format may differ from “an effective amount” in an immediate release design format based upon pharmacokinetic and pharmacodynamic considerations.

The terms “enhance” or “enhancing” refers to an increase or prolongation of either the potency or duration of a desired effect of non-natural Trk receptor agonist, or a diminution of any adverse symptomotology that is consequent upon the administration of the therapeutic agent. Thus, in regard to enhancing the effect of the non-natural Trk receptor agonists disclosed herein, the term “enhancing” refers to the ability to increase or prolong, either in potency or duration, the effect of other therapeutic agents that are used in combination with the non-natural Trk receptor agonist disclosed herein. An “enhancing-effective amount,” as used herein, refers to an amount of non-natural Trk receptor agonist or other therapeutic agent which is adequate to enhance the effect of another therapeutic agent or non-natural Trk receptor agonist of the target auris structure in a desired system. When used in a patient, amounts effective for this use will depend on the severity and course of the disease, disorder or condition, previous therapy, the patient's health status and response to the drugs, and the judgment of the treating physician.

The term “inhibiting” includes preventing, slowing, or reversing the development of a condition, for example, or advancement of a condition in a patient necessitating treatment.

The terms “kit” and “article of manufacture” are used as synonyms.

“Pharmacodynamics” refers to the factors which determine the biologic response observed relative to the concentration of drug at the desired site within the auris media and/or auris interna.

“Pharmacokinetics” refers to the factors which determine the attainment and maintenance of the appropriate concentration of drug at the desired site within the auris media and/or auris interna.

The term “non-natural Trk receptor agonist” includes agents that recognize and bind to one or more epitopes on a Trk receptor. For example, Trk receptors include TrkA, TrkB, or TrkC receptor. In some embodiments, the non-natural Trk receptor agonist is a neurotrophic agent. The non-natural Trk receptor agonists are agents that can promote the growth and/or regeneration of neurons and their processes and connections and/or the hair cells of the auris. In some embodiments, a non-natural Trk receptor agonist provides therapeutic benefit (e.g., alleviation of hearing loss) by promoting the growth and/or regeneration and/or phenotypic maintenance of auris sensory cells and their processes and connections (e.g., neurons and/or the hair cells) of the auris. In some embodiments, a non-natural Trk receptor agonist provides therapeutic benefit (e.g., alleviation of tinnitus due to acoustic trauma) by treating and/or reversing damage to auris sensory cells (e.g., dysfunction of neurons and/or hair cells of the auris) or reducing or delaying further damage (e.g., cell death) to auris sensory cells (e.g., by exerting an otoprotectant effect or atrophic effect).

Non-natural Trk receptor agonists include “non-natural neurotrophic agent” which means a chemically modified analog of a naturally occurring neurotrophic agent (e.g., NGF, BDNF, NT-3, NT-4, NT-5), or a naturally occurring neurotrophic agent with one or more mutations in amino acid residues, that promotes the survival, growth and/or regeneration of auris sensory cells (e.g., neurons and/or the hair cells of the auris). In some embodiments, a non-natural neurotrophic agent reduces or inhibits oxidative damage and/or osteoneogenesis and/or degeneration of auris sensory cells. In some embodiments, a non-natural neurotrophic agent maintains healthy auris sensory cells (e.g., after a surgical implant of a medical device). In some embodiments, a non-natural neurotrophic agent is an immunosuppressant (e.g., an immunosuppressant used during otic surgery). In some embodiments, a non-natural neurotrophic agent is a growth factor (e.g., a growth factor used after an implantation procedure to promote growth of auris cells). In some instances, the non-natural Trk receptor agonist is a modified neurotrophic agent (e.g., NGF, BDNF, NT-3, NT-4, NT-5) comprising one or more mutations in amino acid residues.

The term “mutein” refers to any protein arising as a result of a modification. In some instances, the modification is a mutation. In some embodiments, the “mutein” is a protein with altered amino acid sequences as compared to the wild-type protein from which the mutated protein is derived.

In prophylactic applications, compositions comprising the non-natural Trk receptor agonists described herein are administered to a patient susceptible to or otherwise at risk of a particular disease, disorder or condition. For example, such conditions include and are not limited to ototoxicity, chemotherapy induced hearing loss, excitotoxicity, sensorineural hearing loss, noise induced hearing loss, hidden hearing loss, Meniere's Disease/Syndrome, endolymphatic hydrops, labyrinthitis, Ramsay Hunt's Syndrome, vestibular neuronitis, tinnitus and microvascular compression syndrome, synaptopathy, drug-induced neurodegeneration of otic neurons. Such an amount is defined to be a “prophylactically effective amount or dose.” In this use, the precise amounts also depend on the patient's state of health, weight, and the like.

As used herein, a “pharmaceutical device” includes any composition described herein that, upon administration to an ear, provides a reservoir for extended release of an active agent described herein.

The term “substantially low degradation products” means about 10% by weight of the active agent are degradation products of the active agent. In further embodiments, the term means less than 10% by weight of the active agent are degradation products of the active agent. In further embodiments, the term means less than 9% by weight of the active agent are degradation products of the active agent. In further embodiments, the term means less than 8% by weight of the active agent are degradation products of the active agent. In further embodiments, the term means less than 7% by weight of the active agent are degradation products of the active agent. In further embodiments, the term means less than 6% by weight of the active agent are degradation products of the active agent. In further embodiments, the term means less than 5% by weight of the active agent are degradation products of the active agent. In further embodiments, the term means less than 4% by weight of the active agent are degradation products of the active agent. In further embodiments, the term means less than 3% by weight of the active agent are degradation products of the active agent. In yet further embodiments, the term means less than 2% by weight of the active agent are degradation products of the active agent. In further embodiments, the term means less than 1% by weight of the active agent are degradation products of the active agent. In some embodiments, any individual impurity (e.g., metal impurity, degradation products of active agent and/or excipients, or the like) present in a formulation described herein is less than 5%, less than 2%, or less than 1% by weight of the active agent. In some embodiments the formulation does not contain precipitate during storage or change in color after manufacturing and storage.

The term “otic intervention” means an external insult or trauma to one or more auris structures and includes implants, otic surgery, injections, cannulations, or the like. Implants include auris-interna or auris-media medical devices, examples of which include cochlear implants, hearing sparing devices, hearing-improvement devices, short electrodes, micro-prostheses or piston-like prostheses; needles; stem cell transplants; drug delivery devices; any cell-based therapeutic; or the like. Otic surgery includes middle ear surgery, inner ear surgery, tympanostomy, cochleostomy, labyrinthotomy, mastoidectomy, stapedectomy, stapedotomy, endolymphatic sacculotomy or the like. Injections include intratympanic injections, intracochlear injections, injections across the round window membrane or the like. Cannulations include intratympanic, intracochlear, endolymphatic, perilymphatic or vestibular cannulations or the like.

A “prodrug” refers to a non-natural Trk receptor agonist that is converted into the parent drug in vivo. In certain embodiments, a prodrug is enzymatically metabolized by one or more steps or processes to the biologically, pharmaceutically or therapeutically active form of the compound. To produce a prodrug, a pharmaceutically active compound is modified such that the active compound will be regenerated upon in vivo administration. In one embodiment, the prodrug is designed to alter the metabolic stability or the transport characteristics of a drug, to mask side effects or toxicity, or to alter other characteristics or properties of a drug. Compounds provided herein, in some embodiments, are derivatized into suitable prodrugs.

“Solubilizers” refer to auris-acceptable compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like that assist or increase the solubility of the non-natural Trk receptor agonists disclosed herein.

“Stabilizers” refers to compounds such as any antioxidation agents, buffers, acids, preservatives and the like that are compatible with the environment of the auris interna. Stabilizers include but are not limited to agents that will do any of (1) improve the compatibility of excipients with a container, or a delivery system, including a syringe or a glass bottle, (2) improve the stability of a component of the composition, or (3) improve formulation stability.

“Steady state,” as used herein, is when the amount of drug administered to the auris interna is equal to the amount of drug eliminated within one dosing interval resulting in a plateau or constant levels of drug exposure within the targeted structure.

As used herein, the term “subject” is used to mean an animal, preferably a mammal, including a human or non-human. The terms “patient(s)”, “individual(s)” and “subject(s)” may be used interchangeably. None of the terms require or are limited to situations characterized by the supervision (e.g. constant or intermittent) of a health care worker (e.g. a doctor, a registered nurse, a nurse practitioner, a physician's assistant, an orderly or a hospice worker).

“Surfactants” refer to compounds that are auris-acceptable, such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants are included to enhance physical stability or for other purposes.

The terms “treat,” “treating” or “treatment,” as used herein, include alleviating, abating or ameliorating a disease or condition, for example tinnitus, symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

Other objects, features, and advantages of the methods and compositions described herein will become apparent from the detailed description. It should be understood, however, that the detailed description and the specific examples, while indicating specific embodiments, are given by way of illustration only.

### EXAMPLES

### Example 1—Neurotrophic Effects of Exemplary Neurotrophin Mutants in Rat Spiral Ganglion Neuron Cultures

In some embodiments, NT-3 and BDNF provide trophic support to spiral ganglion neurons in the cochlea through activation of TrkC and TrkB receptors, respectively (Green et al, Anat Rec. 295:1877, 2012). Furthermore, interaction of NT-3 and BDNF with p75 leads to activation of death receptor pathway leading to apoptosis of spiral ganglion neurons (Green et al, Anat Rec. 295:1877, 2012). In some cases, mutations of NT-3 have been observed to modulate TrkB activity and interaction with p75 (Urfer et al, EMBO Journal 13:5896, 1994). In this example, exemplary NT-3 mutants were generated and their ability to provide trophic support to rat spiral ganglion neurons was tested.

Spiral Ganglion Dissection and Culture

Postnatal Sprague Dawley rats (P2-4) of both sexes were anesthetized with isoflurane and decapitated. Temporal bones were removed and transferred to a cell culture dish with the dissection medium containing 1×HBSS (Invitrogen), 1M HEPES (Life Technologies) and 0.35% D-glucose (Fisher Scientific). Under microscopic visualization, the cochlear capsule was carefully removed from the temporal bone using forceps and transferred to a new cell culture dish containing the same buffer mentioned above. The cochlea was then dissected from the cochlear capsule using fine forceps. The stria vascularis and the organ of Corti were removed from the cochlear tissue, and the spiral ganglion neurons were subsequently detached from the modiolus. This strand, containing spiral ganglion neurons, was transferred to a 1.5 mL microcentrifuge tube containing 0.9 mL of the dissection medium. Once ˜12 of these strands (representing 6 animals) were collected in cold dissection medium, enzymatic and mechanical dissociation proceeded as described below.

0.1 ml of Trypsin (Life Technologies) was added to the spiral ganglion neurons in dissection medium and incubated at room temperature for 10 minutes. The cells were then briefly centrifuged, the supernatant discarded and the cells were washed twice with culture medium containing Dulbecco's modified Eagle's medium (Life Technologies), 1×N2 supplement and 10% fetal bovine serum (Thermo Fisher). The cells were resuspended in 0.5 ml of culture medium and mechanically dissociated with a glass pipette, for 4-6 triturations. After 4-6 triturations, the cells were briefly centrifuged and the supernatant applied to a 40-micron cell strainer (Millipore). This was repeated until the tissue was fully dissociated with no visible cell clusters remaining. Surviving cells were counted using the Countess II (Thermo Fisher) using trypan blue, and then seeded into a 96-well plate, pre-coated with poly-L-ornithine and laminin (Corning) at a density of 1.4×10⁴ cells per well.

Treatments with wildtype and mutant NT-3 were conducted for 4 days at 37° C. and 5% CO₂. Immediately after seeding, test agents were added to the culture medium prepared at 10× concentration and the volume was then diluted 10-fold when added to the seeded cells. The next day, the adhered cells were washed once with serum-free culture medium and then refilled with fresh serum-free medium. 10× concentrated treatments were again added to the cells with 10-fold dilution. The cultures were kept in the incubator for an additional 3 days before being fixed, stained, and imaged.

Immunohistochemistry

Cells were fixed in cold 4% paraformaldehyde for 20 minutes, then washed twice in PBS containing 0.5% triton (PBS-T). Cells were then incubated 1-2 hr at room temperature in primary antibody (Anti-200 kD chicken-Neurofilament Heavy antibody; Abcam) in PBS-T containing 10% goat serum. After washing three times in PBS, the cells were then incubated for 1 hr at room temperature with secondary antibody (Goat Anti-Chicken IgY H&L (Alexa Fluor® 488) preadsorbed; Abcam). Cells were then washed twice, treated with DAPI nuclear stain for 5-10 minutes, and then washed two more times with PBS before imaging. Numbers of spiral ganglion neurons (identified by neurofilament staining) surviving in each well were counted.

Results

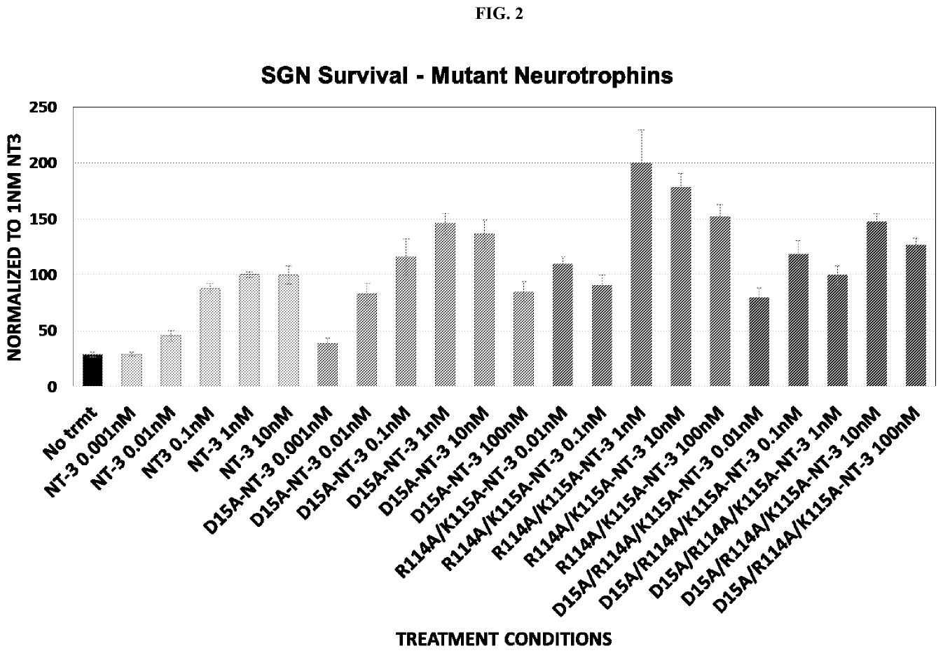

NT-3 and BDNF both supported spiral ganglion neuron survival in culture. Typically, approximately 150-200 neurons/well were present with 1 nM NT-3, compared to 30-40 neurons in the untreated wells. All three mutant neurotrophins showed a dose-dependent trophic effect on spiral ganglion neurons (FIG. 2). Compared with the maximal effect of NT-3 (at 1 nM, normalized to 100%), the maximal effect of D15A-NT-3, R114A/K115A-NT-3 and D15A/R114A/K115A-NT-3 was increased to 150%, 200%, and 150%, respectively (see FIG. 2).

### Example 2—Sustained Release of Exemplary Neurotrophins BDNF and NT-3

BDNF, NT3 in Poloxamer 407

Poloxamer 407 gel at 16% was prepared using the cold method. In brief, a 16% w/w stock solution of poloxamer 407 was prepared by slowly adding it to a cold buffer solution (10 mM PBS, pH 7.4). Sterilization was achieved by filtration. Human recombinant BDNF was suspended with an appropriate amount of poloxamer 407 solution to reach a concentration of 1.05 mg/ml BDNF. Human recombinant NT-3 was suspended with an appropriate amount of poloxamer 407 solution to reach a concentration of 1.05 mg/ml NT-3.

Pharmacokinetics

Female rats (Charles River) weighing 200-300 g of approximately 12-16 weeks of age served as subjects (N=4 per group). Prior to any procedures, animals were anesthetized using a combination of xylazine (10 mg/kg) and ketamine (90 mg/kg) for up to an hour via the intraperitoneal route. If needed, an intraoperative booster was administered intraperitoneally representing a one-tenth of the original dose.

Intratympanic Injection—

Each animal was positioned so that the head was tilted at an angle to favor injection towards the round window niche. Briefly, under visualization with an operating microscope, 20 μL of the formulation was injected using a 25 G (Gauge) 11/2 needle through the tympanic membrane into the superior posterior quadrant. Formulations were delivered using a perfusion pump at the rate of 2 μL/sec. Contact with the round window membrane was maintained for 30 minutes by placing the animal in a recumbent position. During the procedure and until recovery, animals were placed on a temperature controlled (40° C.) heating pad until consciousness was regained at which time they were returned to the vivarium.

Perilymph Sampling Procedure—

The skin behind the ear of anesthetized rats was shaved and disinfected with povidone-iodine. An incision was then made behind the ear, and muscles were carefully retracted from over the bulla. A hole was drilled through the bulla using a dental burr so that the middle ear was exposed and accessed. The cochlea and the round window membrane were visualized under a stereo surgical microscope. The basal turn of bulla was cleaned by using small cotton ball. A unique microhole was hand drilled through the bony shell of the cochlea (cochlear capsule) adjacent to the round window. Perilymph (about 2 μL) was then collected using a microcapillary inserted into the cochlear scala tympani. Perilymph samples were added to a vial containing 18 μL of acetonitrile/water (50/50, v/v), stored at −80° C. until analysis.

Analytical Method

Concentrations of BDNF and NT-3 in perilymph and plasma samples were determined using commercially available ELISA kits.

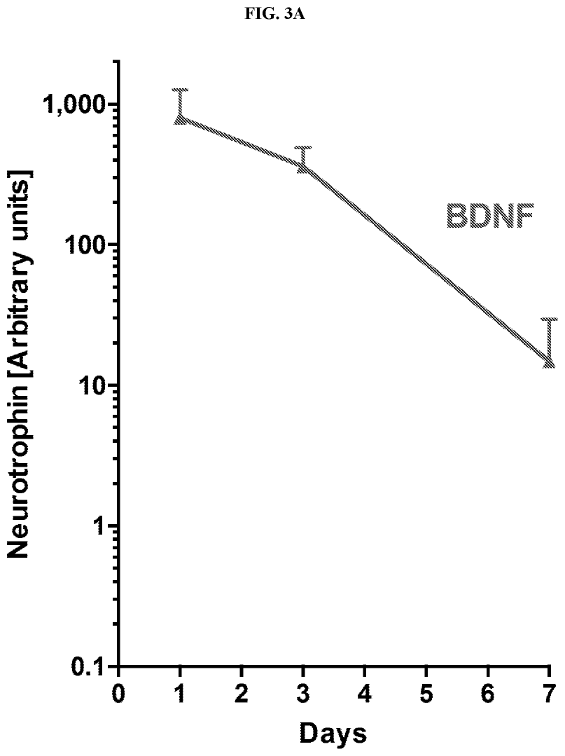

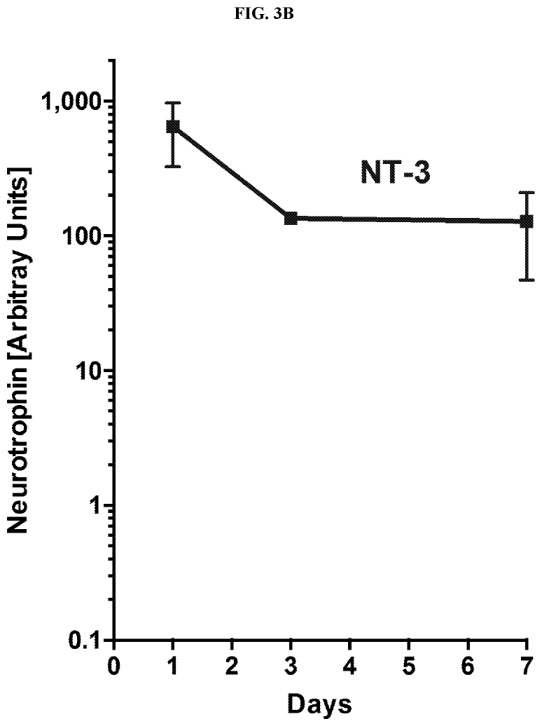

FIG. 3A-FIG. 3B illustrate perilymph concentrations of BDNF and NT-3 following a single IT injection in rats. BDNF was administered at a dose of 0.1% (1.05 mg/ml) (FIG. 3A) and NT-3 was administered at a dose of 0.1% (1.05 mg/ml) (FIG. 3B).

### Example 3—Sustained Release of BDNF in Poloxamer and MCT Formulations

BDNF in Poloxamer 407

Poloxamer 407 solution at 32% was prepared by slowly adding it to a cold buffer solution (50 mM tris buffer with saline, pH 7.4). Sterilization was achieved by filtration. Human recombinant BDNF was either diluted or concentrated with the same tris buffer solution to a concentration twice the target concentration. The solutions were sterilized by filtration. Combining the above two solutions at 1:1 ratio yields a solution or a suspension containing 16% P407 and a concentration range of 0.005% to 0.5% BDNF.

BDNF/PVP (K25) in MCT SiO2

BDNF/PVP (2% or 10%) in MCT/SiO2 (0.5%) formulations were prepared as follows. PVP was dissolved in water for injection at 2 or 10% concentration. The BDNF frozen solution was thawed at 5° C. overnight and was then diluted with water for injection to a concentration of 0.15% BDNF. Combining the above two solutions at a ratio of 1:1 (w/w) yields a solution with 0.075% BDNF and 1% or 5% PVP. The solution was sterile filtered via a 0.22 um filter, filled into vials and then lyophilized. The dry powder was then dispersed into sterile filtered MCT by bead-blasting. Finally, the heat sterilized SiO2 was added. The final suspension contains 0.15% BDNF, 2 or 10% PVP and 0.5% SiO2 in MCT.

BDNF/Carbopol (971P) in MCT/SiO2

BDNF/Carbopol (0.15%) in MCT/SiO2 (0.5%) formulation was prepared as follows. Carbopol was dissolved in water for injection at 0.04% concentration. The BDNF frozen solution was thawed at 5° C. overnight and was then diluted with water for injection to a concentration of 0.04% BDNF. Combining the above two solutions at a ratio of 1:1 (w/w) yields a solution with 0.02% BDNF and 0.02% Carbopol. The solution was sterile filtered via a 0.22 um filter, filled into vials and then lyophilized. The dry powder was then dispersed into sterile filtered MCT by bead-blasting. Finally, the heat sterilized SiO2 was added. The final suspension contains 0.15% BDNF, 0.15% Carbopol and 0.5% SiO2 in MCT.

BDNF/Poloxamer (P407) in MCT/SiO2

BDNF/P407 (10%) in MCT/SiO2 (0.5%) formulation was prepared as follows. P407 was dissolved in water for injection at 10% concentration. The BDNF frozen solution was thawed at 5 C overnight and was then diluted with water for injection to a concentration of 0.15% BDNF. Combining the above two solutions at a ratio of 1:1 (w/w) yields a solution with 0.075% BDNF and 5% P407. The solution was sterile filtered via a 0.22 um filter, filled into vials and then lyophilized. The dry powder was then dispersed into sterile filtered MCT by bead-blasting. Finally, the heat sterilized SiO2 was added. The final suspension contains 0.15% BDNF, 10% P407 and 0.5% SiO2 in MCT.

Pharmacokinetics

Female rats (Charles River) weighing 200-300 g of approximately 12-16 weeks of age served as subjects (N=4 per group). Prior to any procedures, animals were anesthetized using a combination of xylazine (10 mg/kg) and ketamine (90 mg/kg) for up to an hour via the intraperitoneal route. If needed, an intraoperative booster was administered intraperitoneally representing a one-tenth of the original dose.

Intratympanic Injection—

Each animal was positioned so that the head was tilted at an angle to favor injection towards the round window niche. Briefly, under visualization with an operating microscope, 20 μL of the formulation was injected using a 25 G (Gauge) 11/2 needle through the tympanic membrane into the superior posterior quadrant. Formulations were delivered using a perfusion pump at the rate of 2 μL/sec. Contact with the round window membrane was maintained for 30 minutes by placing the animal in a recumbent position. During the procedure and until recovery, animals were placed on a temperature controlled (40° C.) heating pad until consciousness was regained at which time they were returned to the vivarium.

Perilymph Sampling Procedure—

The skin behind the ear of anesthetized rats was shaved and disinfected with povidone-iodine. An incision was then made behind the ear, and muscles were carefully retracted from over the bulla. A hole was drilled through the bulla using a dental burr so that the middle ear was exposed and accessed. The cochlea and the round window membrane were visualized under a stereo surgical microscope. The basal turn of bulla was cleaned by using small cotton ball. A unique microhole was hand drilled through the bony shell of the cochlea (cochlear capsule) adjacent to the round window. Perilymph (about 2 μL) was then collected using a microcapillary inserted into the cochlear scala tympani. Perilymph samples were added to a vial containing 18 μL of acetonitrile/water (50/50, v/v), stored at −80° C. until analysis.

Analytical Method

Concentrations of BDNF in perilymph and samples were determined using commercially available ELISA kits.

The limits of detection of human BDNF were 4 pg/mL.

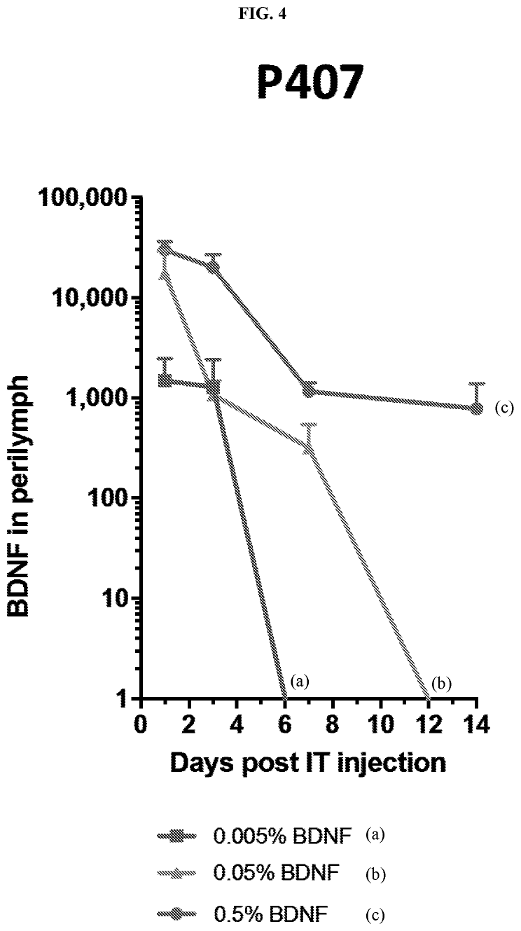

FIG. 4 illustrates perilymph concentrations of BDNF following a single IT injection in rats in poloxamer 407 formulation. BDNF was administered at the indicated doses. Perilymph BDNF is in arbitrary units.

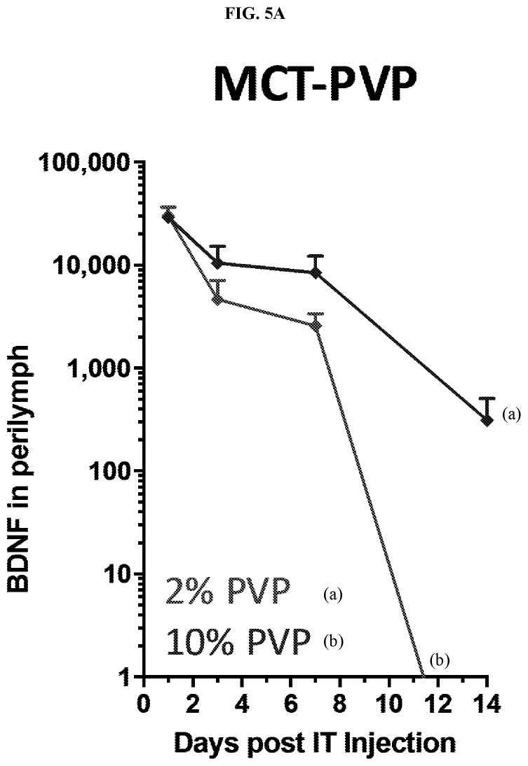

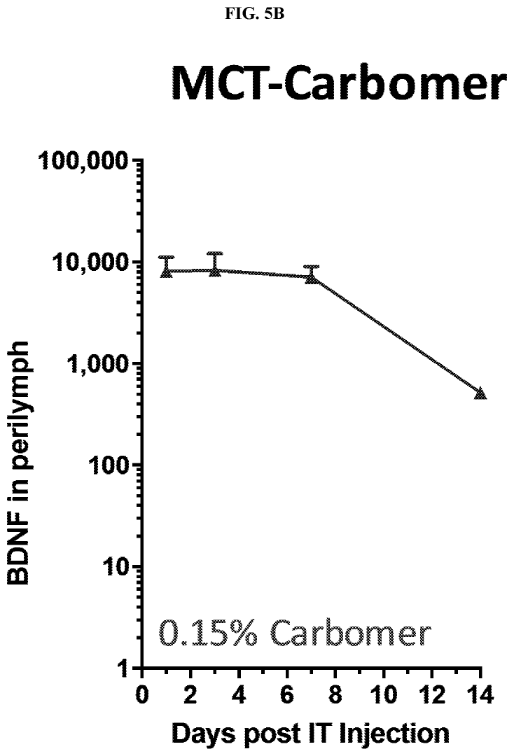

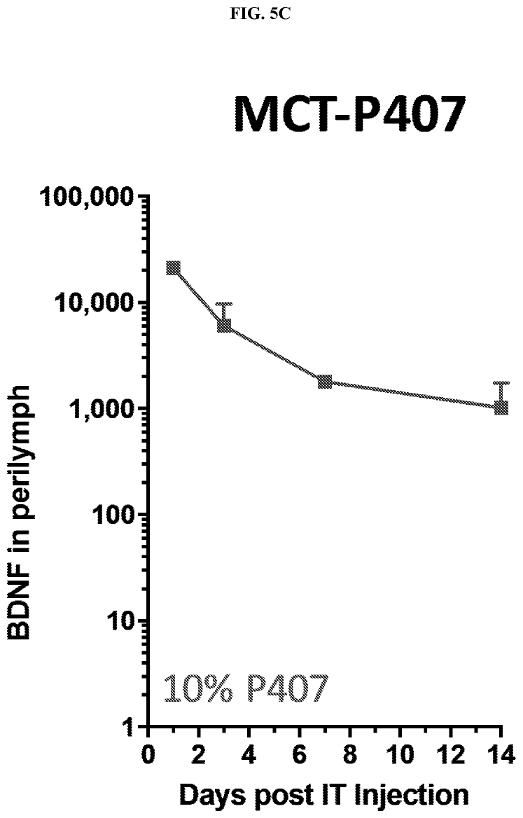

FIG. 5A-FIG. 5C illustrate perilymph concentrations of BDNF following a single IT injection in rats in MCT formulations. BDNF was administered at a dose of 0.15% (1.5 mg/ml) in the MCT formulation containing different polymers: PVP (polyvinylpyrrolidone) at 2 or 10% (FIG. 5A); 0.15% carbomer (FIG. 5B); or P407 (poloxamer 407) at 10% (FIG. 5C). Perilymph BDNF is in arbitrary units.

### Example 4—TrkC Receptor Assays

Cells and Incubation with Test Neurotrophin Mutants:

Cell lines stably expressing human TrkC (in an NIH3T3 cell background) were maintained in culture with Dulbecco's modified Eagle's medium (DMEM) with 10% fetal bovine serum and 1% penicillin and streptomycin. 48 hours prior to assay, cells were transferred to a 96-well plate (2500 cells/well). On the day of the assay, cells were serum-starved by replacing the culture medium with Dulbecco's modified Eagle's medium (DMEM; 100 μl/well) and incubated for 3-4 hours at 37° C. Cells were then incubated (50 μl/well) with NT-3, mutant neurotrophins and for 20 min in phosphate-buffered saline (PBS) at room temperature. After aspiration of the incubation media, cells were lysed by the addition of lysis buffer (1× AlphaSure Lysis Buffer Ultra, Perkin Elmer; 50 μl/well).

Determination of Phospho-ERK (p-ERK) Using AlphaLisa:

10 μl of the cell lysates were placed in a 384-well plate. 10 μl of antibody reagents were added to each well followed by 10 μl of the acceptor/donor bead mix according to the kit instructions (Perkin Elmer kit: ALSU-PERK-A10K). Incubation was continued for 4 hours at room temperature in the dark and the 384-well plates were read at 680/520-620 excitation/emission using an Enspire (Perkin Elmer) plate reader. Values obtained (relative light units) are a quantitative representation of p-ERK in the cells. After subtraction of background from untreated cells, values for NT-3 and test antibodies were expressed relative to that for 10 nM NT-3 (100%) and dose-response curves generated in GraphPad Prism. EC₅₀ and maximum effect values were calculated for individual dose-response curves using a curve fitting program in GraphPad Prism.

Results

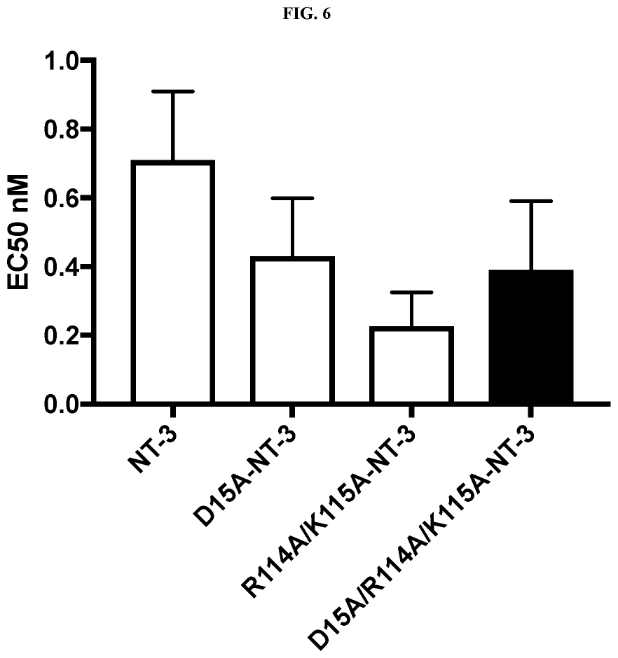

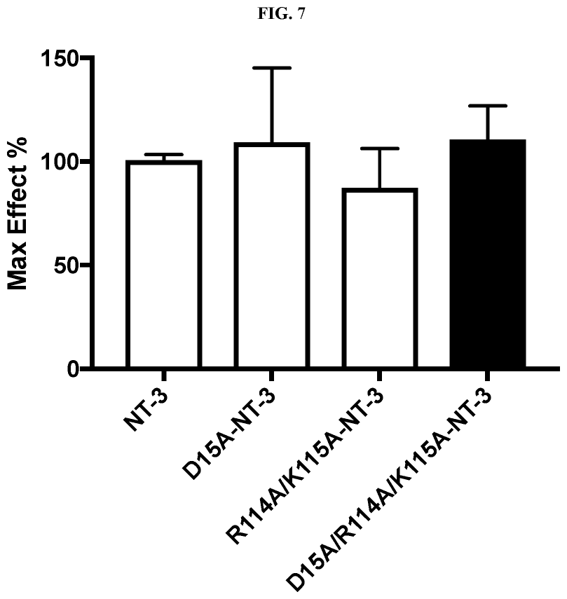

Phosphorylation of ERK is an important downstream consequence of TrkC receptor activation. The natural ligand NT-3 increases intracellular levels of p-ERK in a dose-dependent manner through TrkC. Consequently, an increase of p-ERK in cell lines expressing TrkC is a measure of how well a mutant neurotrophin activates that receptor, relative to NT-3. This can be assessed by determining both the EC₅₀ value (a measure of affinity) and the maximal effect relative to NT-3 or BDNF (a measure of efficacy). As shown in FIG. 6, the EC₅₀ values for D15A-NT-3, R114A/K115A-NT-3 and D15A/R114A/K115A-NT-3 had lower values compared with NT-3, indicating improved affinity. The maximal effects of D15A-NT-3, R114A/K115A-NT-3 and D15A/R114A/K115A-NT-3 were similar to that of NT-3 (FIG. 7). These data indicate that D15A-NT-3, R114A/K115A-NT-3 and D15A/R114A/K115A-NT-3 have improved properties as TrkC agonists compared with NT-3.

### Example 5—Interaction of TrkC with Exemplary NT-3 Mutants

In this study, exemplary NT-3 mutants (SEQ ID NOs: 23, 31, and 32) were profiled for their respective interaction with TrkC and p75 in the presence of wild type NT-3. NT-3 mutants (SEQ ID NOs: 23 and 32) were observed to have reduced binding to p75 relative to wild type NT-3.

Wild type NT-3 was covalently linked to biotin for use as a tracer. In this study, the presence of the covalently linked biotin was identified using a fluorescent-avidin in fluocytometry studies with mean channel fluorescence (MCF) indicating the level of binding. Assays were based on Barcelona, et al., Mol Cell Biol. 35(19):3396-3408 (2015).

Unlabeled wild type NT3 or exemplary mutant NT3 proteins were used as competitors (10-fold excess) as “cold competitors” to competitively block binding of Biotin-NT-3 to receptors. In the absence of competitor, Biotin-NT-3 bound to cells expressing the indicated receptor TrkC or p75 (Table 3 lane 2) and was standardized to 100% binding. Unlabeled wild type NT-3 competitively blocked binding of Biotin-NT-3 to TrkC or to p75 receptors to levels of about 18% and 17% of maximal levels (Table 3 lane 3). An exemplary NT-3 mutant (SEQ ID NO: 31) competed with Biotin-NT-3 for binding to TrkC and to p75 to levels of about 8% and 15% of maximal levels, respectively (Table 3 lane 4). A second NT-3 mutant (SEQ ID NO: 32) competed with Biotin-NT-3 for binding to TrkC to a level of about 2%, and with Biotin-NT-3 for binding to p75 to a level of about 78% of maximal levels (Table 3 lane 5). A third NT-3 mutant (SEQ ID NO: 23) competed with Biotin-NT-3 for binding to TrkC to a level of about 13%, and with Biotin-NT-3 for binding to p75 to a level of about 89% of maximal levels (Table 3 lane 6).

TABLE 3

| | | | | % of | | % of |
|---|---|---|---|---|---|---|
| | Biotin- | | NIH- | maximal | NIH- | maximal |
| | NT3 | Test or cold | TrkC | binding | p75 | binding |
| Lane | (nM) | competitor | MCF | to TrkC | MCF | to p75 |
| 1 | 0 | Background | 10 | 0 | 11 | 0 |
| | | (no biotin- | | | | |
| | | NT3) | | | | |
| 2 | 20 | No competition | 93 | 100 | 76 | 100 |
| 3 | 20 | Wild type NT3 | 25 | 18 | 22 | 17 |
| | | 200 nM | | | | |
| 4 | 20 | NT-3 mutant | 17 | 8 | 21 | 15 |
| | | (SEQ ID NO: 31) | | | | |
| | | at 200 nM | | | | |
| 5 | 20 | NT-3 mutant | 12 | 2 | 62 | 78 |
| | | (SEQ ID NO: 32) | | | | |
| | | at 200 nM | | | | |
| 6 | 20 | NT-3 mutant | 21 | 13 | 69 | 89 |
| | | (SEQ ID NO: 23) | | | | |
| | | at 200 nM | | | | |

### Example 6—Exemplary NT-3 Mutants Activate TrkC and Promote Survival Signals

Wild type NT-3 or NT-3 mutant proteins (SEQ ID NOs: 23, 31, 32) were tested as survival factors for cells that express TrkC. Cells were cultured in serum free media to cause stress and death. Supplementation with a growth factor (NT3 or NT3 mutants for the TrkC-expressing cells) prevented death caused by culture in serum free media. MTT assay as described in Brahimi et al, PLoS One 11(10): e0162307 (2016) was used and the colorimetrical readout was in Optical Density unit.

Without supplementation, TrkC-expressing cells did not survive and did not metabolize MTT (Table 4 lane 1). Addition of supplementation to wild type NT3 promoted cell survival in a dose dependent manner. 1 nM wild type NT3 was standardized as 100% (Table 4 lanes 2 and 3). Addition of supplementation to NT3 mutants (SEQ ID NOs: 23, 31, and 32) promoted cell survival in a dose dependent manner (Table 4 lanes 4, 5 and 6, 7 and 8, 9).

TABLE 4

| | | Concentration | MTT | |
|---|---|---|---|---|
| Lane | Test Growth Factor | (nM) in culture | O.D. | % survival |
| 1 | No treatment (background) | 0 | 0.04 | 0 |
| 2 | Wild type NT3 | 0.1 | 0.30 | 34 |
| 3 | | 1 | 0.81 | 100 |
| 4 | NT-3 mutant | 0.1 | 0.28 | 31 |
| | (SEQ ID NO: 31) | | | |
| 5 | | 1 | 0.87 | 108 |
| 6 | NT-3 mutant | 0.1 | 0.34 | 39 |
| | (SEQ ID NO: 32) | | | |
| 7 | | 1 | 0.92 | 114 |
| 8 | NT-3 mutant | 0.1 | 0.27 | 30 |
| | (SEQ ID NO: 23) | | | |
| 9 | | 1 | 0.89 | 110 |

### Example 7—Interaction of TrkA with Exemplary NT-3 Mutants

In this study, exemplary NT-3 mutants (SEQ ID NOs: 23, 31, and 32) were profiled for their respective interaction with TrkA and were observed to have higher binding interaction with TrkA relative to wild type NT-3.

Wild type NT-3 was covalently linked to biotin for use as a tracer. In this study, the presence of the covalently linked biotin was identified using a fluorescent-avidin in fluocytometry studies with mean channel fluorescence (MCF) indicating the level of binding. Assays were based on Barcelona, et al., Mol Cell Biol. 35(19):3396-3408 (2015).

Unlabeled wild type NT3 or exemplary mutant NT3 proteins were used as competitors (3-fold excess) as “cold competitors” to competitively block binding of Biotin-NT-3 to receptors.

In the absence of competitor, Biotin-NT-3 bound to cells expressing TrkA (Table 5 lane 2) and was standardized to 100% binding. Unlabeled wild type NT-3 competitively blocked binding of Biotin-NT-3 to TrkA receptors and binding was reduced to about 36% of maximal levels (Table 5 lane 3). An exemplary NT-3 mutant (SEQ ID NO: 31) exhibited reduced binding to TrkA in the presence of Biotin-NT-3 and binding remained at 79% of maximal levels (Table 5 lane 4). A second exemplary NT-3 mutant (SEQ ID NO: 32) was observed to compete with Biotin-NT-3 for binding to TrkA and binding was reduced to 46% of maximal levels (Table 5 lane 5). A third exemplary NT-3 mutant (SEQ ID NO: 23) competed with Biotin-NT-3 for binding to TrkA with higher efficacy than wild type NT-3, and binding was reduced to 21% of maximal levels (Table 5 lane 6).

TABLE 5

| | | | | % of |
|---|---|---|---|---|
| | | | | maximal |
| | Biotin-NT3 | Test or | NIH-TrkA | binding |
| Lane | (nM) | cold competitor | MCF | to TrkA |
| 1 | 0 | Background (no biotin-NT3) | 10 | 0 |
| 2 | 20 | No competition | 38 | 100 |
| 3 | 20 | Wild type NT3 | 20 | 36 |
| 4 | 20 | NT-3 mutant (SEQ ID NO: 31) | 32 | 79 |
| 5 | 20 | NT-3 mutant (SEQ ID NO: 32) | 23 | 46 |
| 6 | 20 | NT-3 mutant (SEQ ID NO: 23) | 16 | 21 |

### Example 8—In Vitro Comparison of Gelation Temperature

The effect of Poloxamer 188 and non-natural neurotrophic agent on the gelation temperature and viscosity of Poloxamer 407 formulations is evaluated with the purpose of manipulating the gelation temperature.

A 25% Poloxamer 407 stock solution in PBS buffer and Poloxamer 188NF from BASF are used. An appropriate amount of a non-natural neurotrophic agent is added to the solutions described in Table 6 to provide a 2% formulation of the non-natural neurotrophic agent.

A PBS buffer (pH 7.3) is prepared by dissolving 805.5 mg of sodium chloride (Fisher Scientific), 606 mg of sodium phosphate dibasic anhydrous (Fisher Scientific), 247 mg of sodium phosphate monobasic anhydrous (Fisher Scientific), then QS to 200 g with sterile filtered DI water.

TABLE 6
Preparation of Samples Containing Poloxamer 407/Poloxamer 188

| | 25% P407 Stock | Poloxamer 188 | |
|---|---|---|---|
| Sample | Solution (g) | (mg) | PBS Buffer (g) |
| 16% P407/10% P188 | 3.207 | 501 | 1.3036 |
| 17% P407/10% P188 | 3.4089 | 500 | 1.1056 |
| 18% P407/10% P188 | 3.6156 | 502 | 0.9072 |
| 19% P407/10% P188 | 3.8183 | 500 | 0.7050 |
| 20% P407/10% P188 | 4.008 | 501 | 0.5032 |
| 20% P407/5% P188 | 4.01 | 256 | 0.770 |

Gelation temperature of the above formulations are measured using procedures described herein.

An equation is fitted to the data obtained and is utilized to estimate the gelation temperature of F127/F68 mixtures (for 17-20% F127 and 0-10% F68).

T_{gel}=−1.8(% F127)+1.3(% F68)+53

An equation is fitted to the data obtained and can be utilized to estimate the Mean Dissolution Time (hr) based on the gelation temperature of F127/F68 mixtures (for 17-25% F127 and 0-10% F68), using results obtained in examples above:

MDT=−0.2(T_{gel})+8.

### Example 9—Preparation of Medium Chain Triglyceride Formulations—Non-Natural Neurotrophic Agent

Formulations 1, 2, and 3 are prepared with the appropriate amounts of a non-natural neurotrophic agent and medium chain triglycerides (CRODAMOL, GTCC-LQ-(MV), PhEur) as shown in the below table (Table 7).

The formulations are prepared by diluting the thawed non-natural neurotrophic agent frozen solution with the appropriate amount of water for injection to a target concentration of the agent. The solution is thoroughly mixed and sterilized by filtration through a 0.22 um filter. The sterile solution is then filled into vials and lyophilized aseptically. The lyophilized powder is then dispersed into sterile filtered medium chain triglyceride via beat-blasting. The final suspension is filled into vials for further testing.

TABLE 7

| Component | Formulation 1 | Formulation 2 | Formulation 3 |
|---|---|---|---|
| Non-natural | 10 wt % | 5 wt % | 0.5 wt % |
| Neurotrophic Agent | | | |
| CRODAMOL, GTCC- | QS to 100 mL | QS to 100 mL | QS to 100 mL |
| LQ-(MV), PhEur | | | |

### Example 10—Additional Preparation of Medium Chain Triglyceride Formulations—Non-Natural Neurotrophic Agent

Formulations 4, 5, and 6 are prepared with the appropriate amounts of the non-natural neurotrophic agent and medium chain triglyceride (CRODAMOL, GTCC-LQ-(MV), PhEur) as shown in the below table (Table 8).

The formulations are prepared by diluting the thawed non-natural neurotrophic agent frozen solution with the appropriate amount of water for injection to a target concentration of the agent. The solution is thoroughly mixed and sterilized by filtration through a 0.22 um filter. The sterile solution is then filled into vials and lyophilized aseptically. The lyophilized powder is then dispersed into sterile filtered medium chain triglyceride via beat-blasting. The final suspension is filled into vials for further testing.

TABLE 8

| Component | Formulation 4 | Formulation 5 | Formulation 6 |
|---|---|---|---|
| Non-natural | 5 wt % | 10 wt % | 15 wt % |
| Neurotrophic Agent | | | |
| CRODAMOL, GTCC- | QS to 100 mL | QS to 100 mL | QS to 100 mL |
| LQ-(MV), PhEur | | | |

### Example 11—Preparation of Long Chain Triglyceride Formulations—Non-Natural Neurotrophic Agent

Long chain triglyceride formulations are prepared with the appropriate amounts of a non-natural neurotrophic agent and long chain triglyceride as shown in the below table (Table 9).

The formulations are prepared by diluting the thawed non-natural neurotrophic agent frozen solution with the appropriate amount of water for injection to a target concentration of the agent. The solution is thoroughly mixed and sterilized by filtration through a 0.22 um filter. The sterile solution is then filled into vials and lyophilized aseptically. The lyophilized powder is then dispersed into sterile filtered long chain triglyceride via beat-blasting. The final suspension is filled into vials for further testing.

TABLE 9

| Component | Amount (wt %) |
|---|---|
| Non-natural | 0.1%-10% |
| Neurotrophic Agent | |
| Long Chain | 99.9%-90% |
| Triglyercide | |

### Example 12—Clinical Trial of a Non-Natural Neurotrophic Agent as a Treatment for Tinnitus

Active Ingredient: Non-natural neurotrophic agent

Dosage: 10 ng delivered in 10 μL of a thermoreversible gel. Release of a non-natural neurotrophic agent is controlled release and occurs over thirty (30) days.

Route of Administration: Intratympanic injection

Treatment Duration: 12 weeks

### Methodology

Monocentric
Prospective
Randomized
Double-blind
Placebo-controlled
Parallel group
Adaptive

Inclusion Criteria
Male and female subjects between the 18 and 64 years of age.
Subjects experiencing subjective tinnitus.
Duration of tinnitus is greater than 3 months.
No treatment of tinnitus within 4 weeks.

### Evaluation Criteria

Efficacy (Primary)
   1. Total score of the Tinnitus Questionnaire
Efficacy (Secondary)
   1. Audiometric measurements (mode, frequency, loudness of the tinnitus, pure tone audiogram, speech audiogram)
   2. Quality of Life questionnaire
Safety
   1. Treatment groups are compared with respect to incidence rates of premature termination, treatment-emergent adverse events, laboratory abnormalities, and ECG abnormalities.

### Study Design

Subjects are divided into three treatment groups. The first group is the safety sample. The second group is the intent-to-treat (ITT) sample. The third group is the valid for efficacy (VfE) group.

For each group, one half of subjects to be given a non-natural neurotrophic agent and the remainder to be given placebo.

### Statistical Methods

The primary efficacy analysis is based on the total score of the Tinnitus Questionnaire in the ITT sample. The statistical analysis is based on an analysis of covariance (ANCOVA) with baseline as covariant and the last observation carried forward value as dependent variable. Factor is “treatment”. The homogeneity of regression slopes is tested. The analysis is repeated for the VfE sample.

Audiometric measurements (mode, frequency, loudness of the tinnitus, pure tone audiogram, speech audiogram) as well as quality of life are also analyzed via the aforementioned model. The appropriateness of the model is not tested. P values are exploratory and are not adjusted for multiplicity.

### Example 13—Clinical Trial of a Non-Natural Neurotrophic Agent as a Treatment for Noise Induced Hearing Loss

Active Ingredient: Non-natural neurotrophic agent

Dosage: A composition comprising 4% by weight of a non-natural neurotrophic agent delivered in 10 μL dose of a thermoreversible gel. Release of the non-natural neurotrophic agent is controlled release and occurs over 3 weeks.

Route of Administration: Intratympanic injection

Treatment Duration: 12 weeks, one injection every 3 weeks

### Methodology

Monocentric
Prospective
Randomized
Double-blind
Placebo-controlled
Parallel group
Adaptive

### Inclusion Criteria

Male and female subjects between the 18 and 64 years of age
Acoustic trauma followed by hearing loss that is documented by audiogram and medical report with an inner ear hearing loss of at least 15 dB
Acute tinnitus that has persisted for at least 3 months
No prior treatment of hearing loss within 4 weeks

### Evaluation Criteria

Efficacy (Primary)
   1. Audiometric measurements (pure tone audiogram, speech audiogram)
   2. Quality of Life questionnaire
Safety
   1. Treatment groups were compared with respect to incidence rates of premature termination, treatment-emergent adverse events, laboratory abnormalities, and ECG abnormalities.

### Study Design

Subjects are divided into three treatment groups. The first group is the safety sample. The second group is the intent-to-treat (ITT) sample. The third group is the valid for efficacy (VfE) group.

For each group, one half of subjects to be given a non-natural neurotrophic agent and the remainder to be given placebo.

### Statistical Methods

The primary efficacy analysis is based on the pure tone audiogram in the ITT sample. The statistical analysis is based on an analysis of covariance (ANCOVA) with baseline as covariant and the last observation carried forward value as dependent variable. Factor is “treatment.” The homogeneity of regression slopes is tested. The analysis is repeated for the VfE sample.

### Example 14—Clinical Trial of a Non-Natural Neurotrophic Agent as a Treatment in Combination with Implantation of a Cochlear Hearing Device

Active Ingredient: non-natural neurotrophic agent

Dosage: A composition comprising a non-natural neurotrophic agent, used as a pre-surgical irrigation solution and a post-surgical irrigation solution. Release of a non-natural neurotrophic agent is immediate release.

Study Design

Twenty patients will be enrolled in the study. Ten patients will be in the control group and ten patients will be in the treatment group.

### Eligibility Criteria

Having severe to profound sensorineural hearing impairment in both ears
Having a functioning auditory nerve
Having lived at least a short amount of time without hearing (approximately 70+ decibel hearing loss, on average)
Having good speech, language, and communication skills, or in the case of infants and young children, having a family willing to work toward speech and language skills with therapy
Not benefiting enough from other kinds of hearing aids
Having no medical reason to avoid surgery

Each patient will be subjected to chochleostomy and insertion of electrodes. The treatment group will be subjected to perfusion of the surgical area with the test composition prior to surgery and after surgery. The patients will be monitored for 6 weeks. Intracochlear trauma will be evaluated based on audiometric measurements, speech audiogram, as well as quality of life. Occurrence of secondary infections and/or inflammation will be monitored.

### Example 15—Clinical Trial of a Non-Natural Neurotrophic Agent in Combination with Cisplatin

The purpose of this study is to determine if a composition comprising a non-natural neurotrophic agent administered in combination with cisplatin is safe and effective in preventing and/or treating chemotherapy induced hearing loss in patients.

Study Type: Interventional

Study Design: This will be a non-inferiority open label study to compare the current standard of care versus the use of extended release intratympanic compositions in combination with cisplatin. The study is designed to test whether administration of a sustained release composition in combination with cisplatin prevents and/or treats chemotherapy induced hearing loss.

Inclusion Criteria:
Male and female subjects between the 18 and 64 years of age with hearing loss in one or both ears
Confirmed diagnosis of advanced head and neck cancer or advanced lung cancer
Patient may not have any disease or condition that would negatively affect the conduct of the study
Analgesic use (other than acetaminophen) is not allowed

Exclusion Criteria:
Age
Subjects previously treated with chemotherapy, antibiotics, or diuretics known to cause hearing loss in the last 90 days
History or presence of significant cardiovascular, pulmonary, hepatic, renal, hematologic, gastrointestinal, endocrine, immunologic, dermatologic, neurologic, otologic, or psychiatric disease
Presence of alcoholism or drug abuse
Participation in another investigational drug or device clinical trial within 30 days prior to the study
Female subjects who are pregnant or lactating

Twenty patients will be divided into two groups. The first group of patients will receive an injection of an extended release composition comprising a non-natural neurotrophic agent in combination with cisplatin. The second group of patients will be given placebo in combination with cisplatin.

Patients are monitored with weekly follow up visits for one month. Any differences in treatment outcomes between the two groups are recorded.

Primary Outcome Measures: Reduction and/or cessation of cisplatin induced hearing loss or the severity of cisplatin induced hearing loss; and number of participants with adverse events.

Secondary Outcome Measures: clinical cure rate, treatment failures, recurrence of disease.

While preferred embodiments of the present invention have been shown and described herein, such embodiments are provided by way of example only. Various alternatives to the embodiments described herein are optionally employed in practicing the inventions. It is intended that the following claims define the scope of the invention and that methods and structures within the scope of these claims and their equivalents be covered thereby.

What is claimed is:

1. A method of treating an otic condition in a subject in need thereof, comprising:
administering to the subject in need thereof an otic composition comprising a therapeutically effective amount of a non-natural neurotrophic agent; and
a pharmaceutically acceptable carrier.

2. The method of claim 1, wherein the non-natural neurotrophic agent comprises a modification at amino acid residue position equivalent to amino acid residue 15 set forth in SEQ ID NO: 2, and optionally comprises a modification at one or more amino acid residue positions equivalent to amino acid residues 11, 51, 68, 87, 103, 114, or 115 set forth in SEQ ID NO: 2.

3. The method of claim 2, wherein the non-natural neurotrophic agent comprises nerve growth factor (NGF), a pro-form of nerve growth factor (proNGF), neurotrophin-3 (NT-3), a pro-form of neurotrophin-3 (proNT-3), neurotrophin-4 (NT-4), a pro-form of neurotrophin-4 (proNT-4), neurotrophin-5 (NT-5), a pro-form of neurotrophin-5 (proNT-5), brain-derived neurotrophic factor (BDNF), a pro-form of brain-derived neurotrophic factor (proBDNF), a pan-neurotrophin (PNT), or PNT-1.

4. The method of claim 2, wherein the modification comprises:
a) a mutation to a non-polar residue;
b) a mutation to a polar residue;
c) a mutation to a charged residue;
d) a mutation to a conservative amino acid;
e) a mutation to a non-conservative amino acid;
f) a mutation to a semi-conservative amino acid; or
g) a mutation to alanine.

5. The method of claim 2, wherein the non-natural neurotrophic agent is NT-3 and wherein NT-3 comprises a modification at amino acid residue position equivalent to amino acid residue 15 set forth in SEQ ID NO: 2, and optionally comprises a modification at one or more amino acid residue positions equivalent to amino acid residues 11, 51, 68, 87, 103, 114, or 115 set forth in SEQ ID NO: 2.

6. The method of claim 2, wherein the non-natural neurotrophic agent comprises about 70%, 80%, 85%, 90%, 91%, 92%, 93%, 94%, 95%, 96%, 97%, 98%, or 99% sequence identity to SEQ ID NOs: 22-34.

7. The method of claim 1, wherein the non-natural neurotrophic agent comprises modifications at amino acid residue positions equivalent to amino acid residues 7 and 103 set forth in SEQ ID NO: 1, and optionally comprises:
a) a modification at an amino acid residue position equivalent to amino acid residue 84 set forth in SEQ ID NO: 1;
b) a modification at an amino acid residue position equivalent to amino acid residue 45 set forth in SEQ ID NO: 1; or
c) modifications at amino acid residue positions equivalent to amino acid residues 32, 34, and 35 set forth in SEQ ID NO: 1.

8. The method of claim 7, wherein the non-natural neurotrophic agent comprises nerve growth factor (NGF), a pro-form of nerve growth factor (proNGF), neurotrophin-3 (NT-3), a pro-form of neurotrophin-3 (proNT-3), neurotrophin-4 (NT-4), a pro-form of neurotrophin-4 (proNT-4), neurotrophin-5 (NT-5), a pro-form of neurotrophin-5 (proNT-5), brain-derived neurotrophic factor (BDNF), a pro-form of brain-derived neurotrophic factor (proBDNF), a pan-neurotrophin (PNT), or PNT-1.

9. The method of claim 8, wherein the non-natural neurotrophic agent is NGF, and wherein NGF comprises:
a) modifications at amino acid residue positions equivalent to amino acid residue 7, 84, and 103 set forth in SEQ ID NO: 1;
b) modifications at amino acid residue positions equivalent to amino acid residue 7, 45, and 103 set forth in SEQ ID NO: 1; or
c) about 70%, 80%, 85%, 90%, 91%, 92%, 93%, 94%, 95%, 96%, 97%, 98% or 99% sequence identity to SEQ ID NO: 8, 9, 10 or 11.

10. The method of claim 9, wherein NGF further comprises modifications at amino acid residue positions equivalent to amino acid residues 32, 34, and 35 set forth in SEQ ID NO: 1.

11. The method of claim 7, wherein the non-natural neurotrophic agent comprises a sequence set forth in SEQ ID NO: 6 or a sequence set forth in SEQ ID NO: 7.

12. The method of claim 1, wherein the non-natural neurotrophic agent comprises at least two modifications selected from amino acid residue positions equivalent to amino acid residues 74, 75, and 77 set forth in SEQ ID NO: 5, and optionally comprises a modification at an amino acid residue position equivalent to amino acid residue 32, 34, 115, or 116 set forth in SEQ ID NO: 5.

13. The method of claim 12, wherein the non-natural neurotrophic agent comprises nerve growth factor (NGF), neurotrophin-3 (NT-3), neurotrophin-4 (NT-4), neurotrophin-5 (NT-5), brain-derived neurotrophic factor (BDNF), or a pan-neurotrophin (PNT).

14. The method of claim 13, wherein the non-natural neurotrophic agent is PNT, and wherein PNT comprises modifications at amino acid residue positions equivalent to amino acid residues 74, 75, and 77 set forth in SEQ ID NO: 5, and optionally comprises modifications at amino acid residue positions equivalent to amino acid residues 32, 34, 115, or 116 set forth in SEQ ID NO: 5.

15. The method of claim 14, wherein PNT comprises about 70%, 80%, 85%, 90%, 91%, 92%, 93%, 94%, 95%, 96%, 97%, 98%, or 99% sequence identity to SEQ ID NO: 14 or 16.

16. The method of claim 1, wherein the non-natural neurotrophic agent comprises modifications at amino acid residue positions equivalent to amino acid residues 32 and 34 set forth in SEQ ID NO: 5, and optionally comprises a modification at an amino acid residue position equivalent to amino acid residue 74, 75, 77, 115, or 116 set forth in SEQ ID NO: 5, wherein the non-natural neurotrophic agent is selected from NGF, NT-4, NT-5, BDNF, or PNT.

17. The method of claim 16, wherein the non-natural neurotrophic agent is PNT, and wherein PNT comprises modifications at amino acid residue positions equivalent to amino acid residues 32 and 34 set forth in SEQ ID NO: 5, and optionally comprises modifications at amino acid residue positions equivalent to amino acid residues 74, 75, 77, 115, and 116 set forth in SEQ ID NO: 5.

18. The method of claim 17, wherein PNT comprises about 70%, 80%, 85%, 90%, 91%, 92%, 93%, 94%, 95%, 96%, 97%, 98% or 99% sequence identity to SEQ ID NO: 13 or 16.

19. The method of claim 1, wherein the non-natural neurotrophic agent comprises modifications at amino acid residue positions equivalent to amino acid residues 115 and 116 set forth in SEQ ID NO: 5, and optionally comprises a modification at an amino acid residue position equivalent to amino acid residue 32, 34, 74, 75, or 77 set forth in SEQ ID NO: 5, wherein the non-natural neurotrophic agent is selected from NGF, NT-4, NT-5, BDNF, or PNT.

20. The method of claim 19, wherein the non-natural neurotrophic agent is PNT, and wherein PNT comprises modifications at amino acid residue positions equivalent to amino acid residues 115 and 116 set forth in SEQ ID NO: 5, and optionally comprises modifications at amino acid residue positions equivalent to amino acid residues 32, 34, 74, 75, and 77 set forth in SEQ ID NO: 5.

21. The method of claim 20, wherein the non-natural neurotrophic agent comprises about 70%, 80%, 85%, 90%, 91%, 92%, 93%, 94%, 95%, 96%, 97%, 98%, or 99% sequence identity to SEQ ID NO: 15 or 16.

22. The method of claim 1, wherein the non-natural neurotrophic agent comprises modifications at amino acid residue positions equivalent to amino acid residues 61 and 100 set forth in SEQ ID NO: 5, wherein the non-natural neurotrophic agent is selected from NT-3, NT-4, NT-5, BDNF, or PNT.

23. The method of claim 22, wherein the non-natural neurotrophic agent is PNT, and wherein PNT comprises modifications at amino acid residue positions equivalent to amino acid residues 61 and 100 set forth in SEQ ID NO: 5.

24. The method of claim 22, wherein the non-natural neurotrophic agent comprises about 70%, 80%, 85%, 90%, 91%, 92%, 93%, 94%, 95%, 96%, 97%, 98%, or 99% sequence identity to SEQ ID NO: 20.

25. The method of claim 1, wherein the non-natural neurotrophic agent comprises a modification at amino acid residue position equivalent to amino acid residue 118 set forth in SEQ ID NO: 5, wherein the non-natural neurotrophic agent is selected from NT-3, NT-4, NT-5, BDNF, or PNT.

26. The method of claim 25, wherein the non-natural neurotrophic agent is PNT, and wherein PNT comprises a modification at amino acid residue positions equivalent to amino acid residue 118 set forth in SEQ ID NO: 5.

27. The method of claim 25, wherein the non-natural neurotrophic agent comprises about 70%, 80%, 85%, 90%, 91%, 92%, 93%, 94%, 95%, 96%, 97%, 98%, or 99% sequence identity to SEQ ID NO: 21.

28. The method of claim 1, wherein the non-natural neurotrophic agent comprises a sequence set forth in SEQ ID NOs: 17-19.

29. The method of any one of the claims 1-28, wherein the non-natural neurotrophic agent recognizes:
a) one or more tropomyosin receptor kinase (Trk) receptors;
b) a TrkA receptor;
c) a TrkB receptor; or
d) a TrkC receptor.

30. The method of claim 1, wherein the non-natural neurotrophic agent has an increased binding affinity to one or more Trk receptors relative to the binding affinity of an equivalent wild type neurotrophin, wherein the increase in binding affinity is about 20%, 30%, 40%, 50%, 60%, 70%, 80%, 85%, 90%, 95%, 96%, 97%, 98%, 99%, or more, relative to the binding affinity of an equivalent wild type neurotrophin.

31. The method of claim 1, wherein the non-natural neurotrophic agent has:
a) a decreased binding affinity to p75^{NTR} relative to the binding affinity of an equivalent wild type neurotrophin, wherein the decrease in binding affinity is about 30%, 40%, 50%, 60%, 70%, 80%, 85%, 90%, 95%, 96%, 97%, 98%, 99%, or more, relative to the binding affinity of an equivalent wild type neurotrophin; or
b) a reduced activation of p75^{NTR} relative to the activation of p75^{NTR} by an equivalent wild type neurotrophin, wherein the reduced activation is about 30%, 40%, 50%, 60%, 70%, 80%, 85%, 90%, 95%, 96%, 97%, 98%, 99%, or more, relative to the activation by an equivalent wild type neurotrophin.

32. The method of claim 1, wherein the otic composition further comprises two or more characteristics selected from:
(i) from about 0.0001% to about 60% by weight of the non-natural TrkB or TrkC agonist, or pharmaceutically acceptable prodrug or salt thereof;
(ii) from about 14% to about 21% by weight of a polyoxyethylene-polyoxypropylene triblock copolymer;
(iii) sterile water, q.s., buffered to provide a pH from about 5.5 to about 8.0;
(iv) a gelation temperature between about 19° C. to about 42° C.; and
(v) an apparent viscosity of about 100,000 cP to about 500,000 cP.

33. The method of claim 1, wherein the non-natural neurotrophic agent is released from the composition for a period of at least 1 day or at least 5 days.

34. The method of claim 1, wherein the otic condition:
a) is selected from a group consisting of ototoxicity, chemotherapy induced hearing loss, excitotoxicity, sensorineural hearing loss, noise induced hearing loss, hidden hearing loss, Meniere's Disease/Syndrome, endolymphatic hydrops, labyrinthitis, Ramsay Hunt's Syndrome, vestibular neuronitis, tinnitus, age-related hearing loss (presbycusis), microvascular compression syndrome, genetic hearing loss, and balance disorder; or
b) is characterized by damaged ribbon synapse, neurodegeneration, hair cell loss, or synaptopathy.

35. The method of claim 1, wherein administering the otic composition comprising the non-natural neurotrophic agent treats sensorineural hearing loss by inducing auris neuronal cell growth.

36. The method of claim 1, wherein administering the otic composition comprising the non-natural neurotrophic agent mediates at least one of synaptic modulation, afferent fiber growth, restoration of ribbon synapses, and increased neuronal connection to hair cells.

37. An otic pharmaceutical composition comprising a therapeutically effective amount of a non-natural neurotrophic agent of claims 1-36, and an auris-acceptable vehicle.

38. The otic pharmaceutical composition of claim 37, wherein the auris-acceptable vehicle comprises medium chain fatty acids.

39. The otic pharmaceutical composition of claim 37, wherein the auris-acceptable vehicle comprises a poloxamer.
